SEQUENCE LISTING (160 sequences)

SEQ ID NO: 1 - DNA, 72 bp, synthetic construct
  misc_feature 1..72: note = MbPylTCGA
  source 1..72: mol_type = other DNA
ggaaacctgatcatgtagatcgaatggactcgaaatccgttcagccgggttagattcccggggtttccgcca

SEQ ID NO: 2 - DNA, 72 bp, synthetic construct
  misc_feature 1..72: note = PylTC15CGA
  source 1..72: mol_type = other DNA
gggagagtggccaaggtggccgtgttgactcgaaatcaacacaggggggttcgattcccccctctcccgcca

SEQ ID NO: 3 - DNA, 72 bp, synthetic construct
  misc_feature 1..72: note = PylTM15CGA
  source 1..72: mol_type = other DNA
ggaaacctggtcagggagaccgaacggactcgaaatccgttcagccgggttcgattcccggggtttccgcca

SEQ ID NO: 4 - DNA, 72 bp, synthetic construct
  misc_feature 1..72: note = PylTCM15CGA
  source 1..72: mol_type = other DNA
gggagagtggccaaggtggccgaacggactcgaaatccgttcaggggggttcgattcccccctctcccgcca

SEQ ID NO: 5 - DNA, 72 bp, synthetic construct
  misc_feature 1..72: note = MetPylTCGA
  source 1..72: mol_type = other DNA
ggagacttggccaaggtggccgaacggactcgaaatccgttcaggggggttcgattcccccagtttccgcca

SEQ ID NO: 6 - DNA, 72 bp, synthetic construct
  misc_feature 1..72: note = SpePylTCGA
  source 1..72: mol_type = other DNA
ggaaatctgatcatgtagatcgaatggactcgaaatccgttcagccgggttagattcccggggtttccgcca

SEQ ID NO: 7 - DNA, 72 bp, synthetic construct
  misc_feature 1..72: note = Pyl-O1CGA
  source 1..72: mol_type = other DNA
ggggacctgatcatgtagatcgaatggactcgaaatccgttcagccgggttagattcccggggtcctcgcca

SEQ ID NO: 8 - DNA, 72 bp, synthetic construct
  misc_feature 1..72: note = Pyl-O2CGA
  source 1..72: mol_type = other DNA
gggcggctgatcatgtagatcgaatggactcgaaatccgttcagccgggttagattcccgggctgcccgcca

SEQ ID NO: 9 - DNA, 72 bp, synthetic construct
  misc_feature 1..72: note = Ma-T6CGA
  source 1..72: mol_type = other DNA
gggggacggtccggcgaccagcgggtctcgaaaacctagcatagcggggttcgacaccccggtctctcgcca

SEQ ID NO: 10 - DNA, 71 bp, synthetic construct
  misc_feature 1..71: note = MaPylTCGA
  source 1..71: mol_type = other DNA
gggggacggtccggcgaccagcgggtctcgaaaacctagccagcggggttcgacaccccggtctctcgcca

SEQ ID NO: 11 - DNA, 71 bp, synthetic construct
  misc_feature 1..71: note = G1PylTCGA
  source 1..71: mol_type = other DNA
ggagggcgctccggcgagcaaacgggtctcgaaaacctgtaagcggggttcgaccccccggcctttcgcca

SEQ ID NO: 12 - DNA, 73 bp, synthetic construct
  misc_feature 1..73: note = I2B72CGA
  source 1..73: mol_type = other DNA
ggggtgtagatcggattgatcgcgtggactcgaaatccgcgaacaacgggtgaaactcccgtacacctcgcca

SEQ ID NO: 13 - DNA, 72 bp, synthetic construct
  misc_feature 1..72: note = Int17CGA
  source 1..72: mol_type = other DNA
ggcgaactggtccgggaccaccaggcctcgaaagccacggttagccgggttcaactcccgggttcgtcgcca

SEQ ID NO: 14 - DNA, 72 bp, synthetic construct
  misc_feature 1..72: note = Int5CGA
  source 1..72: mol_type = other DNA
ggtgacatggtccgggaccaccaggcctcgaaagccacggttagccgggttcaactcccggtgtcatcgcca

SEQ ID NO: 15 - DNA, 72 bp, synthetic construct
  misc_feature 1..72: note = Int6BCGA
  source 1..72: mol_type = other DNA
ggtgaactggtccgggaccaccaggcctcgaaagccacggctagccgggttcaactcccgggttcatcgcca

SEQ ID NO: 16 - DNA, 72 bp, synthetic construct
  misc_feature 1..72: note = Int6CCGA
  source 1..72: mol_type = other DNA
ggtgaactggtccgggaccaccaggcctcgaaagccacggttagccgggttcaactcccgggttcatcgcca

SEQ ID NO: 17 - DNA, 72 bp, synthetic construct
  misc_feature 1..72: note = Int13CGA
  source 1..72: mol_type = other DNA
ggtgttctggtccgggaccaccgggcctcgaaagccacggttagccgggttcaactcccgggaacatcgcca

SEQ ID NO: 18 - DNA, 73 bp, synthetic construct
  misc_feature 1..73: note = Alv21CGA
  source 1..73: mol_type = other DNA
gggggacggtccggcgaccagcgggtctcgaaaacctagcataagcggggttcgaccccccggtctctcgcca

SEQ ID NO: 19 - DNA, 72 bp, synthetic construct
  misc_feature 1..72: note = Alv8CGA
  source 1..72: mol_type = other DNA
gggggacggtccggcgaccagcgggtctcgaaaacctagccttgcggggttcgacaccccggtctctcgcca

SEQ ID NO: 20 - DNA, 73 bp, synthetic construct
  misc_feature 1..73: note = Alv17CGA
  source 1..73: mol_type = other DNA
gggggacggtccggcgaccagcgggtctcgaaaacctagcgtaagcggggttcgacaccccggtctctcgcca

SEQ ID NO: 21 - DNA, 72 bp, synthetic construct
  misc_feature 1..72: note = Alv10CGA
  source 1..72: mol_type = other DNA
gggggacggtccggcgaccagcgggtctcgaaaacctagcttagcggggttcgacaccccggtctctcgcca

SEQ ID NO: 22 - DNA, 74 bp, synthetic construct
  misc_feature 1..74: note = Alv22CGA
  source 1..74: mol_type = other DNA
gggggacggtccggcgaccagcgggtctcgaaaacctagctcaaggcggggttcgactccccggtctctcgcca

SEQ ID NO: 23 - DNA, 71 bp, synthetic construct
  misc_feature 1..71: note = G1hybCGA
  source 1..71: mol_type = other DNA
gggggacgctccggcgagcaaacgggtctcgaaaacctgtaagcggggttcgaccccccggtctctcgcca

SEQ ID NO: 24 - DNA, 71 bp, synthetic construct
  misc_feature 1..71: note = IntCGA
  source 1..71: mol_type = other DNA
ggagtgttggtccgggaccaccaggcctcgacagccacggcagccgggttcaactcccgggcacttcgcca

SEQ ID NO: 25 - DNA, 72 bp, synthetic construct
  misc_feature 1..72: note = Therm1CGA
  source 1..72: mol_type = other DNA
ggggggctggtcgggtggccaagggggctcgaaaccctcggttgccgggttcaactcccgggctccccacca

SEQ ID NO: 26 - DNA, 73 bp, synthetic construct
  misc_feature 1..73: note = BH52CGA
  source 1..73: mol_type = other DNA
ggggcgttgatcggattgatcgcgtggactcgaaatccgcggccgacgggtgaaactcccgtacacctctcca

SEQ ID NO: 27 - DNA, 72 bp, synthetic construct
  misc_feature 1..72: note = CM4CGA
  source 1..72: mol_type = other DNA
gggagagtggccaaggtggccgaacggacttcgaatccgttcagccgggttcgattcccggctctcccacca

SEQ ID NO: 28 - DNA, 72 bp, synthetic construct
  misc_feature 1..72: note = AS78CGA
  source 1..72: mol_type = other DNA
ggggaggtggccaaggtggccgaacggactcgaaatccgttcagccgggttcgattcccggcctccccacca

SEQ ID NO: 29 - DNA, 82 bp, synthetic construct
  misc_feature 1..82: note = BsTyrTCGA
  source 1..82: mol_type = other DNA
ggtggggtagcgaagtggctaaacgcggcggactcgaaatccgctccctttgggttcggcggttcgaatccgtcccccacca

SEQ ID NO: 30 - DNA, 82 bp, synthetic construct
  misc_feature 1..82: note = NGS6CGA
  source 1..82: mol_type = other DNA
ggggcggtagcgaagtggctaaacgcggcggactcgaaatccgctccctttgggttcggcggttcgaatccgcgccccacca

SEQ ID NO: 31 - DNA, 87 bp, synthetic construct
  misc_feature 1..87: note = EcLeuTCGA
  source 1..87: mol_type = other DNA
gcccggatggtggaatcggtagacacaagggattcgaaatccctcggcgttcgcgctgtgcgggttcaagtcccgctccgggtacca

SEQ ID NO: 32 - DNA, 87 bp, synthetic construct
  misc_feature 1..87: note = LeuT-G1CGA
  source 1..87: mol_type = other DNA
gcccgtatggtggaatcggtagacacaagggattcgaaatccctcggcgttcgcgctgtgcgggttcaagtcccgctgcgggcacca

SEQ ID NO: 33 - DNA, 87 bp, synthetic construct
  misc_feature 1..87: note = LeuT--G2CGA
  source 1..87: mol_type = other DNA
gggcgtgtggtggaatcggtagacacaagggattcgaaatccctcggcgttcgcgctgtgcgggttcaagtcccgccgcgcccacca

SEQ ID NO: 34 - DNA, 72 bp, synthetic construct
  misc_feature 1..72: note = C15nnn
  misc_feature 31: note = n = a, c, g or t
  misc_feature 32: note = n = a, c, g or t
  misc_feature 33: note = n = a, c, g or t
  source 1..72: mol_type = other DNA
gggagagtggccaaggtggccgtgttgactnnnaatcaacacaggggggttcgattcccccctctcccgcca

SEQ ID NO: 35 - DNA, 72 bp, synthetic construct
  misc_feature 1..72: note = M15nnn
  misc_feature 31: note = n = a, c, g or t
  misc_feature 32: note = n = a, c, g or t
  misc_feature 33: note = n = a, c, g or t
  source 1..72: mol_type = other DNA
ggaaacctggtcagggagaccgaacggactnnnaatccgttcagccgggttcgattcccggggtttccgcca

SEQ ID NO: 36 - DNA, 72 bp, synthetic construct
  misc_feature 1..72: note = CM15nnn
  misc_feature 31: note = n = a, c, g or t
  misc_feature 32: note = n = a, c, g or t
  misc_feature 33: note = n = a, c, g or t
  source 1..72: mol_type = other DNA
gggagagtggccaaggtggccgaacggactnnnaatccgttcaggggggttcgattcccccctctcccgcca

SEQ ID NO: 37 - DNA, 72 bp, synthetic construct
  misc_feature 1..72: note = MbPylTnnn
  misc_feature 31: note = n = a, c, g or t
  misc_feature 32: note = n = a, c, g or t
  misc_feature 33: note = n = a, c, g or t
  source 1..72: mol_type = other DNA
ggaaacctgatcatgtagatcgaatggactnnnaatccgttcagccgggttagattcccggggtttccgcca

SEQ ID NO: 38 - DNA, 72 bp, synthetic construct
  misc_feature 1..72: note = MetPylTnnn
  misc_feature 31: note = n = a, c, g or t
  misc_feature 32: note = n = a, c, g or t
  misc_feature 33: note = n = a, c, g or t
  source 1..72: mol_type = other DNA
ggagacttggccaaggtggccgaacggactnnnaatccgttcaggggggttcgattcccccagtttccgcca

SEQ ID NO: 39 - DNA, 72 bp, synthetic construct
  misc_feature 1..72: note = SpePylTnnn
  misc_feature 31: note = n = a, c, g or t
  misc_feature 32: note = n = a, c, g or t
  misc_feature 33: note = n = a, c, g or t
  source 1..72: mol_type = other DNA
ggaaatctgatcatgtagatcgaatggactnnnaatccgttcagccgggttagattcccggggtttccgcca

SEQ ID NO: 40 - DNA, 72 bp, synthetic construct
  misc_feature 1..72: note = Pyl-O1nnn
  misc_feature 31: note = n = a, c, g or t
  misc_feature 32: note = n = a, c, g or t
  misc_feature 33: note = n = a, c, g or t
  source 1..72: mol_type = other DNA
ggggacctgatcatgtagatcgaatggactnnnaatccgttcagccgggttagattcccggggtcctcgcca

SEQ ID NO: 41 - DNA, 72 bp, synthetic construct
  misc_feature 1..72: note = Pyl-O2nnn
  misc_feature 31: note = n = a, c, g or t
  misc_feature 32: note = n = a, c, g or t
  misc_feature 33: note = n = a, c, g or t
  source 1..72: mol_type = other DNA
gggcggctgatcatgtagatcgaatggactnnnaatccgttcagccgggttagattcccgggctgcccgcca

SEQ ID NO: 42 - DNA, 72 bp, synthetic construct
  misc_feature 1..72: note = Ma-T6nnn
  misc_feature 29: note = n = a, c, g or t
  misc_feature 30: note = n = a, c, g or t
  misc_feature 31: note = n = a, c, g or t
  source 1..72: mol_type = other DNA
gggggacggtccggcgaccagcgggtctnnnaaacctagcatagcggggttcgacaccccggtctctcgcca

SEQ ID NO: 43 - DNA, 71 bp, synthetic construct
  misc_feature 1..71: note = MaPylTnnn
  misc_feature 29: note = n = a, c, g or t
  misc_feature 30: note = n = a, c, g or t
  misc_feature 31: note = n = a, c, g or t
  source 1..71: mol_type = other DNA
gggggacggtccggcgaccagcgggtctnnnaaacctagccagcggggttcgacaccccggtctctcgcca

SEQ ID NO: 44 - DNA, 71 bp, synthetic construct
  misc_feature 1..71: note = G1PylTnnn
  misc_feature 30: note = n = a, c, g or t
  misc_feature 31: note = n = a, c, g or t
  misc_feature 32: note = n = a, c, g or t
  source 1..71: mol_type = other DNA
ggagggcgctccggcgagcaaacgggtctnnnaaacctgtaagcggggttcgaccccccggcctttcgcca

SEQ ID NO: 45 - DNA, 71 bp, synthetic construct
  misc_feature 1..71: note = G1hybnnn
  misc_feature 30: note = n = a, c, g or t
  misc_feature 31: note = n = a, c, g or t
  misc_feature 32: note = n = a, c, g or t
  source 1..71: mol_type = other DNA
gggggacgctccggcgagcaaacgggtctnnnaaacctgtaagcggggttcgaccccccggtctctcgcca

SEQ ID NO: 46 - DNA, 73 bp, synthetic construct
  misc_feature 1..73: note = I2B72nnn
  misc_feature 31: note = n = a, c, g or t
  misc_feature 32: note = n = a, c, g or t
  misc_feature 33: note = n = a, c, g or t
  source 1..73: mol_type = other DNA
ggggtgtagatcggattgatcgcgtggactnnnaatccgcgaacaacgggtgaaactcccgtacacctcgcca

SEQ ID NO: 47 - DNA, 72 bp, synthetic construct
  misc_feature 1..72: note = Int17nnn
  misc_feature 29: note = n = a, c, g or t
  misc_feature 30: note = n = a, c, g or t
  misc_feature 31: note = n = a, c, g or t
  source 1..72: mol_type = other DNA
ggcgaactggtccgggaccaccaggcctnnnaagccacggttagccgggttcaactcccgggttcgtcgcca

SEQ ID NO: 48 - DNA, 72 bp, synthetic construct
  misc_feature 1..72: note = Int5nnn
  misc_feature 29: note = n = a, c, g or t
  misc_feature 30: note = n = a, c, g or t
  misc_feature 31: note = n = a, c, g or t
  source 1..72: mol_type = other DNA
ggtgacatggtccgggaccaccaggcctnnnaagccacggttagccgggttcaactcccggtgtcatcgcca

SEQ ID NO: 49 - DNA, 72 bp, synthetic construct
  misc_feature 1..72: note = Int6Bnnn
  misc_feature 29: note = n = a, c, g or t
  misc_feature 30: note = n = a, c, g or t
  misc_feature 31: note = n = a, c, g or t
  source 1..72: mol_type = other DNA
ggtgaactggtccgggaccaccaggcctnnnaagccacggctagccgggttcaactcccgggttcatcgcca

SEQ ID NO: 50 - DNA, 72 bp, synthetic construct
  misc_feature 1..72: note = Int6Cnnn
  misc_feature 29: note = n = a, c, g or t
  misc_feature 30: note = n = a, c, g or t
  misc_feature 31: note = n = a, c, g or t
  source 1..72: mol_type = other DNA
ggtgaactggtccgggaccaccaggcctnnnaagccacggttagccgggttcaactcccgggttcatcgcca

SEQ ID NO: 51 - DNA, 72 bp, synthetic construct
  misc_feature 1..72: note = Int13nnn
  misc_feature 29: note = n = a, c, g or t
  misc_feature 30: note = n = a, c, g or t
  misc_feature 31: note = n = a, c, g or t
  source 1..72: mol_type = other DNA
ggtgttctggtccgggaccaccgggcctnnnaagccacggttagccgggttcaactcccgggaacatcgcca

SEQ ID NO: 52 - DNA, 73 bp, synthetic construct
  misc_feature 1..73: note = Alv21nnn
  misc_feature 29: note = n = a, c, g or t
  misc_feature 30: note = n = a, c, g or t
  misc_feature 31: note = n = a, c, g or t
  source 1..73: mol_type = other DNA
gggggacggtccggcgaccagcgggtctnnnaaacctagcataagcggggttcgaccccccggtctctcgcca

SEQ ID NO: 53 - DNA, 72 bp, synthetic construct
  misc_feature 1..72: note = Alv8nnn
  misc_feature 29: note = n = a, c, g or t
  misc_feature 30: note = n = a, c, g or t
  misc_feature 31: note = n = a, c, g or t
  source 1..72: mol_type = other DNA
gggggacggtccggcgaccagcgggtctnnnaaacctagccttgcggggttcgacaccccggtctctcgcca

SEQ ID NO: 54 - DNA, 73 bp, synthetic construct
  misc_feature 1..73: note = Alv17nnn
  misc_feature 29: note = n = a, c, g or t
  misc_feature 30: note = n = a, c, g or t
  misc_feature 31: note = n = a, c, g or t
  source 1..73: mol_type = other DNA
gggggacggtccggcgaccagcgggtctnnnaaacctagcgtaagcggggttcgacaccccggtctctcgcca

SEQ ID NO: 55 - DNA, 72 bp, synthetic construct
  misc_feature 1..72: note = Alv10nnn
  misc_feature 29: note = n = a, c, g or t
  misc_feature 30: note = n = a, c, g or t
  misc_feature 31: note = n = a, c, g or t
  source 1..72: mol_type = other DNA
gggggacggtccggcgaccagcgggtctnnnaaacctagcttagcggggttcgacaccccggtctctcgcca

SEQ ID NO: 56 - DNA, 74 bp, synthetic construct
  misc_feature 1..74: note = Alv22nnn
  misc_feature 29: note = n = a, c, g or t
  misc_feature 30: note = n = a, c, g or t
  misc_feature 31: note = n = a, c, g or t
  source 1..74: mol_type = other DNA
gggggacggtccggcgaccagcgggtctnnnaaacctagctcaaggcggggttcgactccccggtctctcgcca

SEQ ID NO: 57 - DNA, 71 bp, synthetic construct
  misc_feature 1..71: note = Intnnn
  misc_feature 29: note = n = a, c, g or t
  misc_feature 30: note = n = a, c, g or t
  misc_feature 31: note = n = a, c, g or t
  source 1..71: mol_type = other DNA
ggagtgttggtccgggaccaccaggcctnnncagccacggcagccgggttcaactcccgggcacttcgcca

SEQ ID NO: 58 - DNA, 72 bp, synthetic construct
  misc_feature 1..72: note = Therm1nnn
  misc_feature 30: note = n = a, c, g or t
  misc_feature 31: note = n = a, c, g or t
  misc_feature 32: note = n = a, c, g or t
  source 1..72: mol_type = other DNA
ggggggctggtcgggtggccaagggggctnnnaaccctcggttgccgggttcaactcccgggctccccacca

SEQ ID NO: 59 - DNA, 73 bp, synthetic construct
  misc_feature 1..73: note = BH52nnn
  misc_feature 31: note = n = a, c, g or t
  misc_feature 32: note = n = a, c, g or t
  misc_feature 33: note = n = a, c, g or t
  source 1..73: mol_type = other DNA
ggggcgttgatcggattgatcgcgtggactnnnaatccgcggccgacgggtgaaactcccgtacacctctcca

SEQ ID NO: 60 - DNA, 72 bp, synthetic construct
  misc_feature 1..72: note = CM4nnn
  misc_feature 32: note = n = a, c, g or t
  misc_feature 33: note = n = a, c, g or t
  misc_feature 34: note = n = a, c, g or t
  source 1..72: mol_type = other DNA
gggagagtggccaaggtggccgaacggacttnnnatccgttcagccgggttcgattcccggctctcccacca

SEQ ID NO: 61 - DNA, 72 bp, synthetic construct
  misc_feature 1..72: note = AS78nnn
  misc_feature 31: note = n = a, c, g or t
  misc_feature 32: note = n = a, c, g or t
  misc_feature 33: note = n = a, c, g or t
  source 1..72: mol_type = other DNA
ggggaggtggccaaggtggccgaacggactnnnaatccgttcagccgggttcgattcccggcctccccacca

SEQ ID NO: 62 - DNA, 82 bp, synthetic construct
  misc_feature 1..82: note = BsTyrTnnn
  misc_feature 35: note = n = a, c, g or t
  misc_feature 36: note = n = a, c, g or t
  misc_feature 37: note = n = a, c, g or t
  source 1..82: mol_type = other DNA
ggtggggtagcgaagtggctaaacgcggcggactnnnaatccgctccctttgggttcggcggttcgaatccgtcccccacca

SEQ ID NO: 63 - DNA, 82 bp, synthetic construct
  misc_feature 1..82: note = NGS6nnn
  misc_feature 35: note = n = a, c, g or t
  misc_feature 36: note = n = a, c, g or t
  misc_feature 37: note = n = a, c, g or t
  source 1..82: mol_type = other DNA
ggggcggtagcgaagtggctaaacgcggcggactnnnaatccgctccctttgggttcggcggttcgaatccgcgccccacca

SEQ ID NO: 64 - DNA, 87 bp, synthetic construct
  misc_feature 1..87: note = EcLeuTnnn
  misc_feature 35: note = n = a, c, g or t
  misc_feature 36: note = n = a, c, g or t
  misc_feature 37: note = n = a, c, g or t
  source 1..87: mol_type = other DNA
gcccggatggtggaatcggtagacacaagggattnnnaatccctcggcgttcgcgctgtgcgggttcaagtcccgctccgggtacca

SEQ ID NO: 65 - DNA, 87 bp, synthetic construct
  misc_feature 1..87: note = L-G1nnn
  misc_feature 35: note = n = a, c, g or t
  misc_feature 36: note = n = a, c, g or t
  misc_feature 37: note = n = a, c, g or t
  source 1..87: mol_type = other DNA
gcccgtatggtggaatcggtagacacaagggattnnnaatccctcggcgttcgcgctgtgcgggttcaagtcccgctgcgggcacca

SEQ ID NO: 66 - DNA, 87 bp, synthetic construct
  misc_feature 1..87: note = L-G2nnn
  misc_feature 35: note = n = a, c, g or t
  misc_feature 36: note = n = a, c, g or t
  misc_feature 37: note = n = a, c, g or t
  source 1..87: mol_type = other DNA
gggcgtgtggtggaatcggtagacacaagggattnnnaatccctcggcgttcgcgctgtgcgggttcaagtcccgccgcgcccacca

SEQ ID NO: 67 - protein, 419 aa, synthetic construct
  REGION 1..419: note = MbPylRS-AA
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMLAPTLYNYLRKLDRILPGPIKIFEVGPCYRKESDGKEHLEEFTMVNFCQMGSGCTRENLEALIKEFLDYLEIDYEIVGDSCMVFGDTLDIMHGDLELSSAVVGPVSLDREWGIDKPWIGAGFGLERLLKVMHGFKNIKRASRSESYYNGISTNL

SEQ ID NO: 68 - protein, 454 aa, synthetic construct
  REGION 1..454: note = MmPylRS-AA
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 69 - protein, 419 aa, synthetic construct
  REGION 1..419: note = chPylRS-IPYE-AA
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRAPKPLENPVSAKASTDTSRSVPSPAKSTPNSPVPTSASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 70 - protein, 275 aa, synthetic construct
  REGION 1..275: note = MaPylRS-AA
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLNLVDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 71 - protein, 273 aa, synthetic construct
  REGION 1..273: note = G1PylRS-AA
MVVKFTDSQIQHLMEYGDNDWSEAEFEDAAARDKEFSSQFSKLKSANDKGLKDVIANPRNDLTDLENKIREKLAARGFIEVHTPIFVSKSALAKMTITEDHPLFKQVFWIDDKRALRPMHAMNLYKVMRELRDHTKGPVKIFEIGSCFRKESKSSTHLEEFTMLNLVEMGPDGDPMEHLKMYIGDIMDAVGVEYTTSREESDVYVETLDVEINGTEVASGAVGPHKLDPAHDVHEPWAGIGFGLERLLMLKNGKSNARKTGKSITYLNGYKLD

SEQ ID NO: 72 - protein, 276 aa, synthetic construct
  REGION 1..276: note = Lum1PylRS-AA
MDTRLTPAQAQRIREMGGTVDPSLAFSSEAERESAFQRISADLQGANLAKIRRCAEAPERHPIGSLENTLACALAAKGFIEVKTPMMIPADGLVKMGIDESHPLWNQVFWVGPKKALRPMLAPNLYFLMRHLRRSVPAPLLLFEIGPCFRKESRGSNHLEEFTMLNLVELAPQADATERLKEHIATVMNAVGLPYELVVEGSEVYGTTIDVEVDGVELASGAVGPLPMDKPHGITEPWAGVGFGLERIALMRTKEQNIKKVGRSLVYVNGARIDIS

SEQ ID NO: 73 - protein, 275 aa, synthetic construct
  REGION 1..275: note = 1R26PylRS-AA
MAEHFTDAQIQRLREYGNGTYKDMEFADVSAREKAFTKLMSDASRDNESALKGMIAHPARQGLSRLMNDIADALVADGFIEVRTPIIISKDALAKMTITPDKPLFKQVFWIDDKRALRPMLAPSLYTVMRSLRDHTDGPVKIFEMGSCFRKESHSGMHLEEFTMLNLVDMGPAGDATESLKKYIGIVMKAAGLPDYQLVHEESDVYKETIDVEINGQEVCSAAVGPHYLDAAHDVHEPWAGAGFGLERLLTIRQGYSTVMKGGASTTYLNGAKMD

SEQ ID NO: 74 - protein, 282 aa, synthetic construct
  REGION 1..282: note = NitraPylRS-AA
MASKIRFTRGQIHRLIELGAEPTELERDFETEAERDKEFNKIAENLARKNLKNIKDFLEQRRKPLVRVIEEKLRTTALRLGFSEVVTPIIIPRLFIKRMGIDEGDPLWKQVMLIDDKRALRPMLAPNLYVLMAKLSNIVRPVKIFEIGPCFRRETGGRYHLEEFTMFNMVELAPEGDPKERLLDYIDTIMRDIGLNYTISVEPSNVYGETLDVVVNGIEVASAAIGPKPIDANWGVREPWIGVGFGVERLAMLVGGYNSIARIAKSLSYLDGSTLSVIKLRW

SEQ ID NO: 75 - protein, 281 aa, synthetic construct
  REGION 1..281: note = DebPylRS-AA
MNSSWTEVQRHRLKELNGAEKDLETAFGDDLQRNRAFQKLEKQLVYQERKRLDRLLDTRFRPLRCELESLLIDALKCEGFTRVETPTIISQNDLERMSIDRSHPFNDQVYRVDSKHCLRPMLAPGLYRLMKDLARIRSGKPVRIFEIGPCFRKETSGARHAGEFTMLNLVEMRIEKGSRRFRIETLAKRIMHAAGIDTYDLVDEPSEVYNTTLDIVCGSDPLEVASCAMGPHPLDAAWGIIDTWVGLGFGLERLLMARENSPGIGKWCKSVSYLDGIRLTL

SEQ ID NO: 76 - protein, 424 aa, synthetic construct
  REGION 1..424: note = EcTyrRS-AA
MASSNLIKQLQERGLVAQVTDEEALAERLAQGPIALYCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYDFACLNKQYGVVLQIGGSDQWGNITSGIDLTRRLHQNQVFGLTVPLITKADGTKFGKTEGGAVWLDPKKTSPYKFYQFWINTARADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 77 - protein, 860 aa, synthetic construct [sequence omitted]

SEQ ID NO: 78 - protein, 555 aa, synthetic construct
  REGION 1..555: note = chPheRS-AA
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRADKELENPVSAKASTDTSRSVPSPAKSTPNSPVPTSASAPALTKSQTDRLEVLLNPKDEISLNSGKPFRELESELLSRRKKDLQQIYAEEREGGGSGGGSGGGSGGGSGGGSQAWGSRPPAAECATQRAPGSVVELLGKSYPQDDHSNLTRKVLTRVGRNLHNQQHHPLWLIKERVKEHFNKQYVDCFGTPLFSVYDNLSPVVTTWQNFDSLLITADHPCRRKGDNYYLNRTHMLRSHTSAHQWDLLHAGLDAFLVVGDVYRRDQIDSQHYPIFHQLEAVRLFTKHELFAGIKDGESQQLFEQSSRSAHKQESHTMEAVKLVEFDLKQTLTRLMAHLFGDEPEIRWVDSYFPFTHPSFEMEINFHGEWLEVLGCGVLEQQLVNSAGAQDRIGWAFGLGLERLAMILYDIPDIRLFWCEDERFLKQFCVSNINQKVKFQPLSK

SEQ ID NO: 79 - protein, 334 aa, synthetic construct
  REGION 1..334: note = EcTrpRS -AA
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLVPVGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 80 - DNA, 15 bp, synthetic construct
  misc_feature 1..15: note = boxB
  source 1..15: mol_type = other DNA
gccctgaaaaagggc

SEQ ID NO: 81 - protein, 22 aa, synthetic construct
  REGION 1..22: note = N22
MDAQTRRRERRAEKQAQWKAAN

SEQ ID NO: 82 - protein, 477 aa, synthetic construct
  REGION 1..477: note = FUS
ASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGG

SEQ ID NO: 83 - protein, 149 aa, synthetic construct
  REGION 1..149: note = MbPylRS-NTD
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPAS

SEQ ID NO: 84 - protein, 149 aa, synthetic construct
  REGION 1..149: note = MmPylRS-NTD
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVP

SEQ ID NO: 85 - protein, 149 aa, synthetic construct
  REGION 1..149: note = ChPylRS-NTD
MDKKPLDVLISATGLWMSRTGTLHKIKHYEISRSKIYIEMACGDHLVVNNSRSCRPARAFRYHKYRKTCKRCRVSDEDINNFLTRSTEGKTSVKVKVVSEPKVKKAMPKSVSRADKELENPVSAKASTDTSRSVPSPAKSTPNSPVPTS

SEQ ID NO: 86 - protein, 60 aa, synthetic construct
  REGION 1..60: note = MaPylRS-NTD
MTVKYTDAQIQRLREYGNGTYEQKVFEDLASRDAAFSKEMSVASTDNEKKIKGMIANPSR

SEQ ID NO: 87 - protein, 60 aa, synthetic construct
  REGION 1..60: note = G1PylRS-NTD
MVVKFTDSQIQHLMEYGDNDWSEAEFEDAAARDKEFSSQFSKLKSANDKGLKDVIANPRN

SEQ ID NO: 88 - protein, 60 aa, synthetic construct
  REGION 1..60: note = 1R26PylRS-NTD
MDTRLTPAQAQRIREMGGTVDPSLAFSSEAERESAFQRISADLQGANLAKIRRCAEAPER

SEQ ID NO: 89 - protein, 60 aa, synthetic construct
  REGION 1..60: note = NitraPylRS-NTD
MASKIRFTRGQIHRLIELGAEPTELERDFETEAERDKEFNKIAENLARKNLKNIKDFLEQ

SEQ ID NO: 90 - protein, 60 aa, synthetic construct
  REGION 1..60: note = DebPylRS-NTD
MNSSWTEVQRHRLKELNGAEKDLETAFGDDLQRNRAFQKLEKQLVYQERKRLDRLLDTRF

SEQ ID NO: 91 - protein, 312 aa, synthetic construct
  REGION 1..312: note = -tRNA -CTD
GSVVELLGKSYPQDDHSNLTRKVLTRVGRNLHNQQHHPLWLIKERVKEHFNKQYVDCFGTPLFSVYDNLSPVVTTWQNFDSLLITADHPCRRKGDNYYLNRTHMLRSHTSAHQWDLLHAGLDAFLVVGDVYRRDQIDSQHYPIFHQLEAVRLFTKHELFAGIKDGESQQLFEQSSRSAHKQESHTMEAVKLVEFDLKQTLTRLMAHLFGDEPEIRWVDSYFPFTHPSFEMEINFHGEWLEVLGCGVLEQQLVNSAGAQDRIGWAFGLGLERLAMILYDIPDIRLFWCEDERFLKQFCVSNINQKVKFQPLSK

SEQ ID NO: 92 - protein, 94 aa, synthetic construct
  REGION 1..94: note = Linker1
ASAPALTKSQTDRLEVLLNPKDEISLNSGKPFRELESELLSRRKKDLQQIYAEEREGGGSGGGSGGGSGGGSGGGSQAWGSRPPAAECATQRAP

SEQ ID NO: 93 - protein, 18 aa, synthetic construct
  REGION 1..18: note = Linker2
KSTRNSVDTSASAPGGGS

SEQ ID NO: 94 - protein, 82 aa, synthetic construct
  REGION 1..82: note = Linker3
ASAPALTKSQTDRLEVLLNPKDEISLNSGKPFRELESELLSRRKKDLQQIYAEEREGGGSAPAPAPAPAPAPAPAPAPGGGS

SEQ ID NO: 95 - DNA, 20 bp, synthetic construct
  misc_feature 1..20: note = TRS-CGA1-1 sgRNA
  source 1..20: mol_type = other DNA
gagcaggattcgaacctgcg

SEQ ID NO: 96 - DNA, 20 bp, synthetic construct
  misc_feature 1..20: note = TRS-CGA2-1 sgRNA
  source 1..20: mol_type = other DNA
gagcaggatttgaacctgcg

SEQ ID NO: 97 - DNA, 21 bp, synthetic construct
  misc_feature 1..21: note = TRS-CGA3-1 sgRNA
  source 1..21: mol_type = other DNA
gagtccaacaccttaaccact

SEQ ID NO: 98 - DNA, 21 bp, synthetic construct
  misc_feature 1..21: note = TRS-CGA4-1 sgRNA
  source 1..21: mol_type = other DNA
gacaggattcgaacctgtgcg

SEQ ID NO: 99 - protein, 17 aa, synthetic construct
  REGION 1..17: note = Nes-AA
ACPVPLQLPPLERLTLD

SEQ ID NO: 100 - DNA, 59 bp, synthetic construct
  misc_feature 1..59: note = Pyl-C15-F
  source 1..59: mol_type = other DNA
atttccccgaaaaatggcgggagaggggggaatcgaacccccctgtgttgatttcgagt

SEQ ID NO: 101 - DNA, 59 bp, synthetic construct
  misc_feature 1..59: note = Pyl-C15-R
  source 1..59: mol_type = other DNA
acgaaacaccgggagagtggccaaggtggccgtgttgactcgaaatcaacacagggggg

SEQ ID NO: 102 - DNA, 59 bp, synthetic construct
  misc_feature 1..59: note = Pyl-M15-F
  source 1..59: mol_type = other DNA
atttccccgaaaaatggcggaaaccccgggaatcgaacccggctgaacggatttcgagt

SEQ ID NO: 103 - DNA, 59 bp, synthetic construct
  misc_feature 1..59: note = Pyl-M15-R
  source 1..59: mol_type = other DNA
acgaaacaccggaaacctggtcagggagaccgaacggactcgaaatccgttcagccggg

SEQ ID NO: 104 - DNA, 59 bp, synthetic construct
  misc_feature 1..59: note = Pyl-CM15-F
  source 1..59: mol_type = other DNA
atttccccgaaaaatggcgggagaggggggaatcgaacccccctgaacggatttcgagt

SEQ ID NO: 105 - DNA, 59 bp, synthetic construct
  misc_feature 1..59: note = Pyl-CM15-R
  source 1..59: mol_type = other DNA
acgaaacaccgggagagtggccaaggtggccgaacggactcgaaatccgttcagggggg

SEQ ID NO: 106 - DNA, 45 bp, synthetic construct
  misc_feature 1..45: note = tRNA-F1/tRNA-v-r
  source 1..45: mol_type = other DNA
caacgaattcggatccggtaccggggttccgcgcacatttccccg

SEQ ID NO: 107 - DNA, 47 bp, synthetic construct
  misc_feature 1..47: note = tRNA-R1
  source 1..47: mol_type = other DNA
gttgaacctgtcacgtcctggcccgtacatcgcgaaggtcgggcagg

SEQ ID NO: 108 - DNA, 42 bp, synthetic construct
  misc_feature 1..42: note = tRNA-F2
  source 1..42: mol_type = other DNA
caggacgtgacaggttcaacggggttccgcgcacatttcccc

SEQ ID NO: 109 - DNA, 43 bp, synthetic construct
  misc_feature 1..43: note = tRNA-R2
  source 1..43: mol_type = other DNA
gttgatcgatccgtagctacgtacatcgcgaaggtcgggcagg

SEQ ID NO: 110 - DNA, 42 bp, synthetic construct
  misc_feature 1..42: note = tRNA-F3
  source 1..42: mol_type = other DNA
gtagctacggatcgatcaacggggttccgcgcacatttcccc

SEQ ID NO: 111 - DNA, 42 bp, synthetic construct
  misc_feature 1..42: note = tRNA-R3
  source 1..42: mol_type = other DNA
gctgagagtgactctgcgtggtacatcgcgaaggtcgggcag

SEQ ID NO: 112 - DNA, 42 bp, synthetic construct
  misc_feature 1..42: note = tRNA-F4
  source 1..42: mol_type = other DNA
cacgcagagtcactctcagcggggttccgcgcacatttcccc

SEQ ID NO: 113 - DNA, 55 bp, synthetic construct
  misc_feature 1..55: note = tRNA-R4
  source 1..55: mol_type = other DNA
ctgaccagatctcaataatcaatgtcaggtaccgtacatcgcgaaggtcgggcag

SEQ ID NO: 114 - DNA, 31 bp, synthetic construct
  misc_feature 1..31: note = EGFP-95TCG-F
  source 1..31: mol_type = other DNA
cgtccagtcgcgcaccatcttcttcaaggac

SEQ ID NO: 115 - DNA, 28 bp, synthetic construct
  misc_feature 1..28: note = EGFP-95TCG-R
  source 1..28: mol_type = other DNA
gcgcgactggacgtagccttcgggcatg

SEQ ID NO: 116 - DNA, 31 bp, synthetic construct
  misc_feature 1..31: note = EGFP-151TCG-F
  source 1..31: mol_type = other DNA
caacgtctcgatcatggccgacaagcagaag

SEQ ID NO: 117 - DNA, 30 bp, synthetic construct
  misc_feature 1..30: note = EGFP-151TCG-R
  source 1..30: mol_type = other DNA
atgatcgagacgttgtggctgttgtagttg

SEQ ID NO: 118 - DNA, 30 bp, synthetic construct
  misc_feature 1..30: note = EGFP-164TCG-F
  source 1..30: mol_type = other DNA
aggtgtcgttcaagatccgccacaacatcg

SEQ ID NO: 119 - DNA, 30 bp, synthetic construct
  misc_feature 1..30: note = EGFP-164TCG-R
  source 1..30: mol_type = other DNA
ttgaacgacaccttgatgccgttcttctgc

SEQ ID NO: 120 - DNA, 28 bp, synthetic construct
  misc_feature 1..28: note = EGFP-172TCG-F
  source 1..28: mol_type = other DNA
caacatctcggacggcagcgtgcagctc

SEQ ID NO: 121 - DNA, 32 bp, synthetic construct
  misc_feature 1..32: note = EGFP-172TCG-R
  source 1..32: mol_type = other DNA
cgtccgagatgttgtggcggatcttgaagttc

SEQ ID NO: 122 - DNA, 28 bp, synthetic construct
  misc_feature 1..28: note = EGFP-182TCG-F
  source 1..28: mol_type = other DNA
accactcgcagcagaacacccccatcgg

SEQ ID NO: 123 - DNA, 28 bp, synthetic construct
  misc_feature 1..28: note = EGFP-182TCG-R
  source 1..28: mol_type = other DNA
tctgctgcgagtggtcggcgagctgcac

SEQ ID NO: 124 - DNA, 30 bp, synthetic construct
  misc_feature 1..30: note = EGFP-200TCG-F1
  source 1..30: mol_type = other DNA
aaccactcgctgagcacccagtccgccctg

SEQ ID NO: 125 - DNA, 26 bp, synthetic construct
  misc_feature 1..26: note = EGFP-200TCG-R1
  source 1..26: mol_type = other DNA
tcagcgagtggttgtcgggcagcagc

SEQ ID NO: 126 - DNA, 26 bp, synthetic construct
  misc_feature 1..26: note = EGFP-95TAG-F
  source 1..26: mol_type = other DNA
tagcgcaccatcttcttcaaggacga

SEQ ID NO: 127 - DNA, 32 bp, synthetic construct
  misc_feature 1..32: note = EGFP-95TAG-R
  source 1..32: mol_type = other DNA
gatggtgcgctactggacgtagccttcgggca

SEQ ID NO: 128 - DNA, 36 bp, synthetic construct
  misc_feature 1..36: note = EGFP-151TAG-F
  source 1..36: mol_type = other DNA
aacagccacaacgtctagatcatggccgacaagcag

SEQ ID NO: 129 - DNA, 27 bp, synthetic construct
  misc_feature 1..27: note = EGFP-151TAG-R
  source 1..27: mol_type = other DNA
gttgtggctgttgtagttgtactccag

SEQ ID NO: 130 - DNA, 32 bp, synthetic construct
  misc_feature 1..32: note = EGFP-164TAG-F
  source 1..32: mol_type = other DNA
aggtgtagttcaagatccgccacaacatctag

SEQ ID NO: 131 - DNA, 29 bp, synthetic construct
  misc_feature 1..29: note = EGFP-164TAG-R
  source 1..29: mol_type = other DNA
ttgaactacaccttgatgccgttcttctg

SEQ ID NO: 132 - DNA, 30 bp, synthetic construct
  misc_feature 1..30: note = EGFP-172TAG-F
  source 1..30: mol_type = other DNA
caacatctaggacggcagcgtgcagctcgc

SEQ ID NO: 133 - DNA, 32 bp, synthetic construct
  misc_feature 1..32: note = EGFP-172TAG-R
  source 1..32: mol_type = other DNA
cgtcctagatgttgtggcggatcttgaactac

SEQ ID NO: 134 - DNA, 59 bp, synthetic construct
  misc_feature 1..59: note = EGFP-182/200TAG-F
  source 1..59: mol_type = other DNA
gcgacggccccgtgctgctgcccgacaaccactagctgagcacccagtccgccctgagc

SEQ ID NO: 135 - DNA, 59 bp, synthetic construct
  misc_feature 1..59: note = EGFP-182/200TAG-R
  source 1..59: mol_type = other DNA
cacggggccgtcgccgatgggggtgttctgctgctagtggtcggcgagctgcacgctgc

SEQ ID NO: 136 - DNA, 44 bp, synthetic construct
  misc_feature 1..44: note = EGFP-182ACG-F1
  source 1..44: mol_type = other DNA
gcgtgcagctcgccgaccacacgcagcagaacacccccatcggc

SEQ ID NO: 137 - DNA, 20 bp, synthetic construct
  misc_feature 1..20: note = EGFP-182ACG-R1
  source 1..20: mol_type = other DNA
gtggtcggcgagctgcacgc

SEQ ID NO: 138 - DNA, 44 bp, synthetic construct
  misc_feature 1..44: note = EGFP-182TCA-F
  source 1..44: mol_type = other DNA
gcgtgcagctcgccgaccactcacagcagaacacccccatcggc

SEQ ID NO: 139 - DNA, 44 bp, synthetic construct
  misc_feature 1..44: note = EGFP-182CGA-F
  source 1..44: mol_type = other DNA
gcgtgcagctcgccgaccaccgacagcagaacacccccatcggc

SEQ ID NO: 140 - DNA, 31 bp, synthetic construct
  misc_feature 1..31: note = EGFP-151ACG-F
  source 1..31: mol_type = other DNA
caacgtcacgatcatggccgacaagcagaag

SEQ ID NO: 141 - DNA, 30 bp, synthetic construct
  misc_feature 1..30: note = EGFP-151ACG-R
  source 1..30: mol_type = other DNA
atgatcgtgacgttgtggctgttgtagttg

SEQ ID NO: 142 - DNA, 43 bp, synthetic construct
  misc_feature 1..43: note = EGFP-84TCG-F
  source 1..43: mol_type = other DNA
acatgaagcagcacgacttctcgaagtccgccatgcccgaagg

SEQ ID NO: 143 - DNA, 23 bp, synthetic construct
  misc_feature 1..23: note = EGFP-84TCG-R
  source 1..23: mol_type = other DNA
gaagtcgtgctgcttcatgtggt

SEQ ID NO: 144 - DNA, 30 bp, synthetic construct
  misc_feature 1..30: note = EGFP-151TAA-F
  source 1..30: mol_type = other DNA
atgatttagacgttgtggctgttgtagttg

SEQ ID NO: 145 - DNA, 31 bp, synthetic construct
  misc_feature 1..31: note = EGFP-151TAA-R
  source 1..31: mol_type = other DNA
caacgtctaaatcatggccgacaagcagaag

SEQ ID NO: 146 - DNA, 34 bp, synthetic construct
  misc_feature 1..34: note = EGFP-200TCG-F2
  source 1..34: mol_type = other DNA
ggtgctcagcgagtggttgtcgggcagcagcacg

SEQ ID NO: 147 - DNA, 35 bp, synthetic construct
  misc_feature 1..35: note = EGFP-200TCG-R2
  source 1..35: mol_type = other DNA
ccgacaaccactcgctgagcacccagtccgccctg

SEQ ID NO: 148 - DNA, 54 bp, synthetic construct
  misc_feature 1..54: note = EGFP-151TCA-F
  source 1..54: mol_type = other DNA
gtctcaatcatggccgacaagcagaagaacggcatcaaggtgaacttcaagatc

SEQ ID NO: 149 - DNA, 43 bp, synthetic construct
  misc_feature 1..43: note = EGFP-151TCA-R
  source 1..43: mol_type = other DNA
gtcggccatgattgagacgttgtggctgttgtagttgtactcc

SEQ ID NO: 150 - DNA, 37 bp, synthetic construct
  misc_feature 1..37: note = EGFP-182ACG-F2
  source 1..37: mol_type = other DNA
cacacgcagcagaacacccccatcggcgacggccccg

SEQ ID NO: 151 - DNA, 48 bp, synthetic construct
  misc_feature 1..48: note = EGFP-182ACG-R2
  source 1..48: mol_type = other DNA
ccgtcgccgatgggggtgttctgctgcgtgtggtcggcgagctgcacg

SEQ ID NO: 152 - DNA, 31 bp, synthetic construct
  misc_feature 1..31: note = EGFP-206TAG-F
  source 1..31: mol_type = other DNA
gtcctagctgagcaaagaccccaacgagaag

SEQ ID NO: 153 - DNA, 29 bp, synthetic construct
  misc_feature 1..29: note = EGFP-206TAG-R
  source 1..29: mol_type = other DNA
gtctttgctcagctaggactgggtgctca

SEQ ID NO: 154 - DNA, 47 bp, synthetic construct
  misc_feature 1..47: note = 1A1-Forward sequence
  source 1..47: mol_type = other DNA
aaaactagtgacgctgtggagaccccattggatttcgagtccaacgc

SEQ ID NO: 155 - DNA, 60 bp, synthetic construct
  misc_feature 1..60: note = 1A1-Reverse sequence
  source 1..60: mol_type = other DNA
aaaactagtgacgctgtgagcaggattcgaacagaccccattggatttcgagtccaacgc

SEQ ID NO: 156 - DNA, 33 bp, synthetic construct
  misc_feature 1..33: note = 2A1-Forward sequence
  source 1..33: mol_type = other DNA
cgaaatccaatgcttctcggtacctctctctct

SEQ ID NO: 157 - DNA, 46 bp, synthetic construct
  misc_feature 1..46: note = 2A1-Reverse sequence
  source 1..46: mol_type = other DNA
cgaaatccaatggggtctccccgcgcttctcggtacctctctctct

SEQ ID NO: 158 - DNA, 67 bp, synthetic construct
  misc_feature 1..67: note = 3C12-Forward sequence
  source 1..67: mol_type = other DNA
acccccattggatttcgagtccaacaccttaactcggccatcacagctccgaggacactttttgttt

SEQ ID NO: 159 - DNA, 41 bp, synthetic construct
  misc_feature 1..41: note = 4C23-Forward sequence
  source 1..41: mol_type = other DNA
ggcgttggactcgaaatcctgttcgtgacggggtttttttt

SEQ ID NO: 160 - DNA, 68 bp, synthetic construct
  misc_feature 1..68: note = 4C23-Reverse sequence
  source 1..68: mol_type = other DNA
ggcgttggactcgaaatccaatggggtttccccgcaacaggttcgaatcctgttcgtgacgggttttt

(19) United States
(12) Patent Application Publication
(10) Pub. No.: US 2025/0327074 A1
(43) Pub. Date: Oct. 23, 2025

(54) THE RARE CODON RECODING PLATFORM FOR NONCANONICAL AMINO ACIDS INCORPORATION IN MAMMALIAN CELLS

(71) Applicant: SHAOXING INSTITUTE, ZHEJIANG UNIVERSITY, Shaoxing City (CN)

(72) Inventors: Shixian LIN, Shaoxing City (CN); Wenlong DING, Shaoxing City (CN); Wei YU, Shaoxing City (CN); Yulin CHEN, Shaoxing City (CN); Yu FANG, Shaoxing City (CN)

(73) Assignee: SHAOXING INSTITUTE, ZHEJIANG UNIVERSITY, Shaoxing City (CN)

(21) Appl. No.: 19/184,330

(22) Filed: Apr. 21, 2025

(30) Foreign Application Priority Data
Apr. 19, 2024 (CN) 202410488274.3

(51) Int. Cl. C12N 15/113; C07K 14/435; C12N 9/00

(52) U.S. Cl. CPC C12N 15/113; C07K 14/43595; C12N 9/93; C12Y 601/01026; C07K 2319/00

(57) ABSTRACT

This application relates to a method for producing a protein containing an unnatural amino acid (UAA), the method comprising culturing a host cell, wherein the host cell is a eukaryotic cell, together with: a nucleotide sequence encoding a first recoding tRNA or a first recoding tRNA, wherein the first recoding tRNA comprises an anticodon complementary to a first codon, and wherein the first codon is a rare codon; and a nucleotide sequence encoding a first aminoacyl-tRNA synthetase or a first aminoacyl-tRNA synthetase, wherein the first aminoacyl-tRNA synthetase is capable of charging the first recoding tRNA with the first unnatural amino acid.

### CROSS-REFERENCE TO RELATED APPLICATIONS

This application claims priority to Chinese Patent Application No. 2024104882743, filed on Apr. 19, 2024, the disclosure of which is hereby incorporated by reference in its entirety.

### INCORPORATION BY REFERENCE OF SEQUENCE LISTING

The content of the electronically submitted sequence listing, file name: Q307948_sequence listing as filed: size: 250,417; and date of creation: Apr. 21, 2025, filed herewith, is incorporated herein by reference in its entirety:

### FIELD OF THE INVENTION

This invention pertains to the field of biomedicine, specifically to systems and methods for expressing proteins containing unnatural amino acids (UAAs) in eukaryotic cells, with a emphasis on mammalian cells. The invention utilizes rare codons to reprogram the genetic code, enabling the site-specific incorporation of UAAs into proteins to produce novel therapeutic, diagnostic, and industrial proteins.

### BACKGROUND OF THE INVENTION

Proteins are fundamental macromolecules that underpin a vast array of biological functions, traditionally synthesized from a set of 20 natural amino acids encoded by the universal genetic code. Recent advances in protein engineering have leveraged unnatural amino acids (UAAs) to expand the functional repertoire of proteins, introducing novel chemical and physical properties that enhance their utility in therapeutic development, diagnostics, and industrial applications.

Genetic code expansion is a well-established technique for incorporating UAAs into proteins at specific sites. This method typically employs orthogonal aminoacyl-tRNA synthetase (aaRS)/tRNA pairs that recognize UAAs and incorporate them in response to designated codons, such as stop codons or rare codons. While this approach has been successfully implemented in prokaryotic systems, its application in eukaryotic cells, particularly mammalian cells, has faced significant hurdles. These challenges stem from the complexity of eukaryotic translation machinery and the competition between orthogonal tRNAs and release factors at stop codons, resulting in low incorporation efficiency and high background noise.

The present invention overcomes these limitations by employing rare codons for genetic code expansion in eukaryotic cells. By reprogramming rare codons, this method avoids the inefficiencies and competition associated with stop codon suppression, enabling the efficient and precise production of UAA-containing proteins in mammalian cells.

### SUMMARY OF THE INVENTION

The present invention overcomes the general barrier that eukaryotic cells cannot utilize rare codons for recoding unnatural amino acids and discloses a method for producing proteins containing unnatural amino acids using rare codons, achieving an efficiency comparable to that of natural protein production in mammalian cells. Through optimization of the recoding strategy, this method unexpectedly enhances recoding efficiency and reduces background incorporation rates significantly: Compared to approaches employing amber stop codons for suppression in eukaryotic cells, the recoding efficiency of rare codons is markedly higher, overcoming the issue of background incorporation inherent in rare codon recoding and attaining the efficiency and level of normal protein production in eukaryotic cells. Moreover, the method disclosed herein does not interfere with ongoing cellular growth, metabolism, or the synthesis of normal proteins, addressing the cytotoxicity challenges posed by the introduction of recoding systems into eukaryotic cells, thus providing a robust foundation for improving protein production yields.

Additionally, the method of this application achieves, for the first time in eukaryotic cells, the site-specific insertion of four or five distinct of unnatural amino acids into target proteins, surpassing the maximum number and types of amino acids encodable by traditional methods. This breakthrough positions the method as a powerful tool for expressing proteins with multiple unnatural amino acids in eukaryotic cells.

This application utilizes rare codons instead of nonsense codons to overcome the competition of release factors, thereby efficiently synthesizing proteins containing unnatural amino acids in eukaryotic cells. Existing strategies for integrating unnatural amino acids in eukaryotic cells rely on introducing unassigned codons or blank codons into the target gene, which is inefficient in generating full-length unnatural amino acid-incorporated proteins, greatly limiting their widespread application.

In the genome, due to the lower abundance of endogenous decoding tRNAs corresponding to rare codons, competing with relatively weak endogenous decoding tRNAs is another strategy for developing site-specific and efficient incorporation of unnatural amino acids in eukaryotes, as opposed to competing with translation release factors. However, a major challenge in developing selective rare codon encoding in eukaryotic cells is that rare codons are more widely distributed in the transcriptome than amber codons, leading to a large amount of erroneous insertion of unnatural amino acids into the proteome. Therefore, it is generally believed in this field that using rare codons to achieve the production of proteins containing unnatural amino acids in eukaryotes is theoretically unfeasible.

This application provides a method for producing proteins containing unnatural amino acids (UAA), which includes culturing host cells together with the following substances, where the host cells are eukaryotic cells: a nucleotide sequence encoding a first recoding tRNA or the nucleotide sequence of the first recoding tRNA, wherein the first recoding tRNA contains an anticodon complementary to the first codon, and the first codon is a rare codon; a nucleotide sequence encoding a first aminoacyl-tRNA synthetase or the first aminoacyl-tRNA synthetase, which can load the first recoding tRNA with the first unnatural amino acid.

In some embodiments, the above method further includes culturing eukaryotic cells together with: a nucleotide sequence encoding a second recoding tRNA or the nucleotide sequence of the second recoding tRNA, wherein the second recoding tRNA contains an anticodon complementary to the second codon; a nucleic acid encoding the nucleotide sequence of a second aminoacyl-tRNA synthetase or the second aminoacyl-tRNA synthetase, which can load the second tRNA with the second unnatural amino acid.

On the other hand, this application provides a translation system for expressing proteins containing unnatural amino acids, which includes host cells, where the host cells are eukaryotic cells: a nucleotide sequence encoding a first recoding tRNA or the nucleotide sequence of the first recoding tRNA, wherein the first recoding tRNA contains an anticodon complementary to the first codon, and the first codon is a first rare codon: a nucleotide sequence encoding a first aminoacyl-tRNA synthetase or the first aminoacyl-tRNA synthetase, which can load the first recoding tRNA with the first unnatural amino acid.

In some embodiments, the above translation system further includes host cells, where the host cells are eukaryotic cells: a nucleotide sequence encoding a first recoding tRNA or the nucleotide sequence of the first recoding tRNA, wherein the first recoding tRNA contains an anticodon complementary to the first codon, and the first codon is a first rare codon; a nucleotide sequence encoding a first aminoacyl-tRNA synthetase or the first aminoacyl-tRNA synthetase, which can load the first recoding tRNA with the first unnatural amino acid.

On the other hand, this application provides a kit including a nucleotide sequence encoding a first recoding tRNA or the nucleotide sequence of the first recoding tRNA and a nucleotide sequence encoding a first aminoacyl-tRNA synthetase or the first aminoacyl-tRNA synthetase, wherein the recoding tRNA includes one or more of anticodons corresponding to the following codons: TCG, ACG, CGA, TCA, CGC, TTG, ATA, GCG, TTA, and TGT.

On the other hand, this application provides a cell including a nucleotide sequence encoding a first recoding tRNA or the nucleotide sequence of the first recoding tRNA and a nucleotide sequence encoding a first aminoacyl-tRNA synthetase or the first aminoacyl-tRNA synthetase, wherein the recoding tRNA includes one or more of anticodons corresponding to the following codons: TCG, ACG, CGA, TCA, CGC, TTG, ATA, GCG, TTA, and TGT. In this application, codons are represented by their coding genes (i.e., DNA). For example, the codons TCG, ACG, CGA, TCA, CGC, TTG, ATA, GCG, TTA, and TGT correspond to UCG, ACG, CGA, UCA, CGC, UUG, AUA, GCG, UUA, and UGU of mRNA, respectively.

In some embodiments, in wild-type host cells, the tRNA decoding rare codons accounts for less than 2% of the tRNA in wild-type host cells, further less than 1.75%, and even further less than 0.5%.

In some embodiments, in wild-type eukaryotic cells, the frequency of rare codons appearing in all codons of wild-type host cells is less than 20 per 1000 codons, further less than 10 per 1000 codons. In some embodiments, the rare codons are one or more of TCG, ACG, CGA, TCA, CGC, TTG, ATA, GCG, TTA, and TGT. In this application, codons are represented by their coding genes (i.e., DNA). For example, the codons TCG, ACG, CGA, TCA, CGC, TTG, ATA, GCG, TTA, and TGT correspond to UCG, ACG, CGA, UCA, CGC, UUG, AUA, GCG, UUA, and UGU of mRNA, respectively:

In some embodiments, the aminoacyl-tRNA synthetase and/or recoding tRNA are derived from prokaryotes or eukaryotes, or variants of enzymes and/or tRNAs derived from prokaryotes or eukaryotes.

In some embodiments, the aminoacyl-tRNA synthetase and/or recoding tRNA are derived from eubacteria or archaea, or variants of enzymes and/or tRNAs derived from eubacteria or archaea.

In some embodiments, the archaea are one or more of Methanococcus jannaschii (Mj), Methanosarcina mazei (Mm), Methanosarcina barkeri (Mb), Methanomethylophilus alvus (Ma), Methanogenic archaeon ISO4-G1 (G1), Methanomassiliicoccus luminyensis (Lum1), Candidatus Methanomethylophilus sp. 1R26 (1R26), Candidatus Methanomassiliicoccus intestinalis (Int), Nitrososphaeria archaeon (Nitra), Deltaproteobacteria bacterium (Deb), Methanobacterium thermoautotrophicum (Mt), Methanococcus maripaludis, Methanopyrus kandleri, Halobacterium, Archaeoglobus fulgidus (Af), Pyrococcus furiosus (Pf), Pyrococcus horikoshii (Ph), Pyrobaculum aerophilum, Pyrococcus abyssi, Sulfolobus solfataricus (Ss), Sulfolobus tokodaii, Aeropyrum pernix (Ap), Thermoplasma acidophilum, and Thermoplasma volcanium.

In some embodiments, the eubacteria are one of Escherichia coli, Thermus thermophilus, Bacillus subtilis, and Geobacillus stearothermophilus.

In some embodiments, the aminoacyl-tRNA synthetase is a chimeric protein derived from enzymes of two or more organisms.

In some embodiments, the aminoacyl-tRNA synthetase comes from one or more of Mm, Mb, Ma, G1, Lum1, 1R26, Int, Nitra, and Deb.

In some embodiments, the aminoacyl-tRNA synthetase includes one or more of TyrRS, LeuRS, PylRS, chPheRS, and EcTrpRS, or variants or functional fragments of the aforementioned enzymes. In some embodiments, PylRS is one or more of MaPyIRS, MbPyIRS, MmPyIRS, G1PylRS, Lum1PylRS, 1R26PylRS, IntPylRS, NitraPylRS, DebPylRS, and chPylRS, or variants or functional fragments of the aforementioned enzymes.

In some embodiments, LeuRS includes EcLeuRS and its variants or functional fragments.

In some embodiments, TyrRS includes EcTyrRS and its variants.

In some embodiments, MmPyIRS is wild-type or a homologous sequence as shown in SEQ ID: 68.

In some embodiments, MmPyIRS has mutations in the amino acid recognition region, preferably, the mutations are selected from one or more of the following: L309A/N346Q/C348S, L301M/Y306L/L309A/C348F, C348T/Y384F, Y384W, Y384F, L301M/L305I/L309A/C348F, L309A/C348S/Y384F, L301M/L305I/Y306L/L309A, Y306A/Y384F, C348V, L301M/L305I/Y306F/L309A/C348F, Y306F/C348T, C348T, L309A/C348F/Y384W, L309A/C348T/Y384W, L309A/C348F/Y384F, L309A/C348A, Y306V/L309A/C348F/Y384F, Y306G/Y384F, Y306M/L309A/C348A, Y306A/L309M, Y306M/L309A/C348A/Y384F, M276F/A302S/Y306C/L309M, Y306I/L309M/C348A, Y306G/L309A/C348F/1405T/P406P/L407I, L301M/L305I/Y306L/L309A/C348F, L309A/C348V/Y384F, L309A/C348S, L309G/C348V/M350A/1405R/1413V, L309T/C348G/Y384F, Y306A/L309V/C348V/M350Y/Y384F/1405R, Y306A/L309M/C348G/Y384F/1405R, Y306G/C348V, L301M/L305I/Y306L/L309A/C348I, L301V/L305I/Y306F/L309A/C348F, A302S/C348V/M350F/D379G, M276/A302/Y306M/L309G/C348A/Y384W, M276/Y306A/L309V/C348V/M350Y/Y384F/1405R, Y306M/L309T/C348A/Y384F, Y306A/L309A/C348S/Y384F, Y306A/Y384F/1413L, C348G/V401C/Y384F, L305I/Y306A/Y384F, L309G/C348V/Y384F, L309C/C348V/1413V, Y306G/C348V/1405R, Y306G/L309V/C348V/M350Y/1405R, Y306A/L309V/C348V/M350Y/1405R, Y306A/L309A/C348V/M350Y/1405R/1413V, C348V/M350L/1405K/1413V, Y306A/C348V/1405L/1413V, C348S/Y384F, L301M/Y306A/L309A/C348F, N346G/C348A, N346A/C348A, Y306L/L309S/N346S/C348M, A302T/N346V/C348W/Y384F/V401L, A302T/N346G/C348T/V4011/W417Y, N346G/C348G/Y384F, A302T/N346A/C348A/V401L/W417A, L305F/L309M/N346G/C348G, Y306L/L309A/N346A/C348M/W417T, A302I/N346T/C348I/Y384L/W417K, L305M/1322T/N346G, A302T/N346A/C348V/Y384F/W417T, L305F/L309M/N346A/C348G, L305F/N346A/C348G, L305F/L309M/N346G/C348G/Y384F, A302T/L309S/N346V/C348G, A302T/L309A/1322T/N346A/C348G, Y306M/L309A/N346A/C348A/Y384F, L305G/N346G/C348A, L305G/L309A/N346G/C348A, L305G/L309L/N346G/C348A, N346G/C348G, 1322V/N346S/C348G/V401H/W417V, N346S/C348A/V401H/W417I, L305A/Y306L/C348A/Y384W/V401S, L305A/Y306F/C348A/Y384W/V401S, N346A/C348M/V401G/W417T, N346G/C348Q, N346Q/C348S/V401G/W417T, L309G/N346A/C348I/V401K/W417I, L305I/Y306F/L309G/C348F/Y384F, A302T/N346T/C348T, L305G/Y306F/N346G/C348F/V401G/W417Y, L305M/Y306M/N346A/C348G/V401G/W417H, N346G/C348Q/V401G, N346G/C348V/V401K, A302Q/N346S/C348W, N346Q/Y384F, L305I/L309G/N346C/C348W/Y384F, L305V/L309G/N346C/C348W/Y384F, N346S/C348G/Y384F, A302D/N346G/C348G, N346S/C348A/Y384F, N346C/C348S/Y384F, A302Y/Y306A/N346T/C348G/Y384F, N346M/C348Q/V401G/W417N, N346Q/C348A/V401M, M241F/A302S/Y306C/L309M, C348W/W417T, Y306C/N346Q/Y384F/V401C, N346S/C348G/V401A/W417T, A302T/N346A/C348G/Y384F/W417T, C348W/W382S, and Y306A/C348V.

In some embodiments, MbPyIRS is wild-type or a homologous sequence as shown in SEQ ID: 67. In some embodiments, MbPyIRS has mutations in the amino acid recognition region, preferably; the mutations are selected from one or more of the following: L274A/N311Q/C313S, L266M/Y271L/L274A/C313F, C313T/Y349F, Y349W, Y349F, L266M/L270I/L274A/C313F, L274A/C313S/Y349F, L266M/L270I/Y271L/L274A, Y271A/Y349F, C313V, L266M/L270I/Y271F/L274A/C313F, Y271F/C313T, C313T, L274A/C313F/Y349W, L274A/C313T/Y349W, L274A/C313F/Y349F, L274A/C313A, Y271V/L274A/C313F/Y349F, Y271G/Y349F, Y271M/L274A/C313A, Y271A/L274M, Y271M/L274A/C313A/Y349F, M241F/A267S/Y271C/L274M, Y271I/L274M/C313A, Y271G/L274A/C313F/V370T/S371P/L372I, L266M/L270I/Y271L/L274A/C313F, L274A/C313V/Y349F, L274A/C313S, L274G/C313V/M315A/V370R/1378V, L274T/C313G/Y349F, Y271A/L274V/C313V/M315Y/Y349F/V370R, Y271A/L274M/C313G/Y349F/V370R, Y271G/C313V, L266M/L270I/Y271L/L274A/C313I, L266V/L270I/Y271F/L274A/C313F, A267S/C313V/M315F/D344G, M241/A267/Y271M/L274G/C313A/Y349W, M241A/Y271A/L274V/C313V/M315Y/Y349F/V370R, Y271M/L274T/C313A/Y349F, Y271A/L274A/C313S/Y349F, Y271A/Y349F/1378L, C313G/V366C/Y349F, L270I/Y271A/Y349F, L274G/C313V/Y349F, L274C/C313V/1378V, Y271G/C313V/V370R, Y271G/L274V/C313V/M315Y/V370R, Y271A/L274V/C313V/M315Y/V370R, Y271A/L274A/C313V/M315Y/V370R/1378V, C313V/M315L/V370K/1378V, Y271A/C313V/V370L/1378V, C313S/Y349F, L266M/Y271A/L274A/C313F, N311G/C313A, N311A/C313A, Y271L/L274S/N311S/C313M, A267T/N311V/C313W/Y349F/V366L, A267T/N311G/C313T/V366I/W382Y, N311G/C313G/Y349F, A267T/N311A/C313A/V366L/W382A, L270F/L274M/N311A/C313G, Y271L/L274A/N311A/C313M/W382T, A2671/N311T/C313I/Y349L/W382K, L270M/I287T/N311G, A267T/N311A/C313V/Y349F/W382T, L270F/N311A/C313G, L270F/L274M/N311G/C313G/Y349F, A267T/L27S/N311V/C313G, A267T/L27A/I287T//N311A/C313G, Y271M/L274A/N311A/C313A/Y349F, L270G/N311G/C313A, L270G/L274A/N311G/C313A, L270G/L274L/N311G/C313A, N311G/C313G, I287V/N311S/C313G/V366H/W382V, N311S/C313A/V366H/W382I, L270A/Y271L/C313A/Y349W/V366S, L270A/Y271F/C313A/Y349W/V366S, N311A/C313M/V366G/W382T, N311G/C313Q, N311Q/C313S/V366G/W382T, L274G/N311A/C313I/V366K/W382I, L270I/Y271F/L274G/C313F/Y349F, A267T/N311T/C313T, L270G/Y271F/N311G/C313F/V366G/W382Y, L270M/Y271M/N311A/C313G/V366G/W382H, N311G/C313Q/V366G, N311G/C313V/V366K, A267Q/N311S/C313W, N311Q/Y349F, L270I/L274G/N311C/C313W/Y349F, L270V/L274G/N311C/C313W/Y349F, N311S/C313G/Y349F, A267D/N311G/C313G, N311S/C313A/Y349F, N311C/C313S/Y349F, A267Y/Y271A/N311T/C313G/Y349F, N311M/C313Q/V366G/W382N, N311Q/C313A/V366M, C313W/W382T, Y271C/N311Q/Y349F/V366C, N311S/C313G/V366A/W382T, A267T/N311A/C313G/Y349F/W382T, C313W/W382S, and Y271A/C313V.

In some embodiments, chPylRS is wild-type or further has an IPYE mutation based on the wild-type, or is a homologous sequence as shown in SEQ ID: 69.

In some embodiments, chPylRS has mutations in the amino acid recognition region, and the mutations are selected from combinations of one or more of the following positions: A267, C313, 1287, 1370, 1378, L266, L270, L274, L27, N311, V366, W382, Y271, and Y349.

In some embodiments, chPylRS has mutations in the amino acid recognition region, preferably, the mutations are selected from one or more of the following: L274A/N311Q/C313S, C313T/Y349F, Y349W, L266M/L270I/L274A/C313F, L274A/C313S/Y349F, Y349F, L266M/L270I/Y271L/L274A, Y271A/Y349F, C313V, L266M/L270I/Y271F/L274A/C313F, Y271F/C313T, C313T, L274A/C313F/Y349W, L274A/C313T/Y349W, L274A/C313F/Y349F, Y271M/L274A/C313A, Y271A/L274M, M241F/A267S/Y271C/L274M, Y271I/L274M/C313A, L274A/C313V/Y349F, L274A/C313S, L274G/C313V/M315A/I370R/I378V, Y271A/L274V/C313V/M315Y/Y349F/I370R, Y271G/C313V, L266M/L270I/Y271L/L274A/C313I, L266V/L270I/Y271F/L274A/C313F, A267S/C313V/M315F/D344G, M241/A267/Y271M/L274G/C313A/Y349W, M241/Y271A/L274V/C313V/M315Y/Y349F/I370R, Y271M/L274T/C313A/Y349F, L270I/Y271A/Y349F, L274G/C313V/Y349F, L274C/C313V/I378V, Y271G/C313V/I370R, Y271G/L274V/C313V/M315Y/I370R, Y271A/L274V/C313V/M315Y/I370R, Y271A/L274A/C313V/M315Y/I370R/I378V, C313V/M315L/V370K/I378V, Y271A/C313V/I370L/I378V, C313S/Y349F, N311G/C313A, L270F/L274M/N311A/C313G, L270F/L274M/N311G/C313G/Y349F, Y271M/L274A/N311A/C313A/Y349F, I287V/N311S/C313G/V366H/W382V, N311S/C313A/V366H/W382I, N311A/C313M/V366G/W382T, L270I/Y271F/L274G/C313F/Y349F, N311G/C313V/V366K, A267Q/N311S/C313W, N311Q/Y349F, N311S/C313G/Y349F, A267D/N311G/C313G, N311S/C313A/Y349F, N311C/C313S/Y349F, A267Y/Y271A/N311T/C313G/Y349F, N311M/C313Q/V366G/W382N, N311Q/C313A/V366M, C313W/W382T, Y271C/N311Q/Y349F/V366C, N311S/C313G/V366A/W382T, C348G/V401C/Y384F, L270I/L274G/N311C/C313W/Y349F, and L270V/L274G/N311C/C313W/Y349F.

In some embodiments, MaPylRS is wild-type or a homologous sequence as shown in SEQ ID: 70.

In some embodiments, MaPylRS has mutations in the amino acid recognition region, and the mutations are selected from combinations of one or more of the following positions: A122, A223, H227, 1142, L121, M129, N166, V168, V235, W239, Y206, and Y126.

In some embodiments, G1PylRS is wild-type or a homologous sequence as shown in SEQ ID: 71.

In some embodiments, G1PylRS has mutations in the amino acid recognition region, and the mutations are selected from combinations of one or more of the following positions: A121, A221, H120, H225, 1141, L124, M128, N165, V167, V233, W237, Y125, and Y204.

In some embodiments, G1PylRS has mutations in the amino acid recognition region, preferably, the mutations are selected from one or more of the following: H120M/Y125L/M128A/V167F, V167T/Y204F, Y204W, Y204F, H120M/L124I/M128A/V167F, M128A/V167S/Y204F, H120M/L124I/Y125L/M128A, Y125A/Y204F, V167V, H120M/L124I/Y125F/M128A/V167F, Y125F/V167T, V167T, M128A/V167F/Y204W, M128A/V167T/Y204W, M128A/V167F/Y204F, M128A/V167A, Y125V/M128A/V167F/Y204F, Y125F/Y204F, Y125M/M128A/V167A, Y125A/M128M, Y125M/M128A/V167A/Y204F, M95F/A121S/Y125C/M128M, Y125I/M128M/V167A, Y125G/M128A/V167F/H225T/K226P/L227I, H120M/L124I/Y125L/M128A/V167F, M128A/V167V/Y204F, M128A/V167S, M128G/M169A/H225R, M128T/V167G/Y204F, Y125A/M128V/M169Y/Y204F/H225R, Y125A/M128M/V167G/Y204F/H225R, Y125G/V167V, H120M/L124I/Y125L/M128A/V167I, H120V/L124I/Y125F/M128A/V167F, A121S/M169F/E199G, M95/A121/Y125M/M128G/V167A/Y204W, M95/Y125A/M128V/M169Y/Y204F/H225R, Y125M/M128T/V167A/Y204F, Y125A/M128A/V167S/Y204F, Y125A/Y204F/V233L, V167G/A221C/Y204F, L124I/Y125A/Y204F, M128G/V167V/Y204F, M128C, Y125G/V167V/H225R, Y125G/M128V/M169Y/H225R, Y125A/M128V/M169Y/H225R, Y125A/M128A/M169Y/H225R, M169L/H225K, Y125A/H225L, V167S/Y204F, H120M/Y125A/M128A/V167F, N165G/V167A, N165A/V167A, Y125L/M128S/N165S/V167M, A121T/N165V/V167W/Y204F/A221L, A121T/N165G/V167T/A221I/W237Y, N165G/V167G/Y204F, A121T/N165A/V167A/A221L/W237A, L124F/M128M/N165G/V167G, Y125L/M128A/N165A/V167M/W237T, A121I/N165T/V167I/Y204L/W237K, L124M/I141T/N165G, A121T/N165A/Y204F/W237T, L124F/M128M/N165A/V167G, L124F/N165A/V167G, L124F/M128M/N165G/V167G/Y204F, A121T/M128S/N165V/V167G, A121T/M128A/N165A/V167G, Y125M/M128A/N165A/V167A/Y204F, L124G/N165G/V167A, L124G/M128A/N165G/V167A, L124G/M128L/N165G/V167A, N165G/V167G, N165S/V167G/A221H/W237V, N165S/V167A/A221H/W237I, L124A/Y125L/V167A/Y204W/A221S, L124A/Y125F/V167A/Y204W/A221S, N165A/V167M/A221G/W237T, N165G/V167Q, N165Q/V167S/A221G/W237T, M128G/N165A/V167I/A221K/W237I, L124I/Y125F/M128G/V167F/Y204F, A121T/N165T/V167T, L124G/Y125F/N165G/V167F/A221G/W237Y, L124M/Y125M/N165A/V167G/A221G/W237H, N165G/V167Q/A221G, N165G/V167V/A221K, A121Q/N165S/V167W, N165Q/Y204F, L124I/M128G/N165C/V167W/Y204F, L124V/M128G/N165C/V167W/Y204F, N165S/V167G/Y204F, A121D/N165G/V167G, N165S/V167A/Y204F, N165C/V167S/Y204F, A121Y/Y125A/N165T/V167G/Y204F, N165M/V167Q/A221G/W237N, N165Q/V167A/A221M, V167W/W237T, Y125C/N165Q/Y204F/A221C, N165S/V167G/A221A/W237T, A121T/N165A/V167G/Y204F/W237T, V167W/W237S, Y125G/M128A/V167F/H226T/K227P/L228I, Y125A/H225I/K226P, L124G/N165G/V167A/M128A, L124A/Y125F/Y204W/A221S/W237Y, and L124G/Y125F/N165G/V167F/Y204W/A221G/W237Y.

In some embodiments, 1R26PylRS is wild-type or a homologous sequence as shown in SEQ ID: 73. In some embodiments, 1R26PylRS has mutations in the amino acid recognition region, and the mutations are selected from combinations of one or more of the following positions: M96, L121, A122, L125, Y126, M129, I142, N166, V168, M170, E201, Y206, A223, H227, Y228, L229, W239, and V235.

In some embodiments, 1R26PylRS has mutations in the amino acid recognition region, preferably, the mutations are selected from one or more of the following: L121M/Y126L/M129A/V168F, V168T/Y206F, Y206W, Y206F, L121M/L125I/M129A/V168F, M129A/V168S/Y206F, L121M/L125I/Y126L/M129A, Y126A/Y206F, V168V, L121M/L125I/Y126F/M129A/V168F, Y126F/V168T, V168T, M129A/V168F/Y206W, M129A/V168T/Y206W, M129A/V168F/Y206F, M129A/V168A, Y126V/M129A/V168F/Y206F, Y126G/Y206F, Y126M/M129A/V168A, Y126A/M129M, Y126M/M129A/V168A/Y206F, M96F/A122S/Y126C/M129M, Y126I/M129M/V168A, Y126G/M129A/V168F/H227T/Y228P/L229I, L121M/L125I/Y126L/M129A/V168F, M129A/V168V/Y206F, M129A/V168S, M129G/M170A/H227R, M129T/V168G/Y206F, Y126A/M129V/M170Y/Y206F/H227R, Y126A/M129M/V170G/Y206F/H227R, Y126G/V168V, L121M/L125I/Y126L/M129A/V168I, L121V/L125I/Y126F/M129A/V168F, A122S/M170F/E201G, M96/A122/Y126M/M129G/V168A/Y206W, M96/Y126A/M129V/M170Y/Y206F/H227R, Y126M/M129T/V168A/Y206F, Y126A/M129A/V168S/Y206F, Y126A/Y206F/V235L, V168G/A223C/Y206F, M129I/Y126A/Y206F, M129G/V168V/Y206F, M129C, Y126G/V168V/L229R, Y126G/M129V/M170Y/H227R, Y126A/M129V/M170Y/H227R, Y126A/M129A/M170Y/H227R, M170L/H227K, Y126A/H227L, V168S/Y206F, L121M/Y126A/M129A/V168F, N166G/V168A, N166A/V168A, Y126L/M129S/N166S/V168M, A122T/N166V/V168W/Y206F/A223L, A122T/N166G/V168T/A223I/W239Y, N166G/V168G/Y206F, A122T/N166A/V168A/A223L/W239A, L125F/M129M/N166G/V168G, Y126L/M129A/N166A/V168M/W239T, A122I/N166T/V168I/Y206L/W239K, L125M/I142T/N166G, A122T/N166A/Y206F/W239T, L125F/M129M/N166A/V168G, L125F/N166A/V168G, L125F/M129M/N166G/V168G/Y206F, A122T/M129S/N166V/V168G, A122T/M129A/N166A/V168G, Y126M/M129A/N166A/V168A/Y206F, L125G/N166G/V168A, L125G/M129A/N166G/V168A, L125G/M129L/N166G/V168A, N166G/V168G, N166S/V168G/A223H/W239V, N166S/V168A/A223H/W239I, L125A/Y126L/V168A/Y206W/A223S, L125A/Y126F/V168A/Y206W/A223S, N166A/V168M/A223G/W239T, N166G/V168Q, N166Q/V168S/A223G/W239T, M129G/N166A/V168I/A223K/W239I, L125I/Y126F/M129G/V168F/Y206F, A122T/N166T/V168T, L125G/Y126F/N166G/V168F/A223G/W239Y, L125M/Y126M/N166A/V168G/A223G/W239H, N166G/V168Q/A223G, N166G/V168V/A223K, A122Q/N166S/V168W, N166Q/Y206F, L125I/M129G/N166C/V168W/Y206F, L125V/M129G/N166C/V168W/Y206F, N166S/V168G/Y206F, A122D/N166G/V168G, N166S/V168A/Y206F, N166C/V168S/Y206F, A122Y/Y126A/N166T/V168G/Y206F, N166M/V168Q/A223G/W239N, N166Q/V168A/A223M, V168W/W239T, Y126C/N166Q/Y206F/A223C, N166S/V168G/A223A/W239T, A122T/N166A/V168G/Y206F/W239T, V168W/W239S, and Y126A/H227I/Y228P.

In some embodiments, Lum1PylRS is wild-type or a homologous sequence as shown in SEQ ID: 72.

In some embodiments, Lum1PylRS has mutations in the amino acid recognition region, and the mutations are selected from combinations of one or more of the following positions: M96, L121, A122, L125, Yl26, M129, L142, N166, V168, L170, M129, E200, Y205, A222, P226, L227, M228, 1234, and W238.

In some embodiments, Lum1PylRS has mutations in the amino acid recognition region, preferably, the mutations are selected from one or more of the following: L121M/Y126L/M129A/V168F, V168T/Y205F, Y205W, Y205F, L121M/L125I/M129A/V168F, M129A/V168S/Y205F, L121M/L125I/Y126L/M129A, Y126A/Y205F, V168V, L121M/L125I/Y126F/M129A/V168F, Y126F/V168T, V168T, M129A/V168F/Y205W, M129A/V168T/Y205W, M129A/V168F/Y205F, M129A/V168A, Y126V/M129A/V168F/Y205F, Y126G/Y205F, Y126M/M129A/V168A, Y126A/M129M, Y126M/M129A/V168A/Y205F, M96F/A122S/Y126C/M129M, Y126I/M129M/V168A, Y126G/M129A/V168F/P226T/L227P/M228I, L121M/L125I/Y126L/M129A/V168F, M129A/V168V/Y205F, M129A/V168S, M129G/L170A/P226R/I234V, M129T/V168G/Y205F, Y126A/M129V/L170Y/Y205F/P226R, Y126A/M129M/V170G/Y205F/P226R, Y126G/V168V, L121M/L125I/Y126L/M129A/V168I, L121V/L125I/Y126F/M129A/V168F, A122S/L170F/E200G, M96/A122/Y126M/M129G/V168A/Y205W, M96/Y126A/M129V/L170Y/Y205F/P226R, Y126M/M129T/V168A/Y205F, Y126A/M129A/V168S/Y205F, Y126A/Y205F/I234L, V168G/A222C/Y205F, Y125I/Y126A/Y205F, M129G/V168V/Y205F, M129C/I234V, Y126G/V168V/P226R, Y126G/M129V/L170Y/P226R, Y126A/M129V/L170Y/P226R, Y126A/M129A/L170Y/P226R, P226K/I234V, Y126A/P226L/I234V, V168S/Y205F, L121M/Y126A/M129A/V168F, N166G/V168A, N166A/V168A, Y126L/M129S/N166S/V168M, A122T/N166V/V168W/Y205F/A222L, A122T/N166G/V168T/A222I/W238Y, N166G/V168G/Y205F, A122T/N166A/V168A/A222L/W238A, L125F/M129/N166G/V168G, Y126L/M129A/N166A/V168M/W238T, A122I/N166T/V168I/Y205L/W238K, L125M/L142T/N166G, A122T/N166A/Y205F/W238T, L125F/M129/N166A/V168G, L125F/N166A/V168G, L125F/M129M/N166G/V168G/Y205F, A122T/M129S/N166V/V168G, A122T/M129A/N166A/V168G, Y126M/M129A/N166A/V168A/Y205F, L125G/N166G/V168A, L125G/M129A/N166G/V168A, L125G/M129L/N166G/V168A, N166G/V168G, N166S/V168G/A222H/W238V, N166S/V168A/A222H/W238I, L125A/Y126L/V168A/Y205W/A222S, L125A/Y126F/V168A/Y205W/A222S, N166A/V168M/A222G/W238T, N166G/V168Q, N166Q/V168S/A222G/W238T, M129G/N166A/V168I/A222K/W238I, L125I/Y126F/M129G/V168F/Y205F, A122T/N166T/V168T, L125G/Y126F/N166G/V168F/A222G/W238Y, L125M/Y126M/N166A/V168G/A222G/W238H, N166G/V168Q/A222G, N166G/V168V/A222K, A122Q/N166S/V168W, N166Q/Y205F, L125I/M129G/N166C/V168W/Y205F, L125V/M129G/N166C/V168W/Y205F, N166S/V168G/Y205F, A122D/N166G/V168G, N166S/V168A/Y205F, N166C/V168S/Y205F, A122Y/Y126A/N166T/V168G/Y205F, N166M/V168Q/A222G/W238N, N166Q/V168A/A222M, V168W/W238T, Y126C/N166Q/Y205F/A222C, N166S/V168G/A222A/W238T, A122T/N166A/V168G/Y205F/W238T, V168W/W238S, and Y126A/P226I/L227P.

In some embodiments, NitraPylRS is wild-type or a homologous sequence as shown in SEQ ID: 74.

In some embodiments, NitraPylRS has mutations in the amino acid recognition region, and the mutations are selected from combinations of one or more of the following positions: M99, L124, A125, L128, Y129, M132, 1144, N168, V170, L172, E202, Y207, A224, W240, K228, and V236.

In some embodiments, NitraPylRS has mutations in the amino acid recognition region, preferably, the mutations are selected from one or more of the following: L124M/Y129L/M132A/V170F, V170T/Y207F, Y207W, Y207F, L124M/L128I/M132A/V170F, M132A/V170S/Y207F, L124M/L128I/Y129L/M132A, Y129A/Y207F, V170V, L124M/L128I/Y129F/M132A/V170F, Y129F/V170T, V170T, M132A/V170F/Y207W, M132A/V170T/Y207W, M132A/V170F/Y207, M132A/V170F/Y207F, M132A/V170A, Y129V/M132A/V170F/Y207F, Y129G/Y207F, Y129M/M132A/V170A, Y129A/M132M, Y129M/M132A/V170A/Y207F, M99F/A125S/Y129C/M132M, Y129I/M132M/V170A, Y129G/M132A/V170F/K228T/P229P/I230I, L124M/L128I/Y129L/M132A/V170F, M132A/V170V/Y207F, M132A/V170S, M132G/L172A/K228R, M132T/V170G/Y207F, Y129A/M132V/L172Y/Y207F/K228R, Y129A/M132M/V170G/Y207F/K228R, Y129G/V170V, L124M/L128I/Y129L/M132A/V170I, L124V/L128I/Y129F/M132A/V170F, A125S/L172F/E202G, M99/A125/Y129M/M132G/V170A/Y207W, M99/Y129A/M132V/L172Y/Y207F/K228R, Y129M/M132T/V170A/Y207F, Y129A/M132A/V170S/Y207F, Y129A/Y207F/V236L, V170G/A224C/Y207F, L128I/Y129A/Y207F, M132G/V170V/Y207F, M132C, Y129G/V170V/K228R, Y129G/M132V/L172Y/K228R, Y129A/M132V/L172Y/K228R, Y129A/M132A/L172Y/K228R, Y129A/K228L, V170S/Y207F, L124M/Y129A/M132A/V170F, N168G/V170A, N168A/V170A, Y129L/M132S/N168S/V170M, A125T/N168V/V170W/Y207F/A224L, A125T/N168G/V170T/A224I/W240Y, N168G/V170G/Y207F, A125T/N168A/V170A/A224L/W240A, L128F/M132/N168G/V170G, Y129L/M132A/N168A/V170M/W240T, A125I/N168T/V170I/Y207L/W240K, L128M/I144T/N168G, A125T/N168A/Y207F/W240T, L128F/M132/N168A/V170G, L128F/N168A/V170G, L128F/M132M/N168G/V170G/Y207F, A125T/M132S/N168V/V170G, A125T/M132A/N168A/V170G, Y129M/M132A/N168A/V170A/Y207F, L128G/N168G/V170A, L128G/M132A/N168G/V170A, L128G/M132L/N168G/V170A, N168G/V170G, N168S/V170G/A224H/W240V, N168S/V170A/A224HI/W240, L128A/Y129L/V170A/Y207W/A224S, L128A/Y129F/V170A/Y207W/A224S, N168A/V170M/A224G/W240T, N168G/V170Q, N168Q/V170S/A224G/W240T, M132G/N168A/V170I/A224K/W240I, L128I/Y129F/M132G/V170F/Y207F, N125T/N168T/V170T, L128G/Y129F/N168G/V170F/A224G/W240Y, L128M/Y129M/N168A/V170G/A224G/W240H, N168G/V170Q/A224G, N168G/V170V/A224K, A125Q/N168S/V170W, N168Q/Y207F, L128I/M132G/N168C/V170W/Y207F, L128V/M132G/N168C/V170W/Y207F, N168S/V170G/Y207F, A125D/N168G/V170G, N168S/V170A/Y207F, N168C/V170S/Y207F, A125Y/Y129A/N168T/V170G/Y207F, N168M/V170Q/A224G/W240N, N168Q/V170A/A224M, V170W/W240T, Y129C/N168Q/Y207F/A224C, N168S/V170G/A224A/W240T, A125T/N168A/V170G/Y207F/W240T, V170W/W240S, and Y129A/K228I.

In some embodiments, DebPylRS is wild-type or a homologous sequence as shown in SEQ ID: 75.

In some embodiments, DebPylRS has mutations in the amino acid recognition region, and the mutations are selected from combinations of one or more of the following positions: L122, M97, I20, A123, L126, Yl27, M130, L134, 1144, N168, V170, M172, E204, Y209, A228, P233, 1240, W244, and H232.

In some embodiments, DebPylRS has mutations in the amino acid recognition region, preferably, the mutations are selected from one or more of the following: L122M/Y127L/M130A/V170F, V170T/Y209F, Y209W, Y209F, L122M/L126I/M130A/V170F, M130A/V170S/Y209F, L122M/L126I/Y127L/M130A, Y127A/Y209F, V170V, L122M/L126I/Y127F/M130A/V170F, Y127F/V170T, V170T, M130A/V170F/Y209W, M130A/V170T/Y209W, M130A/V170F/Y209F, M130A/Y209A, Y127V/M130A/V170F/Y209F, Y127G/Y209F, M130A/V170V/Y209F, Y127M/M130A/V170A, Y127A/M130M, Y127M/M130A/V170A/Y209F, M97F/A123S/Y127C/M130M, Y127I/M130M/V170A, Y127G/M130A/V170F/H232T/P233P/L134I, L122M/L126I/Y127L/M130A/V170F, M130A/V170S, M130G/M172A/H232R/I240V, M130T/V170G/Y209F, Y127A/M130V/M172Y/Y209F/H232R, Y127A/M130M/V170G/Y209F/H232R, Y127G/V170V, L122M/L126I/Y127L/M130A/V170I, L122V/L126I/Y127F/M130A/V170F, A123S/M172F/E204G, M97/A123/Y127M/M130G/V170A/Y209W, M97/Y127A/M130V/M172Y/Y209F/H232R, Y127M/M130T/V170A/Y209F, Y127A/M130A/V170S/Y209F, Y127A/Y209F/I240L, V170G/A228C/Y209F, L126I/Y127A/Y209F, M130G/V170V/Y209F, M130C/I240V, Y127G/V170V/H232R, Y127G/M130V/M172Y/H232R, Y127A/M130V/M172Y/H232R, Y127A/M130A/M172Y/H232R, M172L/H232K/I240V, Y127A/H232L/I20V, V170S/Y209F, L122M/Y127A/M130A/V170F, N168G/V170A, N168A/V170A, Y127L/M130S/N168S/V170M, A123T/N168V/V170W/Y209F/A228L, A123T/N168G/V170T/A228I/W244Y, N168G/V170G/Y209F, A123T/N168A/V170A/A228L/W244A, L126F/M130/N168G/V170G, Y127L/M130A/N168A/V170M/W244T, A123I/N168T/V170I/Y209L/W244K, L126M/I144T/N168G, A123T/N168A/Y209F/W244T, L126F/M130/N168A/V170G, L126F/N168A/V170G, L126F/M130M/N168G/V170G/Y209F, A123T/M130S/N168V/V170G, A123T/M130A/N168A/V170G, Y127M/M130A/N168A/V170A/Y209F, L126G/N168G/V170A, L126G/M130A/N168G/V170A, L126G/M130L/N168G/V170A, N168G/V170G, N168S/V170G/A228H/W244V, N168S/V170A/A228H/W244I, L126A/Y127L/V170A/Y209W/A228S, L126A/Y127F/V170A/Y209W/A228S, N168A/V170M/A228G/W244T, N168G/V170Q, N168Q/V170S/A228G/W244T, M130G/N168A/V170I/A228K/W244I, L126I/Y127F/M130G/V170F/Y209F, A123T/N168T/V170T, L126G/Y127F/N168G/V170F/A228G/W244Y, L126M/Y127M/N168A/V170G/A228G/W244H, N168G/V170Q/A228G, N168G/V170V/A228K, A123Q/N168S/V170W, N168Q/Y209F, L126I/M130G/N168C/V170W/Y209F, L126V/M130G/N168C/V170W/Y209F, N168S/V170G/Y209F, A123D/N168G/V170G, N168S/V170A/Y209F, N168C/V170S/Y209F, A123Y/Y127A/N168T/V170G/Y209F, N168M/V170Q/A228G/W244N, N168Q/V170A/A228M, V170W/W244T, Y127C/N168Q/Y209F/A228C, N168S/V170G/A228A/W244T, A123T/N168A/V170G/Y209F/W244T, V170W/W244S, and Y127A/H232I.

In some embodiments, chPheRS is a fusion protein that includes the tRNA binding domain (NTD) and the amino acid recognition domain (CTD).

In some embodiments, the tRNA binding domain of chPheRS is from the tRNA binding domain of any one of MaPylRS, MbPylRS, MmPylRS, G1PylRS, Lum1PylRS, 1R26PylRS, IntPylRS, NitraPylRS, DebPylRS, and chPylRS.

In some embodiments, the tRNA binding domain of chPheRS is as shown in any one of SEQ ID: 83-90 or its homologous sequence.

In some embodiments, the amino acid recognition domain of chPheRS is from the catalytic domain of phenylalanyl-tRNA synthetase in the mitochondria of eukaryotes.

In some embodiments, the amino acid recognition domain of chPheRS is from the catalytic domain of human mitochondrial phenylalanyl-tRNA synthetase, with a sequence as shown in SEQ ID: 91.

In some embodiments, chPheRS further includes a linker peptide, and the linker peptide includes a sequence as shown in any one of SEQ ID: 92-95 or its homologous sequence.

In some embodiments, chPheRS is a homologous sequence as shown in SEQ ID: 78 or its homologous sequence.

In some embodiments, chPheRS has mutations in the amino acid recognition region, and the mutations are selected from combinations of one or more of the following positions: Q113, E148, V150, F221, T224, L247, and A264.

In some embodiments, chPheRS has mutations in the amino acid recognition region, preferably, the mutations are selected from one or more of the following: T224G/A264G, F221I/T224G/A264G, F221C/T224G/A264G, E148D/T224G/A264G, Q113N/T224S/A264S, Q113G/L247A/E148D/T224G/A264G, Q113F/E148D/V150C/T224G/A264G, E148D/V150G/F221V/T224G/L247V/A264G, F221V/T224G/A264G, E148D/F221V/T224G/A264G, V150G/F221V/Q113F/E148D/L247L/T224G/A264G, V150G/F221C/T224G/A264G, and F221V/Q113G/L247A/E148D/T224G/A264G.

In some embodiments, EcTyrRS is wild-type or a homologous sequence as shown in SEQ ID: 76.

In some embodiments, EcTyrRS has mutations in the amino acid recognition region, and the mutations are selected from combinations of one or more of the following positions: 17, Y37, L56, H70, L71, T76, S120, A121, W129, D158, D165, G180, D182, F183, L186, D192, Q195, and D265.

In some embodiments, EcTyrRS has mutations in the amino acid recognition region, preferably, the mutations are selected from one or more of the following: Y37V/D182S/F183M, Y37G/D182G/L186A, Y37V/D182S/F183Y, Y37I/D192G/F183M/L186A, Y37L/D182S/F183M/L186A, Y37I/D182G/F183M/L186A/D265R, H70A/D158T, Y37V/D182S/F183M/L186C/D165G, Y37G/D182G/L186A/I7F/L71V/G180S, Y37G/L71V/D182C/F183Y/L186C, Y37H/L71V/D182G/L186M, L71V/D182G/, L71V/W129F/D182G, Y37S/D182S/F183M/L186A, Y37S/D182S/F183A/L186E, Y37L/Q195S, Y37A/D182T/F183M, and Y37G/L71H/D182G/L186G/L56E/T76G/S120Y/A121H/F183I.

In some embodiments, EcLeuRS is wild-type or a homologous sequence as shown in SEQ ID: 77.

In some embodiments, EcLeuRS has mutations in the amino acid recognition region, and the mutations are selected from combinations of one or more of the following positions: E20, L38, M40, L41, T252, M40, Y499, Y500, Y527, L538, H529, F531, H537, and A560.

In some embodiments, EcLeuRS has mutations in the amino acid recognition region, preferably, the mutations are selected from one or more of the following: 499C/Y527G/H537T, M40I/Y499I/Y527A/H529G/T252A, M40V/L41S/Y499S/Y527L/H529G/T252R/E20K, M40A/L41N/Y499I/Y527G/H537T, L38F/M40G/L41P/Y499V/Y500L/Y527A/H537G/L538S/F531C/A560V, M40G/L41P/Y499G/Y527A/H537T, M40I/Y499I/Y527A/H537G, M40V/L41M/Y499L/Y527L/H537G, M40L/L41E/Y499R/Y527A/H537G, M40W/L41S/Y499I/Y527A/H537G, M40G/L41Q/Y499L/Y527G/H537F, M40G/L41Q/Y499L/Y527G/H537F/T252A, and E20K/M40V/L41S/T252R/Y499S/Y527L/H537G.

In some embodiments, EcTrpRS is wild-type or a homologous sequence as shown in SEQ ID: 79.

In some embodiments, EcTrpRS has mutations in the amino acid recognition region, and the mutations are selected from combinations of one or more of the following positions: V144, S8, Q109, and V146.

In some embodiments, the recoding tRNA is a chimera of two tRNAs or its variant, where the two different tRNAs come from different organisms.

In some embodiments, the recoding tRNA includes one or more of the anticodons corresponding to the following codons: TCG, ACG, CGA, TCA, CGC, TTG, ATA, and GCG.

In some embodiments, the recoding tRNA is selected from one of orthogonal tyrosyl-tRNA (TyrT), orthogonal leucyl-tRNA (LeuT), orthogonal pyrrolysyl-tRNA (PylT), preferably, the recoding tRNA is selected from one or more of C15, M15, CM15, MbPylT, MetPylT, SpePylT, Pyl-O1, Pyl-O2, MaPylT, G1PylT, G1hyb, Ma-T6, 12B72, Int, Int17, Int5, Int6B, Int6C, Int13, Alv21, Alv8, Alv17, Alv10, Alv22, G1hyb, Therm1, Int, BH52, CM4, AS78, BsTyrT, NGS6, EcLeuT, L-G1, and L-G2.

In some embodiments, when the aminoacyl-tRNA synthetase is PylRS, the orthogonal pyrrolysyl-tRNA is selected from one or more of C15, M15, CM15, MbPylT, MetPylT, SpePylT, Pyl-O1, Pyl-O2, Ma-T6, MaPylT, G1PylT, G1hyb, Ma-T6, 12B72, Int17, Int5, Int6B, Int6C, Int13, Alv21, Alv8, Alv17, Alv10, Alv22, G1hyb, Int, Therm1, and BH52.

In some embodiments, when the aminoacyl-tRNA synthetase is chPheRS, the recoding tRNA is selected from one or more of CM4, CM15, MbPylT, Pyl-O1, Pyl-O2, and AS78.

In some embodiments, when the aminoacyl-tRNA synthetase is EcTyrRS, the recoding tRNA is selected from one or more of BsTyrT and NGS6.

In some embodiments, when the aminoacyl-tRNA synthetase is EcLeuRS, the recoding tRNA is selected from one or more of EcLeuT, L-G1, and L-G2.

In some embodiments, the recoding tRNA is further encoded by a sequence as shown in any one of SEQ ID: 1-66 or its homologous sequence.

In some embodiments, the tRNA is expressed through a recombinant vector, and the recombinant vector is assembled and constructed by the Gibson seamless cloning method.

In some embodiments, the host cell is a modified host cell, and compared to the wild-type host cell, the expression level and/or activity of the endogenous tRNA encoding rare codons in the modified host cell is downregulated.

In some embodiments, the modification includes: gene knockout, gene mutation, and/or gene silencing.

In some embodiments, the modification includes knocking out the alleles of the endogenous tRNA encoding rare codons in the host cell.

In some embodiments, the modification includes administering one or more substances selected from the group consisting of: antisense RNA, siRNA, shRNA, and CRISPR/Cas9 system to the host cell.

In some embodiments, the modification includes administering the CRISPR/Cas9 system to the host cell.

In some embodiments, the modification further includes administering an sgRNA targeting the gene of the endogenous tRNA encoding rare codons to the host cell.

In some embodiments, the sequence of the sgRNA targeting the gene of the endogenous tRNA encoding rare codons includes a sequence as shown in any one of SEQ ID: 95-98.

In some embodiments, the environment in which the host cell is located does not contain the amino acid encoded by the rare codon in the wild-type host cell.

In some embodiments, the host cell includes a transcription template.

In some embodiments, the transcription template includes a first rare codon and/or a second rare codon.

In some embodiments, the transcription template includes a boxB sequence.

In some embodiments, the boxB sequence includes the sequence shown in SEQ ID: 80.

In some embodiments, the first aminoacyl-tRNA synthetase and/or the second aminoacyl-tRNA synthetase include the λN22 protein and functional fragments of the FUS protein.

In some embodiments, the sequence of the λN22 protein functional fragment includes the sequence shown in SEQ ID: 81.

In some embodiments, the sequence of the FUS protein functional fragment includes the sequence shown in SEQ ID: 82.

In some embodiments, the first aminoacyl-tRNA synthetase and/or the second aminoacyl-tRNA synthetase include functional fragments of the FUS protein.

In some embodiments, the unnatural amino acid is selected from one or more of the following: Tetrazine unnatural amino acids; p-acetyl-L-phenylalanine; p-iodo-L-phenylalanine; O-methyl-L-tyrosine; p-propargyloxyphenylalanine; p-propargyl-phenylalanine; L-3-(2-naphthyl)alanine; 3-methyl-phenylalanine; O-4-allyl-L-tyrosine; 4-propyl-L-tyrosine; L-DOPA; fluorinated phenylalanine; isopropyl-L-phenylalanine; p-azido-L-phenylalanine; p-acyl-L-phenylalanine; p-benzoyl-L-phenylalanine; L-phosphoserine; phosphonoserine; phosphonotyrosine; p-bromophenylalanine; p-amino-L-phenylalanine; isopropyl-L-phenylalanine; unnatural analogues of tyrosine amino acids; unnatural analogues of glutamine amino acids; unnatural analogues of phenylalanine amino acids; unnatural analogues of serine amino acids; unnatural analogues of threonine amino acids; amino acids substituted with alkyl, aryl, acyl, azido, cyano, halo, hydrazine, acylhydrazine, hydroxyl, alkenyl, alkynyl, ether, thiol, sulfonyl, seleno, ester, thioacid, borate, boronate, phosphate, phosphonate, phosphine, heterocyclic, enone, imine, aldehyde, hydroxylamine, keto, or amino groups; amino acids with photoactivatable crosslinkers; spin-labeled amino acids; fluorescent amino acids; metal-binding amino acids; metal-containing amino acids; radioactive amino acids; photocaged and/or photoisomerizable amino acids; amino acids containing biotin or biotin analogs; amino acids containing keto groups; amino acids containing polyethylene glycol or polyether; amino acids substituted with heavy atoms; chemically cleavable or photocleavable amino acids; amino acids with elongated side chains; amino acids containing toxic groups; sugar-substituted amino acids; amino acids containing carbon-linked sugars; amino acids with redox activity; amino acids containing α-hydroxy acids; amino thioacids; α,α-disubstituted amino acids; D-amino acids; cyclic amino acids other than proline or histidine; aromatic amino acids other than phenylalanine, tyrosine, or tryptophan, etc.

In some embodiments, the protein containing unnatural amino acids (UAAs) includes one or more of therapeutic proteins, diagnostic proteins, and industrial enzymes.

In some embodiments, the protein containing unnatural amino acids (UAAs) is selected from one or more of transcriptional regulatory proteins, cytokines, growth factor receptors, inflammatory molecules, oncogene products, peptide hormones, signal transduction molecules, and steroid hormone receptors.

In some embodiments, the protein containing unnatural amino acids (UAAs) includes one or more of α-1 antitrypsin, angiostatin, antihemolytic factor, antibodies, apolipoprotein, atrial natriuretic peptide, C-X-C chemokines, Hedgehog proteins, hemoglobin, hepatocyte growth factor (HGF), hirudin, insulin, insulin-like growth factor (IGF), keratinocyte growth factor (KGF), lactoferrin, leukemia inhibitory factor, luciferase, neurturin, neutrophil inhibitory factor (NIF), oncostatin M, osteogenin, parathyroid hormone, PD-ECSF, PDGF, pleiotrophin, protein A, protein G, pyrogenic exotoxins A, B or C, relaxin, renin, SCF, soluble complement receptor I, soluble interleukin receptor, soluble TNF receptor, somatomedin, streptokinase, superantigens, staphylococcal enterotoxins, superoxide dismutase (SOD), toxic shock syndrome toxin, thymosin al, tissue plasminogen activator, tumor growth factor (TGF), tumor necrosis factor and corticosterone, GAL4, erythropoietin (EPO), human growth hormone, T39765, NAP-2, ENA-78, Gro-a, Gro-b, Gro-c, IP-10, GCP-2, NAP-4, SDF-1, PF4, MIG, calcitonin, c-kit ligand, CC chemokines, monocyte chemoattractant protein-1, monocyte chemoattractant protein-2, monocyte chemoattractantprotein-3, monocyte inflammatoryprotein-1α, monocyte inflammatoryprotein-1β, RANTES, 1309, R83915, R91733, HCC1, T58847, D31065, T64262, CD40, CD40 ligand, C-kit ligand, collagen, colony stimulating factor (CSF), complement factor 5a, complement inhibitor, complement receptor 1, DHFR, epithelial neutrophil activating peptide-78, GROa/MGSA, GROβ, GROγ, MIP-1α, MIP-1δ, MCP-1, epidermal growth factor (EGF), epithelial neutrophil activating peptide, erythropoietin (EPO), exfoliating toxin, factor IX, factor VII, factor VIII, factor X, fibroblast growth factor (FGF), fibrinogen, fibronectin, G-CSF, GM-CSF, glucocerebrosidase, gonadotropin, human serum albumin, ICAM-1, ICAM-1 receptor, LFA-1, LFA-1 receptor, IGF-I, IGF-II, IFN-α, IFN-β, IFN-γ, IL-1, IL-2, IL-3, IL-4, IL-5, IL-6, IL-7, IL-8, IL-9, IL-10, IL-11, IL-12, SEA, SEB, SEC1, SEC2, SEC3, SED, SEE, soluble I-CAM1, TGF-α, TGF-β, tumor necrosis factor α, tumor necrosis factor R, tumor necrosis factor receptor (TNFR), VLA-4 protein, VCAM-1 protein, vascular endothelial growth factor (VEGF), urokinase, Mos, Ras, Raf, Met, p53, Tat, Fos, Myc, Jun, Myb, Rel, estrogen receptor, progesterone receptor, testosterone receptor, aldosterone receptor, LDL receptor, SCF/c-Kit, CD40L/CD40, VLA-4/VCAM-1, ICAM-1/LFA-1, and hyaluronan/CD44.

### DESCRIPTION OF THE DRAWINGS

Embodiments of the present invention will now be described further with reference to the accompanying drawing, in which:

FIG. 1 shows characteristics of all the sense codons. Sense codons were plotted based on the frequency in the genome and the abundance of corresponding tRNA decoders in HEK293T cells (right). The sense codons with frequency below 20 (/1000 codons) and corresponding tRNA abundance below 2% were chosen and shown in the left.

FIG. 2 shows schematic of ncAA recoding rate calculation for rare codon recoding. The recoding translation system and the rare codon-bearing POI were co-transfected into HEK293T cells. Then, two methods were used to calculate the ncAA recoding rate: 1) LC-MS analysis: the protein of the POI mixture, including the fraction incorporated with ncAA and the fraction with NAA at rare codons, was purified by Ni2+-NTA beads and subjected to LC-MS analysis. The ncAA recoding rate was determined by the intensity of two fractions quantified by LC-MS. 2) TCO-PEG5000 mass tag labeling analysis: this method was suitable for quantifying the TetBu recoding rate in this work. When the protein mixture was incubated with TCO-PEG5000 mass tag, the protein incorporated with TetBu would be labeled by this mass tag and migrated much slower in SDS-PAGE. The TetBu recoding rate was obtained by quantifying the intensity of the POI band shown in the figure.

FIG. 3. shows the recoding rate and background incorporation in proteome for different rare codons. The TetBu recoding rate into EGFP with different rare codon recoding systems by TetRS. EGFP bearing different rare codons at position 149 were co-expressed with corresponding rare codon recoding systems in the presence of TetBu and the recoding rate was then measured by LC-MS analysis. The background incorporation in the proteome by rare codon recoding was assayed with dot blotting. The anticodon of MmPylT was mutated to decode these rare codons and the amber stop codon. The recoding synthetase was obtained by introducing mutations (L270G/N311G/C313A) into chPylRSIPYE to generate TetBu-chPylRSIPYE (TetRS). The chPylRSIPYE (TetRS)/MmPylTyyy (yyy=anticodons of 7 rare codons and amber codon) pair was expressed in HEK293T cells in the presence or absence of 200 μM TetBu (3-(6-butyl-1,2,4,5-tetrazin-3-yl)-phenylalanine). The cell transfected with blank vector was set as control. The proteome incorporated with TetBu was labelled with TCO-biotin and visualized by dot blotting.

FIG. 4 show optimization of the TCG recoding system. The strategies to increase the efficiency of TCG recoding. The keys to the efficient TCG recoding are to increase the ratio of aminoacylated Rec-tRNACGA and decrease the ratio of aminoacylated Ser-tRNACGA. S1 and S2 were steps to increase the abundance of aminoacylated Rec-tRNACGA, while S3 and S4 were steps to reduce the level of aminoacylated Ser-tRNACGA (left). The trajectory of steps showed increased recoding efficiency compared to progenitor design (S0) (right).

FIG. 5 shows the detailed result of recoding rate for step 1-4. The DNA sequence encoding MbPylT_{CGA}, PylTC15_{CGA}, PylTM15_{CGA}, PylTCM15_{CGA} comprise the sequence of SEQ ID NO: 1-4.

FIG. 6 shows sequence result of monoclonal cell lines selected from tRNA isodecoder knockout. Two alleles of targeting tRNAs were successfully edited and repaired by the non-homologous end joining (NHEJ) pathway with indel generation for the knockout. Heterogeneous indels were generated at two alleles for 1A8 (SEQ ID NO:154-155), 2A3 (SEQ ID NO:156-157) and 4C23 (SEQ ID NO:158) cell lines, and homogeneous indels for the 3A12 (SEQ ID NO:159-160) cell line.

FIG. 7 shows the increased recoding rate in the knockout strains and the reproductive rare of these strains.

FIG. 8 shows the yield of EGFP incorporated with TetBu by the optimized TCG recoding system. The yield by rare codon recoding system were comparable to EGFP-WT.

FIG. 9 shows the decreased background misincorporation when the reporter is co-expressed. a of FIG. 9: The procedure to evaluate background incorporation of ncAA in the proteome for TCG recoding system was shown in the left, the In-gel fluorescence analysis of ncAA background incorporation under the indicated conditions for TCG recoding was shown in the right. b of FIG. 9: The translatome quantification of the same amount of HEK293T cells co-transfected with the TCG recoding system with or without reporter. The figure was plotted with RPKM (Reads Per Kilobase Million) of endogenous gene, TetRS and reporter.

FIG. 10 shows membraneless RTS organelles decrease the background misincorporation in proteome and retain the high recoding rate in the reporter. a of FIG. 10: Schematic of membranelles membraneless RTS organelles. The fusion of assembler protein (Fus) and a mRNA binding domain (λN22) to orthogonal synthetase (TetRS) formed a membranelles membraneless organelle (RTS organelle) and recruited Rec-tRNA to specifically translate the mRNA tagged with boxB motif, which enabled mRNA-specific TCG recoding in mammalian cells. b of FIG. 10: The background incorporation of TetBu in proteome was assayed by in-gel fluorescent labeling of TCO-Cy5 with cell lysate at indicated conditions. Membranelless RTS organelles led to a notable decrease in background incorporation. c of FIG. 10: Mass spectrometry characterization of the TetBu recoding rate into EGFP with membranelles membraneless RTS organelles. TetBu recoding efficiencies were shown in bracket.

FIG. 11 shows the cell expressed with the recoding systems exhibit the similar viability and expression pattern. a of FIG. 11: Expression of the recoding system in cells showed no cytotoxicity compared to cells transfected with empty plasmid control. Cell viability was measured using the CellTiter-Glo Luminescent Cell Viability Assay (Promega). Error bars represent ±s.e.m. (n=4 biologically independent experiments). Statistical significance was quantified with one-way ANOVA. Lip2000 was the transfection agent, and RTS was the abbreviation for recoding translation system. b of FIG. 11: Serine depletion did not show effect on cell viability. The cell viability was measured by Trypan Blue Staining Assay. Error bars represent ±s.e.m. (n=3 biologically independent experiments). c of FIG. 11: The procedure of nascent proteome assay with azidohomoalanine (AHA) labeling. d of FIG. 11: In-gel fluorescence analysis of the nascent proteome under the indicated conditions (left). The loading control was showed with the Coomassie brilliant blue (CBB) staining (right). e of FIG. 11: The expression distribution under the indicated conditions was assayed with transcriptome sequencing. Statistical significance was quantified with t-test; ns, not significant.

FIG. 12 shows the detailed recoding rate and background incorporation of rare codons. The ratios of recoding rate to background incorporation for each rare codon were labeled on the top of the bars. Error bars represent ±s.d. (n=3 biologically independent experiments for recoding rates, n=4 biologically independent experiments for background analysis.)

FIG. 13 shows the recoding rate and protein yield of the recoding systems for different rare codons. a of FIG. 13: The optimization of TetBu recoding efficiency with the increased tRNA copies and the deprived culture mediums. The mediums deprived of serine, threonine and arginine were used for the corresponding TCG/TCA, ACG and CGA recoding systems, respectively. b of FIG. 13: The relative yields of EGFP incorporated with TetBu by the optimized rare codon recoding systems. Data was quantified from Western blotting in fig. S2B and normalized to the expression level of EGFP-WT.

FIG. 14 shows the representative chemical structure used in the patent.

FIG. 15 shows the recoding rate of different recoding system for various unnatural amino acids. a of FIG. 15: Mass spectrometry characterization of the incorporation of indicated ncAAs into EGFP in response to TCG codon at position 151 by PylRS. The expected MW values of EGFP incorporated with Kcr (ε-N-crotonyl-lysine), BocK (ε-N-tert-butyloxycarbonyllysine) and DiZSeK were 27874, 27908 and 28014 Da and the observed MW values were 27876, 27907 and 28012 Da respectively. The peaks of EGFP with ncAAs and serine were labelled. ncAAs recoding rates were shown in brackets. b of FIG. 15: Mass spectrometry characterization of the incorporation of indicated ncAAs into EGFP in response to TCG codon at position 151 by their corresponding aaRSs in mammalian cells. The expected MW values of EGFP incorporated with AcF (4-Acetyl-phenylalanine) and OmeY (O-methyl-tyrosine) were 27869 and 27857 Da and the observed MW values were 27867 and 27856 Da respectively. The peaks of EGFP with ncAAs and serine were labelled. ncAAs recoding rates were shown in brackets. c of FIG. 15: Mass spectrometry characterization of the incorporation of indicated ncAAs into EGFP in response to TCG codon at position 151 by their corresponding aaRSs in mammalian cells. PylRS_Ack (MbPylRSIPYE-L266M/L270I/Y271F/L274A/C313F), PylRS_Kpr (MbPylRSIPYE-Y271F/C313T), PylRS_Bu (MbPylRSIPYE-C313T) and EcTyrRS_sTyr (L71V/D182G) were used. The expected MW values of EGFP incorporated with AcK, Kpr, Kbu and sTyr were 27849, 27863, 27877 and 28922 Da and the observed MW values were 27850, 27863, 27877 and 27922 Da respectively. The peaks of EGFP with ncAAs and serine were labelled. The de-acylation peaks (star) were also detected in samples of Ack, Kpr and Kbu incorporation.

FIG. 16 shows the high recoding rate of TCG recoding system for multi-sites UAAs incorporation. a of FIG. 16: The TCG recoding system for multi-site UAAs incorporation. EGFP reporters with indicated TCG or TAG codons (up) was co-expressed with chPylRSIPYE (TetRS)/MmPylTCGA or chPylRSIPYE (TetRS)/MmPylTcuA in the presence of 200 μM TetBu. Full-length EGFP was detected in the cell lysate by western blotting using anti-His-tag antibody and with HSP90 as loading control (down). The relative yield of EGFP by TCG recoding system was shown in the figure with yield of EGFP by amber suppression set as 1 for data normalization. b of FIG. 16: Mass spectrometry characterization of three-site TetBu incorporation at positions 151/182/200 of EGFP by the TCG recoding system. The peaks of 3*TetBu and 2*TetBu incorporation were labelled and 3*TetBu recoding rate was shown in brackets. c of FIG. 16: Mass spectrometry characterization of three- or six-site ncAAs incorporation into EGFP in response TCG codons at position 151/182/200 or 95/164/151/172/182/200, by chPheRS in the presence of indicated ncAAs.

FIG. 17 shows high recoding rate in functional proteins. a of FIG. 17: The incorporation of TetBu into several interleukin proteins including IL-1B, IL-2, IL-4 and IL-6 by the TCG recoding system. IL-1B, IL-2, IL-4 and IL-6 bearing TCG codon at position 205, 62, 121 and 87 respectively were co-expressed with chPylRSIPYE (TetRS)/MmPylTCGA in presence of 200 μM TetBu. The TetBu-incorporated protein was labelled by TCO-PEG5000 and showed mobility shifted (arrow). The TetBu recoding rate was shown below the figure. The detailed procedure for recoding rate calculation was shown in FIG. 2. b of FIG. 17: Mass spectrometry characterization of TetBu-incorporated IL-1B after reaction with TCO reagents including TCO-amine, TCO-PEG4-Biotin and TCO-Cy5.

FIG. 18 shows the scalability of dual rare codon recoding systems. a of FIG. 18: MS characterization of pAzF and TetPr dual incorporation rate into the EGFP reporter with dual rare codon recoding system. The peak of dual ncAA incorporation was labeled in the figure. The dual recoding rate of pAzF and TetPr was shown in parenthesis. b of FIG. 18: MS characterization of pAzF and TetPr dual incorporation rate into EGFP reporters after flanking context optimization for rare codon. c of FIG. 18: Trajectory of step S1 to S2, showing increased dual recoding efficiency compared with progenitor design (S0). d of FIG. 18: Western blotting showed the yields of dual ncAA incorporation by dual stop codon suppression and dual rare codon recoding strategy. In the assay, GFP antibody (up) was used with β-actin as a loading control (down). e of FIG. 18: MS characterization of dual incorporations of indicated ncAAs into EGFP in response to 84TCG codon and 151ACG codon by corresponding ncAA_aaRS/tRNA pairs. The dual ncAA recoding rates were shown in parentheses.

FIG. 19 shows triple and quadruple distinct UAAs incorporation achieved by combination of rare codon recoding and stop codon suppression. a of FIG. 19: Schematic of incorporation of three distinct ncAAs into a single protein in mammalian cells. Triply orthogonal translation systems were co-expressed with the EGFP reporter bearing 39TGA/151TCG/182TAG in the presence of indicated ncAAs. b of FIG. 19: Mass spectrometry characterization of the incorporation of three distinct ncAAs with design in (a of FIG. 19). The expected MW values of EGFP-(39-5MTP (5-Methoxy-L-tryptophan)/151-TetBu/182-OmeY) in group-1 and EGFP-(39-5MTP/151-AcF/182-BocK) in group-2 were 27844 and 27800 Da and the observed MW values were 27841 and 27798 Da respectively. c of FIG. 19: The schematic design (up) and mass spectrometry characterization (down) of four distinct ncAAs incorporation into a single protein. Quadruply orthogonal translation systems were co-expressed with the EGFP reporter bearing 39TGA/151TAA/182TAG/200TCG in the presence indicated ncAAs.

FIG. 20 shows triple and quintuplet uaas incorporation with multi-type rare codon recoding. a of FIG. 20: Schematic of incorporation of three distinct ncAAs into a single protein in mammalian cells by multi-type rare codon recoding. Triply orthogonal translation systems were co-expressed with the EGFP reporter bearing 84TCA/151ACG/182TCG in the presence of indicated ncAAs. b of FIG. 20: MS characterization of the incorporation of three distinct ncAAs with the design in (A). The expected MW value of EGFP-(840meY/151TetPr/182pAAF (4-(2-azidoacetamido)-phenylalanine)) was 27874 Da; the observed MW value was 27873 Da. The recoding efficiency of triple ncAA incorporation was shown in parenthesis. c of FIG. 20: Western blotting (left) showed the protein expression levels of three distinct ncAA incorporation by triple rare codon recoding and triple stop codon suppression strategy with reporters of EGFP-84TCA/151ACG/182TCG and EGFP-84TAG/151TGA/182TAA, respectively. In the assay, GFP antibody (up) was used with HSP90 as a loading control (down). The relative yields (right) of EGFP-(840meY/151TetPr/182pAAF) in two strategies were quantified from Western blotting shown above. The yield of EGFP by triple stop codon suppression system was set as 1 for data normalization. Error bars represent ±s.d. (n=3 biologically independent experiments). d of FIG. 20: Schematic design of five distinct ncAAs incorporation into a single protein. Quintuply orthogonal translation systems were co-expressed with the EGFP reporter bearing 39TGA/84TCA/151ACG/182TCG/200TAG in the presence of indicated ncAAs. e of FIG. 20: MS characterization of the incorporation of five distinct ncAAs with the design in (d of FIG. 20). The expected MW value of EGFP-(39-5MTP/840meY/151TetPr/182pAAF/200BocK) was 27992 Da; the observed MW value was 27990 Da. The recoding efficiency of quintuple ncAAs incorporation was shown in parenthesis.

FIG. 21 shows the strategy of protein dual labelling with dual rare codon recoding systems. a of FIG. 21: Schematic of protein dual labelling after pAzF and TetPr dual incorporation with dual rare codon recoding systems. Alkyne-Cy3 and TCO-Cy5 were used to label protein dual recoding with pAzF and TetPr via CuAAC and SPIEDAC reaction, respectively b of FIG. 21: In-gel fluorescence analysis of dual labelling of two EGFP variants with dual incorporation of pAzF and TetPr into indicated codon sites. The EGFP expression levels were also shown (down) by Western blotting. C of FIG. 21: In-gel fluorescence analysis of dual labelling of various functional proteins with dual incorporation of pAzF and TetPr. The functional proteins were purified using flag beads and then efficiently dual labelled with Alkyne-Cy3 and (4E)-TCO-PEG3-Cy5. The protein expression levels were also shown (down) by Western blotting.

FIG. 22 shows protein manipulation with TCG recoding system. a of FIG. 22: The sites selection for TCG incorporation in EGFP for activity manipulation. b of FIG. 22: The activity of EGFP mutants of TCG substitute. c of FIG. 22: The EGFP activity is tuned with OmeY addition. d of FIG. 23: The response curve of EGFP-F84TCG with the concentration of OmeY. d of FIG. 22: The time lapse of function activation of EGFP-F84TCG after OmeY addition.

FIG. 23 shows functional manipulation of Kras with the TCG recoding strategy a of FIG. 23: The residues F82 and Y40 around ATP were chosen for TCG codon introduction. b of FIG. 23: The unnatural amino acids chosen for functional simulation of tyrosine and phenylalanine. c of FIG. 23: The functional manipulation of Kras by UAAs with TCG recoding system was measured by the formation of p-ERK2.

FIG. 24 shows protein functional manipulation with dual decaging strategy. a of FIG. 24: Schematic of functional manipulation of protein by dual rare codon recoding and decaging. Two distinct caged ncAAs can be genetically incorporated at two functional sites or proximal sites in the active pocket of protein for temporal activity blockage. The protein function was then rescued by dual decaging triggered by photo (UV) and click chemistry (small molecules or catalysts). b of FIG. 24: The activity of FLuc incorporated with dual caged ncAAs before and after decaging. Protein dual incorporated with ONBY at Y255 and TCOK at K529 was dual decaged. c of FIG. 24: Protein dual incorporated with ONBY at Y255 and PABK at K529 was dual decaged.

FIG. 25 shows plasmid map of pCMV-Nes-chPylRS (TetRS)-12* (U6-CM15_{CGA}).

FIG. 26 shows plasmid map of pEGFP-mCherry-T2A-EGFP151TCG.

FIG. 27 shows plasmid map of pCMV-EcTyrRS (OmeY)-8*(U6-BsTyrT_{CGA}).

FIG. 28 shows plasmid map of pCMV-G1PylRS (TetPr)-8*(U6-G1PylT_{CGT}).

FIG. 29 shows plasmid map of pEGFP-mCherry-T2A-EGFP-84TCG/151ACG.

FIG. 30 shows plasmid map of pEGFP-EGFP-39TGA/151TCG/182TAG-EF1α-EcTrpRS (5MTP)-4*(U6-EcTrpT_{UCA}).

FIG. 31 shows plasmid map of pCMV-Nes-chPylRSIPYE (TetBu)-4*(U6-CM15_{CGA})-EF1α-EcTyrRS (OmeY)-4*(U6-BsTyrT_{CUA}).

FIG. 32 shows plasmid map of pCMV-EcTyrRS (OmeY)-8*(U6-BsTyrT_{UUA}).

FIG. 33 shows plasmid map of pEGFP-EGFP-39TGA/151TAA/182TAG/200TCG-EF1α-EcTrpRS (5MTP)-4*(U6-EcTrpT_{UCA}).

FIG. 34 shows plasmid map of pCMV-Nes-chPylRSIPYE (TetRS)-4*(U6-CM15_{CGA})-EF1α-MaPylRS (BocK)-4*(U6-MaPylT_{CUA}-T6).

### DEFINITION

For the purposes of this specification and the appended claims, the following terms shall have the meanings set forth below unless otherwise indicated:

“Unnatural Amino Acid” refers to an amino acid and/or an amino acid derivative or analog that cannot be biosynthetically produced in any organism. Unnatural amino acids include, but are not limited to, amino acids beyond the 20 naturally occurring amino acids, selenocysteine, pyrrolysine, pyrroline-carboxy-lysine, and modified forms, derivatives, or analogs of the foregoing. In the context of this application, unnatural amino acids can be incorporated into a target protein, polypeptide, or amino acid residue with the assistance of an aminoacyl-tRNA synthetase and a recoding tRNA. The incorporation of an unnatural amino acid may impart specific functionalities to the target protein, polypeptide, or amino acid residue.

“Recoding Aminoacyl-tRNA Synthetase” refers to an enzyme capable of preferentially aminoacylating a recoding tRNA with an amino acid carried by the enzyme. Such preferential aminoacylation may occur within a translation system or in any reaction environment, including, but not limited to, a cell-free protein synthesis system. “Preferential aminoacylation” means that the aminoacyl-tRNA synthetase aminoacylates the recoding tRNA with greater efficiency compared to the aminoacylation of endogenous tRNAs naturally present in a host cell, for example, at an efficiency of at least 70%, 75%, 85%, 90%, 95%, 99%, or higher. The aminoacyl-tRNA synthetase may be an orthogonal aminoacyl-tRNA synthetase or an enzyme homologous to any orthogonal aminoacyl-tRNA synthetase. In some embodiments, the aminoacyl-tRNA synthetase described herein is a variant of any orthogonal aminoacyl-tRNA synthetase. The amino acid loaded onto the recoding tRNA by the aminoacyl-tRNA synthetase may be any amino acid, including, but not limited to, natural amino acids and/or unnatural amino acids, whether modified or unmodified. The aminoacyl-tRNA synthetase described herein can incorporate an unnatural amino acid into a target protein, polypeptide, or amino acid residue with high fidelity, such as at an efficiency greater than 75%, greater than about 80%, greater than about 90%, greater than about 95%, or greater than about 99% with respect to a designated codon.

“Orthogonal” refers to a molecule (e.g., a recoding tRNA and/or aminoacyl-tRNA synthetase) that functions with lower efficiency, or is incapable of functioning, using molecules expressed by the host cell itself, as compared to corresponding endogenous molecules of the cell or translation system. Molecules expressed by the host cell itself are those expressed without any genetic manipulation. In the context of tRNAs and aminoacyl-tRNA synthetases, “orthogonal” may mean that, compared to an endogenous tRNA functioning with an endogenous aminoacyl-tRNA synthetase, a recoding tRNA either cannot function with an endogenous aminoacyl-tRNA synthetase or functions at a reduced efficiency (e.g., less than 20%, less than 10%, less than 5%, or less than 1% efficiency); alternatively, compared to an endogenous aminoacyl-tRNA synthetase functioning with an endogenous tRNA, an orthogonal aminoacyl-tRNA synthetase either cannot function with an endogenous tRNA or functions at a reduced efficiency (e.g., less than 20%, less than 10%, less than 5%, or less than 1% efficiency). Typically, orthogonal molecules lack functional endogenous complementary molecules within the cell. For example, when compared to the aminoacylation of an endogenous tRNA by an endogenous aminoacyl-tRNA synthetase, any endogenous aminoacyl-tRNA synthetase in the cell aminoacylates a recoding tRNA with lower efficiency or no efficiency.

In another embodiment, when compared to the aminoacylation of an endogenous tRNA by an endogenous aminoacyl-tRNA synthetase, a recoding aminoacyl-tRNA synthetase aminoacylates any endogenous tRNA in the host cell with reduced efficiency or no efficiency. First and second orthogonal components may be sequentially introduced into a host cell to exert their functions, such as introducing a recoding tRNA and a recoding aminoacyl-tRNA synthetase into the host cell in sequence.

“Recoding tRNA” refers to a tRNA capable of recognizing and binding both an aminoacyl-tRNA synthetase and a rare codon on a transcription template, thereby incorporating the amino acid carried by the aminoacyl-tRNA synthetase into a protein, polypeptide, or amino acid residue. The recoding tRNA may be preferentially aminoacylated by an aminoacyl-tRNA synthetase carrying an amino acid. The recoding tRNA may be homologous to a recoding tRNA from any species, where “homologous” refers to molecules with different sequences but similar functions, exhibiting sequence homology of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. The recoding tRNA described herein may be a variant of a recoding tRNA from any species. The recoding tRNA may be loaded with an amino acid or may exist in an unloaded (apical) state. The amino acid loaded onto the recoding tRNA may be any amino acid, including, but not limited to, natural amino acids and/or unnatural amino acids. The anticodon loop of the recoding tRNA described herein can recognize and hybridize to a specific codon on a transcription template during translation, thereby incorporating the loaded amino acid into a target protein, polypeptide, or amino acid residue. “Hybridize” refers to the process by which the anticodon loop of a tRNA, following base-pairing complementarity principles, recognizes and binds to a corresponding codon on the transcription template. Through hybridization, the tRNA can incorporate the loaded amino acid into a target protein, polypeptide, or amino acid residue. The codons recognized and hybridized by the anticodon of the recoding tRNA may include nonsense codons (e.g., amber, ochre, or opal stop codons), codons with four or more bases, rare codons, or codons derived from natural or unnatural bases.

“Transcription Template” refers to a molecule synthesized by DNA transcription. The transcription template may include a primary transcription template of pre-messenger RNA (pre-mRNA) prior to processing and/or a mature mRNA molecule post-processing. The mature mRNA molecule is a functional form derived from the modification of pre-mRNA transcribed from DNA, capable of producing various proteins and bioactive components. In some embodiments, the transcription template may include exons, introns, 5′ untranslated regions (5′ UTR), 3′ untranslated regions (3′ UTR), and any regulatory elements. In some embodiments, the transcription template may also include modified transcription templates, such as those encoding spliced fusion proteins or exogenous nucleic acid sequences.

“Wild-Type Host Cell” generally refers to a cell capable of being transformed by a nucleic acid molecule but which has not yet been transformed by any nucleic acid molecule. When the nucleic acid molecule contains a gene of interest, the transformed wild-type host cell can express the transformed gene of interest. The term also encompasses progeny of the wild-type host cell, regardless of whether the progeny are morphologically or genetically identical to the original wild-type host cell, provided the progeny remain untransformed and fall within the scope of wild-type host cells as described herein. Wild-type host cells may include those existing in isolated form, such as in a culture. A wild-type host cell may possess a wild-type translation system, i.e., an untransformed translation system naturally expressed by the wild-type host cell itself. The wild-type host cell's translation system includes components such as endogenous tRNAs, endogenous aminoacyl-tRNA synthetases, amino acids, and/or transcription templates.

“Endogenous tRNA” refers to a tRNA that is endogenous to a host cell and incorporates an amino acid into a protein, polypeptide, or amino acid residue by binding to a codon on a transcription template. In some embodiments, endogenous tRNAs may read through sense codons, such as the 61 common codons, four-base codons, rare codons, and/or codons derived from natural or unnatural base pairs.

“Rare Codon” refers to a codon that can be recognized by an endogenous tRNA of a host cell or by an exogenous recoding tRNA expressed by a non-host cell. An endogenous tRNA of the host cell is one expressed without any genetic modification, while an exogenous recoding tRNA expressed by a non-host cell requires genetic modification of the host cell for expression. The anticodon loops of endogenous tRNAs and exogenous recoding tRNAs may compete to recognize and bind the rare codon. Upon binding the rare codon, an endogenous tRNA or exogenous recoding tRNA can insert an amino acid, such as an unnatural amino acid, at the site on the polypeptide chain encoded by the transcription template corresponding to the rare codon. Rare codons may be codons with low corresponding endogenous tRNA abundance in the host cell, where the abundance of endogenous tRNAs encoding the rare codon may be less than 2%, 1.75%, 1.5%, 1.25%, 1%, 0.5%, or 0.25%.

The rare codons described herein may also be codons with low usage frequency in the host cell, such as a usage frequency of less than 20/1000, 17.5/1000, 15/1000, 12.5/1000, 10/1000, 7.5/1000, or 5/1000 codons.

“Abundance” refers to the proportion of endogenous tRNAs encoding codons on a transcription template within the total tRNA population of a host cell. Typically, the abundance of endogenous tRNAs may fluctuate with the host cell's growth cycle and environmental conditions, but the relative abundance of various endogenous tRNAs within a specific species remains relatively stable.

“Frequency” refers to the usage frequency of a selected codon among all coding codons in a species' genome, a cell's genome, or a gene's corresponding codons. Generally, codon usage frequency correlates positively with endogenous tRNA abundance, meaning codons with low endogenous tRNA abundance also exhibit low usage frequency.

“Translation,” “Production,” or “Expression” are used interchangeably herein and refer to the biosynthetic process of proteins, polypeptides, and/or amino acid residues in a host cell. Translation may occur within a translation system or in any reaction environment, such as a cell-free protein synthesis system. Translation involves the assembly of an amino acid sequence controlled by a transcription template to produce a protein, polypeptide, and/or amino acid residue.

“Translation System” refers to a collection of components capable of incorporating amino acids into a growing protein, polypeptide, and/or amino acid residue. Components of the translation system may include, but are not limited to, ribosomes, tRNAs, aminoacyl-tRNA synthetases, transcription templates, and/or amino acids. In the context of this invention, components of the translation system (e.g., recoding tRNAs, aminoacyl-tRNA synthetases, and/or amino acids) may be introduced into the translation system of a wild-type host cell, such as a eukaryotic cell, including yeast cells, mammalian cells, reptilian cells, avian cells, plant cells, algal cells, fungal cells, and/or insect cells.

“Eubacteria” refers to prokaryotic organisms distinct from archaea. Similarly, “Archaea” refers to prokaryotic organisms distinct from eubacteria. In some embodiments, eubacteria and archaea may be distinguished by morphological and biochemical methods, such as differences in ribosomal RNA sequences, RNA polymerase structure, presence or absence of introns, sensitivity to antibiotics, presence or absence of cell wall peptidoglycan or other cell wall components, branched or unbranched membrane lipids, and presence or absence of histones or histone-like proteins.

Examples of eubacteria described herein include, but are not limited to, Escherichia coli, Thermus thermophilus, Bacillus subtilis, and Bacillus stearothermophilus, or combinations thereof. Examples of archaea described herein include, but are not limited to, Methanococcus jannaschii (Mi), Methanosarcina mazei (Mm), Methanosarcina barkeri (Mb), Methanomethylophilus alvus (Ma), Methanogenic archaeon ISO4-G1 (G1), Methanomassiliicoccus luminyensis (Lum1), Candidatus Methanomethylophilus sp. 1R26 (1R26), Candidatus Methanomassiliicoccus intestinalis (Int), Nitrososphaeria archaeon (Nitra), Deltaproteobacteria bacterium (Deb), Methanobacterium thermoautotrophicum (Mt), Methanococcus maripaludis, Methanopyrus kandleri, Halobacterium, Archaeoglobus fulgidus (Af), Pyrococcus furiosus (Pf), Pyrococcus horikoshii (Ph), Pyrobaculum aerophilum, Pyrococcus abyssi, Sulfolobus solfataricus (Ss), Sulfolobus tokodaii, Aeropyrum pernix (Ap), Thermoplasma acidophilum, and Thermoplasma volcanium, or combinations thereof.

“Vector” generally refers to a nucleic acid delivery vehicle into which any nucleic acid molecule may be inserted. Typically, the inserted nucleic acid molecule may carry a gene of interest, and upon insertion into the vector, the gene of interest can be stably expressed in vivo or in vitro. Vectors may be introduced into a host cell via transformation, transduction, or transfection, enabling expression of the carried gene of interest within the host cell. Examples of vectors include, but are not limited to, plasmids, phagemids, cosmids, artificial chromosomes such as yeast artificial chromosomes (YACs), bacterial artificial chromosomes (BACs), or P1-derived artificial chromosomes (PACs), and phages such as lambda phage, M13 phage, or animal viruses. Animal viruses used as vectors include, but are not limited to, retroviruses (including lentiviruses), adenoviruses, adeno-associated viruses, herpesviruses (e.g., herpes simplex virus), poxviruses, baculoviruses, papillomaviruses, and/or polyomaviruses (e.g., SV40). Typically, the vector may contain one or more expression control elements, including promoter sequences, transcription initiation sequences, enhancer sequences, selection sequences, and reporter genes. Additionally, the vector may include an origin of replication sequence. The vector may also comprise components facilitating its entry into cells, including, but not limited to, viral particles, liposomes, and/or protein shells. Furthermore, the vector may include specific site recombination sequences and/or restriction endonuclease cleavage sites.

“Variant” refers to a molecule that is functionally similar to a pre-modified molecule but has an altered sequence. The pre-modified molecule and/or variant may be a component of a translation system, such as a variant recoding tRNA or a variant aminoacyl-tRNA synthetase. A variant aminoacyl-tRNA synthetase or aminoacyl-tRNA synthetase can aminoacylate a corresponding recoding tRNA and/or variant recoding tRNA with an amino acid, though the recoding tRNA, variant recoding tRNA, variant aminoacyl-tRNA synthetase, and aminoacyl-tRNA synthetase do not share identical sequences. The sequence of a variant may include, but is not limited to, one, two, three, four, or five or more sequence site mutations, provided the variant retains the function of the pre-modified molecule, such as being capable of aminoacylation by the corresponding aminoacyl-tRNA synthetase of a recoding tRNA.

### INCORPORATION BY REFERENCE

The following references are hereby incorporated by reference in their entirety:
Dunkelmann, D. L., Willis, J. C. W., Beattie, A. T., and Chin, J. W. (2020). Engineered triply orthogonal pyrrolysyl-tRNA synthetase/tRNA pairs enable the genetic encoding of three distinct non-canonical amino acids. Nat Chem 12, 535-544.
Beattie, A. T., Dunkelmann, D. L., and Chin, J. W. (2023). Quintuply orthogonal pyrrolysyl-tRNA synthetase/tRNA(Pyl) pairs. Nat Chem 15, 948-959 Suzuki, T., Miller, C., Guo, L. T., Ho, J. M. L., Bryson, D. I., Wang, Y. S., Liu, D. R., and Soll, D. (2017).
Crystal structures reveal an elusive functional domain of pyrrolysyl-tRNA synthetase. Nat Chem Biol 13, 1261-1266.
Chinese Patent Publication No. CN110172467B.

### DETAILED DESCRIPTION

The present application overcomes the technical bias widely held in the art that rare codons cannot be used for recoding in eukaryotic organisms. It provides a highly efficient method for producing proteins containing unnatural amino acids in eukaryotic cells using rare codons, thereby solving the problem of low efficiency in producing such proteins in eukaryotic cells.

Those skilled in the art generally believe that while recoding schemes using rare codons can achieve good results in prokaryotes, they cannot yield significant recoding outcomes in eukaryotes. Due to the significant differences in genetic structure and biological functions between prokaryotes and eukaryotes, the key distinctions affecting the use of rare codons for recoding unnatural amino acids in eukaryotic cells include: 1) Eukaryotes inherently lack the most critical translation components, such as recoding aminoacyl-tRNA synthetases and tRNAs, which must be exogenously introduced for production. 2) The usage frequency of rare codons in eukaryotes is higher than in prokaryotes, leading to a large number of mistranslations and background incorporations. 3) There are differences in post-translational processes between eukaryotes and prokaryotes, making it unpredictable whether proteins containing unnatural amino acids produced using prokaryotic production schemes in eukaryotes will retain normal biological activity. Due to these critical differences affecting recoding efficiency, the outcome of using rare codons for recoding in eukaryotes cannot be predicted, and there has been no corresponding technological breakthrough in using rare codons for recoding unnatural amino acids in eukaryotic cells.

On one hand, the present application provides a method for producing proteins containing unnatural amino acids in eukaryotic organisms, which may include culturing a host cell with:
a nucleotide sequence encoding a first recoding tRNA or the nucleotide sequence of the first recoding tRNA, wherein the first recoding tRNA comprises an anticodon complementary to a first codon, and the first codon is a first rare codon;
a nucleotide sequence encoding a first aminoacyl-tRNA synthetase or the first aminoacyl-tRNA synthetase, wherein the first aminoacyl-tRNA synthetase is capable of charging the first recoding tRNA with a first unnatural amino acid.

In some embodiments, the host cell is a eukaryotic cell.

In some embodiments, the method further includes co-culturing the host cell with the first unnatural amino acid.

In some embodiments, the method for producing proteins containing unnatural amino acids in eukaryotic organisms may further include culturing the eukaryotic cell with:
a nucleotide sequence encoding a second recoding tRNA or the nucleotide sequence of the second recoding tRNA, wherein the second recoding tRNA comprises an anticodon complementary to a second codon;
a nucleic acid encoding a nucleotide sequence of a second aminoacyl-tRNA synthetase or the second aminoacyl-tRNA synthetase, wherein the second aminoacyl-tRNA synthetase is capable of charging the second tRNA with a second unnatural amino acid.

In some embodiments, the method further includes co-culturing the host cell with the second unnatural amino acid.

On the other hand, the present application provides a translation system for efficiently expressing unnatural amino acids in eukaryotic organisms. The system includes:
a host cell, wherein the host cell is a eukaryotic cell;
a nucleotide sequence encoding a first recoding tRNA or the nucleotide sequence of the first recoding tRNA, wherein the first recoding tRNA comprises an anticodon complementary to a first codon, and the first codon is a first rare codon;
a nucleotide sequence encoding a first aminoacyl-tRNA synthetase or the first aminoacyl-tRNA synthetase, wherein the first aminoacyl-tRNA synthetase is capable of charging the first recoding tRNA with a first unnatural amino acid.

In some embodiments, the system further includes the first unnatural amino acid.

In some embodiments, the system further includes:
a nucleotide sequence encoding a second recoding tRNA or the nucleotide sequence of the second recoding tRNA, wherein the second recoding tRNA comprises an anticodon complementary to a second codon;
a nucleic acid encoding a nucleotide sequence of a second aminoacyl-tRNA synthetase or the second aminoacyl-tRNA synthetase, wherein the second aminoacyl-tRNA synthetase is capable of charging the second tRNA with a second unnatural amino acid.

In some embodiments, the system further includes the second unnatural amino acid.

The following are specific embodiments of the components or steps involved in the above method or system.

### Host Cell Expression of Proteins/Polypeptides Containing Unnatural Amino Acids

The host cell can express one or more proteins and/or polypeptides. In some embodiments, the host cell can express a protein and/or polypeptide containing an unnatural amino acid.

The host cell may contain or express a transcription template, such as mRNA. The protein and/or polypeptide containing the unnatural amino acid is expressed based on the transcription template of the host cell.

The transcription template can be produced by the expression of endogenous genes in the host cell. The transcription template can also be produced by the expression of exogenous genes; for example, the transcription template can be introduced into the host cell via a vector, allowing the transcription template to be expressed within the host cell.

The transcription template may have multiple rare codons. For example, the transcription template may have one or more first rare codons. The second codon may be a second rare codon. In some embodiments, the transcription template may further have one or more second rare codons, where the first rare codon may be different from the second rare codon. In this application, the host cell uses the rare codons on the transcription template to introduce unnatural amino acids at the positions of the rare codons, thereby expressing proteins and/or polypeptides containing unnatural amino acids.

For example, the transcription template may have a first rare codon and a second rare codon, and the host cell uses the first rare codon and the second rare codon to express proteins and/or polypeptides containing both the first unnatural amino acid and the second unnatural amino acid.

Alternatively, the transcription template may have multiple first rare codons and/or second rare codons, and the host cell can use multiple first rare codons and/or multiple second rare codons to express proteins and/or polypeptides containing multiple first unnatural amino acids and/or second unnatural amino acids.

In addition to rare codons, the transcription template may have amber codons, ochre codons, or opal codons, or codons with four or more bases.

For example, the second codon may be an amber codon. The transcription template may have a first rare codon and an amber codon. The host cell uses the rare codon and the amber codon on the transcription template to introduce one or more unnatural amino acids at the positions of the rare codon and the amber codon, thereby expressing proteins and/or polypeptides containing unnatural amino acids.

In this application, the host cell introduces unnatural amino acids at the positions of rare codons and/or amber codons on the transcription template based on a system composed of recoding tRNAs and aminoacyl-tRNA synthetases.

In this application, the host cell can be a eukaryotic cell, such as a yeast cell, mammalian cell, reptilian cell, avian cell, plant cell, algal cell, fungal cell, and/or insect cell. The present application enables efficient recoding in mammalian cells to produce therapeutic proteins with unnatural amino acids, which is a promising strategy for synthesizing functionalized therapeutic proteins for drug development.

### Aminoacyl-tRNA Synthetases

In this application, the aminoacyl-tRNA synthetase can be derived from prokaryotes or eukaryotes. For example, the aminoacyl-tRNA synthetase is derived from prokaryotes; further, the aminoacyl-tRNA synthetase can be derived from eubacteria or archaea. For types of eubacteria and archaea, see the “Definitions” section.

In some embodiments, the aminoacyl-tRNA synthetase can be a pyrrolysyl-tRNA synthetase (PylRS). Pyrrolysyl-tRNA synthetase can form aminoacyl-tRNA with unnatural amino acids and pyrrolysyl-tRNA (PylT).

Specifically, the pyrrolysyl-tRNA synthetase can be one or more of the following enzymes: pyrrolysyl-tRNA synthetase from Methanosarcina mazei (MmPylRS), pyrrolysyl-tRNA synthetase from Methanosarcina barkeri (MbPylRS), pyrrolysyl-tRNA synthetase from Methanomethylophilus alvus (MaPylRS), pyrrolysyl-tRNA synthetase from Methanogenic archaeon ISO4-G1 (G1PylRS), pyrrolysyl-tRNA synthetase from Methanomassiliicoccus luminyensis (Lum1PylRS), pyrrolysyl-tRNA synthetase from Candidatus Methanomethylophilus sp. 1R26 (1R26PylRS), pyrrolysyl-tRNA synthetase from Candidatus Methanomassiliicoccus intestinalis (IntPylRS), pyrrolysyl-tRNA synthetase from Nitrososphaeria archaeon (NitraPylRS), pyrrolysyl-tRNA synthetase from Deltaproteobacteria bacterium (DebPylRS), and mutants, functional fragments, fusion enzymes, or chimeric enzymes of the above enzymes.

In some embodiments, the aminoacyl-tRNA synthetase can be a tyrosyl-tRNA synthetase (TyrRS). Tyrosyl-tRNA synthetase can form aminoacyl-tRNA with unnatural amino acids and tyrosyl-tRNA (TyrT).

Specifically, the tyrosyl-tRNA synthetase can be tyrosyl-tRNA synthetase from Escherichia coli (EcTyrRS) and its mutants, functional fragments, fusion enzymes, or chimeric enzymes.

In some embodiments, the aminoacyl-tRNA synthetase can be a leucyl-tRNA synthetase (LeuRS). Leucyl-tRNA synthetase can form aminoacyl-tRNA with unnatural amino acids and leucyl-tRNA (LeuT).

Specifically, the leucyl-tRNA synthetase can be leucyl-tRNA synthetase from Escherichia coli (EcLeuRS) and its mutants, functional fragments, fusion enzymes, or chimeric enzymes.

In some embodiments, the aminoacyl-tRNA synthetase can be a chimeric enzyme, such as chHmPheRS (referred to as chPheRS in this application) as described in patent CN110172467B, or chPylRS as described in the literature “Crystal structures reveal an elusive functional domain of pyrrolysyl-tRNA synthetase.” Nature Chemical Biology, 2017. DOI:10.1038/nchembio.2497. These two references are incorporated herein by reference. Specifically, chPylRS can be a chimera of positions 1-149 of MbPylRS and positions 185-454 of MmPylRS.

In some embodiments, the aminoacyl-tRNA synthetase may include one or more mutations that enhance efficiency. For example, the aminoacyl-tRNA synthetase may include the IPYE mutation (V311/T56P/H62Y/A100E), which improves the enzyme's efficiency. For instance, chPylRS, MmPylRS, or MbPylRS may further include the IPYE mutation (V31I/T56P/H62Y/A100E) compared to their wild-type forms.

In some embodiments, to accommodate different unnatural amino acids, the aminoacyl-tRNA synthetase may have one or more mutations related to binding unnatural amino acids. These mutations enable the aminoacyl-tRNA synthetase to efficiently bind specific unnatural amino acids. For example, for MmPylRS, introducing L305G/M346G/C348A mutations allows it to efficiently bind tetrazine unnatural amino acids (e.g., (S)-2-amino-3-(3-(6-butyl-1,2,4,5-tetrazin-3-yl)phenyl)propanoic acid, hereinafter referred to as TetBu). Similarly, for G1PylRS, introducing L124G/N165G/V167A/M128A mutations enables efficient binding to TetBu.

Specific details are shown in Table 1 below, where “wt” represents the wild-type aminoacyl-tRNA synthetase, “mutation sites” indicate the positions and types of mutations related to specific unnatural amino acid binding on the wild-type aminoacyl-tRNA synthetase, and “unnatural amino acid” represents the unnatural amino acid corresponding to the mutated aminoacyl-tRNA synthetase. In the unnatural amino acid encoding efficiency, “+” indicates a recoding efficiency greater than 40%, “++” greater than 60%, and “+++” greater than 80%.

TABLE 1
Recoding efficiency of all amino acids

| | | | | Recoding |
|---|---|---|---|---|
| No | aaRS | mutation | Unnatural amino acid | rate (%) |
| 1 | G1PylRS | M128A/N165Q/V167S | N6-((2-Azidoacetyl)glycyl)-L-lysine | 64 |
| 2 | G1PylRS | H120M/Y125L/M128A/V167F | N6-(2-Hydroxypropanoyl)-L-lysine | 82 |
| 3 | G1PylRS | V167T/Y204F | N6-(3-Hydroxybutanoyl)-L-lysine | 70 |
| 4 | G1PylRS | Y204W | N6-Benzoyl-L-lysine | 87 |
| 5 | G1PylRS | V167T/Y204F | N6-Isobutanoyl-L-lysine | 81 |
| 6 | G1PylRS | WT | N6-(3-Methylbutanoyl)-L-lysine | 82 |
| 7 | G1PylRS | Y204F | N6-(tert-Butoxycarbonyl)-N6-methyl-L-lysine | 83 |
| 8 | MbPylRS | L266M/L270I/L274A/C313F. | N6-(2,2,2-Trifluoroacetyl)-L-lysine | +++ |
| 9 | G1PylRS | Y204F | N6-(L-Methionyl)-L-lysine | 56 |
| 10 | G1PylRS | Y204F | N6-(L-Threonyl)-L-lysine | 78 |
| 11 | G1PylRS | Y204F | N6-((R)-2-Aminopent-4-ynoyl)-L-lysine | 53 |
| 12 | MbPylRS | L266M/L270I/Y271L/L274A | N6-Ethylsulfanyl-L-lysine | +++ |
| 13 | MbPylRS | Y271A/Y349F | N6-(5-(1,2-Dithiolan-3-yl)pentanoyl)-L-lysine | +++ |
| 14 | G1PylRS | V167T/Y204F | N6-(2-Hydroxy-2-methylpropanoyl)-L-lysine | 83 |
| 15 | MbPylRS | C313V | N6-(L-Cysteinyl)-L-lysine | ++ |
| 16 | MbPylRS | L266M/L270I/Y271F/L274A/ | N6-Acetyl-L-lysine | 78 |
| | | C313F | | |
| 17 | MbPylRS | Y271F/C313T | N6-Propanoyl-L-lysine | 74 |
| 18 | MbPylRS | C313T | N6-Butanoyl-L-lysine | 79 |
| 19 | MbPylRS | L266M/L270I/Y271F/L274A/ | N6-Formyl-L-lysine | ++ |
| | | C313F | | |
| 20 | MbPylRS | L274A/C313F/Y349W | ε-N-Crotonyl-L-lysine | 84 |
| 21 | MmPylRS | Y384F | N6-(2-Fluorobenzoyl)-L-lysine | ++ |
| 22 | MmPylRS | Y384F | N6-(2,5-Difluorobenzoyl)-L-lysine | +++ |
| 23 | MbPylRS | D76G/L266M/L270I/Y271F/ | (S)-2-(3-(3-Acetamidopropyl)-3H-diazirine-3- | +++ |
| | | L274A/C313F | yl)-2-aminoacetic acid | |
| 24 | MbPylRS | D76G/L266M/L270I/Y271F/ | (S)-2-Amino-2-(3-(3-propanamidopropyl)-3H- | +++ |
| | | L274A/C313F | diazirine-3-yl)acetic acid | |
| 25 | MbPylRS | C313T | (S)-2-Amino-2-(3-(3-butanamidopropyl)-3H- | +++ |
| | | | diazirine-3-yl)acetic acid | |
| 26 | MbPylRS | L274A/C313T/Y349W | (S,E)-2-Amino-2-(3-(3-(but-2- | +++ |
| | | | enamido)propyl)-3H-diazirine-3-yl)acetic acid | |
| 27 | MbPylRS | C313T | (S)-2-Amino-2-(3-(3-(2-hydroxy-2- | +++ |
| | | | methylpropanamido)propyl)-3H-diazirine-3- | |
| | | | yl)acetic acid | |
| 28 | MbPylRS | D76S/L274A/C313F/Y349F | (S)-2-Amino-2-(3-(3-((R)-3- | +++ |
| | | | hydroxybutanamido)propyl)-3H-diazirine-3- | |
| | | | yl)acetic acid | |
| 29 | MbPylRS | L274A/C313S/Y349F | (S)-2-Amino-2-(3-(3-((((4- | +++ |
| | | | azidobenzyl)oxy)carbonyl)amino)propyl)-3H- | |
| | | | diazirine-3-yl)acetic acid | |
| 30 | MmPylRS | L309A/C348A | (S)-2-Amino-2-(3-(3-((((4- | +++ |
| | | | nitrobenzyl)oxy)carbonyl)amino)propyl)-3H- | |
| | | | diazirine-3-yl)acetic acid | |
| 31 | MmPylRS | Y306A/Y384F | (S)-2-Amino-6-(3-methyl-1H-1,2,4-triazol-5- | ++ |
| | | | yl)hexanoic acid | |
| 32 | MbPylRS | L274A/C313S/Y349F | N6-((3-(3-Methyl-3H-diazirine-3- | +++ |
| | | | yl)propoxy)carbonyl)-L-lysine | |
| 33 | MmPylRS | Y306V/L309A/C348F/Y384F | N6-(2-(1-Methyl-1H-pyrrol-2-yl)-2H-tetrazole- | |
| | | | 5-carbonyl)-L-lysine | |
| 34 | MmPylRS | Y306V/L309A/C348F/Y384F | N6-(2-(Furan-2-yl)-2H-tetrazole-5-carbonyl)-L- | ++ |
| | | | lysine | |
| 35 | MmPylRS | Y306V/L309A/C348F/Y384F | N6-(2-Phenyl-2H-tetrazole-5-carbonyl)-L- | ++ |
| | | | lysine | |
| 36 | MmPylRS | Y306V/L309A/C348F/Y384F | O-(2-(2-(1-Methyl-1H-pyrrol-2-yl)-2H- | ++ |
| | | | tetrazole-5-carboxamido)ethyl)-L-serine | |
| 37 | MmPylRS | Y306V/L309A/C348F/Y384F | S-(2-(2-(1-Methyl-1H-pyrrol-2-yl)-2H- | ++ |
| | | | tetrazole-5-carboxamido)ethyl)-L-cysteine | |
| 38 | MmPylRS | Y306V/L309A/C348F/Y384F | (R)-2-Amino-3-((2-(2-(1-methyl-1H-pyrrol-2- | ++ |
| | | | yl)-2H-tetrazole-5- | |
| | | | carboxamido)ethyl)selanyl)propanoic acid | |
| 39 | MmPylRS | Y306A/Y384F | N6-(((3-(3-(Trifluoromethyl)-3H-diazirine-3- | +++ |
| | | | yl)benzyl)oxy)carbonyl)-L-lysine | |
| 40 | MmPylRS | Y306G/Y384F | N6-(((4-(3-(Trifluoromethyl)-3H-diazirine-3- | +++ |
| | | | yl)benzyl)oxy)carbonyl)-L-lysine | |
| 41 | G1PylRS | M128A/V167S/Y204F | N6-((2-(3-Methyl-3H-diazirine-3- | 85 |
| | | | yl)ethoxy)carbonyl)-L-lysine | |
| 42 | MbPylRS | L274A/C313S/Y349F | (S)-2-Amino-4-((3-((3-(3-methyl-3H-diazirine- | +++ |
| | | | 3-yl)propyl)amino)-3- | |
| | | | oxopropyl)selanyl)butanoic acid | |
| 43 | MbPylRS | L274A/C313S/Y349F | Nε-3-((3-Methyl-3H-diazirine-3- | 91 |
| | | | yl))propanecarbonyl-y-selanyl-L-lysine | |
| 44 | MmPylRS | Y306A/Y384F | N6-(((3-((Prop-2-yn-1-yloxy)methyl)-3H- | +++ |
| | | | diazirine-3-yl)methoxy)carbonyl)-L-lysine | |
| 45 | PylRS | Y306A/Y384F | N6-((2-(Furan-2-yl)ethoxy)carbonyl)-L-lysine | +++ |
| 46 | MmPylRS | Y306A/Y384F | N6-((((E)-Cyclooct-2-en-1-yl)oxy)carbonyl)-L- | |
| | | | lysine | |
| 47 | MmPylRS | Y384F | N6-(((2-Methylbut-3-yn-2-yl)oxy)carbonyl)-L- | +++ |
| | | | lysine | |
| 48 | MbPylRS | Y271M/L274A/C313A | N6-(2-Phenylacetyl)-L-lysine | +++ |
| 49 | MbPylRS | Y271A/L274M | N6-(((7-Hydroxy-2-oxo-2H-chromen-4- | ++ |
| | | | yl)methoxy)carbonyl)-L-lysine | |
| 50 | MbPylRS | Y271A/L274M | N6-(((6-Bromo-7-hydroxy-2-oxo-2H-chromen- | ++ |
| | | | 4-yl)methoxy)carbonyl)-L-lysine | |
| 51 | MbPylRS | Y271A/L274M | N6-(((7-Amino-2-oxo-2H-chromen-4- | ++ |
| | | | yl)methoxy)carbonyl)-L-lysine | |
| 52 | G1PylRS | Y125M/M128A/V167A/Y204F | N6-(((2-Nitrobenzyl)oxy)carbonyl)-L-lysine | 55 |
| 53 | MbPylRS | M241F/A267S/Y271C/L274M | N6-((1-(6-Nitrobenzo[d][1,3]dioxol-5- | 40 |
| | | | yl)ethoxy)carbonyl)-L-lysine | |
| 54 | MbPylRS | Y271I/L274M/C313A | N6-Methyl-N6-(((2-nitrobenzyl)oxy)carbonyl)- | +++ |
| | | | L-lysine | |
| 55 | MmPylRS | Y306V/L309A/C348F/Y384F | N6-(4-Phenyl-2H-1,2,3-triazole-2-carbonyl)-L- | +++ |
| | | | lysine | |
| 56 | MmPylRS | Y306V/L309A/C348F/Y384F | N6-(4-(4-Fluorophenyl)-2H-1,2,3-triazole-2- | +++ |
| | | | carbonyl)-L-lysine | |
| 57 | MmPylRS | Y306V/L309A/C348F/Y384F | N6-(4-(Thiophen-2-yl)-2H-1,2,3-triazole-2- | +++ |
| | | | carbonyl)-L-lysine | |
| 58 | MmPylRS | Y306V/L309A/C348F/Y384F | N6-(4-(1-Methyl-1H-pyrrol-2-yl)-2H-1,2,3- | +++ |
| | | | triazole-2-carbonyl)-L-lysine | |
| 59 | G1PylRS | Y125G/M128A/V167F/H225T/ | N6-(4-((Fluorosulfonyl)oxy)benzoyl)-L-lysine | 81 |
| | | K226P/L227I | | |
| 60 | MmPylRS | L301M/L305I/Y306L/L309A/ | N6-(2-Fluoroacetyl)-L-lysine | ++ |
| | | C348F | | |
| 61 | MmPylRS | Y384F | N6-(6-Bromohexanoyl)-L-lysine | ++ |
| 62 | MbPylRS | Y271M/L274A/C313A | N6-(7-Bromoheptanoyl)-L-lysine | ++ |
| 63 | MbPylRS | L274A/C313V/Y349F | N6-((But-3-en-1-yloxy)carbonyl)-L-lysine | +++ |
| 64 | MbPylRS | L274A/C313V/Y349F | N6-((Pent-4-en-1-yloxy)carbonyl)-L-lysine | +++ |
| 65 | G1PylRS | WT | N6-((Prop-2-yn-1-yloxy)carbonyl)-L-lysine | 76 |
| 66 | MbPylRS | WT | N6-((But-3-yn-1-yloxy)carbonyl)-L-lysine | +++ |
| 67 | MbPylRS | L274A/C313S | N6-((Pent-4-yn-1-yloxy)carbonyl)-L-lysine | +++ |
| 68 | MbPylRS | L274G/C313V/M315A/V370R/ | N6-((Hept-6-yn-1-yloxy)carbonyl)-L-lysine | +++ |
| | | I378V | | |
| 69 | MmPylRS | Y306A/Y384F | N6-(((2-Ethynylbenzyl)oxy)carbonyl)-L-lysine | +++ |
| 70 | MmPylRS | Y306A/Y384F | N6-(((3-Ethynylbenzyl)oxy)carbonyl)-L-lysine | +++ |
| 71 | MmPylRS | WT | N6-((((2R,3R)-3-Ethynyltetrahydrofuran-2- | ++ |
| | | | yl)oxy)carbonyl)-L-lysine | |
| 72 | MmPylRS | WT | (S)-2-Amino-6-(((R)-2-aminopent-4- | +++ |
| | | | ynoyl)oxy)hexanoic acid | |
| 73 | G1PylRS | M128A/V167S/Y204F | N6-(((4-Azidobenzyl)oxy)carbonyl)-L-lysine | 73 |
| 74 | MmPylRS | Y306A/Y384F | N6-(6-(Azidomethyl)pyridine-2-carbonyl)-L- | +++ |
| | | | lysine | |
| 75 | MmPylRS | Y306A/Y384F | N6-(((3-Azidobenzyl)oxy)carbonyl)-L-lysine | +++ |
| 76 | MbPylRS | L274A/C313V/Y349F | N6-((((1S,2S)-2- | ++ |
| | | | Azidocyclopentyl)oxy)carbonyl)-L-lysine | |
| 77 | MmPylRS | WT | N6-((2-Azidoethoxy)carbonyl)-L-lysine | +++ |
| 78 | MbPylRS | C313V | N6-((3-Azidopropoxy)carbonyl)-L-lysine | +++ |
| 79 | MmPylRS | L309T/C348G/Y384F | (S,E)-2-Amino-6-((((4- | +++ |
| | | | azidobenzyl)oxy)carbonyl)amino)hex-5-enoic | |
| | | | acid | |
| 80 | MmPylRS | Y306A/Y384F | N6-(((2-Azidobenzyl)oxy)carbonyl)-L-lysine | +++ |
| 81 | MbPylRS | Y271A/L274V/C313V/M315Y/ | N6-((Cyclooct-2-yn-1-yloxy)carbonyl)-L-lysine | +++ |
| | | Y349F/V370R | | |
| 82 | MmPylRS | Y306A/Y384F | N6-((((1R,8S,9s)-Bicyclo[6.1.0]non-4-yn-9- | ++ |
| | | | yl)methoxy)carbonyl)-L-lysine | |
| 83 | MbPylRS | Y271A/L274V/C313V/M315Y/ | N6-((Bicyclo[6.1.0]non-4-yn-9- | +++ |
| | | Y349F/V370R | ylmethoxy)carbonyl)-L-lysine | |
| 84 | G1PylRS | WT | N6-((Bicyclo[2.2.1]hept-5-en-2- | 79 |
| | | | yloxy)carbonyl)-L-lysine | |
| 85 | MmPylRS | Y306A/Y384F | N6-((Bicyclo[2.2.1]hept-5-en-2- | ++ |
| | | | ylmethoxy)carbonyl)-L-lysine | |
| 86 | MmPylRS | Y306A/L309M/C348G/Y384F/ | N6-((((S,E)-Cyclooct-4-en-1- | +++ |
| | | I405R | yl)methyl)carbamoyl)-L-lysine | |
| 87 | MmPylRS | Y306A/L309M/C348G/Y384F/ | N6-((((R,E)-Cyclooct-4-en-1- | +++ |
| | | I405R | yl)methyl)carbamoyl)-L-lysine | |
| 88 | MmPylRS | Y306A/L309M/C348G/Y384F/ | N6-((E)-10-Oxo-9-azabicyclo[6.1.1]dec-6-ene- | +++ |
| | | I405R | 9-carbonyl)-L-lysine | |
| 89 | MmPylRS | Y306A/L309M/C348G/Y384F/ | N6-(2-((R,E)-Cyclooct-2-en-1-yl)acetyl)-L- | +++ |
| | | I405R | lysine | |
| 90 | MmPylRS | Y306A/Y384F | N6-((((S,Z)-Cyclooct-4-en-1-yl)oxy)carbonyl)- | +++ |
| | | | L-lysine | |
| 91 | MmPylRS | Y306A/Y384F | N6-((((E)-5,8-Dihydro-4H-1,3-dioxocin-5- | ++ |
| | | | yl)oxy)carbonyl)-L-lysine | |
| 92 | MbPylRS | Y271G/C313V | N6-((2-(6-Methyl-1,2,4,5-tetrazin-3- | ++ |
| | | | yl)ethoxy)carbonyl)-L-lysine | |
| 93 | MbPylRS | Y271G/C313V | N6-(((3-(6-Methyl-1,2,4,5-tetrazin-3- | ++ |
| | | | yl)benzyl)oxy)carbonyl)-L-lysine | |
| 94 | MmPylRS | WT | N6-(((2-Methylcycloprop-2-en-1- | ++ |
| | | | yl)methoxy)carbonyl)-L-lysine | |
| 95 | MmPylRS | WT | N6-((Spiro[2.3]hex-1-en-5- | ++ |
| | | | ylmethoxy)carbonyl)-L-lysine | |
| 96 | MbPylRS | L266M/L270I/Y271L/L274A/ | N6-(1-Methylcycloprop-2-ene-1-carbonyl)-L- | ++ |
| | | C313I | lysine | |
| 97 | G1PylRS | H120M/L124I/Y125F/M128A/ | N6-Acryloyl-L-lysine | 744 |
| | | V167F | | |
| 98 | MbPylRS | D76G/L266V/L270I/Y271F/ | (S)-2-Amino-8-oxononanoic acid | ++ |
| | | L274A/C313F | | |
| 99 | MmPylRS | WT | N6-(D-Cysteinyl)-L-lysine | ++ |
| 100 | MbPylRS | A267S/C313V/M315F/d344g | N6-((R)-Thiazolidine-4-carbonyl)-L-lysine | ++ |
| 101 | MbPylRS | L274A/C313S | N6-(((4-Iodobenzyl)oxy)carbonyl)-L-lysine | +++ |
| 102 | MbPylRS | M241/A267/Y271M/L274G/ | (2S)-2-Amino-5-mercapto-6-((((4- | +++ |
| | | C313A/Y349W | nitrobenzyl)oxy)carbonyl)amino)hexanoic acid | |
| 103 | MbPylRS | M241A/Y271A/L274V/C313V/ | N6-(((5-((3aR,6aS)-2-Oxohexahydro-1H- | ++ |
| | | M315Y/Y349F/V370R | thieno [3,4-d]imidazol-4- | |
| | | | yl)pentyl)oxy)carbonyl)-L-lysine | |
| 104 | MbPylRS | M241A/Y271A/L274V/C313V/ | N6-((4-((3aR,6aS)-2-Oxohexahydro-1H- | ++ |
| | | M315Y/Y349F/V370R | thieno[3,4-d]imidazol-4-yl)butoxy)carbonyl)-L- | |
| | | | lysine | |
| 105 | MbPylRS | M241A/Y271A/L274V/C313V/ | N6-(((5-((3aR,6aS)-2-Iminohexahydro-1H- | ++ |
| | | M315Y/Y349F/V370R | thieno [3,4-d]imidazol-4- | |
| | | | yl)pentyl)oxy)carbonyl)-L-lysine | |
| 106 | MbPylRS | Y271M/L274T/C313A/Y349F | N6-(2-(2,4-Dinitrophenyl)acetyl)-L-lysine | |
| 107 | MbPylRS | Y271A/L274M | N6-((2-(7-Hydroxy-2-oxo-2H-chromen-4- | ++ |
| | | | yl)ethoxy)carbonyl)-L-lysine | |
| 108 | MbPylRS | Y271A/L274M | N6-((2-(7-Amino-2-oxo-2H-chromen-4- | 13 |
| | | | yl)ethoxy)carbonyl)-L-lysine | |
| 109 | MmPylRS | Y306A/L309A/C348S/Y384F | N6-((2-(7-Amino-6-fluoro-2-oxo-2H-chromen- | ++ |
| | | | 4-yl)ethoxy)carbonyl)-L-lysine | |
| 110 | MmPylRS | Y306A/Y384F | N6-(((Trimethylsilyl)methoxy)carbonyl)-L- | |
| | | | lysine | |
| 111 | MmPylRS | Y306A/Y384F/I413L | N6-(((1-Hydroxy-2,2,5,5-tetramethyl-2,5- | ++ |
| | | | dihydro-1H-pyrrol-3-yl)methoxy)carbonyl)-L- | |
| | | | lysine | |
| 112 | MmPylRS | Y306A/Y384F/I413L | N6-(2-(Trimethylsilyl)acetyl)-L-lysine | ++ |
| 113 | chPylRS | C348G/V401C/Y384F | N6-(2-(1,2-Dithiolan-3-yl)acetyl)-L-lysine | ++ |
| 114 | MbPylRS | Y271A/Y349F | N6-(2-(Phenylthio)acetyl)-L-lysine | ++ |
| 115 | MbPylRS | L270I/Y271A/Y349F | N6-(3-(4-Fluorophenyl)propanoyl)-L-lysine | ++ |
| 116 | MbPylRS | L274G/C313V/Y349F | N6-(2-Phenoxyacetyl)-L-lysine | ++ |
| 117 | MbPylRS | L270I/Y271A/Y349F | N6-(D-Prolyl)-L-lysine | +++ |
| 118 | MbPylRS | WT | N6-(L-Prolyl)-L-lysine | ++ |
| 1 | MbPylRS | WT | N6-((R)-Piperidine-2-carbonyl)-L-lysine | ++ |
| 120 | MbPylRS | WT | N6-((S)-Piperidine-2-carbonyl)-L-lysine | ++ |
| 121 | MbPylRS | WT | N6-((Allyloxy)carbonyl)-L-lysine | ++ |
| 122 | MmPylRS | Y384F | N6-((Hex-5-en-1-yloxy)carbonyl)-L-lysine | ++ |
| 123 | MbPylRS | L274C/C313V/1378V | N6-((Hept-6-en-1-yloxy)carbonyl)-L-lysine | ++ |
| 124 | MbPylRS | Y271G/C313V/V370R | N6-((Oct-7-en-1-yloxy)carbonyl)-L-lysine | ++ |
| 125 | MbPylRS | Y271G/L274V/C313V/M315Y/ | N6-((Non-8-en-1-yloxy)carbonyl)-L-lysine | ++ |
| | | V370R | | |
| 126 | MbPylRS | Y271A/L274V/C313V/M315Y/ | N6-((Dec-9-en-1-yloxy)carbonyl)-L-lysine | ++ |
| | | V370R | | |
| 127 | MbPylRS | Y271A/L274A/C313V/M315Y/ | N6-((2,2,2-Trifluoroethoxy)carbonyl)-L-lysine | + |
| | | V370R/I378V | | |
| 128 | MbPylRS | C313V/M315L/V370K/1378V | N6-((2,2,3,3,3-Pentafluoropropoxy)carbonyl)- | ++ |
| | | | L-lysine | |
| 129 | MbPylRS | Y271A/C313V/V370L/1378V | N6-((2,2,3,3,4,4,4- | ++ |
| | | | Heptafluorobutoxy)carbonyl)-L-lysine | |
| 130 | MbPylRS | Y271A/L274V/C313V/M315Y/ | N6-(((2,2,3,3,4,4,5,5,5- | ++ |
| | | Y349F/V370R | Nonafluoropentyl)oxy)carbonyl)-L-lysine | |
| 131 | MbPylRS | Y271A/L274V/C313V/M315Y/ | N6-((4,4,4-Trifluoro-3- | ++ |
| | | Y349F/V370R | (trifluoromethyl)butoxy)carbonyl)-L-lysine | |
| 132 | MbPylRS | Y271A/L274V/C313V/M315Y/ | N6-(((3,4,5-Trifluorobenzyl)oxy)carbonyl)-L- | ++ |
| | | Y349F/V370R | lysine | |
| 133 | MbPylRS | Y271A/L274V/C313V/M315Y/ | N6-(((4- | ++ |
| | | Y349F/V370R | (Trifluoromethyl)benzyl)oxy)carbonyl)-L- | |
| | | | lysine | |
| 134 | MbPylRS | Y271A/L274V/C313V/M315Y/ | (E)-N6-(((3,7-Dimethylocta-2,6-dien-1- | ++ |
| | | Y349F/V370R | yl)oxy)carbonyl)-L-lysine | |
| 135 | MbPylRS | Y271A/L274V/C313V/M315Y/ | (Z)-N6-(((3,7-Dimethylocta-2,6-dien-1- | ++ |
| | | Y349F/V370R | yl)oxy)carbonyl)-L-lysine | |
| 136 | MbPylRS | Y271A/L274V/C313V/M315Y/ | N6-((((2E,4E)-Hexa-2,4-dien-1- | ++ |
| | | Y349F/V370R | yl)oxy)carbonyl)-L-lysine | |
| 137 | MbPylRS | Y271A/L274V/C313V/M315Y/ | N6-((2-(Thiophen-2-yl)ethoxy)carbonyl)-L- | ++ |
| | | Y349F/V370R | lysine | |
| 138 | MbPylRS | Y271A/L274V/C313V/M315Y/ | N6-((Pyridin-2-ylmethoxy)carbonyl)-L-lysine | ++ |
| | | Y349F/V370R | | |
| 139 | MbPylRS | Y271A/L274V/C313V/M315Y/ | N6-(tert-Butoxycarbonyl)-L-lysine | ++ |
| | | Y349F/V370R | | |
| 140 | G1PylRS | Y204F | (S)-6-((tert-Butoxycarbonyl)amino)-2- | 83 |
| | | | hydroxyhexanoic acid | |
| 141 | MbPylRS | L274A/C313V/Y349F | N6-(2-Bromo-2-methylpropanoyl)-L-lysine | + |
| 142 | MbPylRS | C313S/Y349F | N6-Pivaloyl-L-lysine | ++ |
| 143 | MbPylRS | C313S/Y349F | N6-Heptanoyl-L-lysine | ++ |
| 144 | MmPylRS | L301M/Y306A/L309A/C348F | N6-(((4-Vinylbenzyl)oxy)carbonyl)-L-lysine | + |
| 145 | MbPylRS | L274A/C313S/Y349F | N6-((Benzyloxy)carbonyl)-L-lysine | ++ |
| 146 | MmPylRS | Y306A/Y384F | N6-(((2-Chlorobenzyl)oxy)carbonyl)-L-lysine | +++ |
| 147 | G1PylRS | Y125A/Y204F | N6-(((2-Bromobenzyl)oxy)carbonyl)-L-lysine | 76 |
| 148 | MmPylRS | Y306A/Y384F | N6-(((4-Nitrobenzyl)oxy)carbonyl)-L-lysine | +++ |
| 149 | G1PylRS | Y125A/Y204F | N6-(((4-Nitrobenzyl)oxy)carbonyl)-L-lysine | 58 |
| 150 | G1PylRS | Y125A/Y204F | N6-((Cyclopentylmethoxy)carbonyl)-L-lysine | 58 |
| 151 | MbPylRS | WT | N6-((((R)-Tetrahydrofuran-2-yl)oxy)carbonyl)- | +++ |
| | | | L-lysine | |
| 152 | MbPylRS | WT | (S)-2-Amino-2-((3- | ++ |
| | | | (((benzyloxy)carbonyl)amino)propyl)thio)acetic | |
| | | | acid | |
| 153 | MmPylRS | Y306A/Y384F | (S)-2-Amino-2-((3- | ++ |
| | | | (((benzyloxy)carbonyl)amino)propyl)seleno)acetic | |
| | | | acid | |
| 154 | MmPylRS | Y306A/Y384F | (2S)-2-Amino-5-hydroxy-6-((((4- | ++ |
| | | | nitrobenzyl)oxy)carbonyl)amino)hexanoic acid | |
| 155 | MbPylRS | M241/A267/Y271M/L274G/ | (S)-2-Amino-3-(2-fluorophenyl)propanoic acid | ++ |
| | | C313A/Y349W | | |
| 156 | G1PylRS | N165G/V167A | (S)-2-Amino-3-(2-chlorophenyl)propanoic acid | 52 |
| 157 | G1PylRS | N165A/V167A | (S)-2-Amino-3-(2-bromophenyl)propanoic acid | 75 |
| 158 | G1PylRS | N165A/V167A | (S)-2-Amino-3-(2-iodophenyl)propanoic acid | 75 |
| 159 | G1PylRS | N165A/V167A | (S)-2-Amino-3-(2-methylphenyl)propanoic acid | 56 |
| 160 | G1PylRS | N165A/V167A | (S)-2-Amino-3-(2-cyanophenyl)propanoic acid | 82 |
| 161 | G1PylRS | N165A/V167A | (S)-2-Amino-3-(3-chlorophenyl)propanoic acid | 83 |
| 162 | MmPylRS | N346A/C348A | (S)-2-Amino-3-(3-bromophenyl)propanoic acid | + |
| 163 | G1PylRS | N165A/V167A | (S)-2-Amino-3-(3-iodophenyl)propanoic acid | 81 |
| 164 | G1PylRS | N165A/V167A | (S)-2-Amino-3-(3-methylphenyl)propanoic acid | 83 |
| 165 | G1PylRS | N165A/V167A | (S)-2-Amino-3-(4-iodophenyl)propanoic acid | 81 |
| 166 | G1PylRS | N165A/V167A | (S)-2-Amino-3-(4-bromophenyl)propanoic acid | 46 |
| 167 | MmPylRS | L105M/Y306L/L309S/N346S/ | (S)-2-Amino-3-(4-methoxyphenyl)propanoic | |
| | | C348M/ | acid | |
| 168 | G1PylRS | A121T/N165V/V167W/Y204F/ | (S)-2-Amino-3-(2,4-difluorophenyl)propanoic | 46 |
| | | A221L | acid | |
| 169 | G1PylRS | N165G/V167A | (S)-2-Amino-3-(2,5-difluorophenyl)propanoic | 63 |
| | | | acid | |
| 170 | G1PylRS | N165G/V167A | (S)-2-Amino-3-(2,6-difluorophenyl)propanoic | 73 |
| | | | acid | |
| 171 | G1PylRS | N165G/V167A | (S)-2-Amino-3-(4-benzoylphenyl)propanoic | 55 |
| | | | acid | |
| 172 | MmPylRS | A302T/N346G/C348T/V401I/ | (S)-2-Amino-3-(4-(prop-1,2-dien-1- | + |
| | | W417Y | yloxy)phenyl)propanoic acid | |
| 173 | MmPylRS | N346G/C348G/Y384F | (2S)-2-Amino-3-(4-((3-methylpent-1-yn-3- | ++ |
| | | | yl)oxy)phenyl)propanoic acid | |
| 174 | MmPylRS | A302T/N346A/C348A/V401L/ | (S)-2-Amino-3-(4-((6-nitrobenzo[d][1,3]dioxol- | ++ |
| | | W417A | 5-yl)methoxy)phenyl)propanoic acid | |
| 175 | G1PylRS | L124F/N165G/V167G | (2S)-2-Amino-3-(4-(1-(6- | 57 |
| | | | nitrobenzo[d][1,3]dioxol-5- | |
| | | | yl)ethoxy)phenyl)propanoic acid | |
| 176 | MbPylRS | L270F/L274M/N311G/C313G/ | (2S)-2-Amino-3-(4-(2-(2- | ++ |
| | | Y349F | nitrophenyl)propoxy)phenyl)propanoic acid | |
| 177 | MbPylRS | L270F/L274M/N311G/C313G/ | (S)-2-Amino-3-(4-((2- | ++ |
| | | Y349F | nitrobenzyl)oxy)phenyl)propanoic acid | |
| 178 | G1PylRS | N165A/V167A | (S)-2-Amino-3-(3-(fluorosulfonyl)-4- | 53 |
| | | | methoxyphenyl)propanoic acid | |
| 179 | MmPylRS | Y306L/L309A/N346A/C348M/ | (S)-2-Amino-3-(4- | ++ |
| | | W417T | ((fluorosulfonyl)oxy)phenyl)propanoic acid | |
| 180 | MmPylRS | A302I/N346T/C348I/Y384L/W | (S)-2-Amino-3-(3- | 13 |
| | | 417K | ((fluorosulfonyl)oxy)phenyl)propanoic acid | |
| 181 | MmPylRS | L305M/I322T/N346G | (S)-2-Amino-3-(3-fluoro-4- | ++ |
| | | | ((fluorosulfonyl)oxy)phenyl)propanoic acid | |
| 182 | MmPylRS | A302I/N346T/C348I/Y384L/W | (S)-2-Amino-3-(4-(3-bromopropoxy)-3- | ++ |
| | | 417K | ethynylphenyl)propanoic acid | |
| 183 | MmPylRS | A302T/N346A/C348V/Y384F/ | (2R)-2-Amino-3-fluoro-3-(4-((2- | ++ |
| | | W417T | nitrobenzyl)oxy)phenyl)propanoic acid | |
| 184 | MbPylRS | L270F/L274M/N311A/C313G | (S)-2-Amino-3-(3-(fluoromethyl)-4-((2- | ++ |
| | | | nitrobenzyl)oxy)phenyl)propanoic acid | |
| 185 | MaPylRS | L125F/N166A/V168G | (S)-2-Amino-3-(4-(3- | ++ |
| | | | chloropropoxy)phenyl)propanoic acid | |
| 186 | MmPylRS | A302T/N346A/C348V/Y384F/ | (S)-2-Amino-3-(4-(2- | ++ |
| | | W417T | bromoethoxy)phenyl)propanoic acid | |
| 187 | MmPylRS | A302T/N346A/C348V/Y384F/ | (S)-2-Amino-3-(4-(3- | ++ |
| | | W417T | bromopropoxy)phenyl)propanoic acid | |
| 188 | MmPylRS | A302T/N346A/C348V/Y384F/ | (S)-2-Amino-3-(4-(4- | ++ |
| | | W417T | bromobutoxy)phenyl)propanoic acid | |
| 189 | MmPylRS | A302T/N346A/C348V/Y384F/ | (S)-2-Amino-3-(4-((5- | + |
| | | W417T | bromopentyl)oxy)phenyl)propanoic acid | |
| 0 | MmPylRS | A302T/N346A/C348V/Y384F/ | (S)-2-Amino-3-(4-(2- | + |
| | | W417T | iodoethoxy)phenyl)propanoic acid | |
| 1 | MmPylRS | A302T/N346A/C348V/Y384F/ | (S)-2-Amino-3-(4-(3- | ++ |
| | | W417T | iodopropoxy)phenyl)propanoic acid | |
| 2 | MmPylRS | A302T/N346A/C348V/Y384F/ | (S,E)-2-Amino-3-(4-((2,6- | + |
| | | W417T | difluorophenyl)diazenyl)phenyl)propanoic acid | |
| 3 | MbPylRS | L270F/ | (S,E)-2-Amino-3-(4-((2,6- | ++ |
| | | L274M/N311G/C313G/Y349F | difluorophenyl)diazenyl)-3,5- | |
| | | | difluorophenyl)propanoic acid | |
| 4 | MbPylRS | L270F/ | (S,E)-2-Amino-3-(4- | ++ |
| | | L274M/N311G/C313G/Y349F | (phenyldiazenyl)phenyl)propanoic acid | |
| 5 | MbPylRS | L270F/ | (S,E)-2-Amino-3-(4-((3- | ++ |
| | | L274M/N311G/C313G/Y349F | vinylphenyl)diazenyl)phenyl)propanoic acid | |
| 6 | MmPylRS | A302T/L309S/N346V/C348G | (S,E)-3-(4-((3-Acetylphenyl)diazenyl)phenyl)- | ++ |
| | | | 2-aminopropanoic acid | |
| 7 | MmPylRS | A302T/L309A/I322T/N346A/ | (S,E)-2-Amino-3-(4-((3- | ++ |
| | | C348G | (chloromethyl)phenyl)diazenyl)phenyl)propanoic | |
| | | | acid | |
| 8 | MmPylRS | A302T/L309S/N346V/C348G | (S,E)-2-Amino-3-(4- | ++ |
| | | | ((pentafluorophenyl)diazenyl)phenyl)propanoic | |
| | | | acid | |
| 9 | MmPylRS | A302T/L309S/N346V/C348G | (S,E)-2-Amino-3-(4-((3- | ++ |
| | | | cyanophenyl)diazenyl)phenyl)propanoic acid | |
| 200 | MmPylRS | A302T/L309S/N346V/C348G | (S,E)-2-Amino-3-(4-((2,4,6- | 23 |
| | | | trifluorophenyl)diazenyl)phenyl)propanoic acid | |
| 201 | G1PylRS | A121T/M128S/N165V/V167G | (S,E)-2-Amino-3-(4-((1,3,5-trimethyl-1H- | 47 |
| | | | pyrazol-4-yl)diazenyl)phenyl)propanoic acid | |
| 202 | MbPylRS | Y271M/L274A/N311A/C313A/ | (S)-2-Amino-3-(3-(6-methyl-1,2,4,5-tetrazin-3- | ++ |
| | | Y349F | yl)phenyl)propanoic acid | |
| 203 | MmPylRS | L305G/N346G/C348A | (S)-2-Amino-3-(3-(6-ethyl-1,2,4,5-tetrazin-3- | +++ |
| | | | yl)phenyl)propanoic acid | |
| 204 | MmPylRS | L305G/N346G/C348A | (S)-2-Amino-3-(3-(6-isopropyl-1,2,4,5-tetrazin- | +++ |
| | | | 3-yl)phenyl)propanoic acid | |
| 205 | MmPylRS | L305G/N346G/C348A | (S)-2-Amino-3-(3-(6-butyl-1,2,4,5-tetrazin-3- | +++ |
| | | | yl)phenyl)propanoic acid | |
| 206 | MmPylRS | L305G/N346G/C348A | (S)-2-Amino-3-(3-(6-butyl-1,2,4,5-tetrazin-3- | +++ |
| | | | yl)phenyl)propanoic acid | |
| 207 | G1PylRS | L124G/N165G/V167A/M128A | (S)-2-Amino-3-(3-(6-butyl-1,2,4,5-tetrazin-3- | +++ |
| | | | yl)phenyl)propanoic acid | |
| 208 | G1PylRS | L124G/M128A/N165G/V167A | (S)-2-Amino-3-(3-(6-pentyl-1,2,4,5-tetrazin-3- | +++ |
| | | | yl)phenyl)propanoic acid | |
| 209 | G1PylRS | L124G/M128L/N165G/V167A | (S)-2-Amino-3-(3-(6-propyl-1,2,4,5-tetrazin-3- | +++ |
| | | | yl)phenyl)propanoic acid | |
| 210 | G1PylRS | N165G/V167G | (S)-2-Amino-3-(3-(6-isobutyl-1,2,4,5-tetrazin- | +++ |
| | | | 3-yl)phenyl)propanoic acid | |
| 211 | MbPylRS | I287V/N311S/C313G/V366H/ | (S)-2-Amino-3-(6-phenyl-1,2,4,5-tetrazin-3- | ++ |
| | | W382V | yl)propanoic acid | |
| 212 | MbPylRS | N311S/C313A/V366H/W382I | (S)-2-Amino-3-(6-(pyridin-2-yl)-1,2,4,5- | ++ |
| | | | tetrazin-3-yl)propanoic acid | |
| 213 | MmPylRS | L305G/N346G/C348A | (S)-2-Amino-3-(3-(6-(3-azidopropyl)-1,2,4,5- | ++ |
| | | | tetrazin-3-yl)phenyl)propanoic acid | |
| 214 | MmPylRS | N346A/C348A | (S)-2-Amino-3-(3-azidophenyl)propanoic acid | ++ |
| 215 | MmPylRS | N346A/C348A | (S)-2-Amino-3-(4-(2- | ++ |
| | | | azidoethoxy)phenyl)propanoic acid | |
| 216 | MmPylRS | N346A/C348A | (S)-2-Amino-3-(3-ethynylphenyl)propanoic | ++ |
| | | | acid | |
| 217 | G1PylRS | N165A/V167A | (S)-2-Amino-3-(4-(prop-2-yn-1- | 91 |
| | | | yloxy)phenyl)propanoic acid | |
| 218 | MmPylRS | N346A/C348A | (S)-2-Amino-3-(3-formylphenyl)propanoic acid | + |
| 2 | MmPylRS | N346A/C348A | (S)-3-(3-Acetylphenyl)-2-aminopropanoic acid | ++ |
| 220 | G1PylRS | L124I/Y125F/M128G/V167F/ | (S)-2-Amino-3-(2-cyanopyridin-4-yl)propanoic | 79 |
| | | Y204F | acid | |
| 221 | G1PylRS | L124A/Y125F/Y204W/A221S/ | (S)-2-Amino-3-(6-cyanopyridin-3-yl)propanoic | ++ |
| | | W237Y | acid | |
| 222 | MbPylRS | N311A/C313M/v366g/W382T | (S)-2-Amino-3-(2,3,5,6-tetrafluoro-4- | ++ |
| | | | methylphenyl)propanoic acid | |
| 223 | G1PylRS | N165A/V167M/A221G/W237T | (S)-2-Amino-3- | 53 |
| | | | (pentafluorcorrelation)propanoic acid | |
| 224 | MbPylRS | N311A/C313M/v366g/W382T | (S)-2-Amino-3-(2,3,5,6- | ++ |
| | | | tetrafluorophenyl)propanoic acid | |
| 225 | MbPylRS | N311G/C313A | (S)-2-Amino-3-(2,3,4,5- | ++ |
| | | | tetrafluorophenyl)propanoic acid | |
| 226 | G1PylRS | N165G/V167Q | (S)-2-Amino-3-(2,3,6- | 87 |
| | | | trifluorophenyl)propanoic acid | |
| 227 | G1PylRS | N165G/V167Q | (S)-2-Amino-3-(2,3,4- | 87 |
| | | | trifluorophenyl)propanoic acid | |
| 228 | G1PylRS | N165G/V167Q | (S)-2-Amino-3-(2,3-difluorophenyl)propanoic | 80? |
| | | | acid | |
| 229 | G1PylRS | N165G/V167Q | (S)-2-Amino-3-(2,4,5- | 83 |
| | | | trifluorophenyl)propanoic acid | |
| 230 | MmPylRS | N346Q/C348S/V401G/W417T | (S)-2-Amino-3-(quinolin-6-yl)propanoic acid | + |
| 231 | MmPylRS | L309G/N346A/C348I/V401K/ | (S)-2-Amino-3-(4-(4- | ++ |
| | | W417I | hydroxyphenoxy)phenyl)propanoic acid | |
| 232 | G1PylRS | N165A/V167A | (S)-2-Amino-3-(2-nitrophenyl)propanoic acid | 72 |
| 233 | MmPylRS | N346A/C348A | (S)-2-Amino-3-(2-methoxyphenyl)propanoic | |
| | | | acid | |
| 234 | G1PylRS | N165A/V167A | (S)-2-Amino-3-(3- | 81 |
| | | | (trifluoromethyl)phenyl)propanoic acid | |
| 235 | G1PylRS | N165A/V167A | (S)-2-Amino-3-(3-cyanophenyl)propanoic acid | 74 |
| 236 | G1PylRS | N165A/V167A | (S)-2-Amino-3-(3-methoxyphenyl)propanoic | 83 |
| | | | acid | |
| 237 | G1PylRS | N165A/V167A | (S)-2-Amino-3-(3-nitrophenyl)propanoic acid | 85 |
| 238 | MmPylRS | N346A/C348A | (S)-2-Amino-3-(4- | ++ |
| | | | (cyclopentylmethoxy)phenyl)propanoic acid | |
| 239 | MmPylRS | N346A/C348A | (S)-2-Amino-3-(4-(but-2-yn-1- | ++ |
| | | | yloxy)phenyl)propanoic acid | |
| 240 | G1PylRS | N165A/V167A | (S)-2-Amino-3-(4- | 46 |
| | | | (benzyloxy)phenyl)propanoic acid | |
| 241 | G1PylRS | N165A/V167A | (S)-3-(4-(Allyloxy)phenyl)-2-aminopropanoic | 83 |
| | | | acid | |
| 242 | MmPylRS | N346A/C348A | (S)-2-Amino-3-(4-(but-3-en-1- | ++ |
| | | | yloxy)phenyl)propanoic acid | |
| 243 | MmPylRS | N346A/C348A | (S)-2-Amino-3-(4-(pent-4-en-1- | ++ |
| | | | yloxy)phenyl)propanoic acid | |
| 244 | G1PylRS | N165A/V167A | (S)-2-Amino-3-(4-(tert- | 72 |
| | | | butoxy)phenyl)propanoic acid | |
| 245 | G1PylRS | N165G/V167Q | (S)-2-Amino-3-(5-bromo-2- | 75 |
| | | | chlorophenyl)propanoic acid | |
| 246 | G1PylRS | N165G/V167Q | (S)-2-Amino-3-(2,5-dichlorophenyl)propanoic | 84 |
| | | | acid | |
| 247 | G1PylRS | L124I/Y125F/M128G/V167F/ | (S)-2-Amino-3-(pyridin-3-yl)propanoic acid | 83 |
| | | Y204F | | |
| 248 | MmPylRS | N346A/C348A | (S)-2-Amino-3-(benzo[d][1,3]dioxol-5- | + |
| | | | yl)propanoic acid | |
| 249 | MmPylRS | A302T/N346T/C348T | (S)-2-Amino-3-(naphthalen-2-yl)propanoic acid | + |
| 250 | G1PylRS | L124G/Y125F/N165G/V167F/ | (S)-2-Amino-3-(7-fluoro-1H-indol-3- | 74 |
| | | A221G/W237Y | yl)propanoic acid | |
| 251 | G1PylRS | L124M/Y125M/N165A/V167G/ | (S)-2-Amino-3-(1H-pyrrolo[2,3-b]pyridin-3- | 22 |
| | | A221G/W237H | yl)propanoic acid | |
| 252 | G1PylRS | N165G/V167Q | (S)-2-Amino-3-(benzo[b]thiophen-3- | 89 |
| | | | yl)propanoic acid | |
| 253 | G1PylRS | L124I/Y125F/M128G/V167F/ | Np-Methyl-L-histidine | 85 |
| | | Y204F | | |
| 254 | MbPylRS | N311G/C313V/V366K | Nt-(2-Nitrobenzyl)-L-histidine | ++ |
| 255 | MbPylRS | A267Q/N311S/C313W | (S)-2-Amino-3-(3H-124-thiazol-4-yl)propanoic | + |
| | | | acid | |
| 256 | G1PylRS | L124I/Y125F/M128G/V167F/ | (S)-2-Amino-3-(thiophen-3-yl)propanoic acid | 64 |
| | | Y204F | | |
| 257 | G1PylRS | L124I/Y125F/M128G/V167F/ | (S)-2-Amino-3-(thiazol-5-yl)propanoic acid | 91 |
| | | Y204F | | |
| 258 | G1PylRS | L124I/Y125F/M128G/V167F/ | (S)-2-Amino-3-(thiophen-2-yl)propanoic acid | 72 |
| | | Y204F | | |
| 259 | G1PylRS | L124I/Y125F/M128G/V167F/ | (S)-2-Amino-3-(5-bromothiophen-2- | 87 |
| | | Y204F | yl)propanoic acid | |
| 260 | G1PylRS | L124I/Y125F/M128G/V167F/ | (S)-2-Amino-3-(furan-2-yl)propanoic acid | 43 |
| | | Y204F | | |
| 261 | chPylRS | L270I/L274G/N311C/C313W/ | (S)-2-Amino-4-((2-nitrobenzyl)oxy)-4- | 23 |
| | | Y349F | oxobutanoic acid | |
| 262 | chPylRS | L270V/L274G/N311C/C313W/ | (2S)-2-Amino-4-(1-(2-nitrophenyl)ethoxy)-4- | ++ |
| | | Y349F | oxobutanoic acid | |
| 263 | MbPylRS | N311S/C313G/Y349F | (S)-2-Amino-5-((2-nitrobenzyl)oxy)-5- | + |
| | | | oxopentanoic acid | |
| 264 | MbPylRS | A267D/N311G/C313G | (S)-2-Amino-5-((6-nitrobenzo[d][1,3]dioxol-5- | ++ |
| | | | yl)methoxy)-5-oxopentanoic acid | |
| 265 | MbPylRS | N311S/C313A/Y349F | (S)-2-Amino-5-(benzyloxy)-5-oxopentanoic | ++ |
| | | | acid | |
| 266 | G1PylRS | X16 N165C/V167S/Y204F | (S)-2-Amino-4-(benzyloxy)-4-oxobutanoic acid | 75 |
| 267 | MbPylRS | A267Y/Y271A/N311T/C313G/ | (S)-2-Amino-4-((2- | ++ |
| | | Y349F | (((cyclopentyloxy)carbonyl)amino)ethyl)thio)- | |
| | | | 4-oxobutanoic acid | |
| 268 | MbPylRS | N311M/C313Q/V366G/W382N | S-(2-Nitrobenzyl)-L-cysteine | + |
| 269 | MbPylRS | N311Q/C313A/V366M | S-(1-(6-Nitrobenzo[d][1,3]dioxol-5-yl)ethyl)-L- | + |
| | | | cysteine | |
| 270 | MbPylRS | M241F/A267S/Y271C/L274M | S-((((1-(6-Nitrobenzo[d][1,3]dioxol-5- | + |
| | | | yl)ethoxy)carbonyl)amino)methyl)-L-cysteine | |
| 271 | MbPylRS | M241F/A267S/Y271C/L274M | S-((((1-(6-Nitrobenzo[d][1,3]dioxol-5- | + |
| | | | yl)ethoxy)carbonyl)amino)methyl)-L- | |
| | | | homocysteine | |
| 272 | MbPylRS | C313W/W382T | (R)-3-(Allyl selenyl)-2-aminopropanoic acid | + |
| 273 | MbPylRS | C313W/W382T | S-(Prop-2-yn-1-yl)-L-cysteine | + |
| 274 | MbPylRS | Y271M/L274A/C313A | S-((2-Phenylacetamido)methyl)-L- | ++ |
| | | | homocysteine | |
| 275 | MmPylRS | N346Q/C348A/V401M | (2R)-2-Amino-3-((1-(6- | + |
| | | | nitrobenzo[d][1,3]dioxol-5- | |
| | | | yl)ethyl)selanyl)propanoic acid | |
| 276 | MbPylRS | Y271C/N311Q/Y349F/V366C | (2S)-2-Amino-3-(((2-((1-(6- | + |
| | | | nitrobenzo[d][1,3]dioxol-5- | |
| | | | yl)ethyl)thio)ethoxy)carbonyl)amino)propanoic | |
| | | | acid | |
| 277 | G1PylRS | N165S/V167G/A221A/W237T | (2S)-2-Amino-3-(9-oxo-8a,9,10,10a- | 63 |
| | | | tetrahydroacridin-2-yl)propanoic acid | |
| 278 | MmPylRS | A302T/N346A/C348G/Y384F/ | (S)-2-Amino-3- | ++ |
| | | W417T | (dibenzo[b,f][1,4,5]thiadiazepin-2-yl)propanoic | |
| | | | acid | |
| 279 | MmPylRS | WT | 2-Amino-5-(4-(dimethylamino)phenyl)oxazole- | ++ |
| | | | 4-carboxylic acid | |
| 280 | MmPylRS | C348W/W417S | S-Allyl-L-cysteine | + |
| 281 | MaPylRS | Y126G/M129A/V168F/H227T/ | N6-(4- | + |
| | | Y228P/L229I | (((Dimethylamino)fluorophosphoryl)oxy)ben- | |
| | | | zoyl)-L-lysine | |
| 282 | MmPylRS | L305M/I322T/N346G | (2S)-2-Amino-3-(4- | + |
| | | | (((dimethylamino)fluorophosphoryl)oxy)phe- | |
| | | | nyl)propanoic acid | |
| 283 | G1PylRS | Y60_Y125A/H225I/K226P | N6-(4-Oxo-4-(propylthio)butanoyl)-L-lysine | 55 |
| 284 | G1PylRS | Y60_Y125A/H225I/K226P | N6-(5-Oxo-5-(propylthio)pentanoyl)-L-lysine | 57 |
| 285 | chPheRS | E148D/F221V/T224G/A264G | (S)-2-Amino-3-(7,8-dihydro-1H-furo[2,3- | ++ |
| | | | glindol-3-yl)propanoic acid | |
| 286 | chPheRS | E148D/T224G/A264G | (S)-2-Amino-3-(3-iodophenyl)propanoic acid | 61 |
| 287 | chPheRS | E148D/T224G/A264G | (S)-2-Amino-3-(4- | +++ |
| | | | ((fluorosulfonyl)oxy)phenyl)propanoic acid | |
| 288 | chPheRS | E148D/T224G/A264G | (S)-2-Amino-3-(3- | +++ |
| | | | (dimethylamino)phenyl)propanoic acid | |
| 289 | chPheRS | E148D/T224G/A264G | (S)-2-Amino-3-(3-(pyrrolidin-1- | ++ |
| | | | yl)phenyl)propanoic acid | |
| 290 | chPheRS | E148D/T224G/A264G | (S)-2-Amino-3-(6-methyl-1H-indol-3- | 76 |
| | | | yl)propanoic acid | |
| 291 | chPheRS | E148D/T224G/A264G | (S)-2-Amino-3-(6-chloro-1H-indol-3- | 73 |
| | | | yl)propanoic acid | |
| 292 | chPheRS | E148D/T224G/A264G | (S)-2-Amino-3-(6-bromo-1H-indol-3- | 73 |
| | | | yl)propanoic acid | |
| 293 | chPheRS | E148D/T224G/A264G | (S)-2-Amino-3-(6-cyano-1H-indol-3- | 84 |
| | | | yl)propanoic acid | |
| 294 | chPheRS | E148D/V150G/F221V/T224G/ | (S)-2-Amino-3-(6-methoxy-1H-indol-3- | 63 |
| | | L247V/A264G | yl)propanoic acid | |
| 295 | chPheRS | E148D/V150G/F221V/T224G/ | (S)-2-Amino-3-(6,7-dimethyl-1H-indol-3- | +++ |
| | | L247V/A264G | yl)propanoic acid | |
| 296 | chPheRS | E148D/V150G/F221V/T224G/ | (S)-2-Amino-3-(6,7-dimethoxy-1H-indol-3- | +++ |
| | | L247V/A264G | yl)propanoic acid | |
| 297 | chPheRS | F221C/T224G/A264G | (S)-2-Amino-3-(4-iodophenyl)propanoic acid | 43 |
| 298 | chPheRS | F221I/T224G/A264G | (S)-2-Amino-3-(4-azidophenyl)propanoic acid | 88 |
| 299 | chPheRS | F221V/T224G/A264G | (S)-2-Amino-3-(benzo[b]thiophen-3- | 84 |
| | | | yl)propanoic acid | |
| 300 | chPheRS | F221V/T224G/A264G | (S)-2-Amino-3-(7-methyl-1H-indol-3- | 69 |
| | | | yl)propanoic acid | |
| 301 | chPheRS | F221V/T224G/A264G | (S)-2-Amino-3-(7-chloro-1H-indol-3- | 71 |
| | | | yl)propanoic acid | |
| 302 | chPheRS | F221V/T224G/A264G | (S)-2-Amino-3-(7-cyano-1H-indol-3- | 69 |
| | | | yl)propanoic acid | |
| 303 | chPheRS | F221V/T224G/A264G | (S)-2-Amino-3-(7-methoxy-1H-indol-3- | +++ |
| | | | yl)propanoic acid | |
| 304 | chPheRS | F221V/Q113G/L247A/E148D/ | (S)-2-Amino-3-(4-(4-cyclopentylbut-1-yn-1- | ++ |
| | | T224G/A264G | yl)phenyl)propanoic acid | |
| 305 | chPheRS | T224G/A264G | (S)-3-(4-Acetylphenyl)-2-aminopropanoic acid | 92 |
| 306 | chPheRS | T224G/A264G | (S)-2-Amino-3-(3-cyanophenyl)propanoic acid | 75 |
| 307 | chPheRS | T224G/A264G | (S)-2-Amino-3-(naphthalen-2-yl)propanoic acid | 66 |
| 308 | chPheRS | T224G/A264G | (S)-2-Amino-3-(4-methoxyphenyl)propanoic | 68 |
| | | | acid | |
| 309 | chPheRS | Q113F/E148D/V150C/T224G/ | 1-Methyl-L-tryptophan | 63 |
| | | A264G | | |
| 310 | chPheRS | Q113G/L247A/E148D/T224G/ | (S)-2-Amino-3-(4-(hex-1-yn-1- | +++ |
| | | A264G | yl)phenyl)propanoic acid | |
| 311 | chPheRS | Q113G/L247A/E148D/T224G/ | (S)-2-Amino-3-(4-(hept-1-yn-1- | +++ |
| | | A264G | yl)phenyl)propanoic acid | |
| 312 | chPheRS | Q113G/L247A/E148D/T224G/ | (S)-2-Amino-3-(4-(oct-1-yn-1- | ++ |
| | | A264G | yl)phenyl)propanoic acid | |
| 313 | chPheRS | Q113G/L247A/E148D/T224G/ | (S)-2-Amino-3-(4-(non-1-yn-1- | + |
| | | A264G | yl)phenyl)propanoic acid | |
| 314 | chPheRS | Q113G/L247A/E148D/T224G/ | (S)-2-Amino-3-(4-(hex-1-en-1- | ++ |
| | | A264G | yl)phenyl)propanoic acid | |
| 315 | chPheRS | Q113G/L247A/E148D/T224G/ | (S)-2-Amino-3-(4-(hex-1-en-1- | +++ |
| | | A264G | yl)phenyl)propanoic acid | |
| 316 | chPheRS | Q113G/L247A/E148D/T224G/ | (S)-2-Amino-3-(4-(5-bromopent-1-yn-1- | ++ |
| | | A264G | yl)phenyl)propanoic acid | |
| 317 | chPheRS | Q113G/L247A/E148D/T224G/ | (S)-2-Amino-3-(4-(7-bromohept-1-yn-1- | + |
| | | A264G | yl)phenyl)propanoic acid | |
| 318 | chPheRS | Q113G/L247A/E148D/T224G/ | (S)-2-Amino-3-(4-(3-phenylprop-1-yn-1- | +++ |
| | | A264G | yl)phenyl)propanoic acid | |
| 3 | chPheRS | Q113G/L247A/E148D/T224G/ | (S)-2-Amino-3-(4-(4-phenylbut-1-yn-1- | ++ |
| | | A264G | yl)phenyl)propanoic acid | |
| 320 | chPheRS | Q113G/L247A/E148D/T224G/ | (S)-2-Amino-3-(4-(5-phenylpent-1-yn-1- | ++ |
| | | A264G | yl)phenyl)propanoic acid | |
| 321 | chPheRS | Q113G/L247A/E148D/T224G/ | (S)-2-Amino-3-(4-(4-(naphthalen-2-yl)but-1- | ++ |
| | | A264G | en-1-yl)phenyl)propanoic acid | |
| 322 | chPheRS | Q113G/L247A/E148D/T224G/ | (S)-2-Amino-3-(4-(4-(2,3-dihydro-1H-inden-5- | ++ |
| | | A264G | yl)but-1-en-1-yl)phenyl)propanoic acid | |
| 323 | chPheRS | Q113N/T224S/A264S | (S)-2-Amino-3-(3,4- | 48 |
| | | | dihydroxyphenyl)propanoic acid | |
| 324 | chPheRS | V150G/F221C/T224G/A264G | (S)-2-Amino-3-(3-ethynyl-4- | ++ |
| | | | ((fluorosulfonyl)oxy)phenyl)propanoic acid | |
| 325 | chPheRS | V150G/F221V/Q113F/E148D/ | 1-Vinyl-L-tryptophan | 59 |
| | | L247L/T224G/A264G | | |
| 326 | chPheRS | V150G/F221V/Q113F/E148D/ | 1-(Prop-2-yn-1-yl)-L-tryptophan | ++ |
| | | L247L/T224G/A264G | | |
| 327 | chPheRS | E148D/T224G/A264G | (S)-2-Amino-3-(6-methoxy-1H-indol-3- | 80 |
| | | | yl)propanoic acid | |
| 328 | chPheRS | T224G/A264G | (S)-2-Amino-3-(4-bromophenyl)propanoic acid | 51 |
| 329 | chPheRS | T224G/A264G | (S)-2-Amino-3-(3-iodophenyl)propanoic acid | 56 |
| 330 | chPheRS | T224G/A264G | (S)-2-Amino-3-(4-cyanophenyl)propanoic acid | 45 |
| 331 | chPheRS | T224G/A264G | (S)-2-Amino-3-(4-iodophenyl)propanoic acid | 43 |
| 332 | chPheRS | F221I/T224G/A264G | (S)-2-Amino-3-(4-methoxyphenyl)propanoic | 73 |
| | | | acid | |
| 333 | chPheRS | V150G/F221V/Q113F/E148D/ | 1-Methyl-L-tryptophan | 63 |
| | | T224G/A264G | | |
| 334 | EcTyrRS | H70A/D158T | (S)-2-Amino-3-(4-hydroxy-3- | +++ |
| | | | iodophenyl)propanoic acid | |
| 335 | EcTyrRS | L71V/D182G/ | (S)-2-Amino-3-(4-(sulfooxy)phenyl)propanoic | 85 |
| | | | acid | |
| 336 | EcTyrRS | L71V/W129F/D182G | (S)-2-Amino-3-(4-(sulfooxy)phenyl)propanoic | 85 |
| | | | acid | |
| 337 | EcTyrRS | Y37A/D182T/F183M | (S)-2-Amino-3-(4-(2- | 60 |
| | | | bromoethoxy)phenyl)propanoic acid | |
| 338 | EcTyrRS | Y37A/D182T/F183M | (S)-2-Amino-3-(4-(2- | ++ |
| | | | fluoroacetyl)phenyl)propanoic acid | |
| 339 | EcTyrRS | Y37G/D182G/L186A | (S)-2-Amino-3-(4-benzoylphenyl)propanoic | 68 |
| | | | acid | |
| 340 | EcTyrRS | Y37G/D182G/L186A | (S)-2-Amino-3-(4-(3- | ++ |
| | | | fluorobenzoyl)phenyl)propanoic acid | |
| 341 | EcTyrRS | Y37G/D182G/L186A | (S)-2-Amino-3-(4-(3- | ++ |
| | | | chlorobenzoyl)phenyl)propanoic acid | |
| 342 | EcTyrRS | Y37G/D182G/L186A | (S)-2-Amino-3-(4-(4- | ++ |
| | | | fluorobenzoyl)phenyl)propanoic acid | |
| 343 | EcTyrRS | Y37G/D182G/L186A | (S)-2-Amino-3-(4-(4- | ++ |
| | | | chlorobenzoyl)phenyl)propanoic acid | |
| 344 | EcTyrRS | Y37G/D182G/L186A | (S)-2-Amino-3-(4-(4- | ++ |
| | | | bromobenzoyl)phenyl)propanoic acid | |
| 345 | EcTyrRS | Y37G/D182G/L186A | (S)-2-Amino-3-(4-(4- | ++ |
| | | | (trifluoromethyl)benzoyl)phenyl)propanoic acid | |
| 346 | EcTyrRS | Y37G/D182G/L186A/I7F/ | (S)-2-Amino-3-(4-benzoylphenyl)propanoic | 74 |
| | | L71V/G180S | acid | |
| 347 | EcTyrRS | Y37G/L71H/D182G/ | (S)-2-Amino-4-(7-hydroxy-2-oxo-2H-chromen- | ++ |
| | | L186G/L56E/T76G/S120Y/ | 4-yl)butanoic acid | |
| | | A121H/F183I | | |
| 348 | EcTyrRS | Y37G/L71V/D182C/F183Y/ | (S)-2-Amino-3-(4-(2- | ++ |
| | | L186C | azidoethoxy)phenyl)propanoic acid | |
| 349 | EcTyrRS | Y37G/L71V/D182C/F183Y/ | (S)-2-Amino-3-(4-(2- | 84 |
| | | L186C | azidoacetamido)phenyl)propanoic acid | |
| 350 | EcTyrRS | Y37G/L71V/D182C/F183Y/ | (S)-2-Amino-3-(4-(3- | +++ |
| | | L186C | azidopropanamido)phenyl)propanoic acid | |
| 351 | EcTyrRS | Y37G/L71V/D182C/F183Y/ | (S)-2-Amino-3-(4-(pent-4- | +++ |
| | | L186C | ynamido)phenyl)propanoic acid | |
| 352 | EcTyrRS | Y37G/L71V/D182C/F183Y/ | (S)-2-Amino-3-(4-(but-3- | +++ |
| | | L186C | ynamido)phenyl)propanoic acid | |
| 353 | EcTyrRS | Y37G/L71V/D182C/F183Y/ | (S)-2-Amino-3-(4-(pent-4- | +++ |
| | | L186C | enamido)phenyl)propanoic acid | |
| 354 | EcTyrRS | Y37G/L71V/D182C/F183Y/ | (S)-2-Amino-3-(4-(but-3- | +++ |
| | | L186C | enamido)phenyl)propanoic acid | |
| 355 | EcTyrRS | Y37G/L71V/D182C/F183Y/ | (S)-2-Amino-3-(4-(2- | +++ |
| | | L186C | fluoroacetamido)phenyl)propanoic acid | |
| 356 | EcTyrRS | Y37G/L71V/D182C/F183Y/ | (S)-2-Amino-3-(4-(2- | +++ |
| | | L186C | chloroacetamido)phenyl)propanoic acid | |
| 357 | EcTyrRS | Y37H/L71V/D182G/L186M | (S)-2-Amino-3-(4- | ++ |
| | | | (carboxymethyl)phenyl)propanoic acid | |
| 358 | EcTyrRS | Y37I/D182G/F183M/L186A/ | (S)-2-Amino-3-(4-(2- | +++ |
| | | D265R | fluoroacetyl)phenyl)propanoic acid | |
| 359 | EcTyrRS | Y37I/D2G/F183M/L186A | (S)-3-(4-Acetylphenyl)-2-aminopropanoic acid | 78 |
| 360 | EcTyrRS | Y37L/D182S/F183M/L186A | (S)-2-Amino-3-(4-azidophenyl)propanoic acid | 79 |
| 361 | EcTyrRS | Y37L/Q5S | (S)-2-Amino-3-(3-amino-4- | 50 |
| | | | hydroxyphenyl)propanoic acid | |
| 362 | EcTyrRS | Y37S/D182S/F183A/L186E | (S)-2-Amino-3-(4-boronophenyl)propanoic acid | 66 |
| 363 | EcTyrRS | Y37S/D182S/F183M/L186A | (S)-2-Amino-3-(4-(prop-2-yn-1- | 78 |
| | | | yloxy)phenyl)propanoic acid | |
| 364 | EcTyrRS | Y37S/D182S/F183M/L186A | (S)-2-Amino-3-(4-(2- | ++ |
| | | | chloroethoxy)phenyl)propanoic acid | |
| 365 | EcTyrRS | Y37V/D182S/F183M | (S)-2-Amino-3-(4-methoxyphenyl)propanoic | 89 |
| | | | acid | |
| 366 | EcTyrRS | Y37V/D182S/F183M | (S)-2-Amino-3-(4-bromophenyl)propanoic acid | 63 |
| 367 | EcTyrRS | Y37V/D182S/F183M | (S)-2-Amino-3-(4-nitrophenyl)propanoic acid | +++ |
| 368 | EcTyrRS | Y37V/D182S/F183M | (S)-2-Amino-3-(4-cyanophenyl)propanoic acid | +++ |
| 369 | EcTyrRS | Y37V/D182S/F183M/L186C/ | (S)-2-Amino-3-(4-methoxyphenyl)propanoic | 84 |
| | | D165G | acid | |
| 370 | EcTyrRS | Y37V/D182S/F183Y | (S)-2-Amino-3-(4-iodophenyl)propanoic acid | 64 |
| 371 | EcTyrRS | Y37L/Q5S | (S)-2-Amino-3-(4-hydroxy-3- | 73 |
| | | | iodophenyl)propanoic acid | |
| 372 | EcTyrRS | L71V/D182G/ | (S)-2-Amino-3-(4- | 70 |
| | | | ((fluorosulfonyl)oxy)phenyl)propanoic acid | |
| 373 | EcTyrRS | L71V/W129F/D182G | (S)-2-Amino-3-(4- | 68 |
| | | | ((fluorosulfonyl)oxy)phenyl)propanoic acid | |
| 374 | EcLeuRS | Y499C/Y527G/H537T | (S)-2-Amino-5-(4-methoxy-7-nitroindolin-1- | 43 |
| | | | yl)-5-oxopentanoic acid | |
| 375 | EcLeuRS | M40I/Y499I/Y527A/H529G/ | (S)-2-Amino-5-(3-(2- | ++ |
| | | T252A | nitrobenzyl)ureido)pentanoic acid | |
| 376 | EcLeuRS | M40I/Y499I/Y527A/H529G/ | (S)-2-Aminooctanoic acid | ++ |
| | | T252A | | |
| 377 | EcLeuRS | M40I/Y499I/Y527A/H529G/ | S-(4-Oxopentyl)-L-cysteine | ++ |
| | | T252A | | |
| 378 | EcLeuRS | M40I/Y499I/Y527A/H529G/ | S-(Hex-5-en-1-yl)-L-cysteine | ++ |
| | | T252A | | |
| 379 | EcLeuRS | M40I/Y499I/Y527A/H529G/ | S-Hexyl-L-cysteine | ++ |
| | | T252A | | |
| 380 | EcLeuRS | M40V/L41S/Y499S/Y527L/ | S-Butyl-L-cysteine | ++ |
| | | H529G/T252R/E20K | | |
| 381 | EcLeuRS | M40I/Y499I/Y527A/H529G/ | S-Pentyl-L-cysteine | 83 |
| | | T252A | | |
| 382 | EcLeuRS | M40I/Y499I/Y527A/H529G/ | S-Hexyl-L-cysteine | 87 |
| | | T252A | | |
| 383 | EcLeuRS | M40I/Y499I/Y527A/H529G/ | S-Heptyl-L-cysteine | 74 |
| | | T252A | | |
| 384 | EcLeuRS | M40I/Y499I/Y527A/H529G/ | S-Octyl-L-cysteine | ++ |
| | | T252A | | |
| 385 | EcLeuRS | M40I/Y499I/Y527A/H529G/ | S-(Pent-4-yn-1-yl)-L-cysteine | ++ |
| | | T252A | | |
| 386 | EcLeuRS | M40I/Y499I/Y527A/H529G/ | S-(Hex-5-yn-1-yl)-L-cysteine | 77 |
| | | T252A | | |
| 387 | EcLeuRS | M40I/Y499I/Y527A/H529G/ | S-(3-Azidopropyl)-L-cysteine | ++ |
| | | T252A | | |
| 388 | EcLeuRS | M40I/Y499I/Y527A/H529G/ | S-(4-Azidobutyl)-L-cysteine | ++ |
| | | T252A | | |
| 389 | EcLeuRS | M40I/Y499I/Y527A/H529G/ | S-(5-Azidopentyl)-L-cysteine | ++ |
| | | T252A | | |
| 390 | EcLeuRS | M40A/L41N/Y499I/Y527G/ | (S)-2-Amino-3-(((5- | 73 |
| | | H537T | (dimethylamino)naphthalen-1- | |
| | | | yl)sulfonylamino)propanoic acid | |
| 391 | EcLeuRS | L38F/M40G/L41P/Y499V/ | (S)-3-(((6-Acetylnaphthalen-2-yl)amino)-2- | ++ |
| | | Y500L/Y527A/H537G/L538S/ | aminopropanoic acid | |
| | | F531C/A560V | | |
| 392 | EcLeuRS | M40G/L41P/Y499G/Y527A/ | (S)-2-Amino-3-(naphthalen-2- | ++ |
| | | H537T | ylamino)propanoic acid | |
| 393 | EcLeuRS | M40I/Y499I/Y527A/H537G | (S)-2-Amino-6-(ethylthio)hexanoic acid | ++ |
| 394 | EcLeuRS | M40I/Y499I/Y527A/H537G | (S)-2-Amino-5-mercaptopentanoic acid | ++ |
| 395 | EcLeuRS | M40I/Y499I/Y527A/H537G | (S)-2-Amino-6-mercaptohexanoic acid | ++ |
| 396 | EcLeuRS | M40I/Y499I/Y527A/H537G | (S)-2-Aminooctanoic acid | 88 |
| 397 | EcLeuRS | M40I/Y499I/Y527A/H537G | (S)-2-Aminononanoic acid | 92 |
| 398 | EcLeuRS | M40I/Y499I/Y527A/H537G | (S)-2-Aminodecanoic acid | 85 |
| 399 | EcLeuRS | M40V/L41M/Y499L/Y527L/ | (S)-2-Aminooctanoic acid | ++ |
| | | H537G | | |
| 400 | EcLeuRS | M40L/L41E/Y499R/Y527A/ | (S)-2-Amino-3-(4-methoxyphenyl)propanoic | 92 |
| | | H537G | acid | |
| 401 | EcLeuRS | M40W/L41S/Y499I/Y527A/ | S-(2-Nitrobenzyl)-L-cysteine | +++ |
| | | H537G | | |
| 402 | EcLeuRS | M40G/L41Q/Y499L/Y527G/ | S-(4,5-Diethoxy-2-nitrobenzyl)-L-serine | +++ |
| | | H537F | | |
| 403 | EcLeuRS | M40G/L41Q/Y499L/Y527G/ | S-(4,5-Diethoxy-2-nitrobenzyl)-L-cysteine | 84 |
| | | H537F | | |
| 404 | EcLeuRS | M40G/L41Q/Y499L/Y527G/ | (S)-2-Amino-3-(((4,5-diethoxy-2- | 59 |
| | | H537F | nitrobenzyl)selanyl)propanoic acid | |
| 405 | EcLeuRS | E20K/M40V/L41S/T252R/ | (Allylsulfinyl)-D-alanine | +++ |
| | | Y499S/Y527L/H537G | | |
| 406 | EcLeuRS | E20K/M40V/L41S/T252R/ | (S)-2-Aminohept-6-enoic acid | +++ |
| | | Y499S/Y527L/H537G | | |
| 407 | EcLeuRS | E20K/M40V/L41S/T252R/ | (S)-2-Aminooct-7-enoic acid | 81 |
| | | Y499S/Y527L/H537G | | |
| 408 | EcLeuRS | E20K/M40V/L41S/T252R/ | (R)-O-(But-2-en-1-yl)-L-serine | +++ |
| | | Y499S/Y527L/H537G | | |
| 409 | EcLeuRS | E20K/M40V/L41S/T252R/ | O-(Pent-4-en-1-yl)-L-serine | +++ |
| | | Y499S/Y527L/H537G | | |
| 410 | | M40G/Y499A/Y527V/H537G | N6-Acetyl-N6-methyl-L-lysine | 83 |
| 411 | EcTrpRS | S8A/V144G/V146C | (S)-2-Amino-3-(5-hydroxy-1H-indol-3-yl) | 75 |
| | | | propanoic acid | |
| 412 | EcTrpRS | S8A/V144G/V146C | (S)-2-Amino-3-(5-methyl-1H-indol-3-yl) | 37 |
| | | | propanoic acid | |
| 413 | EcTrpRS | S8A/V144G/V146C | (S)-2-Amino-3-(5-methoxy-1H-indol-3- | 85 |
| | | | yl)propanoic acid | |
| 414 | EcTrpRS | S8A/V144G/V146C | (S)-2-Amino-3-(5-bromo-1H-indol-3- | + |
| | | | yl)propanoic acid | |
| 415 | EcTrpRS | S8A/V144G/V146C | (S)-2-Amino-3-(5-(prop-2-yn-1-yloxy)-1H- | + |
| | | | indol-3-yl)propanoic acid | |
| 416 | EcTrpRS | S8A/V144G/V146C | (S)-2-Amino-3-(5-azido-1H-indol-3- | + |
| | | | yl)propanoic acid | |
| 417 | EcTrpRS | S8A/V144S/V146A | (S)-2-Amino-3-(5-amino-1H-indol-3- | + |
| | | | yl)propanoic acid | |
| 418 | EcTrpRS | Q109P | (S)-2-Amino-3-(4-fluoro-1H-indol-3- | 82 |
| | | | yl)propanoic acid | |

For mutations in pyrrolysyl-tRNA synthetases, a particular mutation in one type of pyrrolysyl-tRNA synthetase may have corresponding mutations in other types, achieving the same effect. The corresponding mutations and their specific relationships are as follows.

For example, for TetBu, the corresponding mutations in MmPylRS can be L305G/N346G/C348A, in MbPylRS L270G/N311G/C313A, in chPylRS L270G/N311G/C313A, in MaPylRS L125G/N166GNV168A, in G1PylRS L124G/N165GNV167A, in 1R26PylRS L125G/N166GNV168A, in LumPylRS L125G/N166GNV168A, in N/tPylRS L128G/N168GNV170A, and in DebPylRS L126G/N168GNV170A. These mutations enable the corresponding aminoacyl-tRNA synthetases to efficiently bind TetBu.

The specific corresponding mutations and their relationships are shown in Table 2 below.

“Aminoacyl-tRNA synthetase” represents the reference aminoacyl-tRNA synthetase, “reference mutation” represents the specific mutation type in the reference aminoacyl-tRNA synthetase, and the corresponding mutations in MmPylRS, MbPylRS, chPylRS, MaPylRS, G1PylRS, R26PylRS, LumPylRS, NitPylRS, and DebPylRS represent the mutations corresponding to the reference mutation in each respective enzyme. After undergoing the corresponding mutations, these enzymes can have the same or similar effects as the reference aminoacyl-tRNA synthetase after the reference mutation.

TABLE 2
The mutations in various type of PylRS.

| AaRS | Reference mutation | MbPylRS mutation | chPylRS mutation | MaPylRS mutation |
|---|---|---|---|---|
| MmPylRS | Y306M/L309A/C348A/ | Y271M/L274A/C313A/ | Y271M/L274A/C313A/ | Y126M/M129A/V168A/ |
| | Y384F | Y349F | Y349F | Y206F |
| MmPylRS | A302I/N346T/C348I/ | A267I/N311T/C313I/ | A267I/N311T/C313I/ | A122I/N166T/V168I/ |
| | Y384L/W417K | Y349L/W382K | Y349L/W382K | Y206L/W239K |
| MmPylRS | A302T/L309A/I322T/ | A267T/L27A/I287T// | A267T/L27A/I287T// | A122T/M129A/N166A/ |
| | N346A/C348G | N311A/C313G | N311A/C313G | V168G |
| MmPylRS | A302T/L309S/N346V/ | A267T/L27S/N311V/ | A267T/L27S/N311V/ | A122T/M129S/N166V/ |
| | C348G | C313G | C313G | V168G |
| MmPylRS | A302T/N346A/C348A/ | A267T/N311A/C313A/ | A267T/N311A/C313A/ | A122T/N166A/V168A/ |
| | V401L/W417A | V366L/W382A | V366L/W382A | A223L/W239A |
| MmPylRS | A302T/N346A/C348G/ | A267T/N311A/C313G/ | A267T/N311A/C313G/ | A122T/N166A/V168G/ |
| | Y384F/W417T | Y349F/W382T | Y349F/W382T | Y206F/W239T |
| MmPylRS | A302T/N346A/C348V/ | A267T/N311A/C313V/ | A267T/N311A/C313V/ | A122T/N166A/Y206F/ |
| | Y384F/W417T | Y349F/W382T | Y349F/W382T | W239T |
| MmPylRS | A302T/N346G/C348T/ | A267T/N311G/C313T/ | A267T/N311G/C313T/ | A122T/N166G/V168T/ |
| | V401I/W417Y | V366I/W382Y | V366I/W382Y | A223I/W239Y |
| MmPylRS | A302T/N346T/C348T | A267T/N311T/C313T | A267T/N311T/C313T | A122T/N166T/V168T |
| MmPylRS | A302T/N346V/C348W/ | A267T/N311V/C313W/ | A267T/N311V/C313W/ | A122T/N166V/V168W/ |
| | Y384F/V401L | Y349F/V366L | Y349F/V366L | Y206F/A223L |
| MmPylRS | C348W/W417S | C313W/W382S | C313W/W382S | V168W/W239S |
| MmPylRS | L105M/Y306L/L309S/ | Y271L/L274S/N311S/ | Y271L/L274S/N311S/ | Y126L/M129S/N166S/ |
| | N346S/C348M/ | C313M | C313M | V168M |
| MmPylRS | L301M/L305I/Y306L/ | L266M/L270I/Y271L/ | L266M/L270I/Y271L/ | L121M/L125I/Y126L/ |
| | L309A/C348F | L274A/C313F | L274A/C313F | M129A/V168F |
| MmPylRS | L301M/Y306A/L309A/ | L266M/Y271A/L274A/ | L266M/Y271A/L274A/ | L121M/Y126A/M129A/ |
| | C348F | C313F | C313F | V168F |
| MmPylRS | L301M/Y306L/L309A/ | L266M/Y271L/L274A/ | L266M/Y271L/L274A/ | L121M/Y126L/M129A/ |
| | C348F | C313F | C313F | V168F |
| MmPylRS | L305G/N346G/C348A | L270G/N311G/C313A | L270G/N311G/C313A | L125G/N166G/V168A |
| MmPylRS | L305M/I322T/N346G | L270M/I287T/N311G | L270M/I287T/N311G | L125M/1142T/N166G |
| MmPylRS | L309A/C348A | L274A/C313A | L274A/C313A | M129A/V168A |
| MmPylRS | L309G/N346A/C348I/ | L274G/N311A/C313I/ | L274G/N311A/C313I/ | M129G/N166A/V168I/ |
| | V401K/W417I | V366K/W382I | V366K/W382I | A223K/W239I |
| MmPylRS | L309T/C348G/Y384F | L274T/C313G/Y349F | L274T/C313G/Y349F | M129T/V168G/Y206F |
| MmPylRS | N346A/C348A | N311A/C313A | N311A/C313A | N166A/V168A |
| MmPylRS | N346G/C348G/Y384F | N311G/C313G/Y349F | N311G/C313G/Y349F | N166G/V168G/Y206F |
| MmPylRS | N346G/C348Q | N311G/C313Q | N311G/C313Q | N166G/V168Q |
| MmPylRS | N346G/C348Q/V401G | N311G/C313Q/V366G | N311G/C313QV366G | N166G/V168Q/A223G |
| MmPylRS | N346Q/C348A/V401M | N311Q/C313A/V366M | N311Q/C313A/V366M | N166Q/V168A/A223M |
| MmPylRS | N346Q/C348S/V401G/ | N311Q/C313S/V366G/ | N311Q/C313S/V366G/ | N166Q/V168S/A223G/ |
| | W417T | W382T | W382T | W239T |
| MmPylRS | Y306A/L309A/C348S/ | Y271A/L274A/C313S/ | Y271A/L274A/C313S/ | Y126A/M129A/V168S/ |
| | Y384F | Y349F | Y349F | Y206F |
| MmPylRS | Y306A/L309M/C348G/ | Y271A/L274M/C313G/ | Y271A/L274M/C313G/ | Y126A/M129M/V168G/ |
| | Y384F/I405R | Y349F/V370R | Y349F/1370R | Y206F/H227R |
| MmPylRS | Y306A/Y384F | Y271A/Y349F | Y271A/Y349F | Y126A/Y206F |
| MmPylRS | Y306A/Y384F | Y271A/Y349F | Y271A/Y349F | Y126A/Y206F |
| MmPylRS | Y306A/Y384F/I413L | Y271A/Y349F/I378L | Y271A/Y349F/I378L | Y126A/Y206F/V235L |
| MmPylRS | Y306G/Y384F | Y271G/Y349F | Y271G/Y349F | Y126G/Y206F |
| MmPylRS | Y306G/Y384F | Y271A/Y349F | Y271A/Y349F | Y126A/Y206F |
| MmPylRS | Y306L/L309A/N346A/ | Y271L/L274A/N311A/ | Y271L/L274A/N311A/ | Y126L/M129A/N166A/ |
| | C348M/W417T | C313M/W382T | C313M/W382T | V168M/W239T |
| MmPylRS | Y306V/L309A/C348F/ | Y271V/L274A/C313F/ | Y271V/L274A/C313F/ | Y126V/M129A/V168F/ |
| | Y384F | Y349F | Y349F | Y206F |
| MmPylRS | Y306V/L309A/C348F/ | Y271V/L274A/C313F/ | Y271V/L274A/C313F/ | Y126V/M129A/V168F/ |
| | Y384F | Y349F | Y349F | Y206F |
| MmPylRS | Y384F | Y349F | Y349F | Y206F |
| MmPylRS | Y384F | Y349F | Y349F | Y206F |
| MbPylRS | L270I/Y271F/L274G/ | L270I/Y271F/L274G/ | L270I/Y271F/L274G/ | L125I/Y126F/M129G/ |
| | C313F/Y349F | C313F/Y349F | C313F/Y349F | V168F/Y206F |
| MbPylRS | N311C/C313S/Y349F | N311C/C313S/Y349F | N311C/C313S/Y349F | N166C/V168S/Y206F |
| MbPylRS | A267D/N311G/C313G | A267D/N311G/C313G | A267D/N311G/C313G | A122D/N166G/V168G |
| MbPylRS | A267Q/N311S/C313W | A267Q/N311S/C313W | A267Q/N311S/C313W | A122Q/N166S/V168W |
| MbPylRS | A267S/C313V/M315F/ | A267S/C313V/M315F/ | A267S/C313V/M315F/ | A122S/M170F/E201G |
| | d344g | D344G | D344G | |
| MbPylRS | A267Y/Y271A/N311T/ | A267Y/Y271A/N311T/ | A267Y/Y271A/N311T/ | A122Y/Y126A/N166T/ |
| | C313G/Y349F | C313G/Y349F | C313G/Y349F | V168G/Y206F |
| MbPylRS | C313S/Y349F | C313S/Y349F | C313S/Y349F | V168S/Y206F |
| MbPylRS | C313T | C313T | C313T | V168T |
| MbPylRS | C313T/Y349F | C313T/Y349F | C313T/Y349F | V168T/Y206F |
| MbPylRS | C313T/Y349F | C313T/Y349F | C313T/Y349F | V168T/Y206F |
| MbPylRS | C313V | C313V | C313V | V168V |
| MbPylRS | C313V/M315L/V370K/ | C313V/M315L/V370K/ | C313V/M315L/V370K/ | M170L/H227K |
| | I378V | I378V | I378V | |
| MbPylRS | C313W/W382T | C313W/W382T | C313W/W382T | V168W/W239T |
| MbPylRS | D76G/L266M/L270I/ | L266M/L270I/Y271F/ | L266M/L270I/Y271F/ | L121M/L125I/Y126F/ |
| | Y271F/L274A/C313F | L274A/C313F | L274A/C313F | M129A/V168F |
| MbPylRS | D76G/L266V/L270I/ | L266V/L270I/Y271F/ | L266V/L270I/Y271F/ | L121V/L125I/Y126F/ |
| | Y271F/L274A/C313F | L274A/C313F | L274A/C313F | M129A/V168F |
| MbPylRS | D76S/L274A/C313F/ | L274A/C313F/Y349F | L274A/C313F/Y349F | M129A/V168F/Y206F |
| | Y349F | | | |
| MbPylRS | I287V/N311S/C313G/ | I287V/N311S/C313G/ | I287V/N311S/C313G/ | N166S/V168G/A223H/ |
| | V366H/W382V | V366H/W382V | V366H/W382V | W239V |
| MbPylRS | L266M/L270I/L274A/ | L266M/L270I/L274A/ | L266M/L270I/L274A/ | L121M/L125I/M129A/ |
| | C313F. | C313F. | C313F. | V168F |
| MbPylRS | L266M/L270I/Y271F/ | L266M/L270I/Y271F/ | L266M/L270I/Y271F/ | L121M/L125I/Y126F/ |
| | L274A/C313F | L274A/C313F | L274A/C313F | M129A/V168F |
| MbPylRS | L266M/L270I/Y271L/ | L266M/L270I/Y271L/ | L266M/L270I/Y271L/ | L121M/L125I/Y126L/ |
| | L274A | L274A | L274A | M129A |
| MbPylRS | L266M/L270I/Y271L/ | L266M/L270I/Y271L/ | L266M/L270I/Y271L/ | L121M/L125I/Y126L/ |
| | L274A/C313I | L274A/C313I | L274A/C313I | M129A/V168I |
| MbPylRS | L270F/L274M/N311G/ | L270F/L274M/N311G/ | L270F/L274M/N311G/ | L125F/M129M/N166G/ |
| | C313G/Y349F | C313G/Y349F | C313G/Y349F | V168G/Y206F |
| MbPylRS | L270F/L274M/N311A/ | L270F/L274M/N311A/ | L270F/L274M/N311A/ | L125F/M129M/N166A/ |
| | C313G | C313G | C313G | V168G |
| MbPylRS | L270F/L274M/N311G/ | L270F/L274M/N311A/ | L270F/L274M/N311A/ | L125F/M129M/N166G/ |
| | C313G | C313G | C313G | V168G |
| MbPylRS | L270F/L274M/N311G/ | L270F/L274M/N311A/ | L270F/L274M/N311A/ | L125F/M129M/N166G/ |
| | C313G/Y349F | C313G | C313G | V168G |
| MbPylRS | L270I/Y271A/Y349F | L270I/Y271A/Y349F | L270I/Y271A/Y349F | L125I/Y126A/Y206F |
| MbPylRS | L274A/C313F/Y349W | L274A/C313F/Y349W | L274A/C313F/Y349W | M129A/V168F/Y206W |
| MbPylRS | L274A/C313S | L274A/C313S | L274A/C313S | Y129A/V168S |
| MbPylRS | L274A/C313S | L274A/C313S | L274A/C313S | M129A/V168S |
| MbPylRS | L274A/C313S/Y349F | L274A/C313S/Y349F | L274A/C313S/Y349F | Y129A/V168S/Y206F |
| MbPylRS | L274A/C313S/Y349F | L274A/C313F/Y349F | L274A/C313F/Y349F | M129A/V168F/Y206F |
| MbPylRS | L274A/C313S/Y349F | L274A/C313S/Y349F | L274A/C313S/Y349F | M129A/V168S/Y206F |
| MbPylRS | L274A/C313S/Y349F | L274A/C313S/Y349F | L274A/C313S/Y349F | M129A/V168S/Y206F |
| MbPylRS | L274A/C313S/Y349F | L274A/C313S/Y349F | L274A/C313S/Y349F | Y129A/V168S/Y206F |
| MbPylRS | L274A/C313T/Y349W | L274A/C313T/Y349W | L274A/C313T/Y349W | M129A/V168T/Y206W |
| MbPylRS | L274A/C313V/Y349F | L274A/C313V/Y349F | L274A/C313V/Y349F | M129A/V168V/Y206F |
| MbPylRS | L274A/N311Q/C313S | L274A/N311Q/C313S | L274A/N311Q/C313S | M129A/N166Q/V168S |
| MbPylRS | L274C/C313V/I378V | L274C/C313V/I378V | L274C/C313V/I378V | M129C |
| MbPylRS | L274G/C313V/M315A/ | L274G/C313V/M315A/ | L274G/C313V/M315A/ | M129G/M170A/H227R |
| | V370R/I378V | V370R/I378V | I370R/I378V | |
| MbPylRS | L274G/C313V/Y349F | L274G/C313V/Y349F | L274G/C313V/Y349F | M129G/V168V/Y194F |
| MbPylRS | M241/A267/Y271M/ | M241/A267/Y271M/ | M241/A267/Y271M/ | M96/A122/Y126M/ |
| | L274G/C313A/Y349W | L274G/C313A/Y349W | L274G/C313A/Y349W | M129G/V168A/Y206W |
| MbPylRS | Y271A/Y349F | Y271A/Y349F | Y271A/Y349F | Y126A/Y206F |
| MbPylRS | M241A/Y271A/L274V/ | M241A/Y271A/L274V/ | M241/Y271A/L274V/ | M96A/Y126A/M129V/ |
| | C313V/M315Y/Y349F/ | C313V/M315Y/Y349F/ | C313V/M315Y/Y349F/ | M170Y/Y206F/H227R |
| | V370R | V370R | I370R | |
| MbPylRS | M241F/A267S/Y271C/ | M241F/A267S/Y271C/ | M241F/A267S/Y271C/ | M96F/A122S/Y126C/ |
| | L274M | L274M | L274M | M129M |
| MbPylRS | M241F/A267S/Y271C/ | M241F/A267S/Y271C/ | M241F/A267S/Y271C/ | M95F/A122S/Y126C/ |
| | L274M | L274M | L274M | M129M |
| MbPylRS | N311A/C313M/v366g/ | N311A/C313M/V366G/ | N311A/C313M/V366G/ | N166A/V168M/A223G/ |
| | W382T | W382T | W382T | W239T |
| MbPylRS | N311A/C313M/v366g/ | N311A/C313M/V366G/ | N311A/C313M/V366G/ | N166A/V168M/A223G/ |
| | W382T | W382T | W382T | W239T |
| MbPylRS | N311G/C313A | N311G/C313A | N311G/C313A | N166G/V168A |
| MbPylRS | N311G/C313V/V366K | N311G/C313V/V366K | N311G/C313V/V366K | N166G/V168V/A223K |
| MbPylRS | N311M/C313Q/V366G/ | N311M/C313Q/V366G/ | N311M/C313Q/V366G/ | N166M/V168Q/A223G/ |
| | W382N | W382N | W382N | W239N |
| MbPylRS | N311Q/Y349F | N311Q/Y349F | N311Q/Y349F | N166Q/Y206F |
| MbPylRS | N311Q/C313A/V366M | N311Q/C313A/V366M | N311Q/C313A/V366M | N166Q/V168A/A223M |
| MbPylRS | N311S/C313A/V366H/ | N311S/C313A/V366H/ | N311S/C313A/V366H/ | N166S/V168A/A223H/ |
| | W382I | W382I | W382I | W239I |
| MbPylRS | N311S/C313A/Y349F | N311S/C313A/Y349F | N311S/C313A/Y349F | N166S/V168A/Y206F |
| MbPylRS | N311S/C313G/V366A/ | N311S/C313G/V366A/ | N311S/C313G/V366A/ | N166S/V168G/A223A/ |
| | W382T/ | W382T/ | W382T/ | W239T |
| MbPylRS | N311S/C313G/Y349F | N311S/C313G/Y349F | N311S/C313G/Y349F | N166S/V168G/Y206F |
| MbPylRS | R61K/G130E/Y349F | Y349F | Y349F | Y206F |
| MbPylRS | Y271A/C313V/V370L/ | Y271A/C313V/V370L/ | Y271A/C313V/I370L/ | Y126A/H227L |
| | I378V | I378V | 1378V | |
| MbPylRS | Y271A/L274A/C313V/ | Y271A/L274A/C313V/ | Y271A/L274A/C313V/ | Y126A/M129A/M170Y/ |
| | M315Y/V370R/I378V | M315Y/V370R/I378V | M315Y/I370R/I378V | H227R |
| MbPylRS | Y271A/L274M | Y271A/L274M | Y271A/L274M | Y126A/M129M |
| MbPylRS | Y271A/L274V/C313V/ | Y271A/L274V/C313V/ | Y271A/L274V/C313V/ | Y126A/M129V/M170Y/ |
| | M315Y/V370R | M315Y/V370R | M315Y/I370R | H227R |
| MbPylRS | Y271A/L274V/C313V/ | Y271A/L274V/C313V/ | Y271A/L274V/C313V/ | Y126A/M129V/M170Y/ |
| | M315Y/Y349F/V370R | M315Y/Y349F/V370R | M315Y/Y349F/I370R | Y206F/H227R |
| MbPylRS | Y271A/Y349F | Y271A/Y349F | Y271A/Y349F | Y126A/Y206F |
| MbPylRS | Y271C/N311Q/Y349F/ | Y271C/N311Q/Y349F/ | Y271C/N311Q/Y349F/ | Y126C/N166Q/Y206F/ |
| | V366C | V366C | V366C | A223C |
| MbPylRS | Y271F/C313T | Y271F/C313T | Y271F/C313T | Y126F/V168T |
| MbPylRS | Y271G/C313V | Y271G/C313V | Y271G/C313V | Y126G/V168V |
| MbPylRS | Y271G/C313V/V370R | Y271G/C313V/V370R | Y271G/C313V/I370R | Y126G/V168V/H227R |
| MbPylRS | Y271G/L274V/C313V/ | Y271G/L274V/C313V/ | Y271G/L274V/C313V/ | Y126G/M129V/M170Y/ |
| | M315Y/V370R | M315Y/V370R | M315Y/1370R | H227R |
| MbPylRS | Y271I/L274M/C313A | Y271I/L274M/C313A | Y271I/L274M/C313A | Y126I/M129M/V168A |
| MbPylRS | Y271M/L274A/C313A | Y271M/L274A/C313A | Y271M/L274A/C313A | Y126M/M129A/V168A |
| MbPylRS | Y271M/L274A/N311A/ | Y271M/L274A/N311A/ | Y271M/L274A/N311A/ | Y126M/M129A/N166A/ |
| | C313A/Y349F | C313A/Y349F | C313A/Y349F | V168A/Y206F |
| MbPylRS | Y271M/L274T/C313A/ | Y271M/L274T/C313A/ | Y271M/L274T/C313A/ | Y126M/M129T/V168A/ |
| | Y349F | Y349F | Y349F | Y206F |
| MbPylRS | Y349F | Y349F | Y349F | Y206F |
| MbPylRS | Y349W | Y349W | Y349W | Y206W |
| MaPylRS | L125F/N166A/V168G | L270F/N311A/C313G | L270F/N311A/C313G | L125F/N166A/V168G |
| MaPylRS | Y126A/H227I/Y228P | Y271A/C313V | Y271A/C313V | Y126A/H227I/Y228P |
| MaPylRS | Y126G/M129A/V168F/ | Y271G/L274A/C313F/ | Y271G/L274A/C313F/ | Y126G/M129A/V168F/ |
| | H227T/Y228P/L229I | V370T/S371P/L372I | I370T/P371P/L372I | H227T/Y228P/L229I |
| MaPylRS | Y126G/M129A/V168F/ | Y271G/L274A/C313F/ | Y271G/L274A/C313F/ | Y126G/M129A/V168F/ |
| | H227T/Y228P/L229I | V370T/S371P/L372I | I370T/P371P/L372I | H227T/Y228P/L229I |
| G1PylRS | L124M/Y125M/N165A/ | L270M/Y271M/N311A/ | L270M/Y271M/N311A/ | L125M/Y126M/N166A/ |
| | V167G/A221G/W237H | C313G/V366G/W382H | C313G/V366G/W382H | V168G/A223G/W239H |
| G1PylRS | L124A/Y125L/V167A/ | L270A/Y271L/C313A/ | L270A/Y271L/C313A/ | L125A/Y126L/V168A/ |
| | Y204W/A221S | Y349W/V366S | Y349W/V366S | Y206W/A223S |
| G1PylRS | L124A/Y125F/Y204W/ | L270A/Y271F/C313A/ | L270A/Y271F/C313A/ | L125A/Y126F/V168A/ |
| | A221S/W237Y | Y349W/V366S | Y349W/V366S | Y206W/A223S |
| G1PylRS | L124G/M128A/N165G/ | L270G/L274A/N311G/ | L270G/L274A/N311G/ | L125G/M129A/N166G/ |
| | V167A | C313A | C313A | V168A |
| G1PylRS | L124G/M128L/N165G/ | L270G/L274L/N311G/ | L270G/L274L/N311G/ | L125G/M129L/N166G/ |
| | V167A | C313A | C313A | V168A |
| G1PylRS | L124G/N165G/V167A/ | L270G/L274A/N311G/ | L270G/L274A/N311G/ | L125G/M129A/N166G/ |
| | M128A | C313A | C313A | V168A |
| G1PylRS | L124G/Y125F/N165G/ | L270G/Y271F/N311G/ | L270G/Y271F/N311G/ | L125G/Y126F/N166G/ |
| | V167F/Y204W/A221G/ | C313F/V366G/W382Y | C313F/V366G/W382Y | V168F/A223G/W239Y |
| | W237Y | | | |
| G1PylRS | N165G/V167G | N311G/C313G | N311G/C313G | N166G/V168G |
| chPylRS | C348G/V401C/Y384F | C313G/V366C/Y349F | C313G/V366C/Y349F | V168G/A223C/Y206F |
| chPylRS | L270I/L274G/N311C/ | L270I/L274G/N311C/ | L270I/L274G/N311C/ | L125I/M129G/N166C/ |
| | C313W/Y349F | C313W/Y349F | C313W/Y349F | V168W/Y206F |
| chPylRS | L270V/L274G/N311C/ | L305V/L309G/N346C/ | L270V/L274G/N311C/ | L125V/M129G/N166C/ |
| | C313W/Y349F | C348W/Y384F | C313W/Y349F | V168W/Y206F |
| AaRS | G1PylRS mutation | 1R26PylR mutation | LumPylR mutation | NitPylRS mutation |
| MmPylRS | Y125M/M128A/V167A/ | Y126M/M129A/V168A/ | Y126M/M129A/V168A/ | Y129M/M132A/V170A/ |
| | Y204F | Y206F | Y205F | Y207F |
| MmPylRS | A121I/N165T/V167I/ | A122I/N166T/V168I/ | A122I/N166T/V168I/ | A125I/N168T/V170I/ |
| | Y204L/W237K | Y206L/W239K | Y205L/W238K | Y207L/W240K |
| MmPylRS | A121T/M128A/N165A/ | A122T/M129A/N166A/ | A122T/M129A/N166A/ | A125T/M132A/N168A/ |
| | V167G | V168G | V168G | V170G |
| MmPylRS | A121T/M128S/N165V/ | A122T/M129S/N166V/ | A122T/M129S/N166V/ | A125T/M132S/N168V/ |
| | V167G | V168G | V168G | V170G |
| MmPylRS | A121T/N165A/V167A/ | A122T/N166A/V168A/ | A122T/N166A/V168A/ | A125T/N168A/V170A/ |
| | A221L/W237A | A223L/W239A | A222L/W238A | A224L/W240A |
| MmPylRS | A121T/N165A/V167G/ | A122T/N166A/V168G/ | A122T/N166A/V168G/ | A125T/N168A/V170G/ |
| | Y204F/W237T | Y206F/W239T | Y205F/W238T | Y207F/W240T |
| MmPylRS | A121T/N165A//Y204F/ | A122T/N166A/Y206F/ | A122T/N166A/Y205F/ | A125T/N168A/Y207F/ |
| | W237T | W239T | W238T | W240T |
| MmPylRS | A121T/N165G/V167T/ | A122T/N166G/V168T/ | A122T/N166G/V168T/ | A125T/N168G/V170T/ |
| | A221I/W237Y | A223I/W239Y | A222I/W238Y | A224I/W240Y |
| MmPylRS | A121T/N165T/V167T | A122T/N166T/V168T | A122T/N166T/V168T | N125T/N168T/V170T |
| MmPylRS | A121T/N165V/V167W/ | A122T/N166V/V168W/ | A122T/N166V/V168W/ | A125T/N168V/V170W/ |
| | Y204F/A221L | Y206F/A223L | Y205F/A222L | Y207F/A224L |
| MmPylRS | V167W/W237S | V168W/W239S | V168W/W238S | V170W/W240S |
| MmPylRS | Y125L/M128S/N165S/ | Y126L/M129S/N166S/ | Y126L/M129S/N166S/ | Y129L/M132S/N168S/ |
| | V167M | V168M | V168M | V170M |
| MmPylRS | H120M/L124I/Y125L/ | L121M/L125I/Y126L/ | L121M/L125I/Y126L/ | L124M/L128I/Y129L/ |
| | M128A/V167F | M129A/V168F | M129A/V168F | M132A/V170F |
| MmPylRS | H120M/Y125A/M128A/ | L121M/Y126A/M129A/ | L121M/Y126A/M129A/ | L124M/Y129A/M132A/ |
| | V167F | V168F | V168F | V170F |
| MmPylRS | H120M/Y125L/M128A/ | L121M/Y126L/M129A/ | L121M/Y126L/M129A/ | L124M/Y129L/M132A/ |
| | V167F | V168F | V168F | V170F |
| MmPylRS | L124G/N165G/V167A | L125G/N166G/V168A | L125G/N166G/V168A | L128G/N168G/V170A |
| MmPylRS | L124M/I141T/N165G | L125M/I142T/N166G | L125M/L142T/N166G | L128M/I144T/N168G |
| MmPylRS | M128A/V167A | M129A/V168A | M129A/V168A | M132A/V170A |
| MmPylRS | M128G/N165A/V167I/ | M129G/N166A/V168I/ | M129G/N166A/V168I/ | M132G/N168A/V170I/ |
| | A221K/W237I | A223K/W239I | A222K/W238I | A224K/W240I |
| MmPylRS | M128T/V167G/Y204F | M129T/V168G/Y206F | M129T/V168G/Y205F | M132T/V170G/Y207F |
| MmPylRS | N165A/V167A | N166A/V168A | N166A/V168A | N168A/V170A |
| MmPylRS | N165G/V167G/Y204F | N166G/V168G/Y206F | N166G/V168G/Y205F | N168G/V170G/Y207F |
| MmPylRS | N165G/V167Q | N166G/V168Q | N166G/V168Q | N168G/V170Q |
| MmPylRS | N165G/V167Q/A221G | N166G/V168Q/A223G | N166G/V168Q/A222G | N168G/V170Q/A224G |
| MmPylRS | N165Q/V167A/A221M | N166Q/V168A/A223M | N166Q/V168A/A222M | N168Q/V170A/A224M |
| MmPylRS | N165Q/V167S/A221G/ | N166Q/V168S/A223G/ | N166Q/V168S/A222G/ | N168Q/V170S/A224G/ |
| | W237T | W239T | W238T | W240T |
| MmPylRS | Y125A/M128A/V167S/ | Y126A/M129A/V168S/ | Y126A/M129A/V168S/ | Y129A/M132A/V170S/ |
| | Y204F | Y206F | Y205F | Y207F |
| MmPylRS | Y125A/M128M/V167G/ | Y126A/M129M/M170G/ | Y126A/M129M/V170G/ | Y129A/M132M/V170G/ |
| | Y204F/H225R | Y206F/H227R | Y205F/P226R | Y207F/K228R |
| MmPylRS | Y125A/Y204F | Y126A/Y206F | Y126A/Y205F | Y129A/Y207F |
| MmPylRS | Y125A/Y204F | Y126A/Y206F | Y126A/Y205F | Y129A/Y207F |
| MmPylRS | Y125A/Y204F/V233L | Y126A/Y206F/V235L | Y126A/Y205F/I234L | Y129A/Y207F/V236L |
| MmPylRS | Y125F/Y204F | Y126G/Y206F | Y126G/Y205F | Y129G/Y207F |
| MmPylRS | Y125A/Y204F | Y126A/Y206F | Y126A/Y205F | Y129A/Y207F |
| MmPylRS | Y125L/M128A/N165A/ | Y126L/M129A/N166A/ | Y126L/M129A/N166A/ | Y129L/M132A/N168A/ |
| | V167M/W237T | V168M/W239T | V168M/W238T | V170L/W240T |
| MmPylRS | Y125V/M128A/V167F/ | Y126V/M129A/V168F/ | Y126V/M129A/V168F/ | Y129V/M132A/V170F/ |
| | Y204F | Y206F | Y205F | Y207F |
| MmPylRS | Y125V/M128A/V167F/ | Y126V/M129A/V168F/ | Y126V/M129A/V168F/ | Y129V/M132A/V170F/ |
| | Y204F | Y206F | Y205F | Y207F |
| MmPylRS | Y204F | Y206F | Y205F | Y207F |
| MmPylRS | Y204F | Y206F | Y205F | Y207F |
| MbPylRS | L124I/Y125F/M128G/ | L125I/Y126F/M129G/ | L125I/Y126F/M129G/ | L128I/Y129F/M132G/ |
| | V167F/Y204F | V168F/Y206F | V168F/Y205F | V170F/Y207F |
| MbPylRS | N165C/V167S/Y204F | N166C/V168S/Y206F | N166C/V168S/Y205F | N168C/V170S/Y207F |
| MbPylRS | A121D/N165G/V167G | A122D/N166G/V168G | A122D/N166G/V168G | A125D/N168G/V170G |
| MbPylRS | A121Q/N165S/V167W | A122Q/N166S/V168W | A122Q/N166S/V168W | A125Q/N168S/V170W |
| MbPylRS | A121S/M169F/E199G | A122S/M170F/E201G | A122S/L170F/E200G | A125S/L172F/E202G |
| MbPylRS | A121Y/Y125A/N165T/ | A122Y/Y126A/N166T/ | A122Y/Y126A/N166T/ | A125Y/Y129A/N168T/ |
| | V167G/Y204F | V168G/Y206F | V168G/Y205F | V170G/Y207F |
| MbPylRS | V167S/Y204F | V168S/Y206F | V168S/Y205F | V170S/Y207F |
| MbPylRS | V167T | V168T | V168T | V170T |
| MbPylRS | V167T/Y204F | V168T/Y206F | V168T/Y205F | V170T/Y207F |
| MbPylRS | V167T/Y204F | V168T/Y206F | V168T/Y205F | V170T/Y207F |
| MbPylRS | V167V | V168V | V168V | V170V |
| MbPylRS | M169L/H225K | M170L/H227K | P226K/I234V | WT |
| MbPylRS | V167W/W237T | V168W/W239T | V168W/W238T | V170W/W240T |
| MbPylRS | H120M/L124I/Y125F/ | L121M/L125I/Y126F/ | L121M/L125I/Y126F/ | L124M/L128I/Y129F/ |
| | M128A/V167F | M129A/V168F | M129A/V168F | M132A/V170F |
| MbPylRS | H120V/L124I/Y125F/ | L121V/L125I/Y126F/ | L121V/L125I/Y126F/ | L124V/L128I/Y129F/ |
| | M128A/V167F | M129A/V168F | M129A/V168F | M132A/V170F |
| MbPylRS | M128A/V167F/Y204F | M129A/V168F/Y206F | M129A/V168F/Y205F | M132A/V170F/Y207 |
| MbPylRS | N165S/V167G/A221H/ | N166S/V168G/A223H/ | N166S/V168G/A222H/ | N168S/V170G/A224H/ |
| | W237V | W239V | W238V | W240V |
| MbPylRS | H120M/L124I/M128A/ | L121M/L125I/M129A/ | L121M/L125I/M129A/ | L124M/L128I/M132A/ |
| | V167F | V168F | V168F | V170F |
| MbPylRS | H120M/L124I/Y125F/ | L121M/L125I/Y126F/ | L121M/L125I/Y126F/ | L124M/L128I/Y129F/ |
| | M128A/V167F | M129A/V168F | M129A/V168F | M132A/V170F |
| MbPylRS | H120M/L124I/Y125L/ | L121M/L125I/Y126L/ | L121M/L125I/Y126L/ | L124M/L128I/Y129L/ |
| | M128A | M129A | M129A | M132A |
| MbPylRS | H120M/L124I/Y125L/ | L121M/L125I/Y126L/ | L121M/L125I/Y126L/ | L124M/L128I/Y129L/ |
| | M128A/V167I | M129A/V168I | M129A/V168I | M132A/V170I |
| MbPylRS | L124F/M128M/N165G/ | L125F/M129M/N166G/ | L125F/M129M/N166G/ | L128F/M132M/N168G/ |
| | V167G/Y204F | V168G/Y206F | V168G/Y205F | V170G/Y207F |
| MbPylRS | L124F/M128M/N165A/ | L125F/M129M/N166A/ | L125F/M129/N166A/ | L128F/M132/N168A/ |
| | V167G | V168G | V168G | V170G |
| MbPylRS | L124F/M128M/N165G/ | L125F/M129M/N166G/ | L125F/M129/N166G/ | L128F/M132/N168G/ |
| | V167G | V168G | V168G | V170G |
| MbPylRS | L124F/M128M/N165G/ | L125F/M129M/N166G/ | L125F/M129/N166G/ | L128F/M132/N168G/ |
| | V167G | V168G | V168G | V170G |
| MbPylRS | L124I/Y125A/Y204F | M129I/Y126A/Y206F | Y125I/Y126A/Y205F | L128I/Y129A/Y207F |
| MbPylRS | M128A/V167F/Y204W | M129A/V168F/Y206W | M129A/V168F/Y205W | M132A/V170F/Y207W |
| MbPylRS | M128A/V167S | M129A/V168S | M129A/V168S | M132A/V170S |
| MbPylRS | M128A/V167S | M129A/V168S | M129A/V168S | M132A/V170S |
| MbPylRS | M128A/V167S/Y204F | M129A/V168S/Y206F | M129A/V168S/Y205F | M132A/V170S/Y207F |
| MbPylRS | M128A/V167F/Y204F | M129A/V168F/Y206F | M129A/V168F/Y205F | M132A/V170F/Y207F |
| MbPylRS | M128A/V167S/Y204F | M129A/V168S/Y206F | M129A/V168S/Y205F | M132A/V170S/Y207F |
| MbPylRS | M128A/V167S/Y204F | M129A/V168S/Y206F | M129A/V168S/Y205F | M132A/V170S/Y207F |
| MbPylRS | M128A/V167S/Y204F | M129A/V168S/Y206F | M129A/V168S/Y205F | M132A/V170S/Y207F |
| MbPylRS | M128A/V167T/Y204W | M129A/V168T/Y206W | M129A/V168T/Y205W | M132A/V170T/Y207W |
| MbPylRS | M128A/V167V/Y204F | M129A/V168V/Y206F | M129A/V168V/Y205F | M132A/V170V/Y207F |
| MbPylRS | M128A/N165Q/V167S | M129A/N166Q/V168S | M129A/N166Q/V168S | M132A/N168Q/V170S |
| MbPylRS | M128C | M129C | M129C/I234V | M132C |
| MbPylRS | M128G/M169A/H225R | M129G/M170A/H227R | M129G/L170A/P226R/ | M132G/L172A/K228R |
| | | | I234V | |
| MbPylRS | M128G/V167V/Y204F | M129G/V168V/Y206F | M129G/V168V/Y205F | M132G/V170V/Y207F |
| MbPylRS | M95/A121/Y125M/ | M96/A122/Y126M/ | M96/A122/Y126M/ | M99/A125/Y129M/ |
| | M128G/V167A/Y204W | M129G/V168A/Y206W | M129G/V168A/Y205W | M132G/V170A/Y207W |
| MbPylRS | Y125A/Y204F | Y126A/Y206F | Y126A/Y205F | Y129A/Y207F |
| MbPylRS | M95A/Y125A/M128V/ | M96A/Y126A/M129V/ | M96A/Y126A/M129V/ | M99A/Y129A/M132V/ |
| | M169Y/Y204F/H225R | M170Y/Y206F/H227R | L170Y/Y205F/P226R | L172Y/Y207F/K228R |
| MbPylRS | M95F/A121S/Y125C/ | M96F/A122S/Y126C/ | M96F/A122S/Y126C/ | M99F/A125S/Y129C/ |
| | M128M | M129M | M129M | M132M |
| MbPylRS | M95F/A121S/Y125C/ | M96F/A122S/Y126C/ | M96F/A122S/Y126C/ | M99F/A125S/Y129C/ |
| | M128M | M129M | M129M | M132M |
| MbPylRS | N165A/V167M/A221G/ | N166A/V168M/A223G/ | N166A/V168M/A222G/ | N168A/V170M/A224G/ |
| | W237T | W239T | W238T | W240T |
| MbPylRS | N165A/V167M/A221G/ | N166A/V168M/A223G/ | N166A/V168M/A222G/ | N168A/V170M/A224G/ |
| | W237T | W239T | W238T | W240T |
| MbPylRS | N165G/V167A | N166G/V168A | N166G/V168A | N168G/V170A |
| MbPylRS | N165G/V167V/A221K | N166G/V168V/A223K | N166G/V168V/A222K | N168G/V170V/A224K |
| MbPylRS | N165M/V167Q/A221G/ | N166M/V168Q/A223G/ | N166M/V168Q/A222G/ | N168M/V170Q/A224G/ |
| | W237N | W239N | W238N | W240N |
| MbPylRS | N165Q/Y204F | N166Q/Y206F | N166Q/Y205F | N168Q/Y207F |
| MbPylRS | N165Q/V167A/A221M | N166Q/V168A/A223M | N166Q/V168A/A222M | N168Q/V170A/A224M |
| MbPylRS | N165S/V167A/A221H/ | N166S/V168A/A223H/ | N166S/V168A/A222H/ | N168S/V170A/A224HI/ |
| | W237I | W239I | W238I | W240 |
| MbPylRS | N165S/V167A/Y204F | N166S/V168A/Y206F | N166S/V168A/Y205F | N168S/V170A/Y207F |
| MbPylRS | N165S/V167G/A221A/ | N166S/V168G/A223A/ | N166S/V168G/A222A/ | N168S/V170G/A224A/ |
| | W237T | W2399T | W238T | W240T |
| MbPylRS | N165S/V167G/Y204F | N166S/V168G/Y206F | N166S/V168G/Y205F | N168S/V170G/Y207F |
| MbPylRS | Y204F | Y206F | Y205F | Y207F |
| MbPylRS | Y125A/H225L | Y126A/H227L | Y126A/P226L/I234V | Y129A/K228L |
| MbPylRS | Y125A/M128A/M169Y/ | Y126A/M129A/M170Y/ | Y126A/M129A/L170Y/ | Y129A/M132A/L172Y/ |
| | H225R | H227R | P226R | K228R |
| MbPylRS | Y125A/M128M | Y126A/M129M | Y126A/M129M | Y129A/M132M |
| MbPylRS | Y125A/M128V/M169Y/ | Y126A/M129V/M170Y/ | Y126A/M129V/L170Y/ | Y129A/M132V/L172Y/ |
| | H225R | H227R | P226R | K228R |
| MbPylRS | Y125A/M128V/M169Y/ | Y126A/M129V/M170Y/ | Y126A/M129V/L170Y/ | Y129A/M132V/L172Y/ |
| | Y204F/H225R | Y206F/H227R | Y205F/P226R | Y207F/K228R |
| MbPylRS | Y125A/Y204F | Y126A/Y206F | Y126A/Y205F | Y129A/Y207F |
| MbPylRS | Y125C/N165Q/Y204F/ | Y126C/N166Q/Y206F/ | Y126C/N166Q/Y205F/ | Y129C/N168Q/Y207F/ |
| | A221C | A223C | A222C | A224C |
| MbPylRS | Y125F/V167T | Y126F/V168T | Y126F/V168T | Y129F/V170T |
| MbPylRS | Y125G/V167V | Y126G/V168V | Y126G/V168V | Y129G/V170V |
| MbPylRS | Y125G/V167V/H225R | Y126G/V168V/L229R | Y126G/V168V/P226R | Y129G/V170V/K228R |
| MbPylRS | Y125G/M128V/M169Y/ | Y126G/M129V/M170Y/ | Y126G/M129V/L170Y/ | Y129G/M132V/L172Y/ |
| | H225R | H227R | P226R | K228R |
| MbPylRS | Y125I/M128M/V167A | Y126I/M129M/V168A | Y126I/M129M/V168A | Y129I/M132M/V170A |
| MbPylRS | Y125M/M128A/V167A | Y126M/M129A/V168A | Y126M/M129A/V168A | Y129M/M132A/V170A |
| MbPylRS | Y125M/M128A/N165A/ | Y126M/M129A/N166A/ | Y126M/M129A/N166A/ | Y129M/M132A/N168A/ |
| | V167A/Y204F | V168A/Y206F | V168A/Y205F | V170A/Y207F |
| MbPylRS | Y125M/M128T/V167A/ | Y126M/M129T/V168A/ | Y126M/M129T/V168A/ | Y129M/M132T/V170A/ |
| | Y204F | Y206F | Y205F | Y207F |
| MbPylRS | Y204F | Y206F | Y205F | Y207F |
| MbPylRS | Y204W | Y206W | Y205W | Y207W |
| MaPylRS | L124F/N165A/V167G | L125F/N166A/V168G | L125F/N166A/V168G | L128F/N168A/V170G |
| MaPylRS | Y125A/H225I/K226P | Y126A/H227I/Y228P | Y126A/P226I/L227P | Y129A/K228I |
| MaPylRS | Y125G/M128A/V167F/ | Y126G/M129A/V168F/ | Y126G/M129A/V168F/ | Y129G/M132A/V170F/ |
| | H225T/K226P/L227I | H227T/Y228P/L229I | P226T/L227P/M228I | K228T/P229P/I230I |
| MaPylRS | Y125G/M128A/V167F/ | Y126G/M129A/V168F/ | Y126G/M129A/V168F/ | Y129G/M132A/V170F/ |
| | H226T/K227P/L228I | H227T/Y228P/L229I | P226T/L227P/M228I | K228T/P229P/I230I |
| G1PylRS | L124M/Y125M/N165A/ | L125M/Y126M/N166A/ | L125M/Y126M/N166A/ | L128M/Y129M/N168A/ |
| | V167G/A221G/W237H | V168G/A223G/W239H | V168G/A222G/W238H | V170G/A224G/W240H |
| G1PylRS | L124A/Y125L/V167A/ | L125A/Y126L/V168A/ | L125A/Y126L/V168A/ | L128A/Y129L/V170A/ |
| | Y204W/A221S | Y206W/A223S | Y205W/A222S | Y207W/A224S |
| G1PylRS | L124A/Y125L/V167A/ | L125A/Y126F/V168A/ | L125A/Y126F/V168A/ | L128A/Y129F/V170A/ |
| | Y204W/A221S | Y206W/A223S | Y205W/A2228 | Y207W/A224S |
| G1PylRS | L124G/M128A/N165G/ | L125G/M129A/N166G/ | L125G/M129A/N166G/ | L128G/M132A/N168G/ |
| | V167A | V168A | V168A | V170A |
| G1PylRS | L124G/M128L/N165G/ | L125G/M129L/N166G/ | L125G/M129L/N166G/ | L128G/M132L/N168G/ |
| | V167A | V168A | V168A | V170A |
| G1PylRS | L124G/M128A/N165G/ | L125G/M129A/N166G/ | L125G/M129A/N166G/ | L128G/M132A/N168G/ |
| | V167A | V168A | V168A | V170A |
| G1PylRS | L124G/Y125F/N165G/ | L125G/Y126F/N166G/ | L125G/Y126F/N166G/ | L128G/Y129F/N168G/ |
| | V167F/A221G/W237Y | V168F/A223G/W239Y | V168F/A222G/W238Y | V170F/A224G/W240Y |
| G1PylRS | N165G/V167G | N166G/V168G | N166G/V168G | N168G/V170G |
| chPylRS | V167G/A221C/Y204F | V168G/A223C/Y206F | V168G/A222C/Y205F | V170G/A224C/Y207F |
| chPylRS | L124I/M128G/N165C/ | L125I/M129G/N166C/ | L125I/M129G/N166C/ | L128I/M132G/N168C/ |
| | V167W/Y204F | V168W/Y206F | V168W/Y205F | V170W/Y207F |
| chPylRS | L124V/M128G/N165C/ | L125V/M129G/N166C/ | L125V/M129G/N166C/ | L128V/M132G/N168C/ |
| | V167W/Y204F | V168W/Y206F | V168W/Y205F | V170W/Y207F |

In one embodiment, the aminoacyl-tRNA synthetase may have both the IPYE mutation and specific mutations for recognizing unnatural amino acids. For example, chPylRS can be mutated to chPylRSIPYE with the IPYE mutation, and then L270G/N311G/C313A mutations can be introduced to obtain an aminoacyl-tRNA synthetase specific for TetBu, denoted as TetRS.

Recoding tRNAs

Recoding tRNAs can be used to load unnatural amino acids and, under the action of aminoacyl-tRNA synthetases, form aminoacyl-tRNAs with unnatural amino acids.

The recoding tRNAs described in this application can be exogenous recoding tRNAs.

Recoding tRNAs may include an anticodon loop with an anticodon that can hybridize and pair with rare codons. Recoding tRNAs use the anticodon to insert unnatural amino acids into transcription templates containing rare codons and/or amber codons.

In this application, the aminoacyl-tRNA synthetase can be derived from prokaryotes or eukaryotes.

For example, the aminoacyl-tRNA synthetase is derived from prokaryotes; further, it can be derived from eubacteria or archaea. For types of eubacteria and archaea, see the “Definitions” section.

In some embodiments, the recoding tRNA can be pyrrolysyl-tRNA (PylT).

In some embodiments, the recoding tRNA can be tyrosyl-tRNA (TyrT).

In some embodiments, the recoding tRNA can be leucyl-tRNA (LeuT).

A particular recoding tRNA can pair with a specific aminoacyl-tRNA synthetase to transport one or more specific unnatural amino acids. For example, the first recoding tRNA pairs with the first aminoacyl-tRNA synthetase to transport the first unnatural amino acid, and the second recoding tRNA pairs with the second aminoacyl-tRNA synthetase to transport the second unnatural amino acid. This pairing can be because the recoding tRNA and the specific aminoacyl-tRNA synthetase are both derived from the same organism or because the recoding tRNA is designed for a specific aminoacyl-tRNA synthetase. Specific pairing relationships between recoding tRNAs and specific aminoacyl-tRNA synthetases can be found in Tables 7 and 8 of this application.

To improve the efficiency of unnatural amino acid expression, wild-type recoding tRNAs can be mutated. First, pyrrolysyl-tRNAs have high homology; for example, MmPyltRNA and MbPyltRNA differ by only one nucleotide (T at position 42 in the former and C at position 42 in the latter). Therefore, MbPylT can be used as a representative to predict the mutation effects of other types of pyrrolysyl-tRNAs.

In some embodiments, compared to wild-type pyrrolysyl-tRNA, the pyrrolysyl-tRNA described in this application may have one or more of the following sequence features: G at position 3, G at position 5, A at position 6, G at position 7, G at position 10, C at position 11, A at position 14, G at position 16, T at position 17, G at position 19, C at position 20, G at position 45, G at position 46, C at position 52, C at position 60, C at position 61, C at position 62, T at position 63, C at position 64, and C at position 66. These sequence features can enhance the expression efficiency of unnatural amino acids. In this application, position X can represent either the absolute position of the nucleotide (e.g., with MmPyltRNA as a reference) or the relative position of the nucleotide (e.g., G at position 3 and G at position 5 means there is one nucleotide between G and G).

Furthermore, the same recoding tRNA can have different anticodons in the anticodon region, corresponding to different rare codons. Depending on the rare codon, the anticodon can be one or more of anticodons corresponding to the following codons:TCG, ACG, CGA, TCA, CGC, TTG, ATA, GCG, TTA, and TGT.

In this application, codons are represented by their coding genes (i.e., DNA). For example, the codons TCG, ACG, CGA, TCA, CGC, TTG, ATA, GCG, TTA, and TGT correspond to UCG, ACG, CGA, UCA, CGC, UUG, AUA, GCG, UUA, and UGU of mRNA, respectively.

### Unnatural Amino Acids and Proteins, Polypeptides, or Amino Acid Residues Containing Unnatural Amino Acids

The unnatural amino acids described in this application refer to any amino acids, modified amino acids, or amino acid analogs other than selenocysteine, pyrrolysine, and the following 20 genetically encoded amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

The unnatural amino acids described in this application may contain a first reactive group, and proteins, polypeptides, or amino acid residues containing unnatural amino acids with the first reactive group can be contacted and biochemically reacted with molecules containing a second reactive group (e.g., polymers such as polyethylene glycol derivatives, photocrosslinkers, cytotoxic compounds, affinity labels, biotin derivatives, resins, second proteins or polypeptides, metal chelators, cofactors, fatty acids, carbohydrates, and/or polynucleotides). For example, the first reactive group can be an alkyne and/or azide moiety, and the second reactive group can be an azide and/or alkyne moiety.

In proteins, polypeptides, or amino acid residues containing unnatural amino acids, the unnatural amino acids can provide various benefits and effects to the protein. For instance, based on the properties of ketone functional groups, the protein, polypeptide, or amino acid residue can be modified in vitro and in vivo by one or more hydrazine- and/or hydroxylamine-containing chemical reagents. When the protein, polypeptide, or amino acid residue contains photoreactive unnatural amino acids (e.g., amino acids with benzophenone and azidoaryl side chains), the protein, polypeptide, or amino acid residue can be efficiently photocrosslinked in vivo and in vitro.

The present application also provides proteins, polypeptides, or amino acid residues containing unnatural amino acids obtained through the production method and/or system described herein.

Recoding tRNA and Aminoacyl-tRNA Synthetase System

The method provided by this application for recoding unnatural amino acids using rare codons overcomes the inefficiency caused by different sources of translation components (such as recoding tRNA and aminoacyl-tRNA synthetase pairs), achieving unexpected results in unnatural amino acid insertion.

Aminoacyl-tRNA synthetase/tRNA pairs are core tools in the translation process, and ensuring their orthogonality is key to developing recoding technology and exploring downstream applications. Orthogonality encompasses two levels: first, the orthogonality of tRNA, meaning that the recoding tRNA can only be specifically recognized and aminoacylated by the corresponding aminoacyl-tRNA synthetase and cannot undergo aminoacylation with other endogenous aminoacyl-tRNA synthetases; second, the orthogonality of the aminoacyl-tRNA synthetase, meaning that the aminoacyl-tRNA synthetase can specifically recognize exogenously added unnatural amino acids and cannot transport natural amino acids. These two levels of orthogonality ensure that the codon corresponding to the anticodon on the recoding tRNA, i.e., the codon used for recoding, can only insert the desired unnatural amino acid after binding to the recoding tRNA.

However, aminoacyl-tRNA synthetase/tRNA pairs in eukaryotes often exhibit less strict orthogonality, and eukaryotes inherently lack recoding aminoacyl-tRNA synthetases/tRNAs, requiring the exogenous introduction of orthogonal aminoacyl-tRNA synthetase/recoding tRNA components to produce proteins containing unnatural amino acids. These components can be from prokaryotes or artificially chimeric. Due to the different sources of translation components, their adaptability in eukaryotes is inevitably reduced, causing aminoacyl-tRNA synthetase/tRNA pairs to often exhibit issues with incorporation efficiency and background incorporation in eukaryotic systems such as yeast and mammalian cells, making the insertion results of unnatural amino acids unpredictable.

In prokaryotes, the problems of low incorporation efficiency and background incorporation can be addressed by redesigning the genome, synonymously replacing corresponding rare codons in the genome, and knocking out the corresponding decoding tRNAs. However, in eukaryotes, due to the complexity of the genome and the high cost of genome editing, researchers in the field generally believe that effective reprogramming cannot be achieved using rare codons in eukaryotic cells. Through a series of optimizations, this application overcomes the inefficiency caused by different sources of translation components (such as recoding tRNA and aminoacyl-tRNA synthetase pairs), achieving unexpected results in unnatural amino acid insertion.

In this application, the production of proteins/polypeptides containing unnatural amino acids is based on the successful expression of recoding tRNAs and aminoacyl-tRNA synthetases. First, the unnatural amino acid and ATP condense to form a mixed anhydride intermediate of amino acid and adenylate, which then non-covalently binds to the active site. The second step is the aminoacyl transfer reaction, where the amino acid is esterified to the 3′ end of the recoding tRNA. As a result, the free unnatural amino acid, under the catalysis of the aminoacyl-tRNA synthetase, forms aminoacyl-tRNA with the corresponding recoding tRNA. The aminoacyl-tRNA, with the help of free ribosomes, is inserted into the peptide chain by forming a peptide bond with the previously inserted amino acid, thereby generating proteins and/or polypeptides containing unnatural amino acids.

In the above process, specific unnatural amino acids preferentially interact with specific recoding tRNAs and/or specific aminoacyl-tRNA synthetases. For example, the first unnatural amino acid preferentially forms the first aminoacyl-tRNA with the first recoding tRNA through the first aminoacyl-tRNA synthetase. Similarly, the second unnatural amino acid preferentially forms the second aminoacyl-tRNA with the second recoding tRNA through the second aminoacyl-tRNA synthetase. The first and/or second unnatural amino acids are further inserted into the peptide chain, forming proteins and/or polypeptides containing unnatural amino acids.

In some embodiments, the first and/or second recoding tRNAs can be exogenous tRNAs. For example, the first and/or second recoding tRNAs can be expressed in the host cell via a vector. In some embodiments, the host cell can be genetically modified. For instance, the wild-type host cell may not express the first and/or second recoding tRNAs, while the modified host cell can express the first and/or second orthogonal tRNAs.

In some embodiments, the first and/or second aminoacyl-tRNA synthetases can be exogenous aminoacyl-tRNA synthetases. For example, the first and/or second aminoacyl-tRNA synthetases can be expressed in the host cell via a vector. In some embodiments, the host cell can be genetically modified. For instance, the wild-type host cell may not express the first and/or second aminoacyl-tRNA synthetases, while the modified host cell can express the first and/or second aminoacyl-tRNA synthetases.

When the aminoacyl-tRNA synthetase is PylRS, the orthogonal pyrrolysyl-tRNA is selected from one or more of C15, M15, CM15, MbPylT, MetPylT, SpePylT, Pyl-O1, Pyl-O2, Ma-T6, MaPylT, G1PylT, G1hyb, Ma-T6, 12B72, Int17, Int5, Int6B, Int6C, Int13, Alv21, Alv8, Alv17, Alv10, Alv22, G1hyb, Int, Therm1, and BH52.

For example, when the aminoacyl-tRNA synthetase is MmPylRS, MbPylRS, or chPylRS, the orthogonal pyrrolysyl-tRNA is selected from one or more of C15, M15, CM15, MbPylT, MetPylT, SpePylT, Pyl-O1, and Pyl-O2. When the aminoacyl-tRNA synthetase is MaPylRS, the orthogonal pyrrolysyl-tRNA is selected from one or more of MaPylT, G1PylT, G1hyb, and Ma-T6. When the aminoacyl-tRNA synthetase is G1PylRS, the orthogonal pyrrolysyl-tRNA is selected from one or more of MaPylT, G1PylT, G1hyb, and Ma-T6. When the aminoacyl-tRNA synthetase is Lum1PylRS, the orthogonal pyrrolysyl-tRNA is selected from one or more of MaPylT, G1PylT, G1hyb, and Ma-T6. When the aminoacyl-tRNA synthetase is 1R26PylRS, the orthogonal pyrrolysyl-tRNA is selected from one or more of Alv21, Alv8, Alv17, Alv10, Alv22, and G1hyb.

When the aminoacyl-tRNA synthetase is IntPylRS, the orthogonal pyrrolysyl-tRNA is selected from one or more of Int, Int17, Int5, Int6B, Int6C, and Int13. When the aminoacyl-tRNA synthetase is NitraPylRS, the orthogonal pyrrolysyl-tRNA is selected from one or more of Int, Int17, Int5, Int6B, Int6C, Intl, and Therm1. When the aminoacyl-tRNA synthetase is DebPylRS, the orthogonal pyrrolysyl-tRNA is selected from one or more of Int and BH52. When the aminoacyl-tRNA synthetase is chPheRS, the recoding tRNA is selected from one or more of CM4, CM15, MbPylT, Pyl-O1, Pyl-O2, and AS78. When the aminoacyl-tRNA synthetase is EcTyrRS, the recoding tRNA is selected from one or more of BsTyrT and NGS6. When the aminoacyl-tRNA synthetase is EcLeuRS, the recoding tRNA is selected from one or more of EcLeuT, L-G1, and L-G2.

### Rare Codons

The recoding tRNA contains an anticodon that can pair with the rare codon, allowing the unnatural amino acid loaded on the recoding tRNA to be introduced at the position of the rare codon on the transcription template.

It is important to note that since rare codons are sense codons, they can encode natural amino acids in the host cell. For example, the rare codon TCG can encode serine (Ser) in eukaryotic cells. This application uses rare codons to encode unnatural amino acids, thereby expressing proteins and/or polypeptides containing unnatural amino acids in eukaryotic cells (e.g., mammalian cells). Therefore, at least the wild-type host cell contains endogenous tRNAs capable of decoding rare codons.

The anticodon loops of endogenous tRNAs and exogenous recoding tRNAs can compete to recognize and bind the rare codon. After binding the rare codon, the endogenous tRNA or exogenous recoding tRNA can insert an amino acid, such as an unnatural amino acid, at the site on the polypeptide chain encoded by the transcription template corresponding to the rare codon.

Compared to using stop codons to encode unnatural amino acids in eukaryotes, one advantage of this application is the ability to more easily obtain proteins and/or polypeptides with full-length peptide chains. When using stop codons to encode unnatural amino acids, suppressor tRNAs compete with translation release factors for binding to the stop codon; when using rare codons to encode unnatural amino acids, recoding tRNAs compete with endogenous tRNAs for binding to the rare codon. Since the abundance of endogenous tRNAs is much lower than that of translation release factors, competition for the codon is reduced, thereby increasing the production efficiency of proteins containing unnatural amino acids.

Another advantage of this application compared to using stop codons to encode unnatural amino acids in eukaryotes is the effective reduction of cytotoxicity caused by the recoding system. In addition to the target protein, there are other sites with recoding codons in the proteome. When using stop codons to encode unnatural amino acids, the erroneous insertion of unnatural amino acids in the proteome can prevent other non-target proteins from terminating translation, leading to significant cytotoxicity. When using rare codons to encode unnatural amino acids, the erroneous insertion of unnatural amino acids in the proteome does not cause abnormal elongation of other non-target proteins, resulting in lower cytotoxicity.

In this application, the codons encoding unnatural amino acids are rare codons. In some embodiments, the rare codons can be one or more of TCG, ACG, CGA, AGA, TCA, GCG, ATA, TTA, TGT, CAA, TGG, ACA, and GGA. Further, the rare codons can be one or more of TCG, ACG, CGA, AGA, TCA, and GCG.

For example, the first rare codon and/or the second rare codon can each be one of TCG, ACG, CGA, AGA, TCA, GCG, ATA, TTA, TGT, CAA, TGG, ACA, and GGA, and the first rare codon and the second rare codon are not the same. Alternatively, the first rare codon and/or the second rare codon can each be one or more of TCG, ACG, CGA, AGA, TCA, and GCG, and the first rare codon and the second rare codon are not the same.

The rare codons can be codons that can be recognized by both endogenous tRNAs and recoding tRNAs. Each organism or cell has a certain tRNA abundance, i.e., tRNA content. The anticodon on the tRNA corresponds to the codon on the transcription template. The tRNA content for common codons is much higher than that for rare codons, and the usage frequency of common codons is also higher than that of rare codons, especially in highly expressed genes. The tRNA content is positively correlated with codon usage frequency; the higher the tRNA content corresponding to a codon, the higher its usage frequency, and similarly, when a codon's usage frequency is high in a gene, its corresponding tRNA content will also be relatively high in the cell or organism expressing the gene. Rare codons can be codons whose corresponding encoding endogenous tRNA abundance in the host cell is less than 2%, 1.75%, 1.5%, 1.25%, 1%, 0.5%, or 0.25%. When the host is a multicellular organism, the tRNA abundance can be the total tRNA content in the host cells used.

Rare codons can be codons with a usage frequency in the host cell of less than 20/1000, 17.5/1000, 15/1000, 12.5/1000, 10/1000, 7.5/1000, or 5/1000. The usage frequency can be the usage frequency of codons in the entire transcriptome of the host cell or the usage frequency of codons on the transcription template corresponding to the desired protein in the host cell. The desired protein can be an endogenous protein expressed by the host cell itself or a recombinant protein introduced exogenously, such as green fluorescent protein and interleukin protein.

Rare codons can include one or more of TCG, ACG, CGA, AGA, TCA, GCG, ATA, TTA, TGT, CAA, TGG, ACA, and GGA. In some embodiments, the rare codons can include one or more of TCG, ACG, CGA, AGA, TCA, and GCG.

The rare codons can be present consecutively on the transcription template, meaning there are no nonsense codons and/or blank codons, and/or any sequences that do not encode proteins, polypeptides, or amino acid residues between the rare codons used. Sequences that do not encode proteins, polypeptides, or amino acid residues can be element sequences on the transcription template, such as 5′ cap structures, 5′ UTR, 3′ UTR, and/or polyA sequences, or intron sequences.

The upstream and downstream sequences of the codon can affect the binding of the codon to tRNA. For example, when the upstream and/or downstream sequences of the codon are specific sequences or combinations, such as high G content, high C content, high A content, and/or high U content, the binding efficiency of the codon to tRNA can be significantly improved. For instance, when one, two, three, four, five, six, or more nucleotide sequences upstream and/or downstream of the codon are specific sequences or combinations, the binding efficiency of the codon to tRNA is significantly enhanced. The impact of upstream and downstream sequences on the binding of the codon to tRNA can be associated with the transcription template; on different transcription templates, the effect of upstream and downstream sequences on the binding of the same codon to tRNA can be the same or different.

Modifications to the bases in the codon can affect the binding of the codon to tRNA. Modifications to the bases in the codon mean keeping the codon unchanged, i.e., the sequence of the transcription template remains the same, only introducing modified bases without changing the base pairing between the codon and tRNA. When unstable modifications are introduced into the codon, the binding rate of the codon to tRNA can be significantly reduced, thereby significantly lowering the recoding efficiency. When stable modifications are introduced into the codon, the binding rate of the codon to tRNA can be significantly increased, thereby significantly improving the recoding efficiency. The insertion site of modified nucleotides can also affect the binding of the codon to tRNA; when modified nucleotides are inserted into the 5′ cap structure, 5′ UTR, CDS region, 3′ UTR, and/or polyA sequence, the binding rate of the codon to tRNA can be significantly increased or decreased, thereby significantly improving or lowering the recoding efficiency.

The introduction of modified bases in the codon can affect the binding of the codon to tRNA by influencing the secondary structure of the transcription template. For example, when the transcription template has stable or locally stable secondary structures, the translation efficiency of the transcription template can be significantly improved, i.e., the binding efficiency of the codon to tRNA is significantly enhanced.

Based on the existing technology providing stop codon recoding systems, it is widely believed in the art that at most three unnatural amino acids can be inserted in eukaryotic organisms. This application provides a method for simultaneously inserting four or more unnatural amino acids at six sites in eukaryotic proteins using rare codons.

Without affecting the normal function of the host cell, the number of codons that can be used for unnatural amino acid recoding in eukaryotic cells is limited, and using three stop codons to simultaneously insert three unnatural amino acids is considered the limit for inserting amino acid types. In contrast, prokaryotic genomes have simple structures and mature editing techniques, allowing for the simultaneous insertion of multiple unnatural amino acids in proteins produced by prokaryotic cells through methods such as adding unnatural base pairs, adding quadruplet codons, or even redesigning the entire genome. The method proposed in this application for using rare codons to achieve unnatural amino acid recoding in eukaryotic cells creatively enables the insertion of more than three unnatural amino acids in eukaryotic proteins.

The translation process and post-translational modification methods differ between eukaryotic and prokaryotic cells, making it unpredictable whether proteins containing multiple unnatural amino acids synthesized using translation components to encode rare codons in eukaryotes will have functional activity. Due to the source and adaptability of translation components, prokaryotes can achieve rapid and precise production of simple proteins within cells. For eukaryotes, due to the aforementioned less strict orthogonality, it is difficult to predict whether the translated polypeptides can be processed into functionally active proteins by the translation system. According to the method proposed in this application for using rare codons to achieve unnatural amino acid recoding in eukaryotic cells, by designing target protein encoding genes containing rare codons, the simultaneous insertion of unnatural amino acids at six sites on the target protein was successfully achieved, and the target protein retained complete functional activity.

The codons can insert one, two, three, four, or more unnatural amino acids into the same protein, polypeptide, or amino acid residue. The gene or transcription template encoding the target protein, polypeptide, or amino acid residue may include multiple copies of specified codons to insert one, two, three, four, or more unnatural amino acids, or may include multiple specified codons and/or combinations thereof to insert one, two, three, four, or more unnatural amino acids.

### Optimization of Recoding Efficiency

Given the complexity of the eukaryotic transcriptome, this application first screens codons suitable for unnatural amino acid recoding in eukaryotes from three aspects: codon rarity, high recoding efficiency, and low background incorporation. Then, optimizations are made at the levels of aminoacyl-tRNA synthetases, recoding tRNAs, culture systems, and the host cell genome, achieving a breakthrough in the efficiency of inserting natural amino acids using the rare codons employed in this application.

### Codon Screening

In eukaryotic cells, an ideal rare codon for encoding proteins, polypeptides, or amino acid residues containing unnatural amino acids should meet the following requirements: 1) The rare codon used has a low abundance of endogenous tRNAs in the host cell that compete with the recoding tRNA for binding to the codon, which can reduce the binding of natural amino acids to the codon, thereby increasing the recoding efficiency of unnatural amino acids. 2) The rare codon used has a low usage frequency in the total transcriptome or on the transcription template of the target gene, which can minimize the incorporation of unnatural amino acids into non-target proteins, thereby reducing erroneous insertion of unnatural amino acids in the proteome. 3) The recoding tRNA carrying the anticodon that binds to the rare codon can be efficiently recognized by its homologous recoding aminoacyl-tRNA synthetase, thereby achieving high recoding efficiency for unnatural amino acids. The orthogonality between the enzymes and tRNA tools used in rare codon recoding ensures the feasibility of site-specific insertion of unnatural amino acids.

This application provides a method for analyzing the usage frequency of codons and their corresponding tRNA abundance in eukaryotic cells. Due to the high complexity of eukaryotic genomes, determining the usage frequency of codons in eukaryotic genomes is relatively difficult compared to prokaryotes. This application obtains the usage frequency of all codons by systematically detecting the usage frequency of all codons in the total transcriptome of the host cell or on the transcription template of the target gene. By systematically measuring the tRNA abundance in the host cell, the abundance of endogenous tRNAs corresponding to all codons is obtained. Through the measurement results of usage frequency and endogenous tRNA abundance, 30 codons with lower occurrence frequency and lower endogenous tRNA abundance in eukaryotic cells were screened.

This application provides a method for detecting the recoding efficiency of rare codons in eukaryotic organisms. This application provides a method for determining the recoding efficiency of rare codons in detected proteins by liquid chromatography-mass spectrometry (LC-MS) based on differences in the molecular weight of the detected proteins. This application also provides a mass tag labeling method to determine the recoding efficiency of rare codons in detected proteins by detecting the labeled amount of proteins containing unnatural amino acids. The biochemical reactions involved in the mass tag labeling method may include, but are not limited to, CuAAC reactions, SPAAC reactions, and/or IEDDA reactions. Macromolecules that can be used for mass tag labeling may include, but are not limited to, TCO ligands and their conjugates or analogs, such as TCO-Cy5 and TCO-PEG5000.

The method for expressing unnatural amino acids through rare codon recoding described in this application may include one or more of the following optimization methods: modification of aminoacyl-tRNA synthetases, modification of recoding tRNAs, optimization of culture systems, and/or modification of the host cell genome.

### Modification of Aminoacyl-tRNA Synthetases

The modification of aminoacyl-tRNA synthetases may include modifying their nuclear localization ability. Natural aminoacyl-tRNA synthetase sequences contain amino acid sequences that act as nuclear localization signals, such as sequences in archaeal pyrrolysyl-tRNA synthetases that serve as nuclear localization signals. Mislocalization of expressed aminoacyl-tRNA synthetases to the nucleus in eukaryotic cells can limit the genetic code encoding efficiency based on such synthetases because it restricts the amount of synthetase available in the cytoplasm where translation occurs. During translation, if there is a high concentration of aminoacyl-tRNA synthetases and a high concentration of recoding tRNAs that can bind to aminoacyl-tRNA synthetases for aminoacylation with unnatural amino acids in the cytoplasm of the host cell, particularly near ribosomes, unnatural amino acids are more easily incorporated into the synthesized polypeptide chain.

This application relates to a method for optimizing the encoding efficiency of aminoacyl-tRNA synthetases, including modifying the aminoacyl-tRNA synthetases to alter their nuclear localization ability. The modification may include removing the nuclear localization signal from the synthetase or replacing the nuclear localization signal with a suitable nuclear export signal.

A nuclear localization signal (abbreviated as “NLS,” also known as “nuclear localization sequence” in the art) is an amino acid sequence that can direct a protein, polypeptide, or amino acid residue containing it (e.g., the aminoacyl-tRNA synthetase described in this invention) into the nucleus of a eukaryotic cell. The nuclear localization signal mediates nuclear localization by binding the protein, polypeptide, or amino acid residue containing the NLS to nuclear import proteins to form a complex that passes through the nuclear pore.

A nuclear export signal (abbreviated as “NES”) is an amino acid sequence that can direct a protein, polypeptide, or amino acid residue containing it (e.g., the aminoacyl-tRNA synthetase described in this invention) out of the nucleus of a eukaryotic cell.

The removal of the nuclear localization signal from the aminoacyl-tRNA synthetase described in this application can be achieved through gene knockout, gene mutation, and/or gene silencing. This may include knocking out alleles encoding the NLS signal or administering one or more substances selected from the group consisting of antisense RNA, siRNA, shRNA, and the CRISPR/Cas9 system to the host cell.

The introduction of the nuclear export signal into the aminoacyl-tRNA synthetase described in this application can be achieved by replacing the nuclear localization signal. The nuclear export signal can be a sequence rich in hydrophobic amino acids, such as including one, two, three, four, or more hydrophobic amino acid sequences, like a hydrophobic leucine-rich NES, where the hydrophobic leucine-rich NES can have a sequence with one, two, three, four, or more hydrophobic residues. The nuclear export signal can also be a sequence rich in isoleucine, valine, phenylalanine, and methionine amino acids, where the hydrophobic NES rich in isoleucine, valine, phenylalanine, and methionine amino acids can have a sequence with one, two, three, four, or more hydrophobic residues.

The nuclear export signal introduced in this application can be an endogenous nuclear export signal, meaning a nuclear export signal that the host cell can express without any genetic manipulation, or an exogenous nuclear export signal, meaning a nuclear export signal that the host cell can express only after genetic manipulation.

This application provides an aminoacyl-tRNA synthetase containing a nuclear export signal. The nuclear export signal can be connected to the aminoacyl-tRNA synthetase in any form, as long as the nuclear export signal can be recognized by nuclear receptors and prevent the aminoacyl-tRNA synthetase from entering the nucleus. The nuclear export signal can be introduced at the N-terminus and/or C-terminus of the aminoacyl-tRNA synthetase. Multiple copies of the nuclear export signal can be introduced at the N-terminus and/or C-terminus of the aminoacyl-tRNA synthetase.

Optimization of Recoding tRNAs

This application provides a method for optimizing the structure of recoding tRNAs to improve recoding efficiency. The structural optimization method can improve recoding efficiency by enhancing the stability of the tRNA. The structural optimization method can also improve recoding efficiency by increasing the concentration of the optimized tRNA.

The optimization method can alter the types and/or numbers of bases on the recoding tRNA, with optimization methods including but not limited to one or more of the following: frameshifting, deletion, and substitution. The optimization method can alter the secondary structure of the recoding tRNA, such as increasing the base interactions within the secondary structure of the recoding tRNA. The optimization can be applied to the D-loop, T-loop, anticodon loop, variable loop, and/or amino acid arm of the recoding tRNA. The method can involve adding hinges between various structures of the recoding tRNA.

The optimized recoding tRNA sequences are as shown in any of SEQ ID NO: 4 or 36.

The expression of proteins containing unnatural amino acids in host cells can be carried out without significantly interfering with the eukaryotic host cell. For example, since the recoding efficiency of the amber codon depends on the competition between the recoding tRNA (e.g., a recoding tRNA that binds to the amber stop codon) and vertebrate translation release factors (which bind to stop codons and promote the release of the growing peptide from the ribosome), the recoding efficiency in this application can be adjusted by increasing the expression level, i.e., concentration, of the recoding tRNA in the cytoplasm.

This application provides a method for increasing the expression level of recoding tRNAs. The expression level of recoding tRNAs can be achieved by increasing the number of gene copies encoding the recoding tRNA. To increase the expression level of recoding tRNAs, the number of gene copies of the recoding tRNA on a single expression vector encoding the recoding tRNA can be increased, such as increasing the number of gene copies expressing the recoding tRNA on the vector to 1-12 times that before optimization. The expression level of recoding tRNAs can also be increased by enhancing the copy number of the expression vector encoding the recoding tRNA itself, such as by optimizing the replication ability of the expression vector to increase its own copy number, thereby increasing the expression level of the recoding tRNA.

In the process of inserting unnatural amino acids using rare codons, the endogenous tRNAs corresponding to the rare codons compete with the recoding tRNAs carrying unnatural amino acids for binding to the rare codons, thereby allowing natural amino acids to be inserted at the rare codon sites, reducing the insertion efficiency of unnatural amino acids. This application provides a method for improving the recoding efficiency of unnatural amino acids corresponding to rare codons by weakening the competitive insertion of natural amino acids.

Optimization of Culture Systems and/or Host Cell Genome

This application provides a method for optimizing the host cell culture system to improve the recoding efficiency of rare codons. The method can adjust the content of culture components in the culture system to enhance the recoding efficiency of rare codons, such as reducing the content of natural amino acids in the culture system that compete with unnatural amino acids for binding to rare codons. The natural amino acids with reduced content can be one or more of the following: glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, and histidine. The natural amino acids with reduced content can be associated with the natural amino acids carried by the endogenous tRNAs corresponding to the rare codons used. The method can reduce the content of natural amino acids to, including but not limited to, 50%, 40%, 30%, 20%, 15%, 10%, 8%, 5%, 3%, 2%, 1%, 0.1%, and/or 0.01% of the natural amino acid content in the original complete culture system of the host cell.

Due to the complexity of the tRNA genome in eukaryotic cells, the impact of targeted knockout of endogenous isoacceptor tRNAs on cell survival cannot be predicted, making knockout more difficult. This application provides a method for improving the recoding efficiency of rare codons by optimizing the host cell genome. The method can enhance the recoding efficiency of rare codons by reducing the content of endogenous isoacceptor tRNAs in the host cell, while ensuring that the growth and metabolic capabilities of the knockout cell line are not affected compared to the original cells.

The method can reduce the content of any endogenous isoacceptor tRNA in the host cell, where the endogenous isoacceptor tRNA can be one or more of the following: glycine tRNA, alanine tRNA, valine tRNA, leucine tRNA, isoleucine tRNA, methionine tRNA, proline tRNA, tryptophan tRNA, serine tRNA, tyrosine tRNA, cysteine tRNA, phenylalanine tRNA, asparagine tRNA, glutamine tRNA, threonine tRNA, aspartic acid tRNA, glutamic acid tRNA, lysine tRNA, arginine tRNA, and histidine tRNA. The method includes knockout, silencing, and/or mutation of genes encoding endogenous isoacceptor tRNAs, such as targeted knockout of alleles encoding endogenous isoacceptor tRNAs in the genome.

The method may include administering to the host cell one or more substances selected from the group consisting of antisense RNA, siRNA, shRNA, and the CRISPR/Cas9 system. The method may also include administering sgRNA to the host cell, where the sequence of the sgRNA includes, but is not limited to, any of the sequences shown in SEQ ID NO: 95-98.

This application also provides a method for screening and identifying the genotype of cell lines with endogenous isoacceptor tRNA gene knockouts. The screening method may include antibiotic selection, where the antibiotics may include, but are not limited to, one or more of ampicillin, kanamycin, chloramphenicol, spectinomycin, tetracycline, bleomycin, streptomycin, hygromycin, gentamicin, puromycin, erythromycin, and blasticidin. The identification method may include one or more of first-generation, second-generation, and third-generation gene sequencing methods, such as Sanger sequencing.

### Optimization of Background Misincorporation

This application provides optimization methods to reduce the background incorporation of unnatural amino acids in the proteome, breaking the widely held belief that the background incorporation rate of rare codons in eukaryotes is too high to achieve unnatural amino acid recoding. While reducing the background incorporation rate, it ensures the normal growth, metabolism, and reproduction of cells, i.e., ensures low cytotoxicity.

The erroneous insertion of unnatural amino acids into the host cell's proteome can affect the normal structure and function of other proteins in the host cell, thereby impacting the survival vitality of the host cell. The usage frequency of rare codons in eukaryotes is higher than in prokaryotes, making it difficult to achieve specific site-directed insertion during translation, leading to mistranslation and background incorporation of unnatural amino acids in the host cell's proteome. In protein production activities, a high background incorporation rate in the host cell can lead to cytotoxicity, waste of cellular resources, and reduced protein yield. Taking the amber stop codon-mediated genetic code expansion system as an example, when unnatural amino acids are incorporated into unwanted amber stop codon sites in the proteome, it can prevent other proteins from terminating translation and cause them to continue extending, leading to cytotoxicity, i.e., disruption of cellular function caused by the genetic code expansion translation system.

During protein expression, ribosomes in the host cell are occupied as reaction sites. To reduce the erroneous insertion of unnatural amino acids into the host cell's proteome, the expression of the target protein can be made to occupy more ribosomal resources during the expression process of the protein containing the target gene, while the expression of other non-target proteins in the proteome (host cell-expressed proteins other than the target protein) occupies fewer ribosomal resources. This reduces the insertion of unnatural amino acids into non-target proteins, i.e., the background of the proteome, during the expression of the target gene.

This application provides a method for reducing background incorporation by co-expressing the target protein gene. The method can construct the target protein gene and the recoding system gene on the same expression vector, expressing the target protein gene simultaneously with the recoding system gene. The method can also construct the target protein gene and the recoding system gene on different expression vectors, expressing the target protein gene simultaneously with the recoding system gene.

This application also provides a method for reducing background incorporation by introducing a phase separation system. The method can construct the phase separation system encoding gene and the recoding system gene on the same expression vector, expressing the phase separation system gene simultaneously with the recoding system gene. The method can also construct the phase separation system encoding gene and the recoding system gene on different expression vectors, expressing the phase separation system simultaneously with the recoding system gene.

In some embodiments, the phase separation system may include the fusion expression of λN22 protein and FUS protein with the aminoacyl-tRNA synthetase. Further, the phase separation system may include introducing the boxB sequence, which is efficiently recognized and bound by the λN22 protein, into the transcription template.

This application also provides a method for detecting the survival vitality of host cells containing the recoding system. The method includes, but is not limited to, one or more detection methods from the following group: detection of cellular metabolic vitality, detection of cellular growth vitality, detection of newly synthesized proteins in cells, and transcriptome sequencing.

### Proteins Containing Unnatural Amino Acids

This application also provides proteins containing unnatural amino acids obtained using the rare codon recoding method. The proteins may include one or more unnatural amino acids or one or more types of unnatural amino acids. Typically, protein production also involves post-translational modification and packaging, and this application also provides proteins containing unnatural amino acids that have undergone post-translational modification and packaging.

The proteins containing unnatural amino acids may include therapeutic proteins, diagnostic proteins, and industrial enzymes. The proteins containing unnatural amino acids may be selected from one or more of transcription regulatory proteins, cytokines, growth factor receptors, inflammatory molecules, oncogene products, peptide hormones, signal transduction molecules, and/or steroid hormone receptors.

The proteins containing unnatural amino acids may be selected from one or more of the following: α-1 antitrypsin, angiostatin, antihemolytic factor, antibodies, apolipoproteins, atrial natriuretic peptide, C-X-C chemokines, Hedgehog proteins, hemoglobin, hepatocyte growth factor (HGF), hirudin, insulin, insulin-like growth factor (IGF), keratinocyte growth factor (KGF), lactoferrin, leukemia inhibitory factor, luciferase, neurturin, neutrophil inhibitory factor (NIF), oncostatin M, osteogenin, parathyroid hormone, PD-ECSF, PDGF, pleiotrophin, protein A, protein G, pyrogenic exotoxins A, B, or C, relaxin, renin, SCF, soluble complement receptor I, soluble interleukin receptor, soluble TNF receptor, somatostatin, streptokinase, superantigens, staphylococcal enterotoxins, superoxide dismutase (SOD), toxic shock syndrome toxin, thymosin al, tissue plasminogen activator, tumor growth factor (TGF), tumor necrosis factor, and/or corticosterone.

The proteins containing unnatural amino acids may be selected from one or more of the following: GAL4, erythropoietin (EPO), human growth hormone, T39765, NAP-2, ENA-78, Gro-a, Gro-b, Gro-c, IP-10, GCP-2, NAP-4, SDF-1, PF4, MIG, calcitonin, c-kit ligand, CC chemokines, monocyte chemoattractant protein-1, monocyte chemoattractant protein-2, monocyte chemoattractantprotein-3, monocyte inflammatoryprotein-la, monocyte inflammatoryprotein-10, RANTES, 1309, R83915, R91733, HCC1, T58847, D31065, T64262, CD40, CD40 ligand, C-kit ligand, collagen, colony-stimulating factor (CSF), complement factor 5a, complement inhibitor, complement receptor 1, DHFR, epithelial neutrophil-activating peptide-78, GROα/MGSA, GROβ, GROγ, MIP-1α, MIP-1δ, MCP-1, epidermal growth factor (EGF), epithelial neutrophil-activating peptide, erythropoietin (EPO), exfoliative toxin, factor IX, factor VII, factor VIII, factor X, fibroblast growth factor (FGF), fibrinogen, fibronectin, G-CSF, GM-CSF, glucocerebrosidase, gonadotropin, human serum albumin, ICAM-1, ICAM-1 receptor, LFA-1, LFA-1 receptor, IGF-I, IGF-II, IFN-α, IFN-β, IFN-γ, IL-1, IL-2, IL-3, IL-4, IL-5, IL-6, IL-7, IL-8, IL-9, IL-10, IL-11, IL-12, SEA, SEB, SEC1, SEC2, SEC3, SED, SEE, soluble I-CAM1, TGF-α, TGF-β, tumor necrosis factor α, tumor necrosis factor R, tumor necrosis factor receptor (TNFR), VLA-4 protein, VCAM-1 protein, vascular endothelial growth factor (VEGF), urokinase, Mos, Ras, Raf, Met, p53, Tat, Fos, Myc, Jun, Myb, Rel, estrogen receptor, progesterone receptor, testosterone receptor, aldosterone receptor, LDL receptor, SCF/c-Kit, CD40L/CD40, VLA-4/VCAM-1, ICAM-1/LFA-1, and/or hyaluronan/CD44.

This application also provides industrial enzymes or their biologically active portions containing unnatural amino acids. Examples of industrial enzymes or their biologically active portions include, but are not limited to, one or more of amidases, amino acid racemases, acylases, dehalogenases, dioxygenases, diarylpropane peroxidases, epimerases, epoxide hydrolases, esterases, isomerases, kinases, glucose isomerases, glycosidases, glycosyltransferases, haloperoxidases, monooxygenases, lipases, lignin peroxidases, nitrile hydratases, nitrilases, proteases, phosphatases, subtilisins, transaminases, and nucleases.

This application also provides protein vaccines containing unnatural amino acids. For example, this application may include replacing one or more natural amino acids in one or more protein vaccines with unnatural amino acids, where the protein vaccines may be derived from one or more of the following: infectious fungi, such as Aspergillus, Candida; bacteria serving as pathogen models, especially Escherichia coli, and medically important bacteria such as Staphylococcus or Streptococcus (e.g., Streptococcus pneumoniae); protozoa such as sporozoa (e.g., Plasmodium), rhizopods (e.g., Entamoeba), and flagellates (Trypanosoma, Leishmania, Trichomonas, Giardia, etc.); viruses such as (+) RNA viruses (examples include poxviruses, e.g., vaccinia; picornaviruses, e.g., poliovirus; togaviruses, e.g., rubella virus; flaviviruses, e.g., HCV; and coronaviruses), (−) RNA viruses (e.g., rhabdoviruses, e.g., VSV; paramyxoviruses, e.g., RSV; orthomyxoviruses, e.g., influenza virus; bunyaviruses; and arenaviruses), dsDNA viruses (e.g., reoviruses), RNA to DNA viruses (i.e., retroviruses, e.g., HIV and HTLV), and certain DNA to RNA viruses (such as hepatitis B virus).

This application also provides agriculture-related proteins containing unnatural amino acids. For example, insect resistance proteins (e.g., Cry proteins), starch and lipid production enzymes, plant and insect toxins, toxin resistance proteins, mycotoxin detoxification proteins, plant growth enzymes (e.g., ribulose-1,5-bisphosphate carboxylase/oxygenase, “RUBISCO”), lipoxygenases (LOX), and/or phosphoenolpyruvate (PEP) carboxylase.

This application also provides the use of a method or system for producing proteins containing unnatural amino acids in the production of therapeutic proteins.

This application also provides a kit comprising a nucleotide sequence encoding a first recoding tRNA or the nucleotide sequence of the first recoding tRNA, and a nucleotide sequence encoding a first aminoacyl-tRNA synthetase or the first aminoacyl-tRNA synthetase, wherein the recoding tRNA includes one or more of the following anticodons: TCG, ACG, CGA, TCA, CGC, TTG, ATA, GCG, TTA, and TGT.

In some embodiments, the kit further includes a first unnatural amino acid, and the first aminoacyl-tRNA synthetase is capable of charging the first recoding tRNA with the first unnatural amino acid. This application also provides the use of the kit for producing proteins containing unnatural amino acids in eukaryotic cells.

This application also provides a cell comprising a nucleotide sequence encoding a first recoding tRNA or the nucleotide sequence of the first recoding tRNA, and a nucleotide sequence encoding a first aminoacyl-tRNA synthetase or the first aminoacyl-tRNA synthetase.

In some embodiments, the recoding tRNA includes one or more of the following anticodons: TCG, ACG, CGA, TCA, CGC, TTG, ATA, GCG, TTA, and TGT.

In some embodiments, the expression level and/or activity of endogenous tRNAs encoding rare codons is downregulated.

This application also provides a recombinant vector capable of expressing the nucleotide sequence of a first recoding tRNA and a first aminoacyl-tRNA synthetase in a host cell.

In some embodiments, the recombinant vector can express the nucleotide sequence of a second recoding tRNA and a second aminoacyl-tRNA synthetase. The recombinant vector can have a sequence as shown in any of SEQ ID NO: 95-98.

Without being bound by any theory, the following examples are merely to illustrate the fusion proteins, preparation methods, and uses of this application and are not intended to limit the scope of the invention,

### EXAMPLES

### Example 1: Screening of Rare Codons in Mammalian Cells for UAA Incorporation

This example describes a method for screening recoding codons in mammalian cells based on rarity, high recoding efficiency, and low background incorporation. The following experiments were performed in HEK293T cells.

### 1.1 Codon Rarity Evaluation

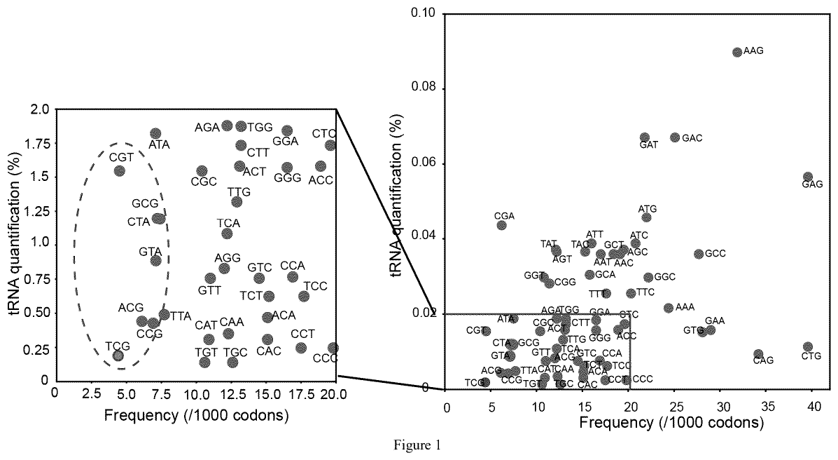

The HEK293T genome contains 61 sense codons, with usage frequencies ranging from 4 to 40 occurrences per 1,000 codons (4/1000-40/1000). Potential rare codons in HEK293T with high recoding efficiency and low background incorporation include: CGT, GTA, CCG, GCG, CTA, ACG, and TCG (FIG. 1). As shown in FIG. 1, these seven rare codons have a usage frequency of less than 20 per 1,000 codons, and their cognate tRNA abundance is below 2%.

### 1.2 Recoding Efficiency Evaluation

### 1.2.1 Method for Evaluating Recoding Efficiency

This study employed the chPylRS_{IPYE}(TetRS)/MmPylT system, derived from the pyrrolysine orthogonal translation system, as the recoding system. This system efficiently recognizes the tetrazine-based noncanonical amino acid (3-(6-butyl-1,2,4,5-tetrazin-3-yl)-phenylalanine, TetBu). All plasmids were constructed using Gibson assembly. The L270G/N311G/C313A mutations for TetBu recognition were introduced into chPylRS_{IPYE} via overlap PCR to generate pCMV-TetRS. Subsequently, PylT_{yyy} (where yyy represents the anticodons for TCG, ACG, GCG, CCG, CGT, GTA, and CTA) was synthesized and inserted into pCMV-TetRS, yielding pCMV-TetRS-PylT_{yyy}.

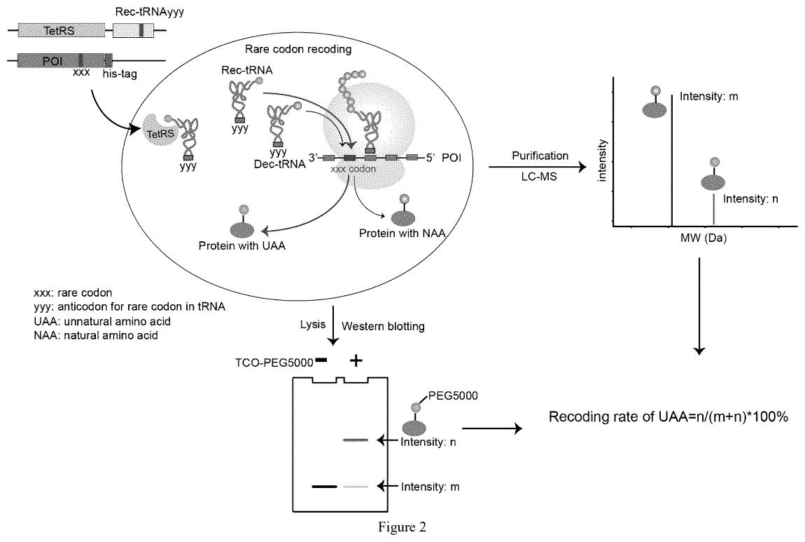

When the recoding system (TetRS/PylT_{yyy}) is co-expressed with a target protein carrying the recoding codon in HEK293T, the recoding codon can be translated into a natural amino acid by endogenous tRNA (Dec-tRNA), or recoding into TetBu by the engineered tRNA (Rec-tRNA), producing a mixture of protein byproducts (with a single-site mutation) and the target protein containing TetBu. Based on this, two methods were established to assess sense codon recoding efficiency: a) Liquid chromatography-mass spectrometry (LC-MS) for differentiates proteins based on molecular weight differences, and b) TCO-PEG5000 macromolecular label target proteins containing TetBu, which increases their molecular weight by 5 kDa. The shift in protein bands is detected via immunoblotting (FIG. 2).

1.2.2 Screening of Rare Codons with High Recoding Efficiency

(1) Transfection: Plasmids encoding the TetBu/PylT_{yyy} recoding system (where xxx represents different codons and yyy represents different anticodons) were co-transfected with the plasmid mCherry-T2A-EGFP^{N149XXX}-6×His at a 1:1 (g:g) ratio. For a 10 cm dish, the total transfection mixture contained 20 μg DNA+60 L Lipofectamine 2000+2 mL Opti-MEM.

(2) Protein expression and purification: After 6 hours, the medium was replaced with fresh DMEM containing 200 μM TetBu for 40 hours to induce protein expression. Cells were lysed in 1 mL Modified RIPA Buffer, and the supernatant was purified using Ni-NTA beads.

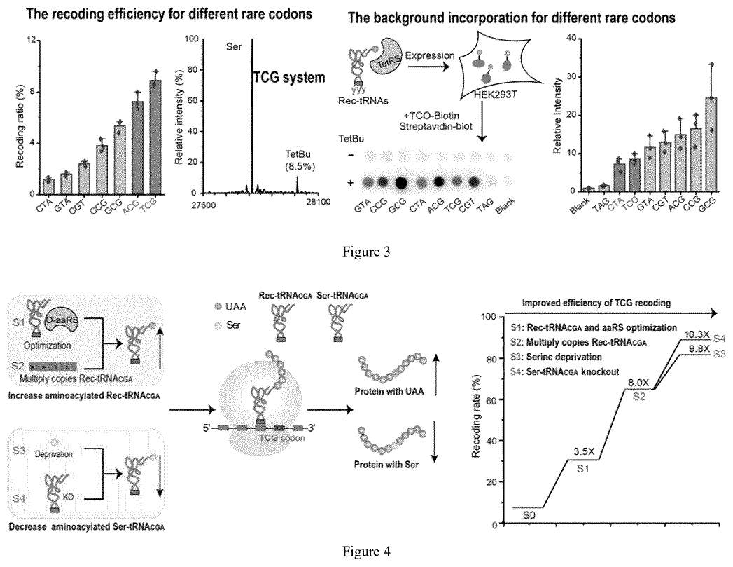

(3) LC-MS analysis of recoding efficiency:Using a Waters Xevo G2-XS TOF Mass Spectrometer, the recoding efficiency of rare codons (CTA, GTA, CGT, CCG, GCG, ACG, and TCG) at the EGFP149 site was evaluated. TCG showed the highest recoding efficiency (8.5%), followed by ACG (7.1%) (FIG. 3, left).

### 1.3 Background Incorporation Analysis

### 1.3.1 Method for Assessing Background Incorporation

When the TetRS/PylT_{yyy} recoding system is expressed in HEK293T, it recodes endogenous genomic codons into TetBu, generating a modified proteome containing TetBu. The IEDDA reaction between TetBu and trans-cyclooctene (TCO) enables biotinylation of TetBu-containing proteins via TCO-Biotin. Streptavidin dot blot detects biotin signals, where signal intensity correlates with TetBu incorporation levels (FIG. 3, right).

1.3.2 Screening of Rare Codons with Low Background Incorporation
(1) Intracellular expression of the recoding system: Plasmids encoding TetBu/PylT_{yyy} (yyy=anticodons for TCG, ACG, GCG, CCG, CGT, GTA, CTA) were transfected using Lipofectamine 2000. After 6 hours, cells were treated with fresh DMEM±200 μM TetBu for 40 hours. Cells were lysed in 100 μL Modified RIPA Buffer to obtain whole-cell lysates.

### (2) Bioorthogonal IEDDA Reaction:

The reaction system was prepared as described in Table 3. Reaction products were mixed with 7 μL bromophenol blue-free 4×SDS loading buffer, boiled at 100° C. for 10 min, and analyzed via streptavidin dot blot.

TABLE 3
IEDDA reaction system

| Component | Volume (μL) |
|---|---|
| Whole-cell lysate supernatant | 19 |
| 1 mM (4E)-TCO-PEG4-biotin | 1 |

Mix and react at 25° C. for 30 min

| | |
|---|---|
| 200 mM TetBu | 1 |

Mix and react at 25° C. for 5 min

### (3) Streptavidin Dot Blot Assay

Samples were spotted onto a nitrocellulose membrane and air-dried at room temperature for 15 min. The membrane was washed with 10 mL TBST (80 rpm, 5 min) three times, discarding the supernatant each time. Blocking was performed with 10 mL TBST containing 5% non-fat milk for 1 h, followed by three additional TBST washes (10 mL, 80 rpm, 5 min each). The membrane was then incubated with streptavidin-HRP overnight at 4° C. Biotin signals were visualized using an Azure Biosystems C400 imaging system. The result show that CTA and TCG codon groups exhibited the lowest biotin signal intensity, indicating that recoding systems based on these codons demonstrated significantly lower proteome-wide background TetBu incorporation (FIG. 3, right).

### Example 2: Optimization of the TetRS/PylT_{CGA} Recoding System

As previously described, the abundance of cognate tRNAs for each sense codon in HEK293T cells determines the rate and efficiency of amino acid incorporation during translation. Here, we propose two strategies to optimize the TetRS/PylT_{CGA} recoding system: On the one hand, enhancing the activity of the TetRS/PylT_{CGA} system. One the other hand, reducing intracellular levels of the Ser-tRNA_{CGA} by serine starvation. FIG. 4 illustrates four strategies to improve the recoding efficiency of the TetRS/PylT_{CGA} System.

### 2.1 Optimization of TetRS/PylT_{CGA} System Components

This section describes methods to systematically enhance the efficiency of the TetRS/PylT_{CGA} system in mammalian cells by optimizing both the aminoacyl-tRNA synthetase (TetRS) and the recoding tRNA (PylT_{CGA}).

### 2.1.1 Optimization of the Aminoacyl-tRNA Synthetase (TetRS)

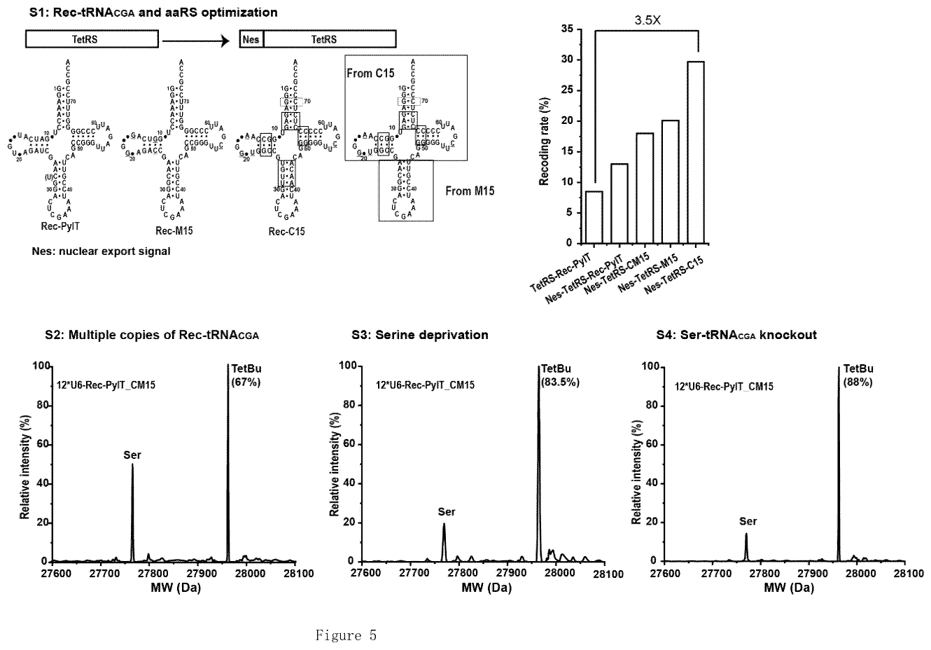

To improve the aminoacylation activity of TetRS in mammalian cells, we fused a nuclear export signal (Nes: ACPVPLQLPPLERLTLD, SEQ ID NO: 99) to the N-terminus of TetRS, increasing its cytoplasmic concentration. Mass spectrometry results showed that Nes-TetRS increased the recoding efficiency of the TetRS/PylT_{CGA} system from 8.5% to 13% (FIG. 5).

2.1.2 Screening and Optimization of Recoding tRNA (PylT_{CGA})

To enhance the decoding activity of PylT_{CGA} in mammalian cells, we designed and constructed a series of PylT_{CGA} mutants based on previously reported PylT^{C15} and PylT^{M15} mutants (sequences shown in SEQ ID NO: 2-4, FIG. 8). When combined with Nes-TetRS, the optimized system achieved a recoding efficiency of 29.7% (FIG. 5, left), representing a 3.5-fold improvement over the original TetRS/PylT_{CGA} system (FIG. 5).

The table 4 lists the nucleotide sequences of the PylT_{CGA} mutants. The table 5 provides the primers used for Gibson assembly to construct the TetRS/PylT_{CGA} mutant expression plasmids.

TABLE 4
The nucleotide sequences of the PylT_{CGA} mutants

| PylT name | Sequence from 5′ to 3′ |
|---|---|
| PylT^{WT}_{CGA} | ggaaacctgatcatgtagatCGAatggactCGAaatc |
| | cgttcagccgggttagattcccggggtttccgcca |
| | (SEQ ID NO: 1) |
| Pyl^{TC15}_{CG}A | gggagagtggccaaggtggccgtgttgactCGAaatc |
| | aacacaggggggttCGAttcccccctctcccgcca |
| | (SEQ ID NO: 2) |
| Pyl^{TM15}_{CGA} | ggaaacctggtcagggagacCGAacggactCGAaatc |
| | cgttcagccgggttCGAttcccggggtttccgcca |
| | (SEQ ID NO: 3) |
| PylT^{CM15}_{CGA} | gggagagtggccaaggtggcCGAacggactCGAaatc |
| | cgttcaggggggttCGAttcccccctctcccgcca |
| | (SEQ ID NO: 4) |

TABLE 5
The primers for PylT_{CGA} variants construction

| Primer name | Sequence from 5′ to 3′ |
|---|---|
| Pyl-C15-F | atttcccCGAaaaaTGGcGGGAGAGgggggaatCGA |
| | acccccctgtgttgatttCGAgt (SEQ ID NO: |
| | 100) |
| Pyl-C15-R | aCGAaacaccggGaGAGTggccaaggtggccgtgtt |
| | gactCGAaatcaacacagggggg (SEQ ID NO: |
| | 101) |
| Pyl-M15-F | atttcccCGAaaaaTGGcggaaaccccgggaatCGA |
| | acccggctgaacggatttCGAgt (SEQ ID NO: |
| | 102) |
| Pyl-M15-R | aCGAaacaccggaaaccTggtcagggagacCGAacg |
| | gactCGAaatccgttcagccggg (SEQ ID NO: |
| | 103) |
| Pyl-CM15-F | atttcccCGAaaaaTGGcGGGAGAGgggggaatCGA |
| | acccccctgAACGGAtttCGAgT (SEQ ID NO: |
| | 104) |
| Pyl-CM15-R | aCGAaacaccggGaGAGTggccaaggtggcCGAACG |
| | GActCGAaaTCCGTTcagggggg (SEQ ID NO: |
| | 105) |

### 2.1.3 Optimization of Recoding PylT_{CGA} Expression Level

To enhance the expression level of PylT_{CGA} in mammalian cells, we constructed expression plasmids containing multicopy tRNA expression cassettes (U6-PylT^{CM15}_{CGA}). Mass spectrometry results demonstrated that increasing the copy number of the recoding tRNA in the plasmid significantly improved recoding efficiency. When HEK293T cells were co-transfected with the Nes-TetRS/12×(U6-PyT^{CM15}_{CGA}) recoding system plasmid and the mCherry-T2A-EGFP^{Y151TCG}-6×His reporter plasmid, the recoding efficiency at the TCG codon (position 151 of EGFP) reached 67% (FIG. 5).

### (1) Construction Method and Primers

Four copies of tRNA were amplified via PCR using primers listed in Table 6 and assembled into a pUC vector using Gibson assembly. The constructed plasmid was digested with BamHI and SpeI to excise the 4×(U6-PylT^{CM15}_{CGA}) fragment. A second digestion was performed with BgIII and SpeI on the vector containing 4×(U6-PylT^{CM15}_{CGA}), followed by T4 ligation to generate the 8×(U6-PylT^{CM15}_{CGA}) plasmid. The same method was used to construct the 12×(U6-PyT^{CM15}C_{GA}) plasmid.

TABLE 6
The primers for multi-copy tRNA cassette
construction

| Primer | |
|---|---|
| name | Sequence from 5′ to 3′ |
| tRNA-F1 | CAACGAATTCggatccGGTACCggggttccgcgcacattt |
| | ccccg (SEQ ID NO: 106) |
| tRNA-R1 | gttgaacctgtcacgtcctggcccgtacatcgCGAaggtc |
| | gggcagg (SEQ ID NO: 107) |
| tRNA-F2 | caggacgtgacaggttcaacggggttccgcgcacatttcc |
| | cc (SEQ ID NO: 108) |
| tRNA-R2 | gttgatCGAtccgtagctacgtacatcgCGAaggtcgggc |
| | agg (SEQ ID NO: 109) |
| tRNA-F3 | gtagctacggatCGAtcaacggggttccgcgcacatttcc |
| | cc (SEQ ID NO: 110) |
| tRNA-R3 | gctgagagtgactctgcgtggtacatcgCGAaggtcgggc |
| | ag (SEQ ID NO: 111) |
| tRNA-F4 | cacgcagagtcactctcagcggggttccgcgcacatttcc |
| | cc (SEQ ID NO: 112) |
| tRNA-R4 | ctgaccagatctCAATAATCAATGTCAGGTACCgtacatc |
| | gCGAaggtcgggcag(SEQ ID NO: 113) |
| tRNA-v-r | CAACGAATTCggatccGGTACCggggttccgcgcacattt |
| | ccccg(SEQ ID NO: 106) |
| tRNA-v-f | ctgaccagatctcaataatcaatgtcaggtaccgtacatc |
| | gCGAaggtcgggcag (SEQ ID NO: 113) |

### (2) the Transfection, Protein Expression, Purification and LC-MS Analysis: Methods as Described in Section 1.2.2.

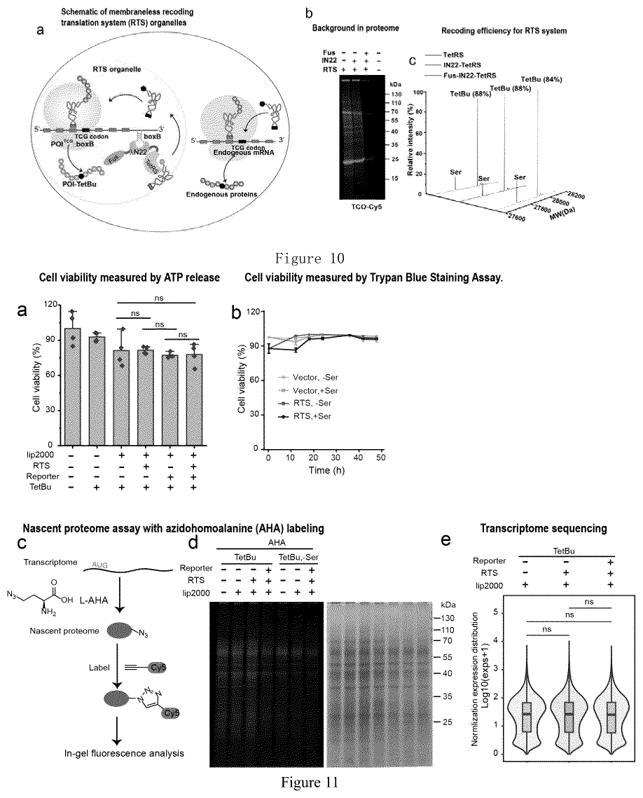

LC-MS analysis confirmed that increasing the tRNA copy number in the recoding plasmid markedly enhanced recoding efficiency. With the Nes-TetRS/12×(U6-PylT^{CM5}_{CGA}) system, the recoding efficiency at EGFP^{Y151TCG} reached 67% (FIG. 10).

### 2.2 Serine Starvation Treatment to Enhance TCG Recoding Efficiency

The following protocol applies to all TCG codon recoding systems, where serine starvation is used to improve the efficiency of non-canonical amino acid incorporation.

This example describes a method to enhance TetBu recoding efficiency at TCG codons in mammalian cells through serine starvation, using the TetRS/PylT system as an example.

Procedure: One day before transfection, HEK293T cells cultured in normal medium (90-100% confluency) were trypsinized and resuspended in 3 mL serine/glycine-free DMEM/F12 medium. Then the cells were seeded at a 1:3 ratio in new dishes or plates and cultured in serine/glycine-free DMEM/F12 for 16-20 hours. When the cells get to 60-70% confluency, cells were co-transfected with the Nes-TetRS/12×(U6-PyT^{CM15}_{CGA}) recoding system plasmid and the mCherry-T2A-EGFP^{Y151TCG}-6*His reporter plasmid. After 6 hours, the medium was replaced with fresh serine/glycine-free DMEM/F12 containing 200 μM TetBu, followed by 40 hours of protein expression. The results show that the optimized Nes-TetRS/12×(U6-PyT^{CM15}_{CGA}) system with the serine starvation could achieve 83.5% recoding efficiency at the TCG codon (FIG. 5).

### 2.3 Reducing Endogenous Decoding Ser-tRNA Levels to Improve TCG Recoding Efficiency

This section describes a strategy to enhance TCG recoding efficiency by knocking out endogenous Ser-tRNA_{CGA} in HEK293T cells using CRISPR/Cas9.

### 2.3.1 Construction and Screening of Ser-tRNACGₐ Knockout Cell Lines

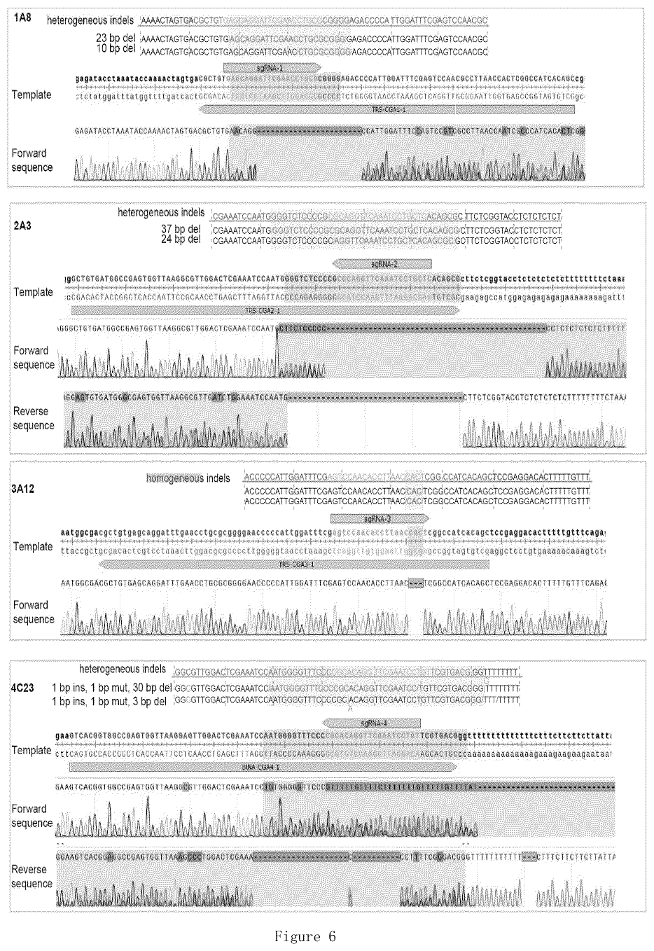

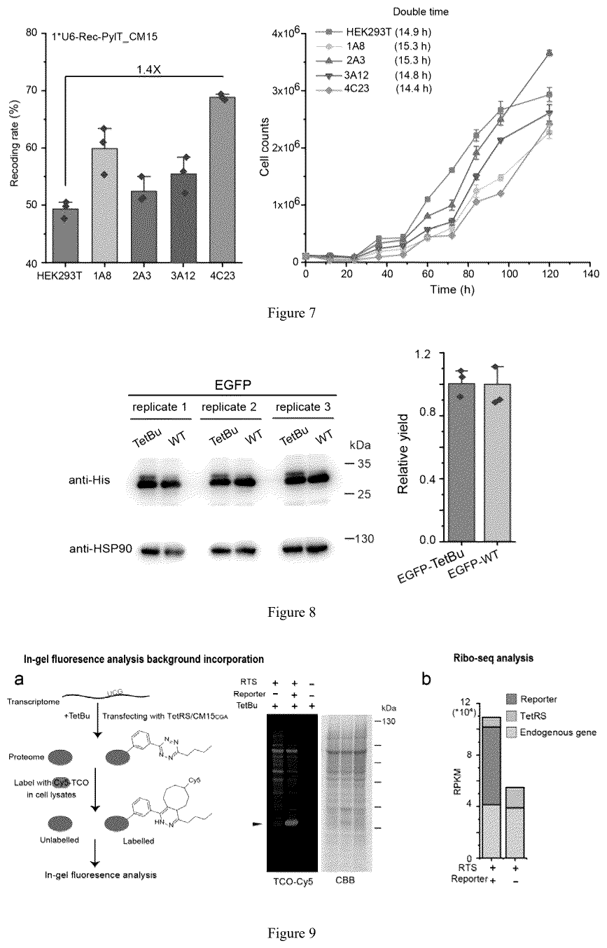

Four sgRNAs targeting Ser-tRNA_{CGA} were designed (Table 6). CRISPR/Cas9 was used to generate knockout polyclonal populations, followed by FACS sorting to isolate monoclonal lines. Sequencing confirmed successful editing in four knockout strains: 1A8, 2A3, 3A12, and 4C23 (FIG. 6). Doubling times of knockout strains (15.3 h, 15.3 h, 14.8 h, and 14.4 h) showed no significant difference from wild-type HEK293T (FIG. 7, right), indicating normal growth.

TABLE 7
The sequence of sgRNA and their targeted genes

| Target gene | sgRNA | |
|---|---|---|
| name | name | sgRNA sequence (5′-3′) |
| TRS-CGA1-1 | sgRNA-1 | gagcaggattCGAacctgcg |
| | | (SEQ ID NO: 95) |
| TRS-CGA2-1 | sgRNA-2 | gagcaggattTgaacctgg |
| | | (SEQ ID NO: 96) |
| TRS-CGA3-1 | sgRNA-3 | GagtccaacAccttaaccact |
| | | (SEQ ID NO: 97) |
| TRS-CGA4-1 | sgRNA-4 | GAcaggattCGAacctgTgcg |
| | | (SEQ ID NO: 98) |

### 2.3.2 TCG Recoding Efficiency in Knockout Strains

This example evaluates the TCG codon recoding capability of the knockout cell lines using the TetRS/PylT_{CGA} recoding system as a model.

LC-MS analysis revealed that the four knockout cell lines (1A8, 2A3, 3A12, and 4C23) exhibited varying efficiencies (61%, 51%, 58%, and 68%, respectively) in recoding the TCG codon at position 151 of EGFP to TetBu (FIG. 7, left) under single-copy of recoding tRNA conditions. Compared to HEK293T cells, all four knockout strains demonstrated enhanced TCG recoding capacity, with strain 4C23 achieving a 1.4-fold improvement (FIG. 7, left), confirming that reducing endogenous decoding tRNA levels boosts TCG recoding efficiency. Further experiments revealed that with 12-copy recoding tRNA, the TetRS/PylT_{CGA} system achieved 88% efficiency for TCG recoding (FIG. 5, bottom right).

### 2.4 Protein Yield Analysis of TCG Recoding System

This example uses the TetRS/PylT_{CGA} recoding system to measure the production yield of TetBu-EGFP protein through TCG recoding.

HEK293T cells were seeded in 12-well plates and transfected at 60%-70% confluency. For the TCG recoding group, 0.8 g of plasmid carrying the Nes-TetRS/12×(U6-PylT_{CGA}) recoding system was co-transfected with 0.8 g of mCherry-T2A-EGFP^{Y151TCG}-6*His plasmid into HEK293T cells. As a WT control, 0.8 μg of Nes-TetRS/12×(U6-PylT_{CGA}) plasmid was co-transfected with 0.8 g of mCherry-T2A-EGFP^{WT}-6*His plasmid. After 6 hours, the medium was replaced with complete medium containing 200 μM TetBu.

Following 40 hours of protein expression, the medium was aspirated and cells were washed with 1 mL PBS. Cells were then lysed with 200 μL 1×SDS loading buffer per well, transferred to 1.5 mL EP tubes, and boiled for 10 minutes in a metal bath for Western blot analysis.

Western blot results demonstrated comparable EGFP protein production between WT and TCG recoding groups was achieved under equal cell loading conditions (FIG. 8). By multiplying the total EGFP signal (m+n, where n represents TetBu-incorporated target protein intensity, and m represents point mutation byproduct as defined in section 1.2.1) with the recoding efficiency, the signal intensity n of TetBu-EGFP was obtained. Quantitative analysis comparing TetBu-EGFP intensity in the TCG recoding group with EGFP-WT intensity in the WT group (FIG. 8, right) confirmed that the TetRS/PylT_{CGA} system can produce TetBu-incorporated EGFP protein at yields equivalent to native EGFP-WT through TCG codon recoding.

### 2.5 Impact of the Optimized TCG Recoding System on Endogenous Protein Translation

This example evaluates the background recoding level of the recoding system in the host cell's endogenous translatome using the TetRS/PylT_{CGA} recoding system as a model.

### 2.5.1 Detection of Noncanonical Amino Acid Incorporation in the Proteome

This example examines the recoding status of endogenous proteins during TCG recoding system operation using TCO fluorescent labeling.

(1) Three transfection systems were established: Sample 1, cells were transfected with Nes-TetRS/12×(U6-PylT_{CGA}) recoding system plasmid; Sample 2, cells were co-transfected with Nes-TetRS/12×(U6-PylT_{CGA}) recoding system plasmid and mCherry-T2A-EGFP^{Y151TCG}-6*His plasmid; Sample 3, cells were transfected with empty vector plasmid (no recoding system).

(2) TCO-Cy5 labeling and imaging of recoding proteome: the (4E)-TCO-PEG4-biotin reaction tag was replaced with TCO-Cy5 to prepare the IEDDA reaction system. Reaction products were mixed with 7 μL 4×SDS loading buffer and heated at 100° C. for 10 min in a metal bath. Then the production samples were separated by 12% SDS-PAGE, and stained with Coomassie Brilliant Blue. The fluorescence images were captured using Azure Biosystems C400. The workflow and principle of this labeling and detection process are illustrated in FIG. 9.

The proteome analysis revealed that: the TCG recoding system lead to detectable background incorporation in HEK293T endogenous proteins (mis-incorporation of noncanonical amino acids in proteome). And when co-expressed with a TCG-containing reporter gene, background incorporation was significantly reduced to nearly negligible levels (FIG. 9, middle)

### 2.5.2 Ribosome Profiling Analysis

This example investigates the molecular mechanism of cellular translation during TCG recoding system operation through ribosome sequencing.

Ribosome sequencing results showed that the volume of ribosomes used to translate the endogenous proteome was basically unchanged in the group that co-expressing with TCG recoding system and the target protein compared with the group only expressing TCG recoding system. The result also showed that half of the ribosomes were used to translate the target gene. This indicates that when the target gene is co-expressed, the expression of the target gene will occupy at least half of the ribosome in the cell, resulting in most of the amino-acylated recoding tRNA being used for the expression of the target gene, thus reducing the proportion of mis-incorporation unnatural amino acids into the proteome. (FIG. 9 right).

The experimental results indicate that the TCG codon recoding system exhibits minimal background incorporation in mammalian cells, and does not affect the normal function of mammalian endogenous translation system. Therefore, the TCG codon recoding system can be applied to the study of biological function in living cells.

### 2.6 Enhancing Recoding Specificity Via Phase Separation System

This example provides a method to improve the specificity of recoding technology through a phase separation system, using the TetRS/PylT_{CGA} recoding system as a model. The λN22 peptide and FUS protein were fused to the N-terminus of TetRS synthetase, while boxB sequences were introduced at the C-terminus of the EGFP transcript. The phase separation system enhanced recoding specificity and reduced misincorporation of non-canonical amino acids in the proteome (b of FIG. 10).

HEK293T cells were co-transfected with: (a) the plasmid containing Nes-TetRS/12×(U6-PylT^{CM15}_{CGA}) recoding system; (b) the plasmid containing 4×λN22-Nes-TetRS/12×(U6-PylT^{CM15}_{CGA}) recoding system; (c) the plasmid containing FUS-4×λN22-Nes-TetRS/12×(U6-PylT^{CM15}_{CGA}) recoding system, and each group was co-transfected with the mCherry-T2A-EGFP^{Y151TCG}-6*His-4*boxB plasmid, along with 200 μM TetBu for protein expression.

The results showed that the system maintained above 80% efficiency under FUS-mediated phase separation conditions (c of FIG. 10). Further analysis showed that although TetBu-EGFP target protein expression slightly decreased, the background incorporation in endogenous proteome was significantly reduced (b of FIG. 10). These findings confirm that the phase separation system enhances the specificity of rare codon recoding systems and minimizes background incorporation of noncanonical amino acids in the proteome.

### 2.7 Assessing the Impact of TCG Recoding System on Cell Viability

This example employs the TetRS/PylT_{CGA} recoding system to investigate the effects of recoding system on cellular viability across four dimensions: cell metabolic activity, cell growth activity, nascent protein synthesis and transcriptome profiles.

### 2.7.1 Measurement of Cellular Metabolic Activity

Cells were transfected with the corresponding recoding system, and after 6 hours, the medium was replaced with complete medium containing 200 μM TetBu. Cells without transfection were served as the control group. After 24 hours of expression, all groups were washed with PBS and treated with the CellTiter-Glo Luminescent Cell Viability Assay kit (Promega) to obtain lysed cell suspensions. The suspensions were then transferred to 96-well plates, and cellular viability was measured using a BioTek Synergy NEO2 microplate reader.

As shown in a of FIG. 11, the intracellular ATP levels in cells transfected with the TCG recoding system showed no significant difference compared to cells transfected with empty vector plasmids. This result demonstrates that cells harboring the TCG recoding system maintain normal levels of cellular metabolic activity.

### 2.7.2 Cell Growth Activity Measurement

HEK293T cells were transfected with plasmid carrying Nes-TetRS/12*(U6-PylT^{CM15}_{CGA}) recoding system and vector plasmid without recoding system, and cultured in complete medium and serine/glycine-free DMEM/F12 medium, respectively. Six hours after transfection, 1×10⁵ cells from each of the above four groups were inoculated into each well of the 12-well plate and continued to be cultured in complete medium containing 200 μM TetBu and serine/glycine free DMEM/F12 medium, respectively. The time point of inoculation with non-natural amino acids was set to t=0. Cells were harvested at different time points, mixed with Trypan Blue solution at a 1:1 volume ratio, and added to the hemocytometer. Cell viability was measured by Countstar BioLab and analyzed by Origin software.

Cell viability assay results showed that cells transfected with TCG recoding system and treated with serine deficiency maintained a survival rate of more than 90% within 48 hours of the recoding system operation, and showed normal cell growth viability (b of FIG. 11), indicating that serine deprivation in the medium did not affect the growth viability of TCG recoding system cells.

### 2.7.3 Nascent Protein Profiling

The corresponding recoding system was transfected and cultured in complete medium containing 200 μM TetBu and serine/glycine-free DMEM/F12 medium, respectively. After 24 hours of transfection, 1 mL DMEM medium without L-methionine was added and continued for 2 hours to deplete intracellular methionine. The medium was then aspirated and added to DMEM medium without L-methionine containing 500 μM of azide hyperalanine (AHA), and continued to be cultured at 37C° for 4 hours to label the cells. The results showed similar pattern of nascent protein expression with or without TCG recoding system (c-d of FIG. 11).

### 2.7.4 RNA Sequencing

The results showed that there was no significant difference in gene expression among the three groups of samples, further indicating that the expression of the TCG recoding system did not affect intracellular transcription (e of FIG. 11).

Based on the above experimental results, it is confirmed that TCG recoding system does not affect cell metabolism, growth and proteome expression from four aspects, including cell metabolic activity, cell growth activity, nascent protein synthesis and transcriptome, and it also indicates that rare codon recoding strategy can be used to study the biological function of living cells in mammalian cells.

### Example 3: Screening Additional Rare Codons for Recoding Using Optimized TCG Recoding System

In Examples 1-2, the recoding efficiency of TCG codon was significantly improved (88%), and experimental evidence confirmed minimal impact of the recoding system on intrinsic cellular physiological activities. Therefore, we further utilized the optimized conditions of the TCG recoding system to screen additional rare codons, aiming to identify other usable rare codons. This would expand the repertoire of codons available for unnatural amino acid incorporation, ultimately enabling simultaneous incorporation of four or more distinct unnatural amino acids.

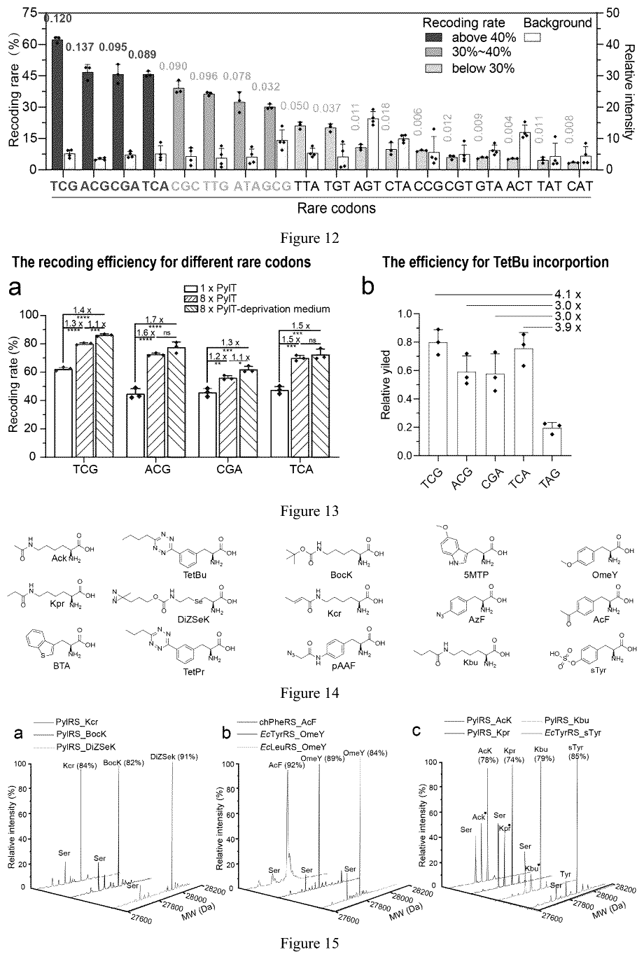

From mammalian cell synonymous codons with usage frequencies below 50%, we selected codons with aminoacyl-tRNA levels less than 3.0% and their codon usage frequency less than 1.5% during translation. We constructed the Nes-TetRS/(U6-PylTCM5_{yyy}) plasmids and pEGFP-mCherry-T2A-EGFP^{Y151xxx}-6*His plasmid (where yyy represents the anticodon for corresponding codon xxx). The two plasmids were co-transfected into HEK293T cells, and the transfection, expression purification, and recoding efficiency quantification by mass spectrometry were the same as in example 1. The experimental results showed the recoding efficiency of ACG, TCA, and CGA was higher than 40% under the condition of recoding tRNA with one copy. The recoding efficiency of CGC, TTG, ATA and GCG codons is higher than 30%, and that of TTA and TGT codons is higher than 15% (FIG. 12a). When tested with 12-copy recoding tRNAs, all ten codons showed substantially improved recoding efficiency (FIG. 13).

Through this screening, we identified 10 codons (from 30 low-frequency mammalian codons) as suitable for our recoding system: TCG, ACG, CGA, TCA, CGC, TTG, ATA, GCG, TTA, and TGT. While the following examples focus on TCG codon, the methodologies are equally applicable to other identified rare codons.

### Example 4: The Scalability of TCG Codon Recoding Strategy

This example uses the rare codon TCG to demonstrate that the rare codon recoding strategy is applicable to multiple orthogonal translation systems, enabling efficient incorporation of various unnatural amino acids in mammalian cells.

### 4.1 Universality of TCG Codon Recoding Strategy Across Multiple Orthogonal Translation Systems

In addition to the chPylRS/MmPylT orthogonal translation system (used in Example 2), this study tested three other common orthogonal translation systems—chPheRS/chPheT, EcTyrRS/EcTyrT, and EcLeuRS/EcLeuT—to evaluate the efficiency of TCG codon recoding for incorporating unnatural amino acids (FIG. 14), and the yield of protein incorporated with UAA.

### 4.1.1 Efficiency Measurement of TCG Codon Recoding for Three Orthogonal Translation Systems

(1) Plasmid construction: In this study, the anticodons of tRNA components in three orthogonal translation systems (chPheRS/chPheT, EcTyrRS/EcTyrT, and EcLeuRS/EcLeuT) were mutated to CGA. Corresponding aminoacyl-tRNA synthetases were also engineered to recognize specific unnatural amino acids—chPheRS for AcF, and both EcTyrRS and EcLeuRS for OmeY. Following the optimized expression strategy described for PylT_{CGA}, we constructed three expression cassettes: 12×(U6-chPheT_{CGA}), 12×(U6-EcTyrT_{CGA}), and 12×(U6-EcLeuT_{CGA}).

(2) Cell transfection and protein expression purification: HEK293T cells were seeded in 10 cm culture dishes and transfected at 60-70% confluency. For each transfection, 10 g of plasmid carrying mCherry-T2A-EGFPY151TCG-6×His was co-transfected with one of the following recoding system plasmids: chPhe_AcFRS/12×(U6-chPheT_{CGA}), EcTyr_OmeYRS/12×(U6-EcTyrT_{CGA}), or EcLeu_OmeYRS/12×(U6-EcLeuT_{CGA}). After 6 hours, the medium was replaced with serine/glycine-free DMEM/F12 containing 500 μM of the respective unnatural amino acid. Following protein expression and purification, LC-MS was used to determine TCG recoding efficiency at position 151 of EGFP. Detailed transfection and purification protocols following section 2.2.1.2, with cell culture conditions in serine-free medium as described in Example 2.

Mass spectrometry results demonstrated that under the optimized conditions combining Examples 2 and 3, all three orthogonal systems achieved efficient incorporation of unnatural amino acids: The EcTyr_OmeYRS/EcTyrT_{CGA} system has a recoding efficiency of 89%, the EcLeu_OmeYRS/EcLeuT_{CGA} system has a recoding efficiency of 84%, and the chPhe_AcFRS/chPheT_{CGA} system has an even higher recoding efficiency of 92% (FIG. 15).

### 4.1.2 Protein Yield Analysis of Three TCG Recoding Systems

Compared with EGFP-WT protein expression signals in WT group, AcF-EGFP and AzF-EGFP proteins expressed by chPheRS/chPheT_{CGA} recoding system, OmeY-EGFP proteins expressed by EcTyrRS/EcTyrT_{CGA} recoding system, OmeY-EGFP protein expressed by EcLeuRS/EcLeuT_{CGA} recoding system has the same protein yields, indicating that the three TCG recoding systems can produce proteins with ncAAs equivalent to the expression of wild-type proteins.

These results demonstrate that the TCG codon recoding strategy is scalable for a variety of orthogonal translation systems to produce proteins with unnatural amino acids.

### 4.2 TCG Recoding Strategy for Efficient Encoding of Multiple Unnatural Amino Acids

In this embodiment, the chPylRS/PylT_{CGA} recoding system, which recognizes unnatural amino acids Kcr, BocK, DiZSeK, AcK, Kbu, and Kpr (FIG. 14), and the EcTyrRS/EcTyrT_{CGA} system, which recognizes sTyr, were used to evaluate the recoding efficiency of various unnatural amino acids.

As described in section 4.1.1, recoding systems recognizing different unnatural amino acids (KcrRS/PylT_{CGA}, BocKRS/PylT_{CGA}, DiZSeKRS/PylT_{CGA}) were co-transfected with the mCherry-T2A-EGFP^{Y151TCG}-6*His plasmid into HEK293T cells, and different unnatural amino acids (500 μM) were supplied. Following protein expression and purification, the recoding efficiencies of the various unnatural amino acids were determined by liquid chromatography-mass spectrometry (LC-MS).

Mass spectrometry results revealed that the chPylRS/PylT_{CGA} recoding system achieved recoding efficiencies of 84%, 82%, and 91% for the unnatural amino acids Kcr, BocK, and DiZSeK, respectively (FIG. 15); efficiencies of 78%, 74%, and 79% for AcK, Kbu, and Kpr, respectively; and an efficiency of 85% for sTyr.

These results demonstrate that the TCG codon recoding strategy is applicable to a variety of unnatural amino acids and orthogonal translation systems, enabling efficient encoding of unnatural amino acids in mammalian cells with broad applicability.

### 4.3 Systems Applicable to the Recoding Strategy

Section 4.1 has demonstrated that the recoding strategy can be applied to four systems: PylRS/PylT, chPheRS/chPheT, EcTyrRS/EcTyrT, and EcLeuRS/EcLeuT. It should be noted that, for the pyrrolysyl system, there exist subsystems from multiple species, and these subsystems remain compatible with the codon recoding strategy. These pyrrole lysine subsystems include those derived from Methanosarcina mazei (MM), Methanosarcina barkeri (Mb), Methanomethylophilus alvus (Ma), Methanogenic archaeon ISO4-G1 (G1), Methanomassiliicoccus luminyensis1 (Lum1), Methanomethylophilus sp.1R26 (1R26), Candidatus Methanomassiliicoccus intestinalis (Int), CA-Nitrososphaeria archaeon (Nitra), S+-Deltaproteobacteria bacterium (Deb). Also included are chPyMRS and its IPYE mutants. The corresponding combinations of aminoacyl-tRNA synthetase and recoding tRNA for the pyrrolysine system are shown in Table 8.

TABLE 8
Pyrrolysine systems for TCG codon recoding

| PylRS | PylT | DNA sequence of PylT |
|---|---|---|
| Mm/Mb/ | C15_{CGA} | gggagagtggccaaggtggccgtgttgactCGAaatcaacacaggggggttCGAttcccccctc |
| ch | | tcccgcca (SEQ ID NO: 2) |
| | M15_{CGA} | ggaaacctggtcagggagacCGAacggactCGAaatccgttcagccgggttCGAttcccgg |
| | | ggtttccgcca (SEQ ID NO: 3) |
| | CM15_{CGA} | gggagagtggccaaggtggcCGAacggactCGAaatccgttcaggggggttCGAttccccc |
| | | ctctoccgcca (SEQ ID NO: 4) |
| | MbPylT_{CGA} | ggaaacctgatcatgtagatCGAatggactCGAaatccgttcagccgggttagattcccggggttt |
| | | ccgcca (SEQ ID NO: 1) |
| | MetPylT_{CGA} | ggagacttggccaaggtggcCGAacggactCGAaatccgttcaggggggttCGAttccccca |
| | | gtttccgcca (SEQ ID NO: 5) |
| | SpePylT_{CGA} | ggaaatctgatcatgtagatCGAatggactCGAaatccgttcagccgggttagattcccggggttt |
| | | ccgcca (SEQ ID NO: 6) |
| | Pyl- | ggggacctgatcatgtagatCGAatggactCGAaatccgttcagccgggttagattcccggggtc |
| | O1_{CGA} | ctcgcca (SEQ ID NO: 7) |
| | Pyl- | gggcggctgatcatgtagatCGAatggactCGAaatccgttcagccgggttagattcccgggctg |
| | O2_{CGA} | cccgcca (SEQ ID NO: 8) |
| Ma | Ma- | gggggacggtccggCGAccagcgggtctCGAaaacctagcatagcggggttCGAcacccc |
| | T6_{CGA} | ggtctctcgcca (SEQ ID NO: 9) |
| | MaPylT_{CGA} | gggggacggtccggCGAccagcgggtctCGAaaacctagccagcggggttCGAcaccccg |
| | | gtctctcgcca (SEQ ID NO: 10) |
| | G1PylT_{CGA} | ggagggcgctccggCGAgcaaacgggtctCGAaaacctgtaagcggggttCGAccccccg |
| | | gcctttcgcca (SEQ ID NO: 11) |
| | Glhyb_{CGA} | gggggacgctccggCGAgcaaacgggtctCGAaaacctgtaagcggggttCGAccccccg |
| | | gtctctcgcca (SEQ ID NO: 23) |
| G1 | Ma- | gggggacggtccggCGAccagcgggtctCGAaaacctagcatagcggggttCGAcacccc |
| | T6_{CGA} | ggtctctcgcca (SEQ ID NO: 9) |
| | MaPylT_{CGA} | gggggacggtccggCGAccagcgggtctCGAaaacctagccagcggggttCGAcaccccg |
| | | gtctctcgcca (SEQ ID NO: 10) |
| | G1PylT_{CGA} | ggagggcgctccggCGAgcaaacgggtctCGAaaacctgtaagcggggttCGAccccccg |
| | | gcctttcgcca (SEQ ID NO: 11) |
| | Glhyb_{CGA} | gggggacgctccggCGAgcaaacgggtctCGAaaacctgtaagcggggttCGAccccccg |
| | | gtctctcgcca (SEQ ID NO: 23) |
| Lum1 | I2B72_{CGA} | ggggtgtagatcggattgatcgcgtggactCGAaatccgCGAacaacgggtgaaactcccgtac |
| | | acctcgcca (SEQ ID NO: 12) |
| | Int17_{CGA} | ggCGAactggtccgggaccaccaggcctCGAaagccacggttagccgggttcaactcccgggt |
| | | tcgtcgcca (SEQ ID NO: 13) |
| | Int5_{CGA} | ggtgacatggtccgggaccaccaggcctCGAaagccacggttagccgggttcaactcccggtgtc |
| | | atcgcca (SEQ ID NO: 14) |
| | Int6B_{CGA} | ggtgaactggtccgggaccaccaggcctCGAaagccacggctagccgggttcaactcccgggttc |
| | | atcgcca (SEQ ID NO: 15) |
| | Int6C_{CGA} | ggtgaactggtccgggaccaccaggcctCGAaagccacggttagccgggttcaactcccgggttc |
| | | atcgcca (SEQ ID NO: 16) |
| | Int13_{CGA} | ggtgttctggtccgggaccaccgggcctCGAaagccacggttagccgggttcaactcccgggaac |
| | | atcgcca (SEQ ID NO: 17) |
| 1R26 | Alv21_{CGA} | gggggacggtccggCGAccagcgggtctCGAaaacctagcataagcggggttCGAccccc |
| | | cggtctctcgcca (SEQ ID NO: 18) |
| | Alv8_{CGA} | gggggacggtccggCGAccagcgggtctCGAaaacctagccttgcggggttCGAcacccc |
| | | ggtctctcgcca (SEQ ID NO: 19) |
| | Alv17_{CGA} | gggggacggtccggCGAccagcgggtctCGAaaacctagcgtaagcggggttCGAcaccc |
| | | cggtctctcgcca (SEQ ID NO: 20) |
| | Alv10_{CGA} | gggggacggtccggCGAccagcgggtctCGAaaacctagcttagcggggttCGAcacccc |
| | | ggtctctcgcca (SEQ ID NO: 21) |
| | Alv22_{CGA} | gggggacggtccggCGAccagcgggtctCGAaaacctagctcaaggcggggttCGActcc |
| | | ccggtctctcgcca (SEQ ID NO: 22) |
| | G1hyb_{CGA} | gggggacgctccggCGAgcaaacgggtctCGAaaacctgtaagcggggttCGAccccccg |
| | | gtctctcgcca (SEQ ID NO: 23) |
| Int | Int_{CGA} | ggagtgttggtccgggaccaccaggcctCGAcagccacggcagccgggttcaactcccgggcac |
| | | ttcgcca (SEQ ID NO: 24) |
| | Int17_{CGA} | ggCGAactggtccgggaccaccaggcctCGAaagccacggttagccgggttcaactcccgggt |
| | | tcgtcgcca (SEQ ID NO: 13) |
| | Int5_{CGA} | ggtgacatggtccgggaccaccaggcctCGAaagccacggttagccgggttcaactcccggtgtc |
| | | atcgcca (SEQ ID NO: 14) |
| | Int6B_{CGA} | ggtgaactggtccgggaccaccaggcctCGAaagccacggctagccgggttcaactcccgggttc |
| | | atcgcca (SEQ ID NO: 15) |
| | Int6C_{CGA} | ggtgaactggtccgggaccaccaggcctCGAaagccacggttagccgggttcaactcccgggttc |
| | | atcgcca (SEQ ID NO: 16) |
| | Int13_{CGA} | ggtgttctggtccgggaccaccgggcctCGAaagccacggttagccgggttcaactcccgggaac |
| | | atcgcca (SEQ ID NO: 17) |
| Nitra | Int_{CGA} | ggagtgttggtccgggaccaccaggcctCGAcagccacggcagccgggttcaactcccgggcac |
| | | ttcgcca (SEQ ID NO: 24) |
| | Int17_{CGA} | ggCGAactggtccgggaccaccaggcctCGAaagccacggttagccgggttcaactcccgggt |
| | | tcgtcgcca (SEQ ID NO: 13) |
| | Int5_{CGA} | ggtgacatggtccgggaccaccaggcctCGAaagccacggttagccgggttcaactcccggtgtc |
| | | atcgcca (SEQ ID NO: 14) |
| | Int6B_{CGA} | ggtgaactggtccgggaccaccaggcctCGAaagccacggctagccgggttcaactcccgggttc |
| | | atcgcca (SEQ ID NO: 15) |
| | Int6C_{CGA} | ggtgaactggtccgggaccaccaggcctCGAaagccacggttagccgggttcaactcccgggttc |
| | | atcgcca (SEQ ID NO: 16) |
| | Int13_{CGA} | ggtgttctggtccgggaccaccgggcctCGAaagccacggttagccgggttcaactcccgggaac |
| | | atcgcca (SEQ ID NO: 17) |
| | Therm1_{CGA} | ggggggctggtcgggtggccaagggggctCGAaaccctcggttgccgggttcaactcccgggct |
| | | ccccacca (SEQ ID NO: 25) |
| Deb | Int_{CGA} | ggagtgttggtccgggaccaccaggcctCGAcagccacggcagccgggttcaactcccgggcac |
| | | ttcgcca (SEQ ID NO: 24) |
| | BH52_{CGA} | ggggcgttgatcggattgatcgcgtggactCGAaatccgcggcCGAcgggtgaaactcccgta |
| | | cacctctcca (SEQ ID NO: 26) |

The TCG codon recoding systems described above can also be adapted to recoding systems for the following codons by altering the anticodon: ACG, CGA, TCA, CGC, TTG, ATA, GCG, TTA, and TGT. The underlined portions can be substituted with the corresponding anticodons.

For the three systems—chPheRS/chPheT, EcTyrRS/EcTyrT, and EcLeuRS/EcLeuT—combinations with various tRNAs are also possible, with the corresponding combinations detailed in Table 9.

TABLE 9
Combinations of various recoding systems

| aaRS | aaT | DNA sequence of aaT |
|---|---|---|
| chPhe | CM4_{CGA} | ggGAGAGTggccaaggtggccgAACGGActCGaaaTCCGTTcagccgggttcgat |
| RS | | tcccggCTCTCccACCA (SEQ ID NO: 27) |
| | CM15_{CGA} | gggagagtggccaaggtggcCGAacggactCGAaatccgttcaggggggttCGAttcccccc |
| | | tctcccgcca (SEQ ID NO: 4) |
| | MbPyIT_{CGA} | ggaaacctgatcatgtagatCGAatggactCGAaatccgttcagccgggttagattcccggggtttc |
| | | cgcca (SEQ ID NO: 1) |
| | Pyl- | ggggacctgatcatgtagatCGAatggactCGAaatccgttcagccgggttagattcccggggtcc |
| | O1_{CGA} | tcgcca (SEQ ID NO: 7) |
| | Pyl- | gggcggctgatcatgtagatCGAatggactCGAaatccgttcagccgggttagattcccgggctg |
| | O2_{CGA} | cccgcca (SEQ ID NO: 8) |
| | AS78_{CGA} | ggggaggtggccaaggtggcCGAacggactCGAaatccgttcagccgggttCGAttcccggc |
| | | ctccccacca (SEQ ID NO: 28) |
| EcTyr | BsTyrT_{CGA} | ggtggggtagCGAagtggctaaacgcggcggactCGAaatccgctccctttgggttcggcggtt |
| RS | | CGAatccgtcccccacca (SEQ ID NO: 29) |
| | NGS6_{CGA} | ggGGCGgtagCGAagtggctaaacgcggcggactCGAaatccgctccctttgggttcggcgg |
| | | ttCGAatccgCGCCccacca (SEQ ID NO: 30) |
| EcLeu | EcLeuT_{CGA} | gcccggatggtggaatcggtagacacaagggattCGAaatccctcggcgttcgcgctgtgcgggtt |
| RS | | caagtcccgctccgggtacca (SEQ ID NO: 31) |
| | L- | gcccgtatggtggaatcggtagacacaagggattCGAaatccctcggcgttcgcgctgtgcgggttc |
| | G1_{CGA} | aagtcccgctgcgggcacca (SEQ ID NO: 32) |
| | L- | gggcgtgtggtggaatcggtagacacaagggattCGAaatccctcggcgttcgcgctgtgcgggttc |
| | G2_{CGA} | aagtcccgccgcgcccacca (SEQ ID NO: 33) |

The TCG codon recoding systems described above can also be converted into recoding systems for the following codons by modifying the anticodon: ACG, CGA, TCA, CGC, TTG, ATA, GCG, TTA, and TGT. For example, CGA can be replaced with CGT, TCG, TGA, GCG, CAA, TAT, CGC, TAA, or ACA. The underlined portions can be substituted with the corresponding anticodons.

### 4.4 Application of the TCG Recoding Strategy for Various Unnatural Amino Acids

The above examples demonstrate that the rare codon recoding strategy can be extended to all unnatural amino acids previously introduced into mammalian cell systems. Accordingly, this application compiles all amino acids reported in the literature that are recognized by orthogonal aminoacyl-tRNA synthetases in mammalian systems and evaluates their recoding efficiency using the TCG codon as an example. Corresponding data are presented in Table 1 (Recoding efficiency of all amino acids).

The introduction of all unnatural amino acids listed in the respective tables can also utilize other rare codons, such as ACG, CGA, TCA, CGC, TTG, ATA, GCG, TTA, and TGT.

### Example 5: Application of the TCG Recoding System for Encoding Unnatural Amino Acids at Multiple Sites in Proteins

This example uses the chPyl_TetRS/PylT_{CGA} recoding system and the chPhe_AcFRS/chPheT_{CGA} recoding system as examples to illustrate a method for expressing proteins with multiple-site insertions of unnatural amino acids in mammalian cells.

(1) Plasmid construction: Using the primers shown in Tables 10 and 11, TCG or TAG mutations were introduced into the EGFP protein gene sequence at positions 95, 151, 164, 172, 182, and 200 via the Gibson seamless cloning method. This resulted in the construction of EGFP protein expression plasmids with multiple TCG mutations: mCherry-T2A-EGFP^{151/182/200TCG}-6His and mCherry-T2A-EGFP^{95/151/164/172/182/200TCG}-6His; or EGFP protein expression plasmids with multiple TAG mutations: mCherry-T2A-EGFP^{151/182/200TAG}-6His and mCherry-T2A-EGFP^{95/151/164/172/182/200TAG}-6His.

TABLE 10
Primer sequences for EGFP-TCG mutations

| Primer name | Sequence from 5′ to 3′ |
|---|---|
| EGFP-95TCG-F | CGTCCAGtcGCGCACCATCTTCTTCAAGGAC |
| | (SEQ ID NO: 114) |
| EGFP-95TCG-R | GCGCGACTGGACGTAGCCTTCGGGCATG |
| | (SEQ ID NO: 115) |
| EGFP-151TCG-F | CAACGTCTCGATCATGGCCGACAAGCAGAAG |
| | (SEQ ID NO: 116) |
| EGFP-151TCG-R | ATGATCGAGACGTTGTGGCTGTTGTAGTTG |
| | (SEQ ID NO: 117) |
| EGFP-164TCG-F | AGGTGtcgTTCAAGATCCGCCACAACATCG |
| | (SEQ ID NO: 118) |
| EGFP-164TCG-R | TTGAACGACACCTTGATGCCGTTCTTCTGC |
| | (SEQ ID NO: 119) |
| EGFP-172TCG-F | CAACATCtcGGACGGCAGCGTGCAGCTC |
| | (SEQ ID NO: 120) |
| EGFP-172TCG-R | CGTCCGAGATGTTGTGGCGGATCTTGAAGTTC |
| | (SEQ ID NO: 121) |
| EGFP-182TCG-F | ACCACTcgCAGCAGAACACCCCCATCGG |
| | (SEQ ID NO: 122) |
| EGFP-182TCG-R | TCTGCTGCGAGTGGTCGGCGAGCTGCAC |
| | (SEQ ID NO: 123) |
| EGFP-200TCG-F | AACCACTcgCTGAGCACCCAGTCCGCCCTG |
| | (SEQ ID NO: 124) |
| EGFP-200TCG-R | TCAGCGAGTGGTTGTCGGGCAGCAGC |
| | (SEQ ID NO: 125) |

TABLE 11
Primer sequences for EGFP-TAG mutations

| Primer | |
|---|---|
| name | Sequence from 5′ to 3′ |
| EGFP- | TAGCGCACCATCTTCTTCAAGGACGA(SEQ ID NO: |
| 95TAG-F | 126) |
| EGFP- | GATGGTGCGCTaCTGGACGTAGCCTTCGGGCA(SEQ |
| 95TAG-R | ID NO: 127) |
| EGFP- | AACAGCCACAACGTCTAgATCATGGCCGACAAGCAG |
| 151TAG- | (SEQ ID NO: 128) |
| F | |
| EGFP- | GTTGTGGCTGTTGTAGTTGTACTCCAG(SEQ ID NO: |
| 151TAG- | 129) |
| R | |
| EGFP- | AGGTGtAgTTCAAGATCCGCCACAACATCtAG(SEQ |
| 164TAG- | ID NO: 130) |
| F | |
| EGFP- | TTGAAcTaCACCTTGATGCCGTTCTTCTG(SEQ ID |
| 164TAG- | NO: 131) |
| R | |
| EGFP- | CAACATCtAGGACGGCAGCGTGCAGCTCGC(SEQ ID |
| 172TAG- | NO: 132) |
| F | |
| EGFP- | CGTCCTaGATGTTGTGGCGGATCTTGAAcTaC(SEQ |
| 172TAG- | ID NO: 133) |
| R | |
| EGFP- | GCGACGGCCCCGTGCTGCTGCCCGACAACCACTAGCTG |
| 182/200T | AGCACCCAGTCCGCCCTGAGC(SEQ ID NO: 134) |
| AG-F | |
| EGFP- | CACGGGGCCGTCGCCGATGGGGGTGTTCTGCTGCTAGT |
| 182/200T | GGTCGGCGAGCTGCACGCTGC(SEQ ID NO: 135) |
| AG-R | |

(2) The above plasmids were transfected into HEK293T cells respectively, supplemented with unnatural amino acids (200 Wt TetBu or 500 μM AcF), to induce protein expression. Following purification according to the methods described in the previous embodiments, LC-MS analysis was performed. Referring to the transfection method in Example “4.1.1 Determination of TCG codon recoding efficiency in three translation systems”, plasmids containing the chPyl_TetRS/PylT_{CGA} recoding system or the chPhe_AcFRS/chPheT_{CGA} recoding system were co-transfected into HEK293T cells with plasmids harboring either three TCG mutations (mCherry-T2A-EGFP^{151/182/200TCG}-61His) or six TCG mutations (mCherry-T2A-EGFP^{95/151/164/172/182/200TCG}-61His), along with unnatural amino acids (200 Wt TetBu or 500 μM AcF) to induce protein expression. Similarly, plasmids containing the amber suppression system based on the TAG codon (chPyl_TetRS/PylT_{CUA}) were co-transfected with plasmids harboring either three TAG mutations (mCherry-T2A-EGFP^{151/182/200IAG}-6His) or six TAG mutations (mCherry-T2A-EGFP^{95/151/164/172/182/200TAG}-61His) into HEK293T cells, with 200 μM TetBu provided for protein expression. Cells were harvested 40 hours post-transfection for subsequent experiments.

(3) Using the protein purification and LC-MS analysis methods described above, quantitative analysis was performed to determine the number and proportion of unnatural amino acids incorporated into EGFP proteins with three or six TCG sites.

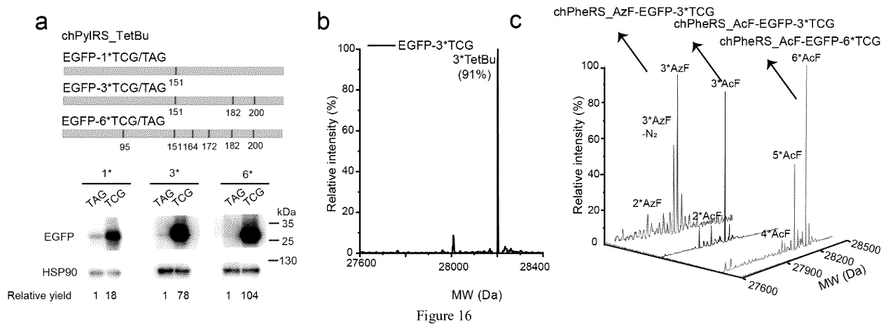

Mass spectrometry results showed that, for EGFP proteins with three TCG mutations, the proportion of proteins with unnatural amino acids inserted at all three sites-expressed via the chPyl_TetRS/PylT_{CGA} and chPhe_AcFRS/chPheT_{CGA} recoding systems-accounted for over 90% of the total expressed protein (b of FIG. 16). For EGFP proteins with six TCG mutations, all proteins expressed via the chPhe_AcFRS/chPheT_{CGA} recoding system exhibited recoding of at least five TCG sites to AcF, with the proportion of proteins having unnatural amino acids inserted at all six sites exceeding 70% of the total expressed protein (c of FIG. 16).

Western blot analysis revealed that the protein yields of EGFP with unnatural amino acids at three sites and six sites, expressed via the TCG recoding system, were 78-fold and 104-fold higher, respectively, than those expressed via the TAG amber suppression system (a of FIG. 16).

These results confirm that the TCG codon recoding system enables highly efficient expression of proteins with multiple-site insertions of unnatural amino acids, achieving exceptional fidelity and yield. The rare codon recoding strategy represents a powerful tool for producing proteins with multi-site unnatural amino acid incorporation in mammalian cells.

### Example 6: Application of the Recoding System in Therapeutic Proteins

This example uses the TetRS/PylT_{CGA} recoding system as an example to illustrate the application of the recoding system in interleukin proteins.

6.1 Intracellular Expression of Interleukin Proteins with Unnatural Amino Acids

In this example, four interleukin proteins—IL-1B, IL-2, IL-4, and IL-6—are used as examples to describe the efficient expression of proteins bearing the unnatural amino acid (TetBu) via the TCG recoding system.

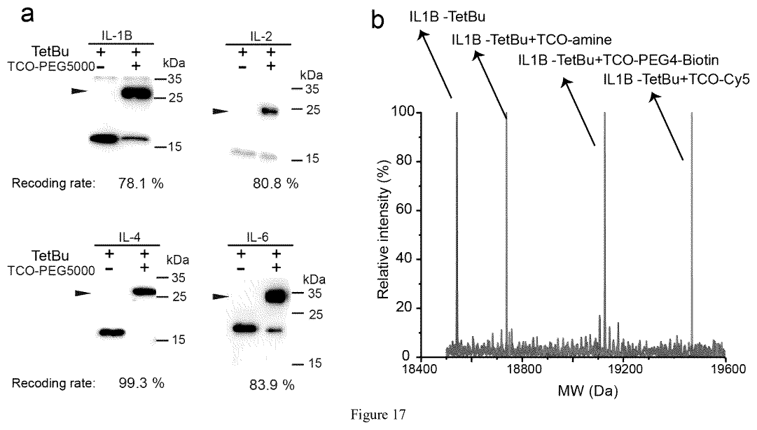

The recoding system and plasmids containing the TCG codon for the cytokines were co-transfected into HEK293T cells. Six hours post-transfection, the medium was replaced with serine/glycine-free DMEM/F12 medium supplemented with 200 μM TetBu. After 40 hours of protein expression, 500 μL of Modified RIPA lysis buffer was added to each well to lyse the cells. The TCO-PEG5000 labeling method was used to assess the recoding efficiency. Based on the definition and description in the 1.2.1, this application calculated that, under these experimental conditions, the TetRS/PylT_{CGA} recoding system achieved a TetBu recoding efficiency of 78.10% at position 205 of the IL-1B protein, 80.8% at position 62 of the IL-2 protein, 99.3% at position 121 of the IL-4 protein, and 83.9% at position 87 of the IL-6 protein (a of FIG. 17).

These results confirm that, under the optimized conditions of Embodiment 2, the TCG recoding system can produce therapeutic proteins with unnatural amino acids in mammalian cells with a recoding efficiency exceeding 70%.

6.2: Application of TetBu-Interleukin Protein Conjugation with TCO Reagents

In this example, TetBu-modified IL-1B protein is used as an example to demonstrate the high efficiency of conjugating TetBu-interleukin proteins—expressed via the TCG recoding system—with various TCO reagents, as well as their potential for multifaceted applications.

The reaction system for conjugating TetBu-IL1B protein with TCO reagents was prepared according to Table 12. The TCO reagents include, but are not limited to, TCO-amine, TCO-PEG4-Biotin, and TCO-Cy5. The reaction products were directly analyzed by LC-MS.

TABLE 12
Reaction system for TetBu-IL1B protein
conjugation with TCO reagents

| Component | Volume (μL) |
|---|---|
| Purified TetBu-IL1B protein solution (40 μM) | 50 |
| 5 mM TCO reagent | 0.5 |

Mix and react at 25° C. for 60 min

| | |
|---|---|
| 200 mM TetBu | 2.5 |

Mix and react at 25° C. for 10 min

Mass spectrometry results clearly showed that TetBu-IL1B can conjugate with various TCO reagents with nearly 100% efficiency (b of FIG. 17), endowing the TetBu-IL1B protein with diverse properties and functionalities.

The above results indicate that the TCG recoding system enables efficient recoding in mammalian cells to produce therapeutic proteins bearing unnatural amino acids. This represents a promising strategy for synthesizing functionalized therapeutic proteins for drug development.

### Example 7: Application of Rare Codon Recoding System for Encoding Multiple Unnatural Amino Acids on a Single Protein

This example demonstrates methods for simultaneously encoding two, three, four or five distinct UAAs on the same protein in mammalian cells based on TCG rare codon recoding system.

### 7.1 Plasmid Construction

### 7.1.1 Plasmids Construction for Introducing Two Distinct of UAAs Using Dual Rare Codon Recoding System

First, plasmids bearing reporter genes with rare codons at different sites were constructed according to the primers listed in Table 13, based on the pEGFP-mCherry-T2A-EGFP151TCG backbone, including: pEGFP-mCherry-T2A-EGFP^{151TCG/182ACG}, pEGFP-mCherry-T2A-EGFP^{51TCG/182TCA}, pEGFP-mCherry-T2A-EGFP^{51TCG/182CGA} and pEGFP-mCherry-T2A-EGFP^{84TCG/151ACG}, Subsequently, plasmids bearing different rare codon recoding systems for distinct UAAs incorporation were constructed, including: pCMV-G1PylRS (TetPr)-8*(U6-G1PylT_{CGT}), pCMV-G1PylRS (TetPr)-8*(U6-G1PylT_{TGA}), pCMV-G1PylRS (TetPr)-8*(U6-G1PylT_{TCG}).

TABLE 13
Primer Sequences for Site-Directed Mutagenesis

| Primer Name | Sequence (5′→3′) |
|---|---|
| EGFP-182ACG-F1 | GCGTGCAGCTCGCCGACCACACGCAGCAGAAC |
| | ACCCCCATCGGC(SEQ ID NO: 136) |
| EGFP-182ACG-R1 | GTGGTCGGCGAGCTGCACGC(SEQ ID NO: |
| | 137) |
| EGFP-182TCA-F | GCGTGCAGCTCGCCGACCACTCACAGCAGAAC |
| | ACCCCCATCGGC(SEQ ID NO: 138) |
| EGFP-182TCA-R | GTGGTCGGCGAGCTGCACGC(SEQ ID NO: |
| | 137) |
| EGFP-182CGA-F | GCGTGCAGCTCGCCGACCACCGACAGCAGAAC |
| | ACCCCCATCGGC(SEQ ID NO: 139) |
| EGFP-182CGA-R | GTGGTCGGCGAGCTGCACGC(SEQ ID NO: |
| | 137) |
| EGFP-151ACG-F | CAACGTCACGATCATGGCCGACAAGCAGAAG |
| | (SEQ ID NO: 140) |
| EGFP-151ACG-R | ATGATCGTGACGTTGTGGCTGTTGTAGTTG |
| | (SEQ ID NO: 141) |
| EGFP-84TCG-F | ACATGAAGCAGCACGACTTCTCGAAGTCCGCC |
| | ATGCCCGAAGG(SEQ ID NO: 142) |
| EGFP-84TCG-R | GAAGTCGTGCTGCTTCATGTGGT(SEQ ID |
| | NO: 143) |

### 7.1.2 Plasmids Construction for Triple Distinct UAAs Incorporation

For the plasmids used for three distinct of UAAs incorporation: two groups were designed with group-1 (EcTrpRS/EcTrpT_{UCA}, chPylRS/MmPylT_{CGA} and EcTyrRS/BsTyrT_{CUA}) and group-2 EcTrpRS/EcTrpT_{UCA}, chPheRS/chPheT_{CGA} and MaPylRS/MaPylT_{CUA}. Sequences encoding EGFP-39TGA/151TCG/182TAG were synthesized and cloned into the pEGFP vector. The fragments of EF1α promoter, EcTrpRS (5MTP) and 4*(U6-EcTrpT_{UCA}) were synthesized and inserted into pEGFP-EGFP-39TGA/151TCG/182TAG to generate plasmid pEGFP-EGFP-39TGA/151TCG/182TAG-EF1α-EcTrpRS (5MTP)-4*(U6-EcTrpT_{UCA}) For group1, the fragments of chPylRS_{IPYE} (TetRS), 4*U6-CM15_{CGA}, EcTyrRS (OmeY) and 4*U6-BsTyrT_{CUA} were synthesized and cloned into pCMV vector to generate plasmid pCMV-Nes-chPylRS_{IPYE} (TetRS)-4*(U6-CM15_{CGA})-EF1α-EcTyrRS (OmeY)-4*(U6-BsTyrT_{CUA}) For group2, the plasmid pCMV-chPheRS (AcF)-4*chPheT_{CGA}-EF1α-MaPylRS (BocK)-4*(U6-MaPylT_{CUA}-T6) for group-2 was constructed as described above.

### 7.1.3 Plasmids Construction for Quadruple Distinct UAAs Incorporation

For the plasmids used in four distinct UAAs incorporation: fragment of EGFP carrying 39TGA/151TAA/182TAG/200TCG was synthesized and inserted into the pEGFP vector carrying EcTrpRS/EcTrpT_{UCA} to generate plasmid pEGFP-EGFP-39TGA/151TAA/182TAG/200TCG-EF1α-EcTrpRS (5MTP)-4*(U6-EcTrpT_{UCA}) The fragments of MaPylRS (BocK), 4*(U6-MaPylT_{CUA}-T6) were inserted into the vector carrying chPylRS_{IPYE} (TetRS)/MmPylT_{CGA} to generate plasmid pCMV-Nes-chPylRS_{IPYE} (TetRS)-4*(U6-CM15_{CGA})-EF1α-MaPylRS (BocK)-4*(U6-MaPylT_{CUA}-T6). The fragment of EcTyrRS (OmeY) and 8*(U6-BsTyrT_{UUA}) were inserted into the pCMV vector to generate pCMV-EcTyrRS (OmeY)- 8*(U6-BsTyrT_{UUA})

TABLE 14
Primer sequences for site-directed mutagenesis

| Primer Name | Sequence (5′→3′) |
|---|---|
| EGFP-151TAA- | ATGATttaGACGTTGTGGCTGTTGTAGTTG |
| F | (SEQ ID NO 144) |
| EGFP-151TAA- | CAACGTCtaaATCATGGCCGACAAGCAGAAG |
| R | (SEQ ID NO 145) |
| EGFP-200TCG- | GGTGCTCAGcgaGTGGTTGTCGGGCAGCAGCACG |
| F | (SEQ ID NO 146) |
| EGFP-200TCG- | CCGACAACCACtcgCTGAGCACCCAGTCCGCCCTG |
| R | (SEQ ID NO 147) |

### 7.1.4 Plasmids Construction for Quintuplet Distinct UAAs Incorporation

To construct the plasmids for the incorporation of quintuplet distinct UAAs, the fragment of EGFP carrying 39TGA/84TCA/151ACG/182TCG/200TAG was also amplified by overlap PCR and inserted into the plasmid pEGFP-EGFP-EF1α-5MTP EcTrpRS-4*(U6-EcTrpT_{UCA}) described in 7.1.2 to generate plasmid pEGFP-EGFP-39TGA/84TCA/151ACG/182TCG/200TAG-6*His-EF1α-EcTrpRS (5MTP)-4*(U6-EcTrpT_{UCA}). The fragments of EF1α promoter, chPheRS (OmeY), 4*(U6-chPheT_{UGA}), EcTyrRS (pAAF) and 4*(U6-BsTyrT_{CGA}) were synthesized and cloned into pCMV vector to generate plasmid pCMV- chPheRS (OmeY)-4*(U6-chPheT_{UGA})-EF1α-EcTyrRS (pAAF)-4*(U6-BsTyrT_{CGA}). The plasmid pCMV-LumPylRS (BocK)-4*(U6-Int17C10_{CUA})-EF1α- G1PylRS (TetPr)-4*(U6-G1PylT_{CGU}) was also constructed as described above.

TABLE 15
Primer sequences for site-directed mutagenesis

| Primer Name | Sequence (5′→3′) |
|---|---|
| EGFP-151TCA- | GTCTCAATCATGGCCGACAAGCAGAAGAACGGCAT |
| F | CAAGGTGAACTTCAAGATC(SEQ ID NO: 148) |
| EGFP-151TCA- | GTCGGCCATGATTGAGACGTTGTGGCTGTTGTAGT |
| R | TGTACTCC(SEQ ID NO: 149) |
| EGFP-182ACG- | CACacgCAGCAGAACACCCCCATCGGCGACGGCCC |
| F2 | CG(SEQID NO: 150) |
| EGFP-182ACG- | CCGTCGCCGATGGGGGTGTTCTGCTGcgtGTGGTC |
| R2 | GGCGAGCTGCACG(SEQ ID NO: 151) |
| EGFP-206TAG- | GTCCtagCTGAGCAAAGACCCCAACGAGAAG(SEQ |
| F | ID NO: 152) |
| EGFP-206TAG- | GTCTTTGCTCAGctaGGACTGGGTGCTCA(SEQ |
| R | ID NO: 153) |

7.2 Protein Expression and Characterization with Multiple UAA Incorporation

The plasmids for site-specifically incorporation of multiple distinct UAAs were transfected into HEK293T cells with corresponding UAAs addition for protein expression. Protein purification was carried out as described above. The recoding rates of multiple UAA incorporation in EGFP proteins containing 2, 3, 4, and 5 site-specific rare codon mutations were determined with the same procedure as described above.

For expression of POIs incorporated with dual distinct UAAs: taking EGFP-151pAzF/182TetPr for example, plasmid pCMV-pAzF_chPheRS-8*(U6-chPheT_{CGA}), pCMV-TetPr G1PylRS-8*(U6-G1PylT_{CGU}) and pEGFP-mCherry-T2A-EGFP-151TCG/182ACG were transfected into the HEK 293T cells line with ratio (1:1:1) in the presence of 500 μM pAzF and 200 μM TetPr. Other POIs incorporated with dual distinct UAAs were expressed with the same procedure, which the final concentration of distinct UAAs were BTA (1 mM), OmeY (500 μM), pAcF (1 mM), sTyr (2 mM), BocK (2 mM), Kcr (4 mM).

For expression of EGFP-incorporated with three distinct UAAs, group 1 plasmids (pCMV-Nes-chPylRS_{IPYE} (TetRS)-4*(U6-CM15_{CGA})-EF1α-EcTyrRS (OmeY)-4*(U6-BsTyrT_{CUA}), pEGFP-EGFP-39TGA/151TCG/182TAG-EF1α-EcTrpRS (5MTP)-4*(U6-EcTrpT_{UCA})), and group 2 plasmids (pCMV-chPheRS (AcF)-4*chPheT_{CGA}-EF1α-MaPylRS (BocK)-4*(U6-MaPylT_{CUA}-T6), pEGFP-EGFP-39TGA/151TCG/182TAG-EF1α-EcTrpRS (5MTP)-4*(U6-EcTrpT_{UCA})) were transfected into the HEK293T cell line in the presence of indicated UAAs. For expression of EGFP-incorporated with four distinct UAAs, plasmids of pEGFP-EGFP-39TGA/151TAA/182TAG/200TCG-EF1α-EcTrpRS (5MTP)-4*(U6-EcTrpT_{UCA}), pCMV-Nes-chPylRS_{IPYE} (TetRS)-4*(U6-CM15_{CGA})-EF1α-MaPylRS (BocK)-4*(U6-MaPylT_{CUA}-T6) and pCMV-EcTyrRS (OmeY)- 8*(U6-BsTyrT_{UUA}) were co-transfected into HEK293T at a 1:1:1 ratio in the presence of indicated UAAs.

For the expression of EGFP incorporated with five distinct UAAs, plasmid pEGFP-EGFP-39TGA/84TCA/151ACG/182TCG/200TAG-6*His-EF1α-EcTrpRS (5MTP)-4*(U6-EcTrpT_{UCA}), pCMV-chPheRS (OmeY)-4*(U6-chPheT_{UGA})-EF1α-EcTyrRS (pAAF)-4*(U6-BsTyrT_{CGA}) and pCMV-LumPylRS (BocK)-4*(U6-Int17C10_{CUA})-EF1α-G1PylRS (TetPr)-4*(U6-G1PylT_{CGU}) were co-transfected into HEK 293T cells in the presence of the indicated orthogonal UAAs (500 μM 5MTP, 500 μM OmeY, 200 μM TetPr, 500 μM pAAF and 500 μM BocK).

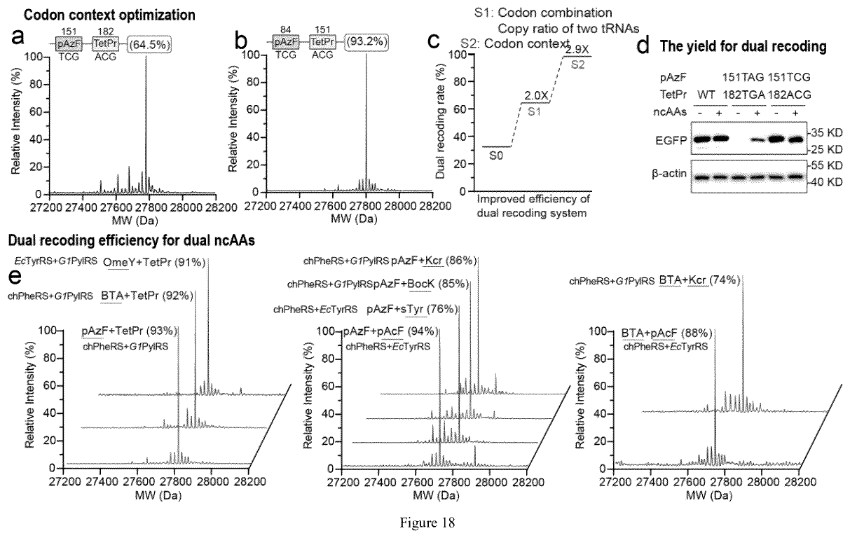

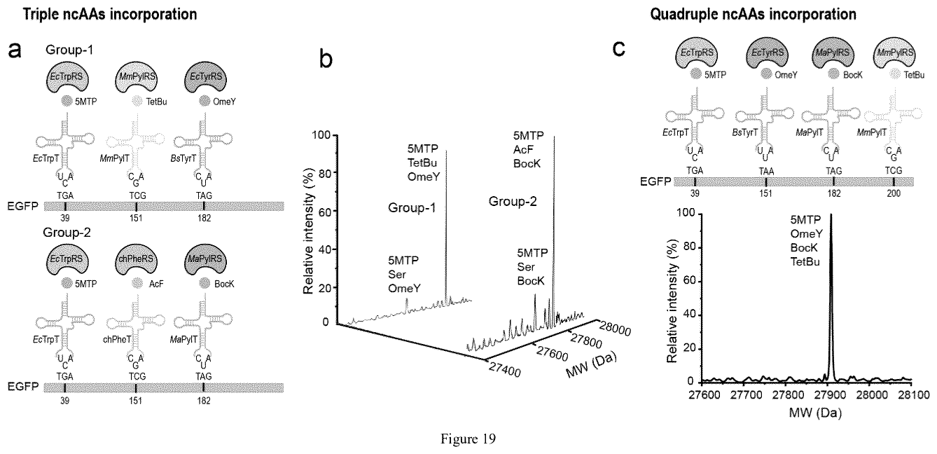

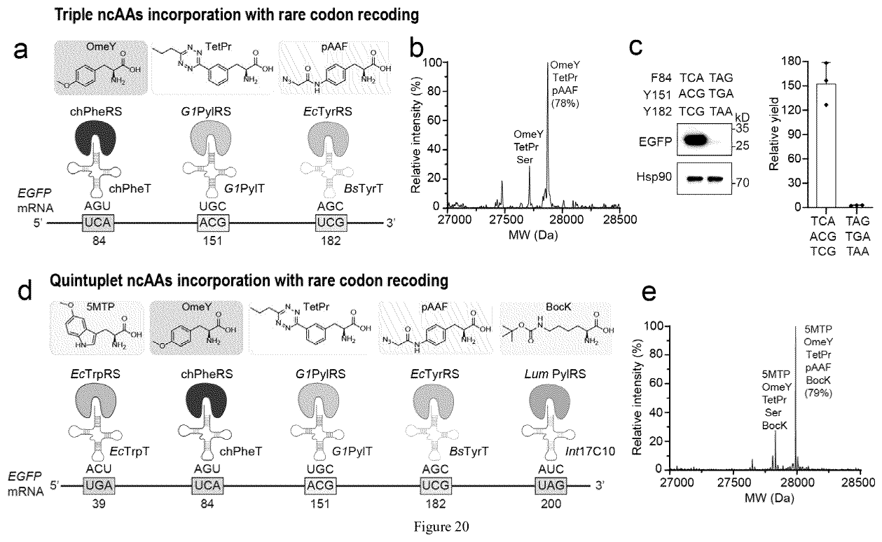

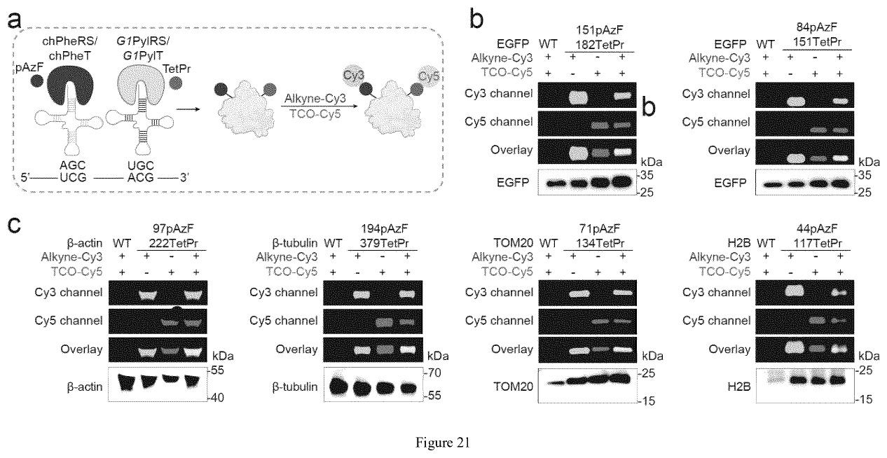

Protein purification and LC-MS analysis were carried out as described above to determine the recoding rate during multiple UAA incorporation. The results of LC-MS analysis demonstrated that two distinct UAAs were incorporated into target proteins with high efficiency and fidelity with two rare codons (FIG. 18), and both UAAs could be simultaneously labeled with bioorthogonal reagents with high efficiency (FIG. 21). Utilizing three rare codons (a-c of FIG. 20) or the combined one rare codon with two termination codons (a-b of FIG. 19), three different UAAs were successfully incorporated into proteins with high efficiency and fidelity. Further combination of rare codon systems with termination codons enabled the synthesis of proteins containing four (c of FIG. 19) or even five (d-e of FIG. 20) distinct UAAs while achieving excellent fidelity. These results confirm that rare codon recoding systems can significantly expand the variety of multiple UAAs incorporation, providing numerous possibilities for engineering proteins with novel and enhanced properties.

### Example 8: Application of Recoding Systems for Functional Activation and Regulation of Proteins in Living Cells

This example utilizes the rare codon recoding systems with TCG and ACG to demonstrate a strategy for achieving rapid and efficient functional activation and regulation of multiple proteins in living cells through rare codon recoding.

### 8.1 Protein Functional Activation and Regulation Strategy Based on the TCG Recoding System

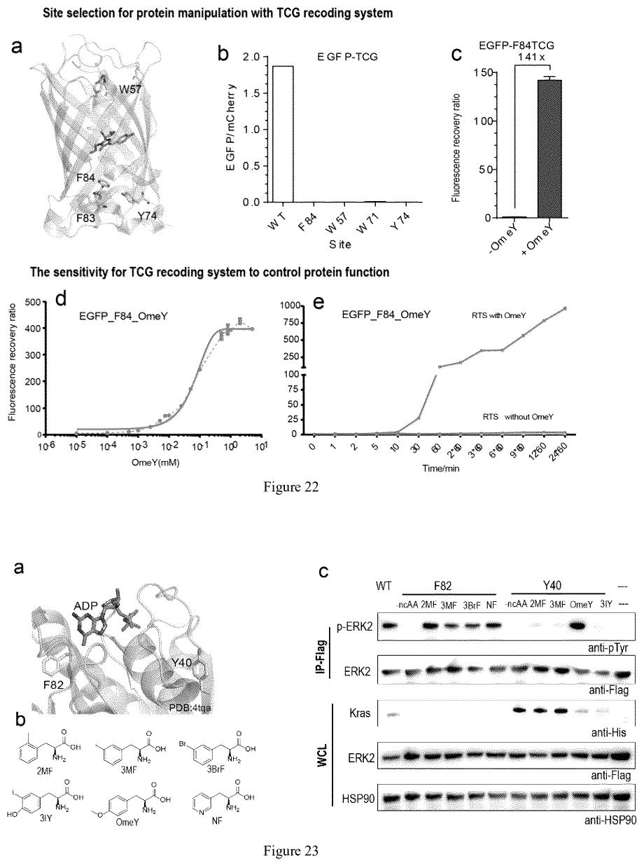

Using EGFP as an example, this example describes a general method for screening recoding sites and UAA types in protein activation regulated by UAA incorporation. In the absence of UAAs, the TCG recoding system decodes the TCG codon as serine (Ser). To maximize disruption towards protein function, we screened phenylalanine (Phe), tyrosine (Tyr), and tryptophan (Trp)—amino acids with the greatest structural divergence from Ser for potential incorporation. Following identification of Ser-substituted inactivation sites, we screened UAAs capable of rescuing protein activity, yielding optimal recoding system/UAA pairs (a-b of FIG. 22). For the EGFP F84TCG mutant, fluorescence quantification revealed that transfection with a TCG-recoding system decoding OmeY resulted in >100-fold fluorescence enhancement upon OmeY addition compared to the control group without UAA addition (c of FIG. 22). This demonstrates that the OmeY effectively rescues fluorescence in EGFP with TCG-recoding. Further optimization of tRNA copy, UAA concentration, and activation time showed high sensitivity with detectable EGFP activation at ultralow OmeY concentrations (0.00001 mM) (d of FIG. 22) and rapid response with fluorescence activation observed within 1 minute of UAA delivery (e of FIG. 22). This strategy was successfully extended to diverse proteins including fluorescent proteins (e.g., mCherry), enzymes (e.g., luciferase, kinases, peroxidases), and translation regulators (e.g., initiation factors, post-translational modification readers). The results (FIG. 23) showed significant increase in phosphorylated substrate yield upon UAA addition for kinases, confirming the generalizability of TCG-recoding-based functional regulation.

### 8.2 Dual Activation Strategy Via Rare Codon Pairing

This example using firefly luciferase (FLuc) illustrates the protein functional activation and regulation strategy involving the incorporation of caged UAAs through the dual rare codon recoding approach, specifically elucidating the protein sites for caged UAA incorporation via recoding systems and demonstrating the strategy's exceptional performance characteristics in protein functional regulation, including undetectable background activity and highly efficient activation.

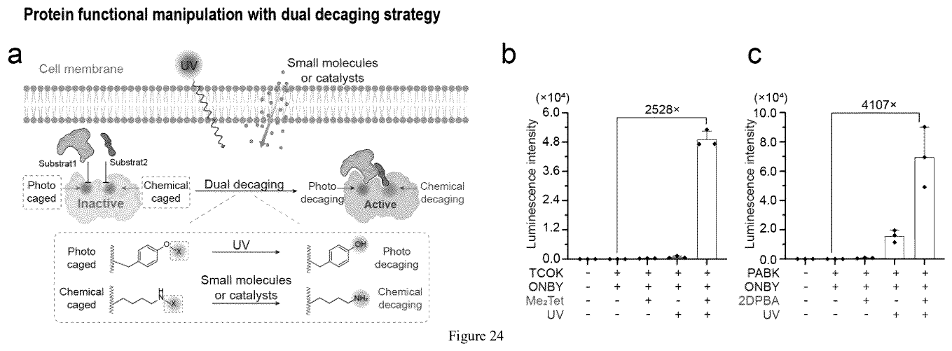

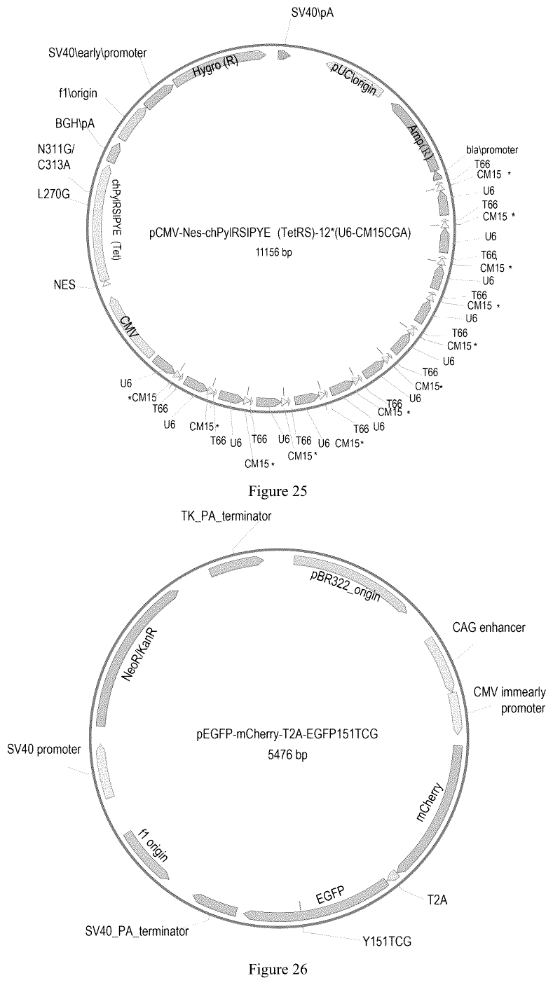

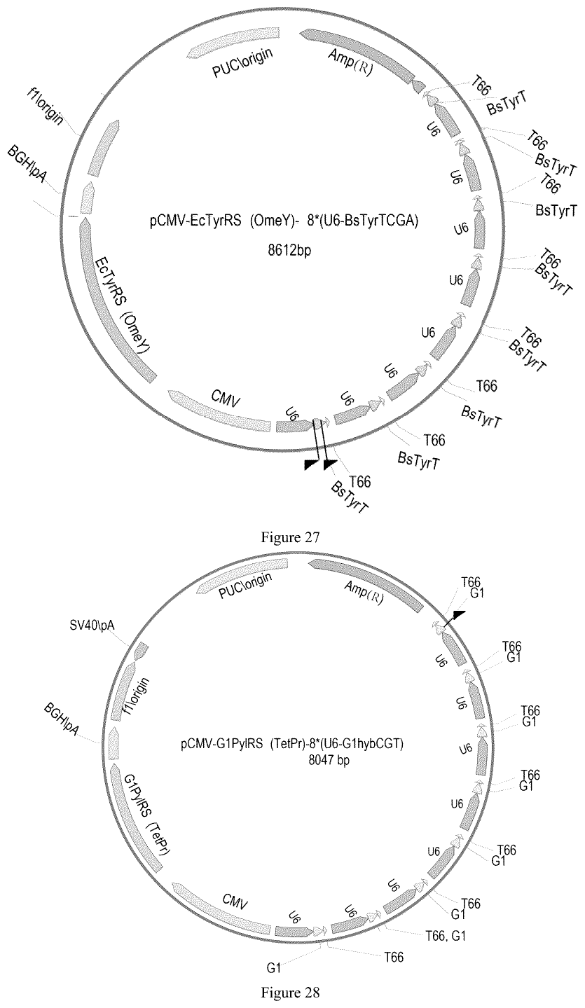

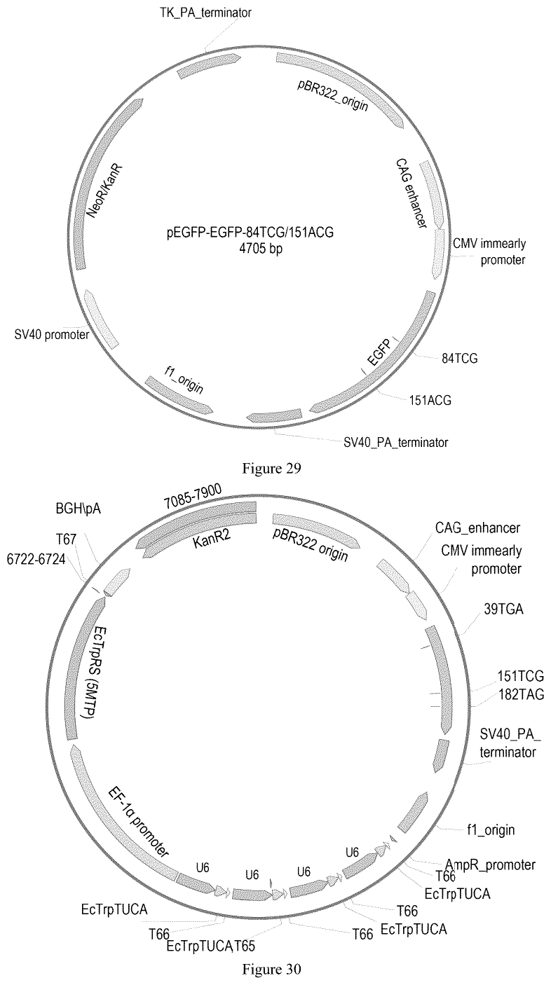

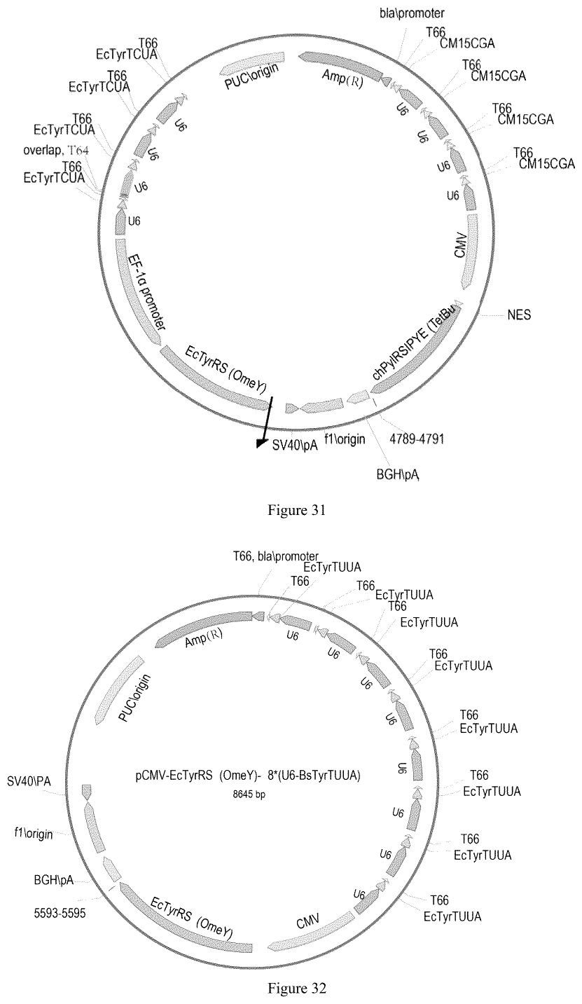

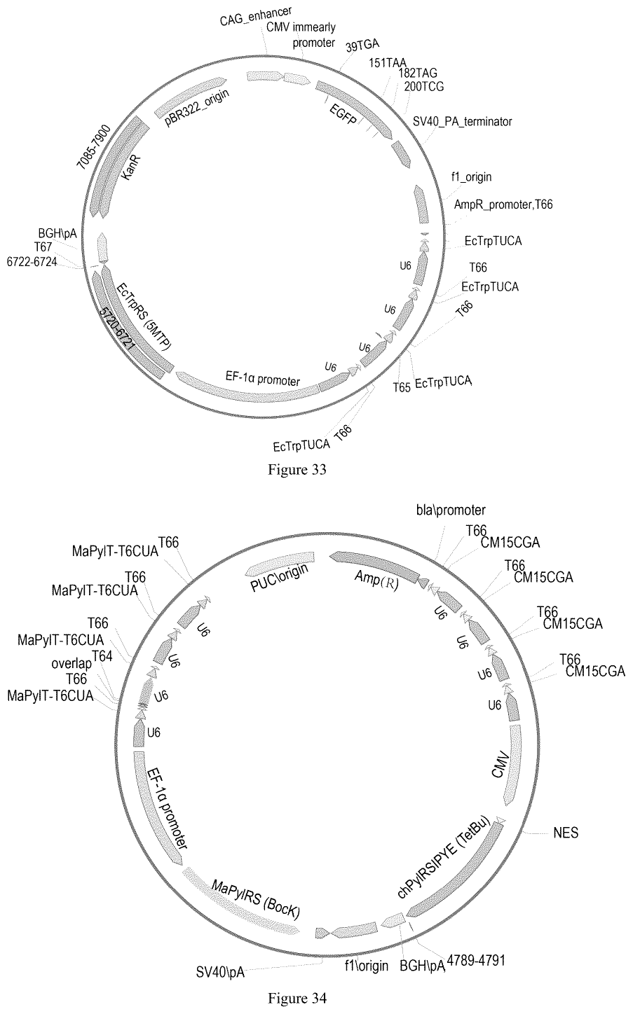

To established a method for orthogonal activation control of proteins, two caged UAAs are incorporated site-specifically into the active pocket or adjacent sites of target proteins through the dual rare codon recoding strategy, wherein steric hindrance leads to the protein inactivation, followed by protein activation through photochemical or chemical decaging strategies. The TCG/ACG dual recoding system decodes TCG/ACG sites as serine/threonine in the absence of UAAs. During the screening of FLuc, the K529 and Y255 site are identified with minimal background upon serine incorporation and threonine incorporation respectively, which were subsequently used for dual caged UAA incorporation. Using the FLucY255ACG/K529TCG mutant as an example, the TCG recoding system decoding ONBY, the ACG recoding system decoding TCOK/PABK, and reporter genes were co-transfected into cells, followed by luminescence assay to evaluate protein activation. The results demonstrated nearly complete inactivation of FLuc in the absence of UAAs or in the presence of both caged UAAs, while achieving over 2500-fold protein activation through photochemical or chemical decaging in live cells (FIG. 24). This strategy exhibits minimal background, excellent biocompatibility, and superior orthogonality of decaging strategies, with efficient protein activation occurring only when both caged UAAs are decaged (FIG. 24).

Given the broad applicability of the TCG/ACG dual recoding system in combinations of dual UAAs and selection of target proteins, the present application demonstrates that this dual rare codon strategy possesses universal applicability in regulating the activity of various functional proteins.

1. A method for producing a protein containing at least one unnatural amino acid (UAA), the method comprising culturing a eukaryotic host cell with:
a. a nucleotide sequence encoding a first recoding tRNA or the first recoding tRNA, wherein the first recoding tRNA comprises an anticodon complementary to a first codon, wherein the first codon is a first rare codon;
b. a nucleotide sequence encoding a first aminoacyl-tRNA synthetase (aaRS) or the first aaRS, wherein the first aaRS is capable of charging the first recoding tRNA with a first unnatural amino acid (UAA).

2. The method of claim 1, further comprising culturing the eukaryotic host cell with:
a. a nucleotide sequence encoding a second recoding tRNA or the second recoding tRNA, wherein the second recoding tRNA comprises an anticodon complementary to a second rare codon distinct from the first rare codon;
b. a nucleotide sequence encoding a second aaRS or the second aaRS, wherein the second aaRS charges the second recoding tRNA with a second UAA.

3. The method of claim 2, wherein the second rare codon is a stop codon or rare codon.

4. The method of claim 2, wherein the first rare codon and/or the second rare codon is selected from the group consisting of TCG, ACG, CGA, TCA, CGC, TTG, ATA, and GCG, wherein in a wild-type host cell, the abundance of aminoacylated tRNAs decoding the rare codon is less than 3% of total tRNAs, and the rare codon occurs in the translatome at a frequency of less than 1.5%.

5. The method of claim 1, wherein the aaRS and/or the recoding tRNA is derived from a prokaryote or a eukaryote, or is a variant thereof.

6. The method of claim 1, wherein the aaRS is a chimeric protein derived from enzymes of two or more organisms, wherein the recoding tRNA is a chimera of two tRNAs or a variant thereof, wherein the two different tRNAs are derived from different organisms.

7. The method of claim 1, wherein the aaRS is selected from the group consisting of TyrRS, LeuRS, PylRS, chPheRS, EcTrpRS, or variants or functional fragments thereof, wherein wild-type TyrRS, LeuRS, PylRS, chPheRS, EcTrpRS comprises the amino acid sequences of SEQ ID NO: 76-79.

8. The method of claim 7, wherein the PylRS is selected from the group consisting of MaPyIRS, MbPyIRS, MmPyIRS, G1PylRS, Lum1PylRS, 1R26PylRS, IntPylRS, NitraPylRS, DebPylRS, chPylRS, or variants thereof, wherein wild-type MaPyIRS, MbPyIRS, MmPyIRS, G1PylRS, Lum1PylRS, 1R26PylRS, IntPylRS, NitraPylRS, DebPylRS, chPylRS comprise the amino acid sequences of SEQ ID NO: 67-75.

9. The method of claim 8, wherein the MmPyIRS comprises a mutation in the amino acid recognition region, the mutation being selected from a combination of one or more of the following positions: L105, M276, L301, A302, L305, Y306, L309, I322, N346, C348, M350, D379, Y384, V401, I405, L407, I413, and W417.

10. The method of claim 8, wherein the G1PylRS comprises a mutation in the amino acid recognition region, the mutation being selected from a combination of one or more of the following positions: A121, A221, H120, H225, I141, L124, M128, N165, V167, V233, W237, Y125, and Y204.

11. The method of claim 7, wherein the chPheRS is a fusion protein comprising a tRNA-binding domain (NTD) from a PylRS variant and an amino acid recognition domain (CTD) from a mitochondrial phenylalanyl-tRNA synthetase of a eukaryote.

12. The method of claim 11, wherein the chPheRS comprises a mutation in the amino acid recognition region, the mutation being selected from a combination of one or more of the following positions: Q113, E148, V150, F221, T224, L247, and A264.

13. The method of claim 1, wherein the recoding tRNA comprises one or more anticodons complementary to codons selected from the group consisting of TCG, ACG, CGA, TCA, CGC, TTG, ATA, and GCG.

14. The method of claim 1, wherein the recoding tRNA is selected from:
a. when the aaRS is PylRS: recoding tRNA is selected from one or more of C15, M15, CM15, MbPylT, MetPylT, SpePylT, Pyl-O1, Pyl-O2, Ma-T6, MaPylT, G1PylT, G1hyb, Ma-T6, I2B72, Int17, Int5, Int6B, Int6C, Int13, Alv21, Alv8, Alv17, Alv10, Alv22, G1hyb, Int, Therm1, and BH52: C15, M15, CM15, MbPylT, MaPylT, G1PylT, or variants thereof;
b. when the aaRS is chPheRS: the recoding tRNA is selected from one or more of CM4, CM15, MbPylT, Pyl-O1, Pyl-O2, and AS78, or variants thereof;
c. when the aaRS is EcTyrRS: the recoding tRNA is selected from BsTyrT, NGS6, or variants thereof;
d. when the aaRS is EcLeuRS: the recoding tRNA is selected from EcLeuT, LeuT-G1, LeuT-G2, or variants thereof.

15. The method of claim 1, wherein the recoding tRNA is further encoded by a sequence as set forth in any one of SEQ ID Nos: 1-66 or a homologous sequence thereof.

16. The method of claim 1, wherein the host cell comprises a transcription template, wherein the transcription template comprises the first rare codon and/or the second rare codon.

17. The method of claim 1, wherein the unnatural amino acid is selected from one or more of the following: tetrazine unnatural amino acids: p-acetyl-L-phenylalanine; p-iodo-L-phenylalanine; O-methyl-L-tyrosine; p-propargyloxyphenylalanine; p-propargyl-phenylalanine; L-3-(2-naphthyl)alanine; 3-methyl-phenylalanine; O-4-allyl-L-tyrosine: 4-propyl-L-tyrosine; L-Dopa; fluorinated phenylalanine; isopropyl-L-phenylalanine; p-azido-L-phenylalanine; p-acyl-L-phenylalanine; p-benzoyl-L-phenylalanine; L-phosphoserine; phosphonoserine; phosphonotyrosine; p-bromophenylalanine; p-amino-L-phenylalanine; isopropyl-L-phenylalanine; unnatural analogs of tyrosine amino acids; unnatural analogs of glutamine amino acids: unnatural analogs of phenylalanine amino acids; unnatural analogs of serine amino acids; unnatural analogs of threonine amino acids; amino acids substituted with alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino groups: amino acids with photoactivatable cross-linkers; spin-labeled amino acids: fluorescent amino acids; metal-binding amino acids; metal-containing amino acids; radioactive amino acids: photocaged and/or photoisomerizable amino acids; amino acids containing biotin or biotin analogs: keto-containing amino acids; amino acids containing polyethylene glycol or polyether; heavy atom-substituted amino acids: chemically cleavable or photocleavable amino acids: amino acids with elongated side chains; amino acids containing toxic groups: sugar-substituted amino acids: amino acids containing carbon-linked sugars; redox-active amino acids; α-hydroxy-containing acids; amino thioacids; α,α-disubstituted amino acids: β-amino acids; cyclic amino acids other than proline or histidine; and aromatic amino acids other than phenylalanine, tyrosine, or tryptophan.

18. The method of claim 1, wherein the protein containing the UAA is a therapeutic protein, a diagnostic protein, or an industrial enzyme.

19. A kit comprising:
a. a nucleotide sequence encoding a first recoding tRNA or the first recoding tRNA;
b. a nucleotide sequence encoding a first aaRS or the first aaRS;
wherein the first recoding tRNA comprises an anticodon complementary to a rare codon selected from the group consisting of TCG, ACG, CGA, TCA, CGC, TTG, ATA, and GCG.

20. The kit of claim 19, further comprising a first UAA, wherein the first aaRS is capable of charging the first recoding tRNA with the first UAA.

21. A cell comprising:
a. a nucleotide sequence encoding a first recoding tRNA or the first recoding tRNA;
b. a nucleotide sequence encoding a first aaRS or the first aaRS;
wherein the first recoding tRNA comprises an anticodon complementary to a rare codon selected from the group consisting of TCG, ACG, CGA, TCA, CGC, TTG, ATA, and GCG.

22. The method of claim 1, wherein the protein contains multiple UAAs incorporated via distinct rare codons.
